(19) United States
(12) Patent Application Publication
(10) Pub. No.: US 2025/0060667 A1
(43) Pub. Date: Feb. 20, 2025

(54) SALT, ACID GENERATOR, RESIST COMPOSITION AND METHOD FOR PRODUCING RESIST PATTERN

(71) Applicant: SUMITOMO CHEMICAL COMPANY, LIMITED, Tokyo (JP)

(72) Inventors: Shohei Terahigashi, Osaka-shi (JP); Issei Kitamura, Osaka-shi (JP); Koji Ichikawa, Osaka-shi (JP)

(73) Assignee: SUMITOMO CHEMICAL COMPANY, LIMITED, Tokyo (JP)

(21) Appl. No.: 18/787,627

(22) Filed: Jul. 29, 2024

(30) Foreign Application Priority Data
Aug. 2, 2023 (JP) 2023-126643

(51) Int. Cl. G03F 7/004; C07C 309/12; C07C 309/17; C07C 381/12; C07D 307/00; C08F 220/18; G03F 7/038; G03F 7/039

(52) U.S. Cl. CPC G03F 7/0045; C07C 309/12; C07C 309/17; C07C 381/12; C07D 307/00; C08F 220/1808; G03F 7/038; G03F 7/039

(57) ABSTRACT

A salt represented by formula (I), a salt or resin including a structural unit represented by formula (IP), an acid generator including the salt, an acid generator including the resin, or a resist composition including the acid generator.

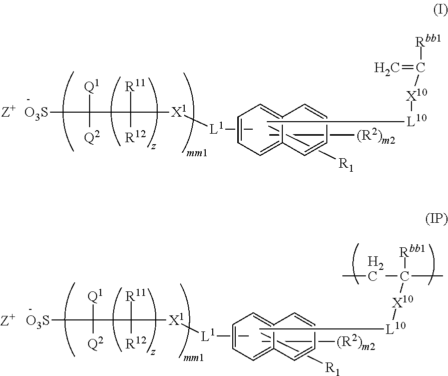

### TECHNICAL FIELD

The present invention relates to a salt, an acid generator, a resist composition and a method for producing a resist pattern.

### BACKGROUND ART

Patent Document 1 mentions a resist composition comprising a resin including a structural unit derived from a salt represented by the following formula.

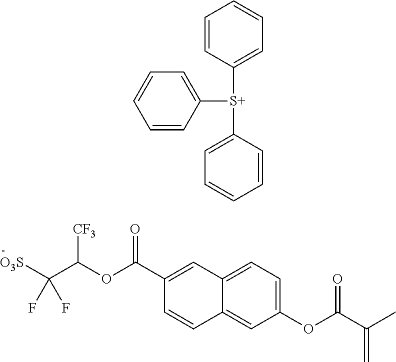

### PRIOR ART DOCUMENT

### Patent Document

[Patent Document 1] JP 2010-077404 A

### DISCLOSURE OF THE INVENTION

### Problems to be Solved by the Invention

It is an object of the present invention to provide a salt forming a resist pattern with CD uniformity (CDU) which is better than that of the above resist composition comprising a salt.

### Means for Solving the Problems

The present invention includes the following inventions.

[1] A salt represented by formula (I):

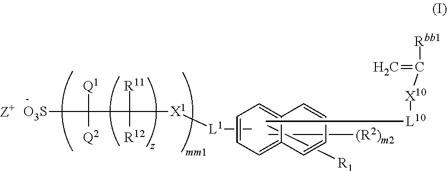

wherein, in formula (I),
Q¹ and Q² each independently represent a hydrogen atom, a fluorine atom, an alkyl group having 1 to 6 carbon atoms, or a perfluoroalkyl group having 1 to 6 carbon atoms,
R¹¹ and R¹² each independently represent a hydrogen atom, a fluorine atom, an alkyl group having 1 to 6 carbon atoms, or a perfluoroalkyl group having 1 to 6 carbon atoms,
z represents an integer of 0 to 6, and when z is 2 or more, a plurality of R¹¹ and R¹² may be the same or different from each other,
X¹ represents *—CO—O—, *—O—CO—, *—O—CO—O— or *—O—, and * represents a bonding site to C(R¹¹) (R¹²) or C(Q¹) (Q²),
mm1 represents 0 or 1,
L¹ represents a single bond or a hydrocarbon group having 1 to 40 carbon atoms which may have a substituent, and —CH₂— included in the hydrocarbon group may be replaced by —O—, —S—, —SO₂— or —CO—,
R¹ represents a halogen atom or a haloalkyl group having 1 to 12 carbon atoms,
R² represents a halogen atom, a hydroxy group, a haloalkyl group having 1 to 12 carbon atoms, or an alkyl group having 1 to 12 carbon atoms, and —CH₂— included in the haloalkyl group and the alkyl group may be replaced by —O— or —CO—,
m2 represents an integer of 0 to 5, and when m2 is 2 or more, a plurality of R² may be the same or different from each other,
R^{bb1} represents a hydrogen atom, a halogen atom, or an alkyl group having 1 to 6 carbon atoms which may have a halogen atom,
X¹⁰ represents a single bond, *—O—**, *—CO—O—**, *—O—CO—O—**, *—CO—NR³—**, *—NR³—CO—O—**, *—O—CO—NR³—** or *-Ax-Ph-Ay-**,
R³ represents a hydrogen atom or an alkyl group having 1 to 6 carbon atoms,
Ph represents a phenylene group which may have a substituent,
Ax and Ay each independently represent one or more groups selected from the group consisting of a single bond, an ether bond, a thioether bond, an ester bond, an amide bond and a carbonic acid ester bond,
* and ** represent a bonding site, and * represents a bonding site to carbon atoms to which R^{bb1} is bonded,
L¹⁰ represents a single bond or a hydrocarbon group having 1 to 36 carbon atoms which may have a substituent, and —CH₂— included in the hydrocarbon group may be replaced by —O—, —S—, —SO₂— or —CO—, and
Z⁺ represents an organic cation.
   [2] The salt according to [1], wherein X¹⁰ is a single bond or a group represented by any one of formula (X¹⁰-1) to formula (X¹⁰-10):

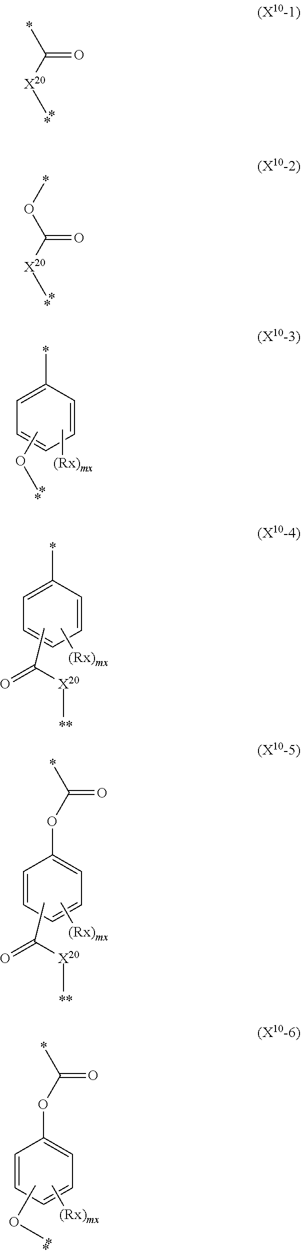

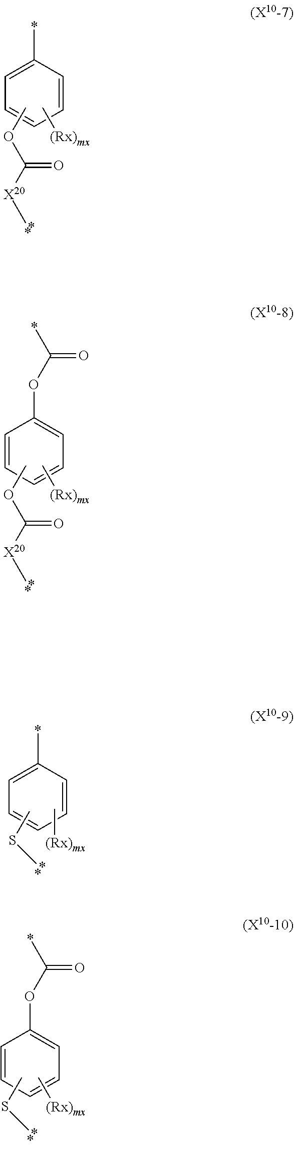

wherein, in formula (X¹⁰-1) to formula (X¹⁰-10),
* and ** are bonding sites, and * represents a bonding site to carbon atoms to which R^{bb1} is bonded,
Rx represents a halogen atom, a hydroxy group, an alkyl fluoride group having 1 to 6 carbon atoms, an alkyl group having 1 to 18 carbon atoms or an alkoxy group having 1 to 6 carbon atoms,
mx represents an integer of 0 to 4,
X²⁰ represents —O— or —NR³—, and
R³ represents a hydrogen atom or an alkyl group having 1 to 6 carbon atoms.
   [3] The salt according to [1] or [2], wherein R¹ is an iodine atom, a fluorine atom or a perfluoroalkyl group having 1 to 3 carbon atoms.
   [4] The salt according to any one of [1] to [3], wherein L¹ is a single bond, *-L²- or *-L²-X³-L³-(L² represents a chain hydrocarbon group having 1 to 10 carbon atoms which may have a substituent, a cyclic hydrocarbon group having 3 to 24 carbon atoms which may have a substituent, or groups obtained by combining a cyclic hydrocarbon group having 3 to 24 carbon atoms which may have a substituent with a chain hydrocarbon group having 1 to 8 carbon atoms which may have a substituent, —CH₂— included in the cyclic hydrocarbon group may be replaced by —O—, —S—, —CO— or —SO₂—, —CH₂— included in the chain hydrocarbon group may be replaced by —O— or —CO—, * represents a bonding site to X¹, X³ represents **—CO—O—, **—O—CO—, **—O—CO—O— or **—O—, ** represents a bonding site to X¹, L³ represents a single bond or a chain hydrocarbon group having 1 to 8 carbon atoms which may have a substituent, and —CH₂— included in the chain hydrocarbon group may be replaced by —O— or —CO—).
   [5] The salt according to any one of [1] to [4], wherein when m2 is an integer of 1 or more, R² is an iodine atom, a fluorine atom, a hydroxy group, a haloalkyl group having 1 to 6 carbon atoms or an alkyl group having 1 to 6 carbon atoms (—CH₂— included in the haloalkyl group and the alkyl group may be replaced by —O— or —CO—).
   [6] A resin comprising a structural unit represented by formula (IP):

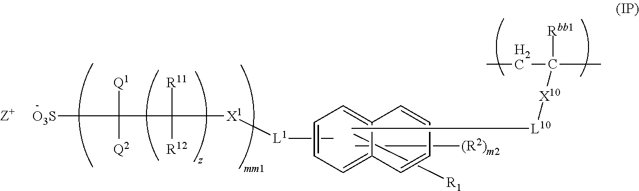

wherein, in formula (IP),
Q¹ and 02 each independently represent a hydrogen atom, a fluorine atom, an alkyl group having 1 to 6 carbon atoms or a perfluoroalkyl group having 1 to 6 carbon atoms,
R¹¹ and R¹² each independently represent a hydrogen atom, a fluorine atom, an alkyl group having 1 to 6 carbon atoms, or a perfluoroalkyl group having 1 to 6 carbon atoms,
z represents an integer of 0 to 6, and when z is 2 or more, a plurality of R¹¹ and R¹² may be the same or different from each other,
X¹ represents *—CO—O—, *—O—CO—, *—O—CO—O— or *—O—, and * represents a bonding site to C(R¹¹) (R¹²) or C(Q¹) (Q²),
mm1 represents 0 or 1,
L¹ represents a single bond or a hydrocarbon group having 1 to 40 carbon atoms which may have a substituent, and —CH₂— included in the hydrocarbon group may be replaced by —O—, —S—, —SO₂— or —CO—,
R¹ represents a halogen atom or a haloalkyl group having 1 to 12 carbon atoms,
R² represents a halogen atom, a hydroxy group, a haloalkyl group having 1 to 12 carbon atoms or an alkyl group having 1 to 12 carbon atoms, and —CH₂— included in the haloalkyl group and the alkyl group may be replaced by —O— or —CO—,
m2 represents an integer of 0 to 5, and when m2 is 2 or more, a plurality of R² may be the same or different from each other,
R^{bb1} represents a hydrogen atom, a halogen atom, or an alkyl group having 1 to 6 carbon atoms which may have a halogen atom,
X¹⁰ represents a single bond, *—O—**, *—CO—O—**, *—O—CO—O—**, *—CO—NR³—**, *—NR³—CO—O—**, *—O—CO—NR³—** or *-Ax-Ph-Ay-**,
R³ represents a hydrogen atom or an alkyl group having 1 to 6 carbon atoms,
Ph represents a phenylene group which may have a substituent,
Ax and Ay each independently represent one or more bonds selected from the group consisting of a single bond, an ether bond, a thioether bond, an ester bond, an amide bond and a carbonic acid ester bond,
* and ** represent a bonding site, and * represents a bonding site to carbon atoms to which R^{bb1} is bonded,
L¹⁰ represents a single bond or a hydrocarbon group having 1 to 36 carbon atoms which may have a substituent, and —CH₂— included in the hydrocarbon group may be replaced by —O—, —S—, —SO₂— or —CO—, and
Z⁺ represents an organic cation.
   [6-a] The resin according to [6], wherein X¹⁰ is a single bond or a group represented by any one of the formula (X¹⁰-1) to formula (X¹⁰-10).
   [6-b] The resin according to [6] or [6-a], wherein R¹ is an iodine atom, a fluorine atom or a perfluoroalkyl group having 1 to 3 carbon atoms.
   [6-c] The resin according to any one of [6], [6-a] and [6-b], wherein L′ is a single bond, *-L²- or *-L²-X³-L³-(L² represents a chain hydrocarbon group having 1 to 10 carbon atoms which may have a substituent, a cyclic hydrocarbon group having 3 to 24 carbon atoms which may have a substituent, or a group obtained by combining a cyclic hydrocarbon group having 3 to 24 carbon atoms which may have a substituent with a chain hydrocarbon group having 1 to 8 carbon atoms which may have a substituent, —CH₂— included in the cyclic hydrocarbon group may be replaced by —O—, —S—, —CO— or —SO₂—, —CH₂— included in the chain hydrocarbon group may be replaced by —O— or —CO—, * represents a bonding site to X¹, X³ represents **—CO—O—, **—O—CO—, **—O—CO—O— or **—O—, ** represents a bonding site to L², L³ represents a single bond or a chain hydrocarbon group having 1 to 8 carbon atoms which may have a substituent, and —CH₂— included in the chain hydrocarbon group may be replaced by —O— or —CO—).
   [6-d] The resin according to any one of [6] and [6-a] to [6-c], wherein when m2 is an integer of 1 or more, R² is an iodine atom, a fluorine atom, a hydroxy group, a haloalkyl group having 1 to 6 carbon atoms or an alkyl group having 1 to 6 carbon atoms (—CH₂— included in the haloalkyl group and the alkyl group may be replaced by —O— or —CO—).
   [7] The resin according to any one of [6] and [6-a] to [6-d], further comprising a structural unit having an acid-labile group.
   [8] An acid generator comprising the salt according to any one of [1] to [5].
   [9] An acid generator comprising the resin according to any one of [6] and [6-a] to [6-d].
   [10] A resist composition comprising the salt according to any one of [1] to [5], and a quencher.
   [11] A resist composition comprising the resin any one of [6] and [6-a] to [6-d], and a quencher.
   [12] A resist composition comprising the salt according to any one of [1] to [5], and the resin according to any one of [6] and [6-a] to [6-d].
   [13] A resist composition comprising the resin according to [7] and a quencher.
   [14] The resist composition according to [13], wherein the structural unit having an acid-labile group includes at least one selected from the group consisting of a structural unit represented by formula (a1-0), a structural unit represented by formula (a1-1), a structural unit represented by formula (a1-2), a structural unit represented by formula (a1-4), a structural unit represented by formula (a1-5) and a structural unit represented by formula (a1-6):

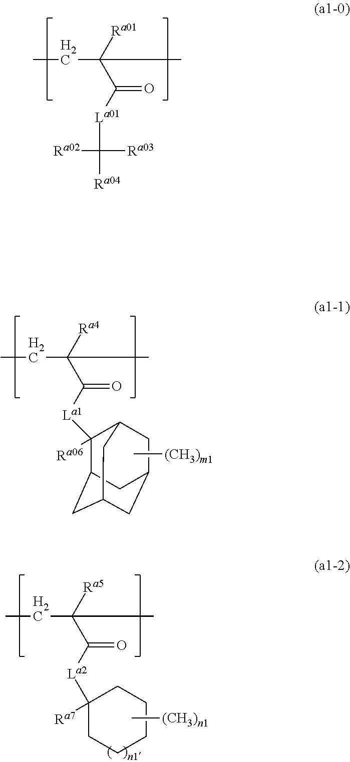

wherein, in formula (a1-0), formula (a1-1) and formula (a1-2),
L^{a01}, L^{a1} and L^{a2} each independently represent-O— or *—O—(CH₂)ₖ₁—CO—O—, k1 represents an integer of 1 to 7, and * represents a bonding site to —CO—,
R^{a01}, R^{a4} and R^{a5} each independently represent a hydrogen atom, a halogen atom, or an alkyl group having 1 to 6 carbon atoms which may have a halogen atom,
R^{a02}, R^{a03} and R^{a04} each independently represent an alkyl group having 1 to 8 carbon atoms, an alicyclic hydrocarbon group having 3 to 18 carbon atoms, an aromatic hydrocarbon group having 6 to 18 carbon atoms, or groups obtained by combining these groups, and the alkyl group, the alicyclic hydrocarbon group and the aromatic hydrocarbon group may have a halogen atom,
R^{a6} and R^{a7} each independently represent an alkyl group having 1 to 8 carbon atoms, an alkenyl group having 2 to 8 carbon atoms, an alicyclic hydrocarbon group having 3 to 18 carbon atoms, an aromatic hydrocarbon group having 6 to 18 carbon atoms, or groups formed by combining these groups, and the alkyl group, the alicyclic hydrocarbon group and the aromatic hydrocarbon group may have a halogen atom,
m1 represents an integer of 0 to 14,
n1 represents an integer of 0 to 10, and
n1′ represents an integer of 0 to 3:

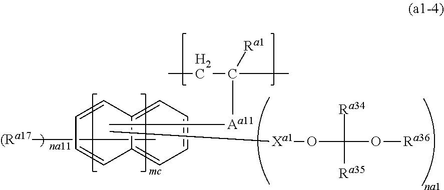

wherein, in formula (a1-4),
R^{a1} represents a hydrogen atom, a halogen atom, or an alkyl group having 1 to 6 carbon atoms which may have a halogen atom,
R^{a17} represents a halogen atom, a hydroxy group, a carboxy group, an alkyl group having 1 to 6 carbon atoms which may have a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group or a methacryloyloxy group,
A^{a11} represents a single bond or an alkanediyl group having 1 to 12 carbon atoms, and —CH₂— included in the alkanediyl group may be replaced by —O—, —CO— or —NR^{a18}—,
R^{a18} represents a hydrogen atom or an alkyl group having 1 to 6 carbon atoms,
X^{a1} represents a single bond or a carbonyl group,
R^{a34} and R^{a35} each independently represent a hydrogen atom or a hydrocarbon group having 1 to 12 carbon atoms, R^{a36} represents a hydrocarbon group having 1 to 20 carbon atoms, or R^{a35} and R^{a36} may be bonded to each other to form a divalent hydrocarbon group having 2 to 20 carbon atoms together with —C—O— to which R^{a35} and R^{a36} are bonded, and —CH₂— included in the hydrocarbon group and the divalent hydrocarbon group may be replaced by —O— or —S—,
na1 represents an integer of 1 to 4, and when na1 is 2 or more, a plurality of groups in parentheses may be the same or different from each other,
na11 represents an integer of 0 to 4 and satisfies 1≤ na1+na11≤5, and when na11 is 2 or more, a plurality of R^{a1}7 may be the same or different from each other, and
mc represents an integer of 0 to 2:

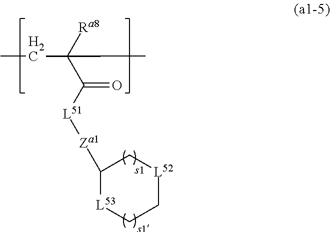

wherein, in formula (a1-5),
R^{a8} represents an alkyl group having 1 to 6 carbon atoms which may have a halogen atom, a hydrogen atom or a halogen atom,
Z^{a1} represents a single bond or *—(CH₂)ₕ₃—CO-L⁵⁴-, h3 represents an integer of 1 to 4, and * represents a bonding site to L⁵¹,
L⁵¹, L⁵², L⁵³ and L⁵⁴ each independently represent-O— or —S—,
s1 represents an integer of 1 to 3, and
s1′ represents an integer of 0 to 3, and:

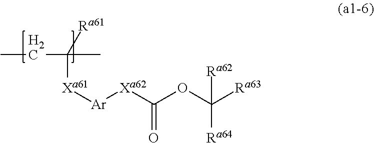

wherein, in formula (a1-6),
R^{a61} represents a hydrogen atom, a halogen atom, or an alkyl group having 1 to 6 carbon atoms which may have a halogen atom,
R^{a62}, R^{a63} and R^{a64} each independently represent an alkyl group having 1 to 6 carbon atoms or a cyclic hydrocarbon group having 3 to 18 carbon atoms which may have a substituent, or R^{a62} and R^{a63} may be bonded to each other to form a ring having 3 to 20 carbon atoms together with carbon atoms to which R^{a62} and R^{a63} are bonded,
X^{a61} represents a single bond, —CO—O—* or —CO—NR⁵—*, * represents a bonding site to —Ar, and R⁵ represents a hydrogen atom or an alkyl group having 1 to 4 carbon atoms,
X^{a62} represents a single bond, *—O-L^{a61}- or *—CO—O— L^{a62}-, * represents a bonding site to —Ar, and L^{a61} and L^{a62} each independently represent an alkanediyl group having 1 to 4 carbon atoms, and
Ar represents an aromatic hydrocarbon group having 6 to 20 carbon atoms which may have a substituent.
   [15] The resist composition according to or [14], wherein the resin according to any one of [6] and [6-a] to [6-d] includes a structural unit represented by formula (a2-A):

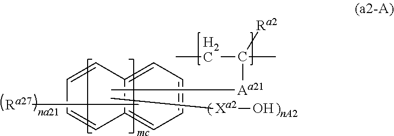

wherein, in formula (a2-A),
R^{a2} represents a hydrogen atom, a halogen atom, or an alkyl group having 1 to 6 carbon atoms which may have a halogen atom,
R^{a27} represents a halogen atom, an alkyl group having 1 to 6 carbon atoms which may have a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group or a methacryloyloxy group,
A^{a21} represents a single bond or an alkanediyl group having 1 to 12 carbon atoms, and —CH₂— included in the alkanediyl group may be replaced by —O—, —CO— or —NR^{a28}—,
R^{a28} represents a hydrogen atom or an alkyl group having 1 to 6 carbon atoms,
X^{a2} represents a single bond or a carbonyl group,
nA2 represents an integer of 1 to 4, and when nA2 is 2 or more, a plurality of groups in parentheses may be the same or different from each other,
na21 represents an integer of 0 to 4 and satisfies 1≤nA2+na21≤5, and when na21 is 2 or more, a plurality of R^{a27} may be the same or different from each other, and mc represents an integer of 0 to 2.
   [16] The resist composition according to any one of to [15], wherein the resin according to [7] further includes a structural unit represented by formula (a3-1), formula (a3-2), formula (a3-3) or formula (a3-4):

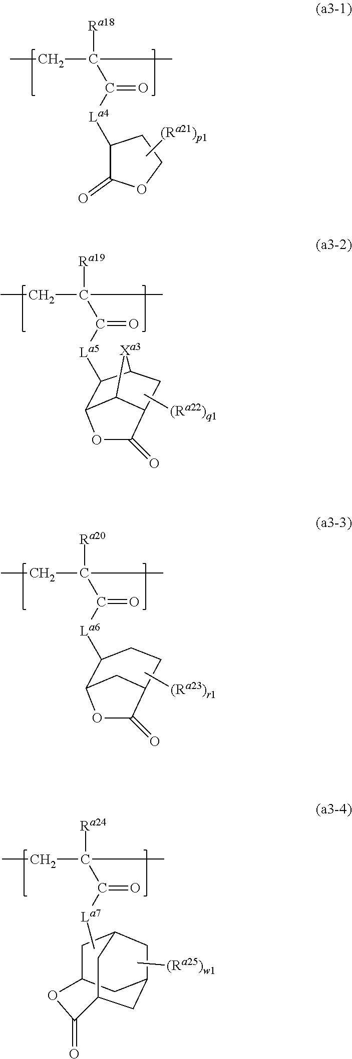

wherein, in formula (a3-1), formula (a3-2), formula (a3-3) and formula (a3-4),
L^{a4}, L^{a5} and L^{a6} each independently represent a group represented by —O— or *—O—(CH₂)ₖ₃—CO—O— (k3 represents an integer of 1 to 7),
L^{a7} represents —O—, *—O-L^{a8}-O—, *—O-L^{a8}-CO—O—, *—O— L^{a8}-CO—O-L^{a9}-CO—O— or *—O-L^{a8}-O—CO-L^{a9}-O—,
L^{a8} and L^{a9} each independently represent an alkanediyl group having 1 to 6 carbon atoms,
* represents a bonding site to a carbonyl group,
R^{a18}, R^{a19}, R^{a20} and R^{a24} each independently represent an alkyl group having 1 to 6 carbon atoms which may have a halogen atom, a hydrogen atom or a halogen atom,
X^{a3} represents —CH₂— or an oxygen atom,
R^{a21} represents an aliphatic hydrocarbon group having 1 to 4 carbon atoms,
R^{a22}, R^{a23} and R^{a25} each independently represent a carboxy group, a cyano group or an aliphatic hydrocarbon group having 1 to 4 carbon atoms,
p1 represents an integer of 0 to 5,
q1 represents an integer of 0 to 3,
r1 represents an integer of 0 to 3,
w1 represents an integer of 0 to 8, and
when p1, q1, r1 and/or w1 is/are 2 or more, a plurality of R^{a21}, R^{a22}, R^{a23} and/or R^{a25} may be the same or different from each other.
   [17] The resist composition according to any one of to [16], wherein the quencher (C) further includes a salt generating an acid having an acidity lower than that of an acid generated from an acid generator.
   [18] A method for producing a resist pattern, which comprises:
(1) a step of applying the resist composition according to any one of to on a substrate,
(2) a step of drying the applied resist composition to form a composition layer,
(3) a step of exposing the composition layer,
(4) a step of heating the exposed composition layer, and
(5) a step of developing the heated composition layer.

### Effects of the Invention

It is possible to produce a resist pattern with satisfactory CD uniformity (CDU) by using a resist composition including a salt or a structural unit of the present invention.

### MODE FOR CARRYING OUT THE INVENTION

As used herein, “(meth)acrylic monomer” means “at least one of acrylic monomer and methacrylic monomer”. Notations such as “(meth)acrylate” and “(meth)acrylic acid” mean the same thing. In groups mentioned herein, regarding groups capable of having both a linear structure and a branched structure, they may have either the linear or branched structure. When —CH₂— included in the hydrocarbon group or the like is replaced by —O—, —S—, —CO— or —SO₂—, the same examples shall apply for each group. “Combined group” means a group in which two or more exemplified groups are bonded, and valences of those groups may be appropriately varied by bonding forms. “Derived” means that a polymerizable C═C bond included in the molecule becomes a —C—C— group (single bond) by polymerization. When stereoisomers exist, all stereoisomers are included. Hydrogen atoms at any position and any number of hydrogen atoms included in each group may be sometimes replaced by a bond depending on the number of substituents or the like. The number of carbon atoms in the substituents is not included in the number of carbon atoms in the group to be substituted.

As used herein, “solid content of the resist composition” means the total amount of components in which the below-mentioned solvent (E) is removed from the total amount of the resist composition.

### [Salt Represented by Formula (I)]

The present invention relates to a salt represented by formula (I) (hereinafter sometimes referred to as “salt (I)”).

Of the salt (I), the side having negative charge is sometimes referred to as “anion (I)”, and the side having positive charge is sometimes referred to as “cation (I)”:

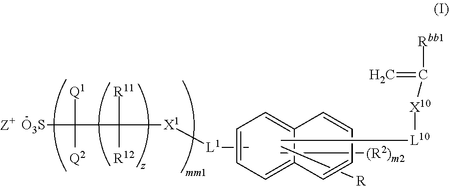

wherein all symbols are the same as defined above.

In formula (I), examples of the perfluoroalkyl group as for Q¹, Q², R¹¹ and R¹² include a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group, a perfluorohexyl group and the like. The number of carbon atoms of the perfluoroalkyl group is preferably 1 to 4, and more preferably 1 to 3.

Examples of the alkyl group as for Q¹, Q², R¹¹ and R¹² include a methyl group, an ethyl group, a propyl group, an isopropyl group, a butyl group, a sec-butyl group, a tert-butyl group, a pentyl group, a hexyl group and the like. The number of carbon atoms of the alkyl group is preferably 1 to 4, and more preferably 1 to 3.

Preferably, at least one of Q¹ and Q² includes a fluorine atom or a perfluoroalkyl group, more preferably at least one is a fluorine atom or a perfluoroalkyl group, still more preferably at least one is each independently a trifluoromethyl group or a fluorine atom, and yet more preferably both are fluorine atoms.

Preferably, R¹¹ and R¹² are each independently a hydrogen atom, a fluorine atom, a methyl group or a trifluoromethyl group, and more preferably a hydrogen atom or a trifluoromethyl group.

Z is preferably an integer of 0 to 4, more preferably an integer of 0 to 3, and still more preferably 0 or 1.

X¹ is preferably *—CO—O—, *—O—CO—O— or *—O—CO—, and more preferably *—CO—O— or *—O—CO—(* represents a bonding site to C(R¹¹) (R¹²) or C(Q¹) (Q²)).

Examples of the hydrocarbon group in L¹ include divalent chain hydrocarbon groups such as an alkanediyl group, monocyclic or polycyclic (including a spiro ring) divalent alicyclic hydrocarbon groups, and divalent cyclic hydrocarbon groups such as a divalent aromatic hydrocarbon groups, and the hydrocarbon group may be groups obtained by combining two or more of these groups (e.g., a divalent hydrocarbon group formed from an alicyclic hydrocarbon group or an aromatic hydrocarbon group and an alkanediyl group). The number of carbon atoms of the hydrocarbon group is preferably 1 to 38, more preferably 1 to 36, still more preferably 1 to 32, and yet more preferably 1 to 28.

Examples of the alkanediyl group include linear alkanediyl groups such as a methylene group, an ethylene group, a propane-1,3-diyl group, a butane-1,4-diyl group, a pentane-1,5-diyl group, a hexane-1,6-diyl group, a heptane-1,7-diyl group, a octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group and a heptadecane-1,17-diyl group, and

branched alkanediyl groups such as an ethane-1,1-diyl group, a propane-1,1-diyl group, a propane-1,2-diyl group, a propane-2,2-diyl group, a pentane-2,4-diyl group, a 2-methylpropane-1,3-diyl group, a 2-methylpropane-1,2-diyl group, a pentane-1,4-diyl group and a 2-methylbutane-1,4-diyl group.

The number of carbon atoms of the chain hydrocarbon group is preferably 1 to 18, more preferably 1 to 12, still more preferably 1 to 10, yet more preferably 1 to 8, further preferably 1 to 7, still further preferably 1 to 6, yet still further preferably 1 to 5, and particularly preferably 1 to 4.

Examples of the monocyclic or polycyclic divalent alicyclic hydrocarbon group include the following alicyclic hydrocarbon groups and the like. The bonding site can be any position.

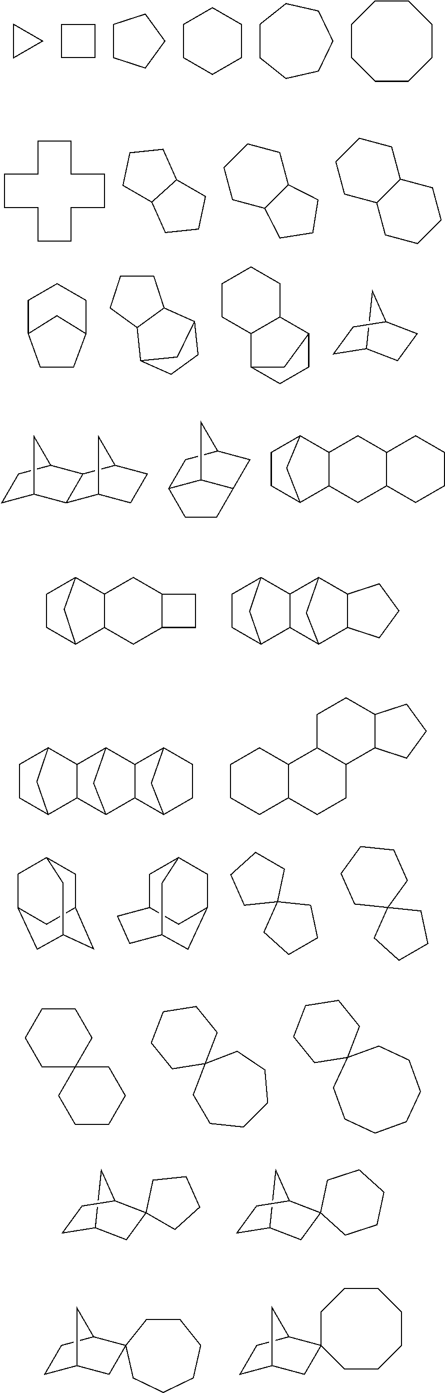

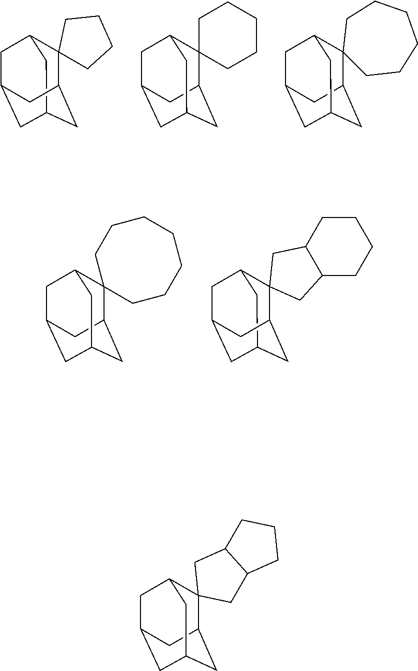

Specific examples thereof include monocyclic divalent alicyclic hydrocarbon groups which are cycloalkanediyl groups, such as a cyclobutane-1,3-diyl group, a cyclopentane-1,3-diyl group, a cyclohexane-1,4-diyl group and a cyclooctane-1,5-diyl group, and

polycyclic divalent alicyclic hydrocarbon groups, for example, spiro rings having a cycloalkanediyl group, a norbornyl group or an adamantyl group and a cycloalkanediyl group spiro-bonded to each group, such as a norbornane-1,4-diyl group, a norbornane-2,5-diyl group, an adamantane-1,5-diyl group, an adamantane-2,6-diyl group, a spirocyclohexane-1,2′-cyclopentane-diyl group and a spiroadamantane-2,3′-cyclopentane-diyl group.

The number of carbon atoms of the alicyclic hydrocarbon group is preferably 3 to 24, more preferably 3 to 20, still more preferably 3 to 18, yet more preferably 3 to 16, and further preferably 3 to 12.

Examples of the divalent aromatic hydrocarbon group include aromatic hydrocarbon groups, for example, arylene groups such as a phenylene group, a naphthylene group, an anthrylene group, a biphenylene group and a phenanthrene group. The number of carbon atoms of the aromatic hydrocarbon group is preferably 6 to 24, more preferably 6 to 20, still more preferably 6 to 18, yet more preferably 6 to 14, and further preferably 6 to 10.

Examples of the groups obtained by combining two or more groups include a group obtained by combining an alicyclic hydrocarbon group with a chain hydrocarbon group, a group obtained by combining an aromatic hydrocarbon group with a chain hydrocarbon group, a group obtained by combining an alicyclic hydrocarbon group with an aromatic hydrocarbon group, a group obtained by combining an alicyclic hydrocarbon group, a chain hydrocarbon group and an aromatic hydrocarbon group and the like. In combination, two or more of alicyclic hydrocarbon groups, aromatic hydrocarbon groups and chain hydrocarbon groups may be respectively combined. Any group may also be bonded to X¹.

Examples of the group obtained by combining an alicyclic hydrocarbon group with a chain hydrocarbon group include an -alicyclic hydrocarbon group-chain hydrocarbon group-, a -chain hydrocarbon group-alicyclic hydrocarbon group-chain hydrocarbon group-, a -chain hydrocarbon group-alicyclic hydrocarbon group- and the like.

Examples of the group obtained by combining the aromatic hydrocarbon group with the chain hydrocarbon group include an -aromatic hydrocarbon group-chain hydrocarbon group-, a -chain hydrocarbon group-aromatic hydrocarbon group-chain hydrocarbon group-, a -chain hydrocarbon group-aromatic hydrocarbon group- and the like.

Examples of the group obtained by combining an alicyclic hydrocarbon group with an aromatic hydrocarbon group include an -aromatic hydrocarbon group-alicyclic hydrocarbon group-, an -alicyclic hydrocarbon group-aromatic hydrocarbon group-, an -alicyclic hydrocarbon group-aromatic hydrocarbon group-alicyclic hydrocarbon group-, a group obtained by fusing an alicyclic hydrocarbon group and an aromatic hydrocarbon group and the like.

—CH₂— included in the hydrocarbon group having 1 to 40 carbon atoms as for L¹ may be replaced by —O—, —S—, —SO₂— or —CO—.

When —CH₂— included in the hydrocarbon group having 1 to 40 carbon atoms as for L¹ is replaced by —O—, —S—, —SO₂— or —CO—, the number of carbon atoms before replacement is taken as the number of carbon atoms of the hydrocarbon group.

Examples of the group in which —CH₂— included in the hydrocarbon group is replaced by —O—, —S—, —SO₂— or —CO— include a hydroxy group (a group in which —CH₂— included in the methyl group is replaced by —O—), a carboxy group (a group in which —CH₂—CH₂— included in the ethyl group is replaced by —O—CO—), a thiol group (a group in which —CH₂— included in the methyl group is replaced by —S—), an alkoxy group (a group in which —CH₂— at any position included in the alkyl group is replaced by —O—), an alkoxycarbonyl group (a group in which —CH₂—CH₂— at any position included in alkyl group is replaced by —O—CO—), an alkylsulfonyl group (a group in which —CH₂— at any position included in the alkyl group is replaced by —SO₂—), an alkylcarbonyl group (a group in which —CH₂— at any position included in the alkyl group is replaced by —CO—), an alkylcarbonyloxy group (a group in which —CH₂—CH₂— at any position included in the alkyl group is replaced by —CO—O—), an alkanediyloxy group (a group in which —CH₂— at any position included in the alkanediyl group is replaced by —O—), an alkanediyloxycarbonyl group (a group in which —CH₂—CH₂— at any position included in the alkanediyl group is replaced by —O—CO—), an alkanediylcarbonyl group (a group in which —CH₂— at any position included in the alkanediyl group is replaced by —CO—), an alkanediylcarbonyloxy group (a group in which —CH₂—CH₂— at any position included in the alkanediyl group is replaced by —CO—O—), an alkanediylsulfonyl group (a group in which —CH₂— at any position included in the alkanediyl group is replaced by —SO₂—), an alkylthio group (a group in which —CH₂— at any position included in the alkyl group is replaced by —S—), an oxy group (a group in which —CH₂— included in the methylene group is replaced by —O—), a carbonyl group (a group in which —CH₂— included in the methylene group is replaced by —CO—), a thio group (a group in which —CH₂— included in the methylene group is replaced by —S—), a sulfonyl group (a group in which —CH₂— included in the methylene group is replaced by —SO₂—), an alkanediylthio group (a group in which —CH₂— at any position included in the alkanediyl group is replaced by —S—), a cycloalkoxy group, a cycloalkylalkoxy group, an alkoxycarbonyloxy group, an aromatic hydrocarbon group-carbonyloxy group, an aromatic hydrocarbon group-carbonyl group, an aromatic hydrocarbon group-oxy group, and groups obtained by combining two or more of these groups.

Examples of the alkoxy group include alkoxy groups having 1 to 39 carbon atoms, for example, a methoxy group, an ethoxy group, a propoxy group, a butoxy group, a pentyloxy group, a hexyloxy group, an octyloxy group, a 2-ethylhexyloxy group, a nonyloxy group, a decyloxy group, an undecyloxy group and the like. The number of carbon atoms of the alkoxy group is preferably 1 to 17, more preferably 1 to 11, still more preferably 1 to 6, yet more preferably 1 to 4, and further preferably 1 to 3.

Examples of the alkoxycarbonyl group include alkoxycarbonyl groups having 2 to 39 carbon atoms, for example, a methoxycarbonyl group, an ethoxycarbonyl group, a butoxycarbonyl group and the like. Examples of the alkylcarbonyl group include alkylcarbonyl groups having 2 to 40 carbon atoms, for example, an acetyl group, a propionyl group and a butyryl group. Examples of the alkylcarbonyloxy group include alkylcarbonyloxy groups having 2 to 39 carbon atoms, for example, an acetyloxy group, a propionyloxy group, a butyryloxy group and the like. The number of carbon atoms of the alkoxycarbonyl group is preferably 1 to 17, more preferably 1 to 11, still more preferably 1 to 6, yet more preferably 1 to 4, and further preferably 1 to 3. The number of carbon atoms of the alkylcarbonyl group is preferably 2 to 18, more preferably 2 to 12, still more preferably 2 to 6, yet more preferably 2 to 4, and further preferably 2 or 3. The number of carbon atoms of the alkylcarbonyloxy group is preferably 2 to 17, more preferably 2 to 11, still more preferably 2 to 6, yet more preferably 2 to 4, and further preferably 2 or 3.

Examples of the alkylsulfonyl group include alkylsulfonyl groups having 1 to 39 carbon atoms, for example, a methylsulfonyl group, an ethylsulfonyl group, a propylsulfonyl group, a butylsulfonyl group, a pentylsulfonyl group, a hexylsulfonyl group, an octylsulfonyl group, a 2-ethylhexylsulfonyl group, a nonylsulfonyl group, a decylsulfonyl group, an undecylsulfonyl group and the like. The number of carbon atoms of the alkylsulfonyl group is preferably 1 to 17, more preferably 1 to 11, still more preferably 1 to 6, yet more preferably 1 to 4, and further preferably 1 to 3.

Examples of the alkanediyloxy group include alkanediyloxy groups having 1 to 39 carbon atoms, for example, a methyleneoxy group, an ethyleneoxy group, a propanediyloxy group, a butanediyloxy group, a pentanediyloxy group and the like. The number of carbon atoms of the alkanediyloxy group is preferably 1 to 17, more preferably 1 to 11, still more preferably 1 to 6, yet more preferably 1 to 4, and further preferably 1 to 3.

Examples of the alkanediyloxycarbonyl group include alkanediyloxycarbonyl groups having 2 to 39 carbon atoms, for example, a methyleneoxycarbonyl group, an ethyleneoxycarbonyl group, a propanediyloxycarbonyl group, a butanediyloxycarbonyl group and the like. Examples of the alkanediylcarbonyl group include alkanediylcarbonyl groups having 2 to 40 carbon atoms, for example, a methylenecarbonyl group, an ethylenecarbonyl group, a propanediylcarbonyl group, a butanediylcarbonyl group, a pentanediylcarbonyl group and the like. Examples of the alkanediylcarbonyloxy group include alkanediylcarbonyloxy groups having 2 to 39 carbon atoms, for example, a methylenecarbonyloxy group, an ethylenecarbonyloxy group, a propanediylcarbonyloxy group, a butanediylcarbonyloxy group and the like. The number of carbon atoms of the alkanediyloxycarbonyl group is preferably 2 to 17, more preferably 2 to 11, still more preferably 2 to 6, yet more preferably 2 to 4, and further preferably 2 or 3. The number of carbon atoms of the alkanediylcarbonyl group is preferably 2 to 18, more preferably 2 to 12, still more preferably 2 to 6, yet more preferably 2 to 4, and further preferably 2 or 3. The number of carbon atoms of the alkanediylcarbonyloxy group is preferably 2 to 17, more preferably 2 to 11, yet more preferably 2 to 6, further preferably 2 to 4, and still further preferably 2 or 3.

Examples of the alkylthio group includes alkylthio groups having 1 to 39 carbon atoms, for example, a methylthio group, an ethylthio group, a propylthio group, a pentylthio group, a hexylthio group, an octylthio group, a 2-ethylhexylthio group, a nonylthio group, a decylthio group, an undecylthio group and the like. The number of carbon atoms of the alkylthio group is preferably 1 to 17, more preferably 1 to 11, still more preferably 1 to 6, yet more preferably 1 to 4, and further preferably 1 to 3.

Examples of the alkanediylsulfonyl group include alkanediylsulfonyl groups having 1 to 39 carbon atoms, for example, a methylenesulfonyl group, an ethylenesulfonyl group, a propylenesulfonyl group and the like. The number of carbon atoms of the alkanediylsulfonyl group is preferably 1 to 17, more preferably 1 to 11, still more preferably 1 to 6, yet more preferably 1 to 4, and further preferably 1 to 3.

Examples of the alkanediylthio group include alkanediylthio groups having 1 to 39 carbon atoms, for example, a methylenethio group, an ethylenethio group, a propylenethio group and the like. The number of carbon atoms of the alkanediylthio group is preferably 1 to 17, more preferably 1 to 11, still more preferably 1 to 6, yet more preferably 1 to 4, and further preferably 1 to 3.

Examples of the cycloalkoxy group include cycloalkoxy groups having 3 to 39 carbon atoms, for example, a cyclohexyloxy group and the like. Examples of the cycloalkylalkoxy group include cycloalkylalkoxy groups having 4 to 39 carbon atoms, for example, a cyclohexylmethoxy group and the like. Examples of the alkoxycarbonyloxy group include alkoxycarbonyloxy groups having 2 to 16 carbon atoms, for example, a butoxycarbonyloxy group and the like. Examples of the aromatic hydrocarbon group-carbonyloxy group include aromatic hydrocarbon group-carbonyloxy group having 7 to 39 carbon atoms, for example, a benzoyloxy group and the like. Examples of the aromatic hydrocarbon group-carbonyl group include aromatic hydrocarbon group-carbonyl group having 7 to 39 carbon atoms, for example, a benzoyl group and the like. Examples of the aromatic hydrocarbon group-oxy group include aromatic hydrocarbon group-oxy group having 6 to 16 carbon atoms, for example, a phenyloxy group and the like.

Examples of the group in which —CH₂— included in the alicyclic hydrocarbon group is replaced by —O—, —S—, —CO— or —SO₂— include the following groups and the like. —O— or —CO— shown below may be replaced by —S— or —SO₂—. The bonding site can be any position.

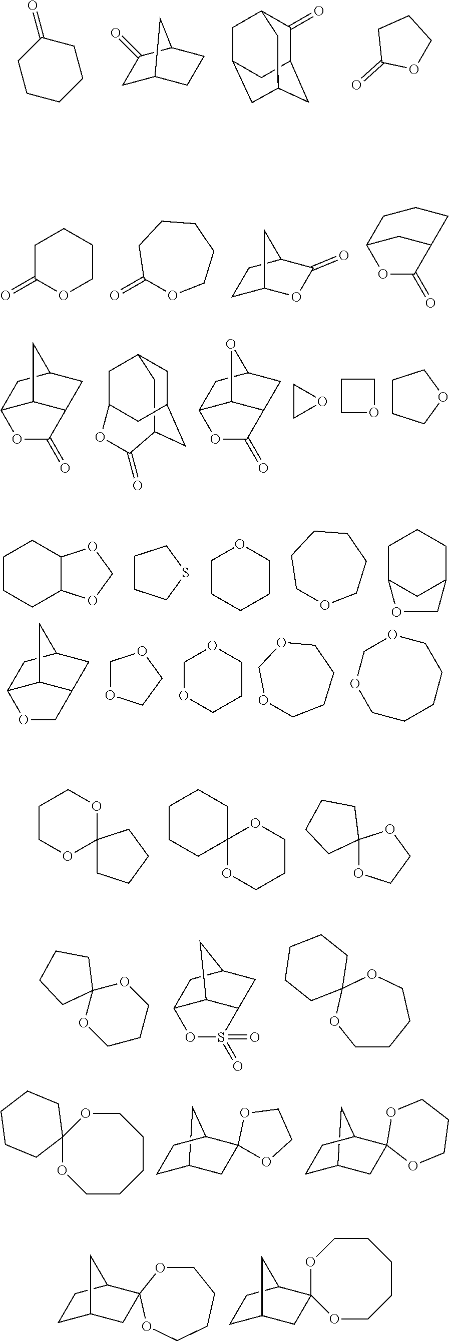

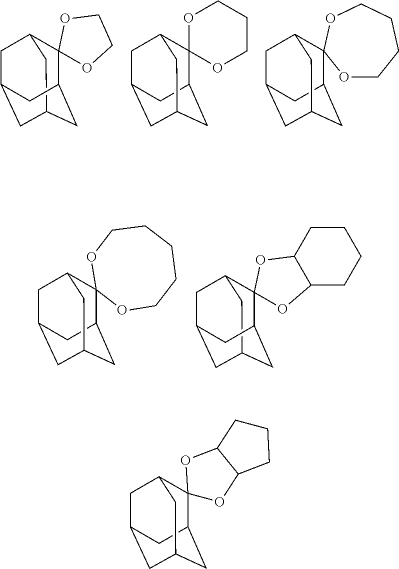

Examples of the substituent which may be possessed by L¹ include a halogen atom, a cyano group, a nitro group and the like. When L¹ is a group obtained by combining an alicyclic hydrocarbon group or an aromatic hydrocarbon group with an alkyl group, the alkyl group may be substantially a substituent of an alicyclic hydrocarbon group or an aromatic hydrocarbon group. By replacing —CH₂— included in the alkanediyl group as for L¹ by —O—, —CO— or —SO₂—, L¹⁰ can substantially have a substituent such as a hydroxy group, a carboxyl group, an alkoxy group, an alkylcarbonyl group, an alkoxycarbonyl group, an alkylcarbonyl group, an alkylcarbonyloxy group, a thiol group or a sulfonyl group.

Examples of the halogen atom include a fluorine atom, a chlorine atom, a bromine atom and an iodine atom.

The substituent is preferably a hydroxy group or a halogen atom.

L¹ is preferably a single bond, a chain hydrocarbon group having 1 to 10 carbon atoms which may have a substituent (in which —CH₂— included in the chain hydrocarbon group may be replaced by —O— or —CO—), a cyclic hydrocarbon group having 3 to 24 carbon atoms which may have a substituent (in which —CH₂— included in the cyclic hydrocarbon group may be replaced by —O—, —S—, —CO— or —SO₂—), or a group obtained by combining a chain hydrocarbon group having 1 to 8 carbon atoms which may have a substituent with a cyclic hydrocarbon group having 3 to 24 carbon atoms which may have a substituent (in which —CH₂— included in the chain hydrocarbon group may be replaced by —O— or —CO—, and —CH₂— included in the cyclic hydrocarbon group may be replaced by —O—, —S—, —SO₂— or —CO—), more preferably a single bond, a chain hydrocarbon group having 1 to 8 carbon atoms which may have a substituent (in which —CH₂— included in the chain hydrocarbon group may be replaced by —O— or —CO—), a cyclic hydrocarbon group having 3 to 20 carbon atoms which may have a substituent (in which —CH₂— included in the cyclic hydrocarbon group may be replaced by —O—, —S—, —CO— or —SO₂—), or a group obtained by combining a chain hydrocarbon group having 1 to 6 carbon atoms which may have a substituent with a cyclic hydrocarbon group having 3 to 20 carbon atoms which may have a substituent (in which —CH₂— included in the chain hydrocarbon group may be replaced by —O— or —CO—, and —CH₂— included in the cyclic hydrocarbon group may be replaced by —O—, —S—, —SO₂— or —CO—), and still more preferably a single bond, a chain hydrocarbon group having 1 to 7 carbon atoms which may have a substituent (in which —CH₂— included in the chain hydrocarbon group may be replaced by —O— or —CO—), a cyclic hydrocarbon group having 3 to 18 carbon atoms which may have a substituent (in which —CH₂— included in the cyclic hydrocarbon group may be replaced by —O— or —CO—), or a group obtained by combining a chain hydrocarbon group having 1 to 5 carbon atoms which may have a substituent with a cyclic hydrocarbon group having 3 to 18 carbon atoms which may have a substituent (in which —CH₂— included in the chain hydrocarbon group may be replaced by —O— or —CO—, and —CH₂— included in the cyclic hydrocarbon group may be replaced by —O— or —CO—).

L¹ is also preferably a single bond, *-L²- or *-L²-X³-L³- (L² represents a chain hydrocarbon group having 1 to 10 carbon atoms which may have a substituent, a cyclic hydrocarbon group having 3 to 24 carbon atoms which may have a substituent, or a group obtained by combining a cyclic hydrocarbon group having 3 to 24 carbon atoms which may have a substituent with a chain hydrocarbon group having 1 to 8 carbon atoms which may have a substituent, —CH₂— included in the cyclic hydrocarbon group may be replaced by —O—, —S—, —CO— or —SO₂—, —CH₂— included in the chain hydrocarbon group may be replaced by —O— or —CO—, * represents a bonding site to X¹, X³ represents **—CO—O—, **—O—CO—, **—O—CO—O— or **—O—, ** represents a bonding site to L², L³ represents a single bond or a chain hydrocarbon group having 1 to 8 carbon atoms which may have a substituent, and —CH₂— included in the chain hydrocarbon group may be replaced by —O— or —CO—).

Examples of the combination of the chain hydrocarbon group and the cyclic hydrocarbon group include a *-chain hydrocarbon group-alicyclic hydrocarbon group or an aromatic hydrocarbon group-, an *-alicyclic hydrocarbon group or an aromatic hydrocarbon group-chain hydrocarbon group-, a *-chain hydrocarbon group-alicyclic hydrocarbon group or an aromatic hydrocarbon group-chain hydrocarbon group- and the like. * represents a bonding site to X¹.

Examples of the substituent which may be possessed by L² and L³ include those which are the same as the substituents which may be possessed by L¹ mentioned above.

Examples of the cyclic hydrocarbon group as for L² include an alicyclic hydrocarbon group, an aromatic hydrocarbon group, or groups obtained by fusing these groups, and include those which are the same as mentioned as for L¹.

Examples of the chain hydrocarbon group as for L² and L³ include those which are the same as mentioned as for L¹.

L² is preferably a chain hydrocarbon group having 1 to 8 carbon atoms which may have a substituent (—CH₂— included in the chain hydrocarbon group may be replaced by —O— or —CO—), a cyclic hydrocarbon group having 3 to 20 carbon atoms which may have a substituent (—CH₂— included in the cyclic hydrocarbon group may be replaced by —O—, —S—, —CO— or —SO₂—), or a group obtained by combining a cyclic hydrocarbon group having 3 to 20 carbon atoms which may have a substituent with a chain hydrocarbon group having 1 to 6 carbon atoms which may have a substituent (—CH₂— included in the cyclic hydrocarbon group may be replaced by —O—, —S—, —CO— or —SO₂—, and —CH₂— included in the chain hydrocarbon group may be replaced by —O— or —CO—), and more preferably a chain hydrocarbon group having 1 to 6 carbon atoms which may have a substituent (—CH₂— included in the chain hydrocarbon group may be replaced by —O— or —CO—), a cyclic hydrocarbon group having 3 to 18 carbon atoms which may have a substituent (—CH₂— included in the cyclic hydrocarbon group may be replaced by —O—, —S—, —CO— or —SO₂—), or a group obtained by combining a cyclic hydrocarbon group having 3 to 18 carbon atoms which may have a substituent with a chain hydrocarbon group having 1 to 4 carbon atoms which may have a substituent (—CH₂— included in the cyclic hydrocarbon group may be replaced by —O—, —S—, —CO— or —SO₂—, and —CH₂— included in the chain hydrocarbon group may be replaced by —O— or —CO—).

L³ is preferably a single bond or a chain hydrocarbon group having 1 to 6 carbon atoms which may have a substituent, more preferably a single bond or a chain hydrocarbon group having 1 to 4 carbon atoms, still more preferably a single bond, a methylene group or an ethylene group, and yet more preferably a single bond or a methylene group.

Examples of the halogen atom as for R¹ and R² include a fluorine atom, a chlorine atom, a bromine atom and an iodine atom.

The haloalkyl group having 1 to 12 carbon atoms in R¹ and R² represents an alkyl group having 1 to 12 carbon atoms which has a halogen atom, and examples thereof include an alkyl fluoride group having 1 to 12 carbon atoms, an alkyl chloride group having 1 to 12 carbon atoms, an alkyl bromide group having 1 to 12 carbon atoms, an alkyl iodide group having 1 to 12 carbon atoms and the like. Examples of the haloalkyl group include a perfluoroalkyl group having 1 to 12 carbon atoms (a trifluoromethyl group, a pentafluoroethyl group, a heptafluoropropyl group, a nonafluorobutyl group, etc.), a difluoromethyl group, a 2,2,2-trifluoroethyl group, a 3,3,3-trifluoropropyl group, a 4,4,4-trifluorobutyl group, a 3,3,4,4,4-pentafluorobutyl group, a chloromethyl group, a bromomethyl group, an iodomethyl group and the like. The number of carbon atoms of the haloalkyl group is preferably 1 to 9, more preferably 1 to 6, still more preferably 1 to 4, and yet more preferably 1 to 3.

Examples of the alkyl group having 1 to 12 carbon atoms as for R² include alkyl groups such as a methyl group, an ethyl group, a propyl group, an isopropyl group, a butyl group, an isobutyl group, a tert-butyl group, a pentyl group, a hexyl group, an octyl group and a nonyl group. The number of carbon atoms of the alkyl group is preferably 1 to 10, more preferably 1 to 8, still more preferably 1 to 6, yet more preferably 1 to 4, and further preferably 1 to 3.

When —CH₂— included in the haloalkyl group or the alkyl group represented by R² is replaced by —O— or —CO—, the number of carbon atoms before replacement is taken as the total number of the haloalkyl group or the alkyl group. Examples of the replaced group include a hydroxy group (a group in which —CH₂— included in the methyl group is replaced by —O—), a carboxy group (a group in which —CH₂—CH₂— included in the ethyl group is replaced by —O—CO—), an alkoxy group (a group in which —CH₂— at any position included in the alkyl group is replaced by —O—), an alkoxycarbonyl group (a group in which —CH₂—CH₂— at any position included in the alkyl group is replaced by —O—CO—), an alkylcarbonyl group (a group in which —CH₂— at any position included in the alkyl group is replaced by —CO—), an alkylcarbonyloxy group (a group in which —CH₂—CH₂— at any position included in the alkyl group is replaced by —CO—O—), an alkoxyalkoxy group (two —CH₂-alkyl groups at any position included in the alkyl group are replaced by —O—), a haloalkoxy group (a group in which —CH₂— at any position included in the haloalkyl group is replaced by —O—), a haloalkoxycarbonyl group (a group in which —CH₂—CH₂— at any position included in the haloalkyl group is replaced by —O—CO—), a haloalkylcarbonyl group (a group in which —CH₂— at any position included in the haloalkyl group is replaced by —CO—), a haloalkylcarbonyloxy group (a group in which —CH₂—CH₂— at any position included in the haloalkyl group is replaced by —CO—O—), groups obtained by combining these two or more groups and the like.

Examples of the alkoxy group include alkoxy groups having 1 to 11 carbon atoms, for example, a methoxy group, an ethoxy group, a propoxy group, a butoxy group, a pentyloxy group, a hexyloxy group, an octyloxy group, a 2-ethylhexyloxy group, a nonyloxy group, a decyloxy group, an undecyloxy group and the like. The number of carbon atoms of the alkoxy group is preferably 1 to 6, more preferably 1 to 4, and still more preferably 1 to 3.

Examples of the alkoxycarbonyl group include alkoxycarbonyl groups having 2 to 11 carbon atoms, for example, a methoxycarbonyl group, an ethoxycarbonyl group, a butoxycarbonyl group and the like, examples of the alkylcarbonyl group include alkylcarbonyl groups having 2 to 12 carbon atoms, for example, an acetyl group, a propionyl group and a butyryl group, examples of the alkylcarbonyloxy group include alkylcarbonyloxy groups having 2 to 11 carbon atoms, for example, an acetyloxy group, a propionyloxy group, a butyryloxy group and the like. The number of carbon atoms of the alkoxycarbonyl group is preferably 2 to 6, more preferably 2 to 4, and still more preferably 2 or 3. The number of carbon atoms of the alkylcarbonyl group is preferably 2 to 6, more preferably 2 to 4, and still more preferably 2 or 3. The number of carbon atoms of the alkylcarbonyloxy group is preferably 2 to 6, more preferably 2 to 4, and still more preferably 2 or 3.

Examples of the alkoxyalkoxy group include alkoxycarbonyl groups having 2 to 10 carbon atoms, for example, a methoxymethoxy group, a methoxyethoxy group, an ethoxymethoxy group, an ethoxyethoxy group, a propoxymethoxy group, an isopropoxymethoxy group, a butoxymethoxy group, a sec-butoxymethoxy group and a tert-butoxymethoxy group. The number of carbon atoms of the alkoxyalkoxy group is preferably 2 to 8, more preferably 2 to 6, still more preferably 2 to 4, and yet more preferably 2 or 3.

Examples of the haloalkoxy group, the haloalkoxycarbonyl group, the haloalkylcarbonyl group and the haloalkylcarbonyloxy group include a haloalkoxy group having 1 to 11 carbon atoms, haloalkoxycarbonyl groups having 2 to 11 carbon atoms, haloalkylcarbonyl groups having 2 to 12 carbon atoms and haloalkylcarbonyloxy groups having 2 to 11 carbon atoms, for example, groups in which one or more hydrogen atoms of the groups mentioned above are substituted with a halogen atom.

R¹ is preferably an iodine atom, a fluorine atom or a haloalkyl group having 1 to 6 carbon atoms, more preferably an iodine atom, a fluorine atom or a perfluoroalkyl group having 1 to 3 carbon atoms, still more preferably an iodine atom or a trifluoromethyl group, and yet more preferably an iodine atom.

m2 is preferably an integer of 0 to 5, more preferably an integer of 0 to 4, still more preferably an integer of 0 to 3, yet more preferably an integer of 0 to 2, and further preferably 0 or 1.

R² is preferably an iodine atom, a fluorine atom, hydroxy group, a haloalkyl group having 1 to 8 carbon atoms or an alkyl group having 1 to 8 carbon atoms (—CH₂— included in the haloalkyl group and the alkyl group may be replaced by —O— or —CO—), more preferably an iodine atom, a fluorine atom, a hydroxy group, a haloalkyl group having 1 to 6 carbon atoms or an alkyl group having 1 to 6 carbon atoms (—CH₂— included in the haloalkyl group and the alkyl group may be replaced by —O— or —CO—), still more preferably an iodine atom, a fluorine atom, a hydroxy group, an alkyl group having 1 to 6 carbon atoms, a haloalkyl group having 1 to 6 carbon atoms or an alkoxy group having 1 to 5 carbon atoms or an alkoxyalkoxy group having 2 to 4 carbon atoms, yet more preferably an iodine atom, a fluorine atom, a hydroxy group, a trifluoromethyl group, an alkoxy group having 1 to 3 carbon atoms or an alkoxyalkoxy group having 2 to 3 carbon atoms, and further preferably an iodine atom, a fluorine atom, a trifluoromethyl group, a hydroxy group or an ethoxymethoxy group.

m2 is preferably 0 to 4, more preferably 0 to 3, still more preferably 0 to 2, and yet more preferably 0 or 1.

Examples of the halogen atom as for R^{bb1} include those which are the same as the halogen atom mentioned as for R². Examples of the alkyl group which may have a halogen atom as for R^{bb1} include those which are the same as the alkyl group and the haloalkyl group mentioned as for R² as long as the upper limit of the number of carbon atoms permit.

R^{bb1} is preferably a hydrogen atom or a methyl group, and more preferably a methyl group.

R³ is preferably a hydrogen atom or an alkyl group having 1 to 4 carbon atoms, more preferably a hydrogen atom or an alkyl group having 1 to 3 carbon atoms, still more preferably a hydrogen atom, a methyl group or an ethyl group, and yet more preferably a hydrogen atom or a methyl group.

When X¹⁰ is a group represented by *-Ax-Ph-Ay-**, preferred is a linking group represented by the following formula (X¹⁰)

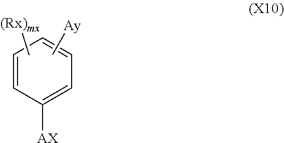

wherein, in formula (X¹⁰),
Ax represents bond species bonded to carbon atoms to which R^{bb1} is bonded, and represents one bond species selected from the group consisting of a single bond, an ether bond, a thioether bond, an ester bond, an amide bond and a carbonic acid ester bond,
Ay represents bond species to which L¹⁰ or a naphthalene ring is bonded, and represents one bond species selected from the group consisting of a single bond, an ether bond, a thioether bond, an ester bond, an amide bond and a carbonic acid ester bond,
Rx represents a halogen atom, a hydroxy group, an alkyl fluoride group having 1 to 6 carbon atoms, or an alkyl group having 1 to 18 carbon atoms, an alkoxy group having 1 to 6 carbon atoms, and
mx represents an integer of 0 to 4, and when mx is an integer of 2 or more, a plurality of Rx may be the same or different from each other.

When one of Ax and Ay is a single bond, the other is preferably one selected from the group consisting of an ether bond, a thioether bond, an ester bond, an amide bond and a carbonic acid ester bond.

When either Ax or Ay is an amide bond, a bond represented by —CO—NR³— is preferable.

Rx is preferably a fluorine atom, an iodine atom, a trifluoromethyl group, av methyl group or an ethyl group.

mx is preferably 0, 1 or 2.

The bonding site of Ay in the phenylene group is preferably the m-position or the p-position, and more preferably the p-position, with respect to the bonding site of Ax.

Examples of X¹⁰ include a group represented by any one of the following formula (X¹⁰-1) to formula (X¹⁰-10). * represents a bonding site to carbon atoms to which —R^{bb1} is bonded. ** represents a bonding site to L¹⁰ or a naphthalene ring:

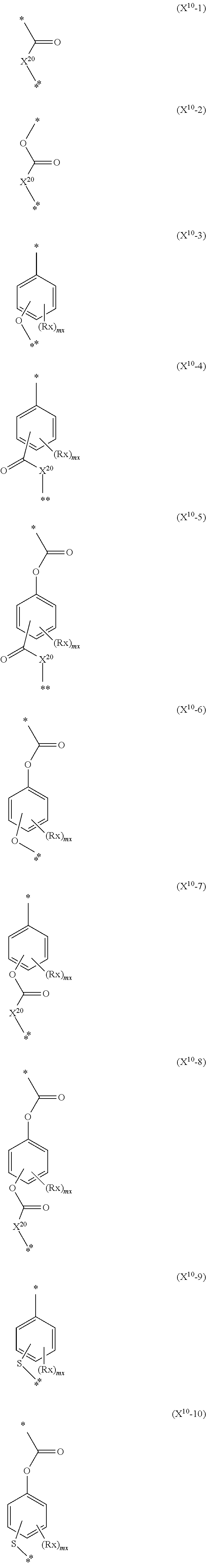

wherein, in formula (X¹⁰-1) to formula (X¹⁰-10),
* and ** are bonding sites, * represents a bonding site to carbon atoms to which R^{bb1} is bonded,
Rx represents a halogen atom, a hydroxy group, an alkyl fluoride group having 1 to 6 carbon atoms, an alkyl group having 1 to 18 carbon atoms or an alkoxy group having 1 to 6 carbon atoms,
mx represents an integer of 0 to 4,
X²⁰ represents —O— or —NR³—, and
R³ represents a hydrogen atom or an alkyl group having 1 to 6 carbon atoms
Specific examples of the groups represented by formula (X¹⁰-1) to formula (X¹⁰-10) include the following groups.

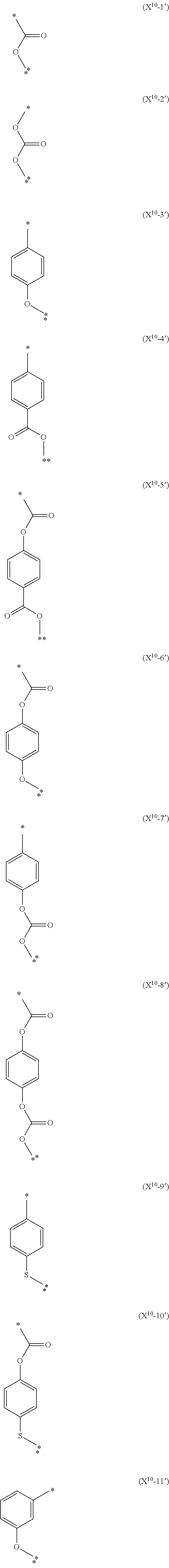

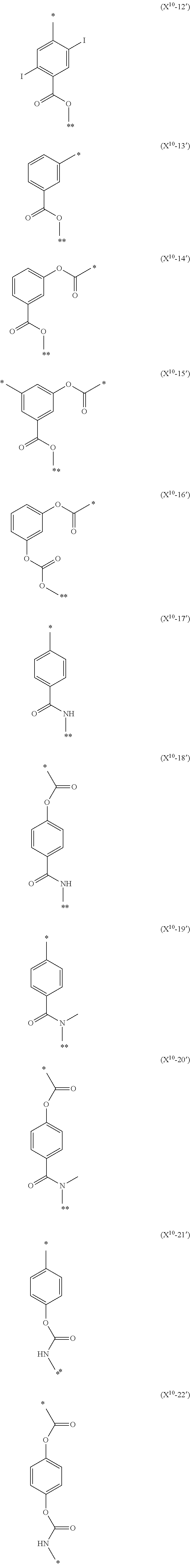

Particularly, X¹⁰ is preferably a single bond or a group represented by any one of formula (X¹⁰-1′) and formula (X¹⁰-3′) to formula (X¹⁰-9′), more preferably a single bond or a group represented by any one of formula (X¹⁰-1′), formula (X¹⁰-4′), formula (X¹⁰-5′), formula (X¹⁰-6′) and formula (X¹⁰-9′), and still more preferably a single bond, a group represented by formula (X¹⁰-1′), a group represented by formula (X¹⁰-5′) or a group represented by formula (X¹⁰-6′) or formula (X¹⁰-9′).

The hydrocarbon group having 1 to 36 carbon atoms as for L¹⁰ includes aliphatic hydrocarbon groups (chain hydrocarbon groups and alicyclic hydrocarbon groups, such as an alkanediyl group, an alkenediyl group and an alkynediyl group), aromatic hydrocarbon groups and the like, and may be hydrocarbon groups obtained by combining two or more of these groups.

Examples of the alkanediyl group include linear alkanediyl groups such as a methylene group, an ethylene group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group and a dodecane-1,12-diyl group; and

branched alkanediyl groups such as an ethane-1,1-diyl group, a propane-1,1-diyl group, a propane-1,2-diyl group, a propane-2,2-diyl group, a pentane-2,4-diyl group, a 2-methylpropane-1,3-diyl group, a 2-methylpropane-1,2-diyl group, a pentane-1,4-diyl group and a 2-methylbutane-1,4-diyl group. The end of the branched alkanediyl group may be a methyl group.

Examples of the alkenediyl group include an ethenediyl group, a propenediyl group, an isopropenediyl group, a butenediyl group, an isobutenediyl group, a tert-butenediyl group, a pentenediyl group, a hexenediyl group, a heptenediyl group, an octynediyl group, an isooctynediyl group, a nonenediyl group and the like.

Examples of the alkynediyl group include an ethynediyl group, a propynediyl group, an isopropynediyl group, a butynediyl group, an isobutynediyl group, a tert-butynediyl group, a pentynediyl group, a hexynediyl group, an octynediyl group, a nonynediyl group and the like.

The number of carbon atoms of the chain hydrocarbon group is preferably 1 to 12, more preferably 1 to 9, still more preferably 1 to 6, yet more preferably 1 to 4, and further preferably 1 to 3.

The divalent alicyclic hydrocarbon group may be monocyclic, polycyclic or spiro ring. Examples of the divalent alicyclic hydrocarbon group include groups shown below. The bonding site can be any position.

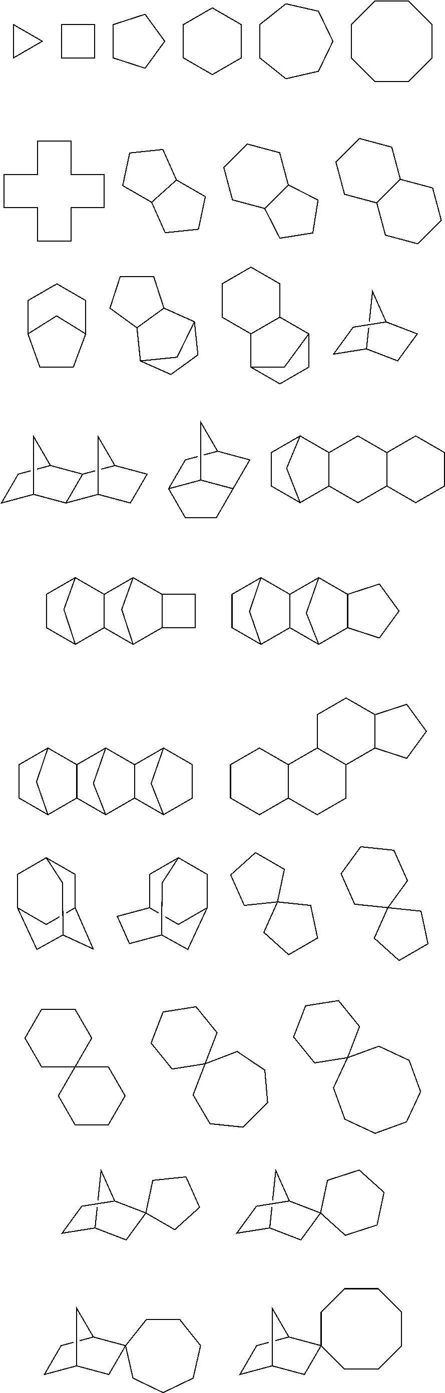

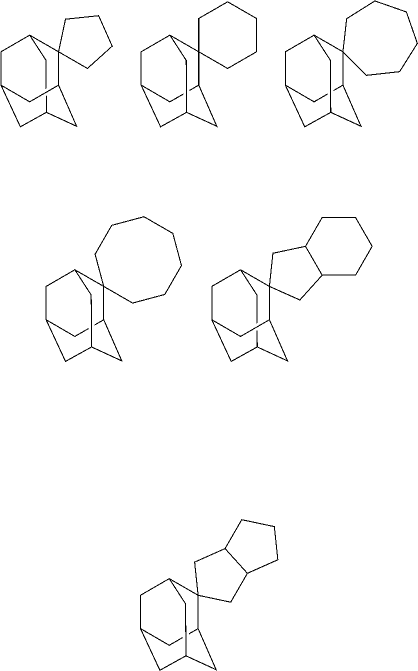

Specifically, examples of the monocyclic divalent alicyclic hydrocarbon group include monocyclic cycloalkanediyl groups such as a cyclobutane-1,3-diyl group, a cyclopentane-1,3-diyl group, a cyclohexane-1,4-diyl group, a cyclohexene-3,6-diyl group and a cyclooctane-1,5-diyl group; and examples of the polycyclic divalent alicyclic hydrocarbon group include polycyclic cycloalkanediyl groups such as a norbornane-1,4-diyl group, a norbornane-2,5-diyl group, a 5-norbornene-2,3-diyl group, an adamantane-1,5-diyl group and an adamantane-2,6-diyl group.

The number of carbon atoms of the divalent alicyclic hydrocarbon group is preferably 3 to 18, more preferably 3 to 16, and still more preferably 3 to 12.

Examples of the divalent aromatic hydrocarbon group include a phenylene group, a naphthylene group, an anthrylene group, a biphenylene group, a phenanthrylene group and the like. The number of carbon atoms of the aromatic hydrocarbon group is preferably 6 to 18, more preferably 6 to 14, and still more preferably 6 to 10.

Examples of the hydrocarbon group obtained by combining two or more groups include groups obtained by combining an alkanediyl group, an alicyclic hydrocarbon group and/or an aromatic hydrocarbon group, and examples thereof include an -alicyclic hydrocarbon group-alkanediyl group-, an -alkanediyl group-alicyclic hydrocarbon group-, an -alkanediyl group-alicyclic hydrocarbon group-alkanediyl group-, an -alkanediyl group-aromatic hydrocarbon group-, an -aromatic hydrocarbon group-alkanediyl group- and the like. In the case of groups obtained by combining an alicyclic hydrocarbon group or an aromatic hydrocarbon group with an alkanediyl group, the end of the alkanediyl group may be a methyl group.

—CH₂— included in the hydrocarbon group having 1 to 36 carbon atoms as for L¹⁰ may be replaced by —O—, —S—, —CO— or —SO₂—.

When the hydrocarbon group having 1 to 36 carbon atoms as for L¹⁰ has a substituent, or when —CH₂— included in the hydrocarbon group is replaced by —O—, —S—, —CO— or —SO₂—, the number of carbon atoms before replacement is taken as the number of carbon atoms of the hydrocarbon group.

Examples of the group in which —CH₂— included in the hydrocarbon group is replaced by —O—, —S—, —SO₂— or —CO— include a hydroxy group (a group in which —CH₂— included in the methyl group is replaced by —O—), a carboxy group (a group in which —CH₂—CH₂— included in the ethyl group is replaced by —O—CO—), a thiol group (a group in which —CH₂— included in the methyl group is replaced by —S—), a carbonyl group (a group in which —CH₂— included in the methylene group is replaced by —CO—), a sulfonyl group (a group in which —CH₂— included in the methylene group is replaced by —SO₂—), an oxy group (a group in which —CH₂— included in the methylene group is replaced by —O—), a thio group (a group in which —CH₂— included in the methylene group is replaced by —S—), an alkoxy group (a group in which —CH₂— at any position included in the alkyl group is replaced by —O—), an alkoxycarbonyl group (a group in which —CH₂—CH₂— at any position included in the alkyl group is replaced by —O—CO—), an alkylcarbonyl group (a group in which —CH₂— at any position included in the alkyl group is replaced by —CO—), an alkylcarbonyloxy group (a group in which —CH₂—CH₂— at any position included in the alkyl group is replaced by —CO—O—), an alkanediyloxy group (a group in which —CH₂ at any position included in the alkanediyl group is replaced by —O—), an alkanediyloxycarbonyl group (a group in which —CH₂—CH₂— at any position included in the alkanediyl group is replaced by —O—CO—), an alkanediylcarbonyl group (a group in which —CH₂— at any position included in the alkanediyl group is replaced by —CO—), an alkanediylcarbonyloxy group (a group in which —CH₂—CH₂— at any position included in the alkanediyl group is replaced by —CO—O—), an alkylthio group (a group in which —CH₂—CH₂— at any position included in the alkyl group is replaced by —S—), an alkylsulfonyl group (a group in which —CH₂— at any position included in the alkyl group is replaced by —SO₂—), an alkanediylsulfonyl group (a group in which —CH₂— at any position included in the alkanediyl group is replaced by —SO₂—), an alkanediylthio group (a group in which —CH₂— at any position included in the alkanediyl group is replaced by —S—), a cycloalkoxy group, a cycloalkylalkoxy group, an alkoxycarbonyloxy group, an aromatic hydrocarbon group-carbonyloxy group, an aromatic hydrocarbon group-carbonyl group, an aromatic hydrocarbon group-oxy group, groups obtained by combining two or more of these groups and the like.

Examples of the alkoxy group include alkoxy groups having 1 to 35 carbon atoms, for example, a methoxy group, an ethoxy group, a propoxy group, a butoxy group, a pentyloxy group, a hexyloxy group, an octyloxy group, a 2-ethylhexyloxy group, a nonyloxy group, a decyloxy group, an undecyloxy group and the like. The number of carbon atoms of the alkoxy group is preferably 1 to 11, more preferably 1 to 6, still more preferably 1 to 4, and yet more preferably 1 to 3.

Examples of the alkoxycarbonyl group include alkoxycarbonyl groups having 2 to 35 carbon atoms, for example, a methoxycarbonyl group, an ethoxycarbonyl group, a butoxycarbonyl group and the like. Examples of the alkylcarbonyl group include alkylcarbonyl groups having 2 to 18 carbon atoms, for example, an acetyl group, a propionyl group and a butyryl group. Examples of the alkylcarbonyloxy group include alkylcarbonyloxy groups having 2 to 35 carbon atoms, for example, an acetyloxy group, a propionyloxy group, a butyryloxy group and the like. The number of carbon atoms of the alkoxycarbonyl group, the alkylcarbonyl group and the alkylcarbonyloxy group is preferably 2 to 11, more preferably 2 to 6, still more preferably 2 to 4, and yet more preferably 2 to 3.

Examples of the alkanediyloxy group include alkanediyloxy groups having 1 to 35 carbon atoms, for example, a methyleneoxy group, an ethyleneoxy group, a propanediyloxy group, a butanediyloxy group, a pentanediyloxy group and the like. The number of carbon atoms of the alkanediyloxy group is preferably 1 to 11, more preferably 1 to 6, still more preferably 1 to 4, and yet more preferably 1 to 3.

Examples of the alkanediyloxycarbonyl group include alkanediyloxycarbonyl groups having 2 to 35 carbon atoms, for example, a methyleneoxycarbonyl group, an ethyleneoxycarbonyl group, a propanediyloxycarbonyl group, a butanediyloxycarbonyl group and the like. Examples of the alkanediylcarbonyl group include alkanediylcarbonyl groups having 2 to 36 carbon atoms, for example, a methylenecarbonyl group, an ethylenecarbonyl group, a propanediylcarbonyl group, a butanediylcarbonyl group, a pentanediylcarbonyl group and the like. Examples of the alkanediylcarbonyloxy group include alkanediylcarbonyloxy groups having 2 to 35 carbon atoms, for example, a methylenecarbonyloxy group, an ethylenecarbonyloxy group, a propanediylcarbonyloxy group, a butanediylcarbonyloxy group and the like. The number of carbon atoms of the alkanediyloxycarbonyl group, the alkanediylcarbonyl group and the alkanediylcarbonyloxy group is preferably 2 to 11, more preferably 2 to 6, still more preferably 2 to 4, and yet more preferably 2 to 3.

Examples of the alkylthio group include alkylthio groups having 1 to 35 carbon atoms, for example, a methylthio group, an ethylthio group, a propylthio group and the like. The number of carbon atoms of the alkylthio group is preferably 1 to 11, more preferably 1 to 6, still more preferably 1 to 4, and yet more preferably 1 to 3.

Examples of the alkylsulfonyl group include alkylsulfonyl groups having 1 to 35 carbon atoms, for example, a methylsulfonyl group, an ethylsulfonyl group, a propylsulfonyl group and the like. The number of carbon atoms of the alkylsulfonyl group is preferably 1 to 11, more preferably 1 to 6, still more preferably 1 to 4, and yet more preferably 1 to 3.

Examples of the alkanediylsulfonyl group include alkanediylsulfonyl groups having 1 to 35 carbon atoms, for example, a methylenesulfonyl group, an ethylenesulfonyl group, a propylenesulfonyl group and the like. The number of carbon atoms of the alkanediylsulfonyl group is preferably 1 to 11, more preferably 1 to 6, still more preferably 1 to 4, and yet more preferably 1 to 3.

Examples of the alkanediylthio group include alkanediylthio groups having 1 to 35 carbon atoms, for example, a methylenethio group, an ethylenethio group, a propylenethio group and the like. The number of carbon atoms of the alkanediylthio group is preferably 1 to 11, more preferably 1 to 6, still more preferably 1 to 4, and yet more preferably 1 to 3.

Examples of the cycloalkoxy group include cycloalkoxy groups having 3 to 35 carbon atoms, for example, a cyclohexyloxy group and the like. Examples of the cycloalkylalkoxy group include cycloalkylalkoxy groups having 4 to 17 carbon atoms, for example, a cyclohexylmethoxy group and the like. Examples of the alkoxycarbonyloxy group include alkoxycarbonyloxy groups having 2 to 34 carbon atoms, for example, a butoxycarbonyloxy group and the like. The number of carbon atoms of the cycloalkoxy group and the cycloalkylalkoxy group is preferably 3 to 11, and more preferably 3 to 6. The number of carbon atoms of the alkoxycarbonyloxy group is preferably 2 to 11, more preferably 2 to 6, still more preferably 2 to 4, and yet more preferably 2 to 3. Examples of the aromatic hydrocarbon group-carbonyloxy group include aromatic hydrocarbon group-carbonyloxy groups having 7 to 35 carbon atoms, for example, a benzoyloxy group and the like. Examples of the aromatic hydrocarbon group-carbonyl group include aromatic hydrocarbon group-carbonyl groups having 7 to 36 carbon atoms, for example, a benzoyl group and the like. Examples of the aromatic hydrocarbon group-oxy group include aromatic hydrocarbon group-oxy groups having 6 to 35 carbon atoms, for example, a phenyloxy group and the like.

Examples of the group in which —CH₂— included in the alicyclic hydrocarbon group is replaced by —O—, —S—, —CO— or —SO₂— include the following groups. —O— or —CO— of the following groups may be replaced by —S— or —SO₂—. The bonding site can be any position.

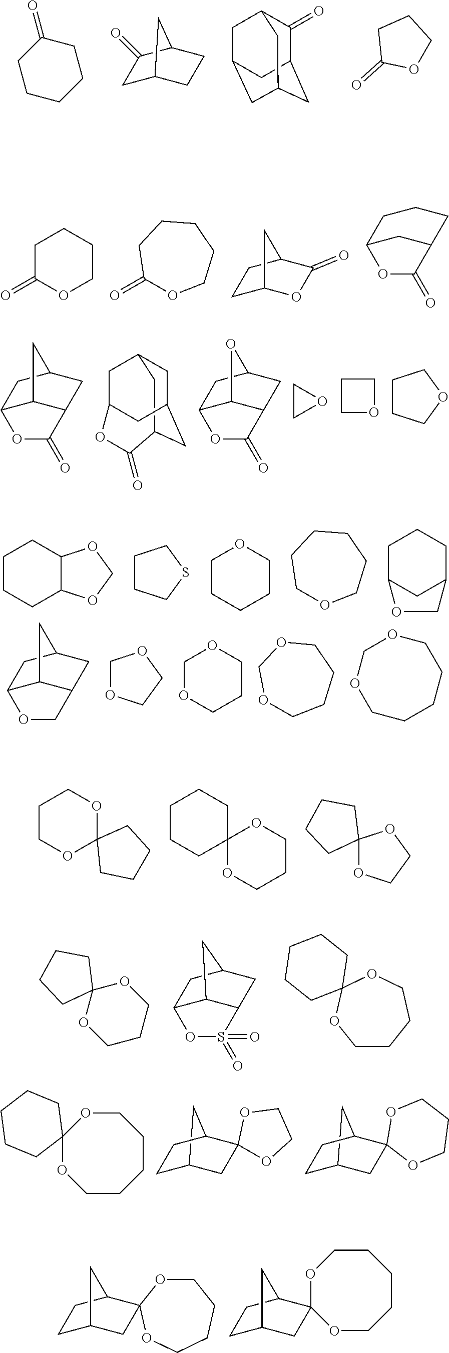

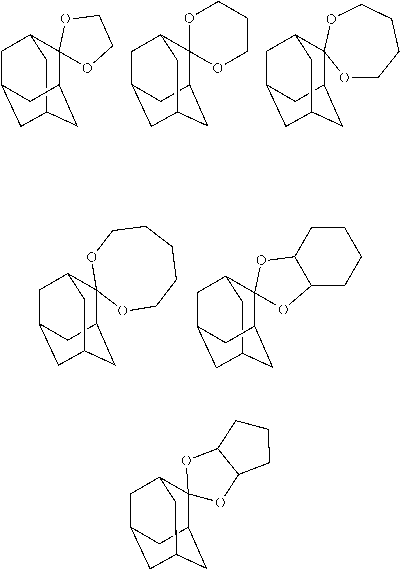

Examples of the substituent which may be possessed by the hydrocarbon group as for L¹⁰ include a halogen atom, a cyano group, a nitro group and the like.

When L¹⁰ includes an alicyclic hydrocarbon group or an aromatic hydrocarbon group, the alkanediyl group (the end of the alkanediyl group may be a methyl group) bonded to an alicyclic hydrocarbon group or an aromatic hydrocarbon group can be substantially regarded as the substituent of the alicyclic hydrocarbon group or the aromatic hydrocarbon group. By the group in which —CH₂— included in the hydrocarbon group as for L¹⁰ is replaced by —O— or —CO—, L¹⁰ can substantially have substituents such as a hydroxy group, a carboxy group, an alkoxy group, an alkoxycarbonyl group, an alkylcarbonyl group and an alkylcarbonyloxy group.

Examples of the halogen atom include a fluorine atom, a chlorine atom, a bromine atom and an iodine atom.

The hydrocarbon group having 1 to 36 carbon atoms as for L¹⁰ may have one substituent or a plurality of substituents.

L¹⁰ is preferably a single bond, an alkanediyl group having 1 to 6 carbon atoms (in which —CH₂— included in the alkanediyl group may be replaced by —O— or —CO—), a cyclic hydrocarbon group having 3 to 18 carbon atoms (in which the cyclic hydrocarbon group may have a substituent, and —CH₂— included in the cyclic hydrocarbon group may be replaced by —O—, —S—, —SO₂— or —CO—) or a group obtained by combining an alkanediyl group having 1 to 6 carbon atoms with a cyclic hydrocarbon group having 3 to 18 carbon atoms (in which the cyclic hydrocarbon group may have a substituent, —CH₂— included in the alkanediyl group may be replaced by —O— or —CO—, and —CH₂— included in the cyclic hydrocarbon group may be replaced by —O—, —S—, —SO₂— or —CO—), more preferably a single bond, an alkanediyl group having 1 to 4 carbon atoms (in which —CH₂— included in the alkanediyl group may be replaced by —O— or —CO—), an alicyclic hydrocarbon group having 3 to 18 carbon atoms (in which —CH₂— included in the alicyclic hydrocarbon group may be replaced by —O—, —S—, —SO₂— or —CO—), a group obtained by combining an aromatic hydrocarbon group having 6 to 18 carbon atoms which may have a substituent, an alkanediyl group having 1 to 4 carbon atoms and an alicyclic hydrocarbon group having 3 to 18 carbon atoms (in which —CH₂— included in the alkanediyl group may be replaced by —O— or —CO—, and —CH₂— included in the alicyclic hydrocarbon group may be replaced by —O—, —S—, —SO₂— or —CO—) or a group obtained by combining an alkanediyl group having 1 to 4 carbon atoms with an aromatic hydrocarbon group having 6 to 18 carbon atoms which may have a substituent (—CH₂— included in the alkanediyl group may be replaced by —O— or —CO—), still more preferably a single bond, an alkanediyl group having 1 to 4 carbon atoms (in which —CH₂— included in the alkanediyl group may be replaced by —O— or —CO—), an alicyclic hydrocarbon group having 3 to 18 carbon atoms (in which —CH₂— included in the alicyclic hydrocarbon group may be replaced by —O— or —CO—), a group obtained by combining a phenylene group which may have a substituent, an alkanediyl group having 1 to 4 carbon atoms and an alicyclic hydrocarbon group having 3 to 18 carbon atoms (in which —CH₂— included in the alkanediyl group may be replaced by —O— or —CO—, and —CH₂— included in the alicyclic hydrocarbon group may be replaced by —O— or —CO—) or a group obtained by combining an alkanediyl group having 1 to 4 carbon atoms with a phenylene group which may have a substituent (in which —CH₂— included in the alkanediyl group may be replaced by —O— or —CO—), and yet more preferably a single bond or alkanediyl group having 1 to 4 carbon atoms (in which —CH₂— included in the alkanediyl group may be replaced by —O— or —CO—).

Examples of the anion (I) include the following anions. Of these, anions represented by formula (Ia-1) to formula (Ia-6), formula (Ia-11) to formula (Ia-16), formula (Ia-20) to formula (Ia-24) and formula (Ia-31) to formula (Ia-35) are preferable.

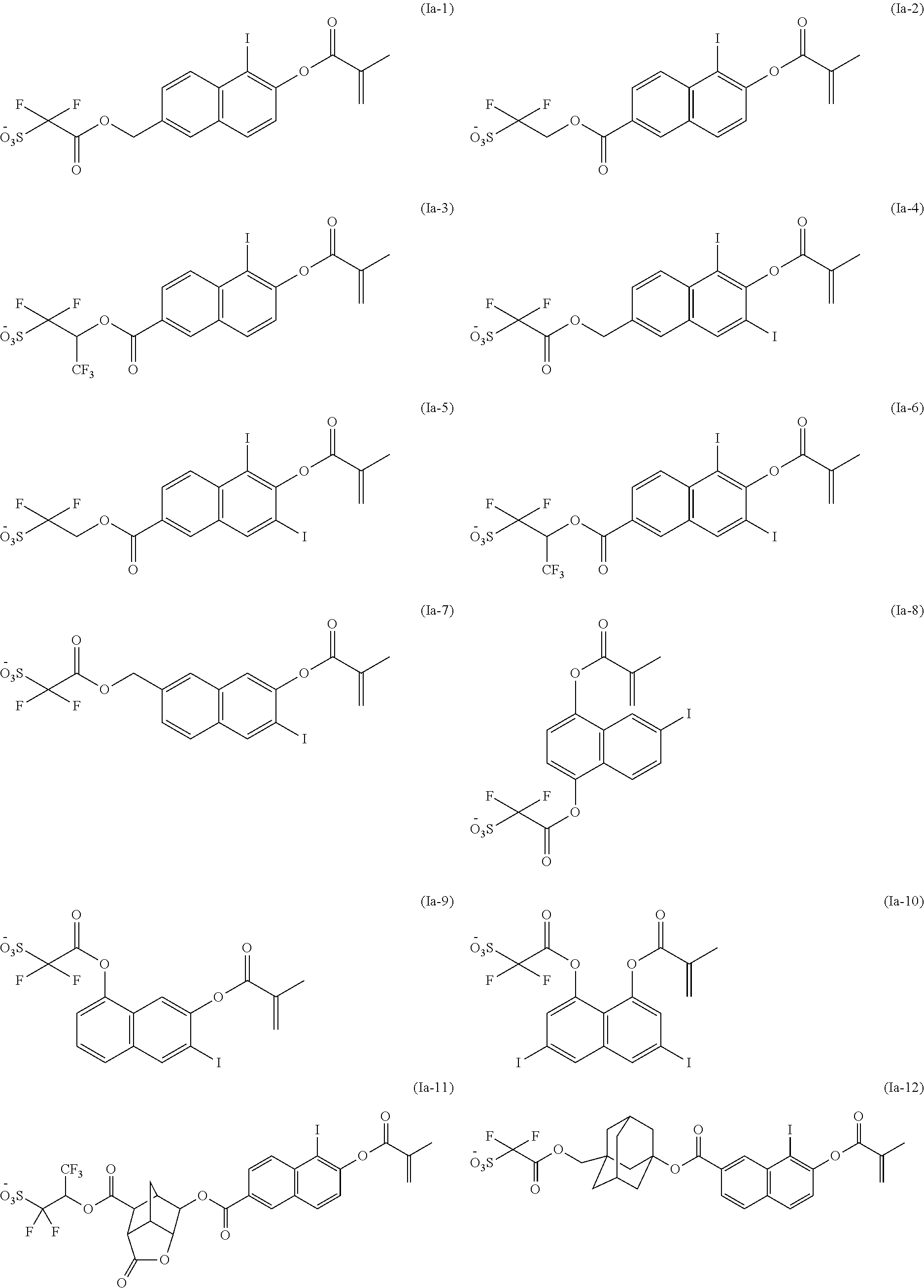

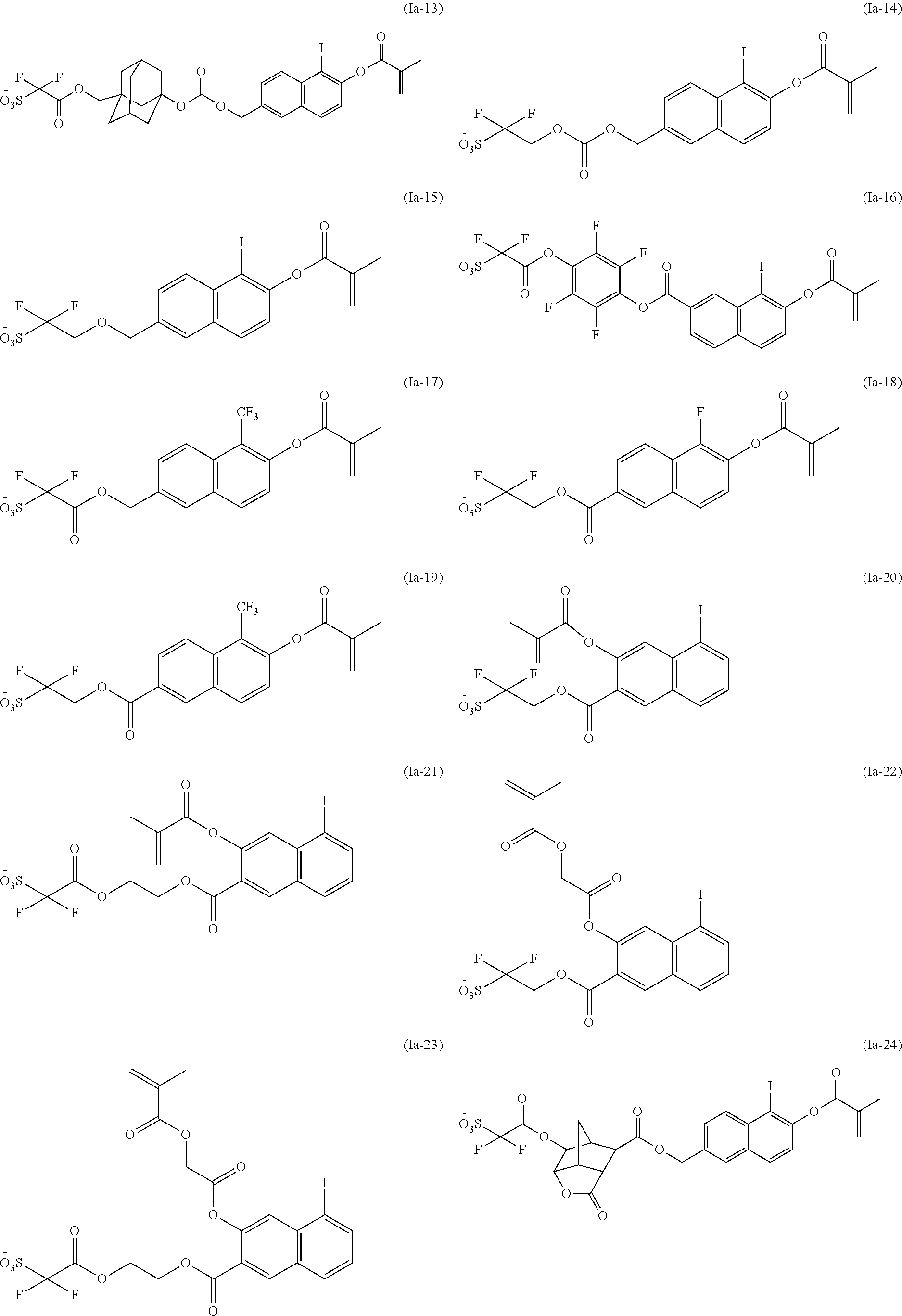

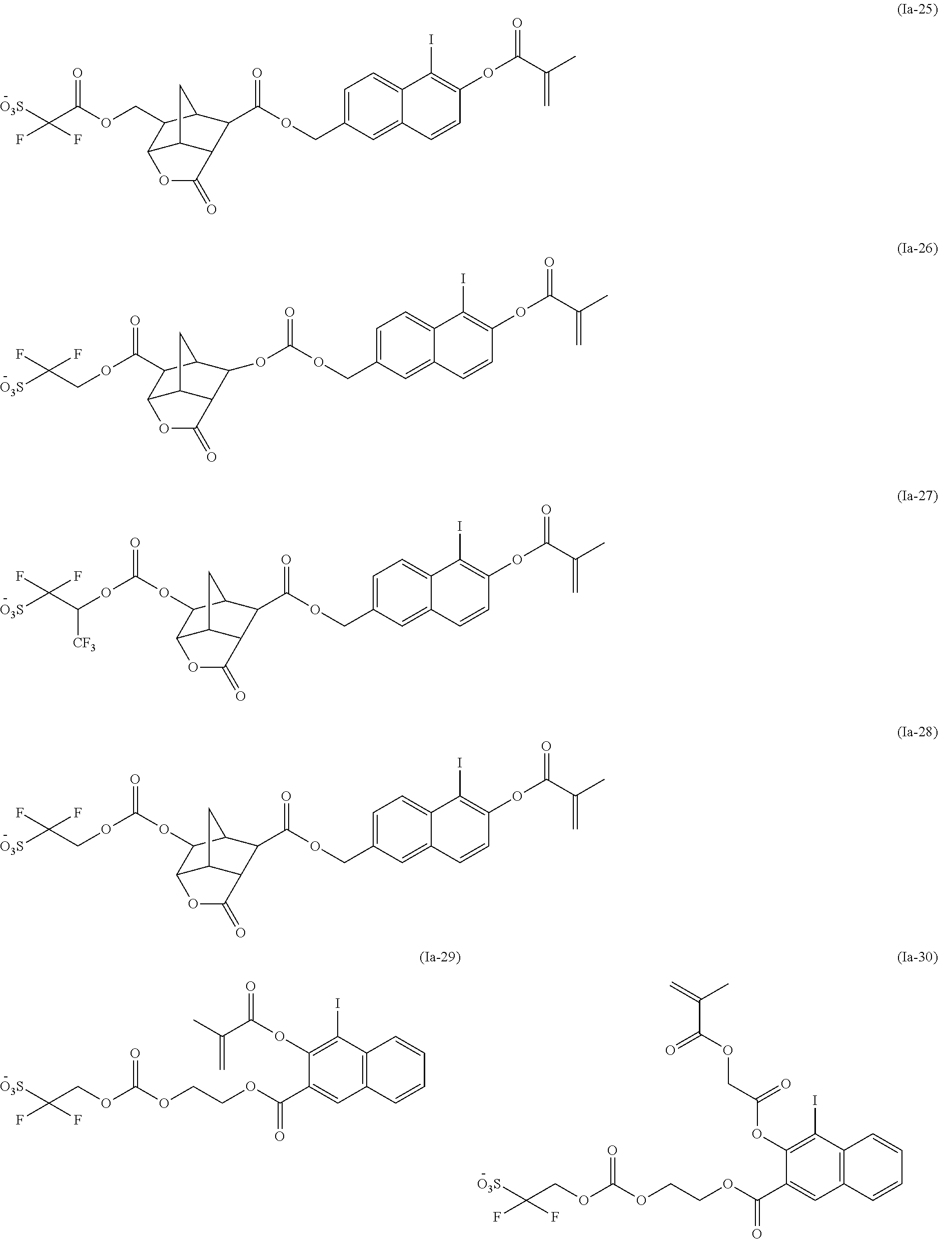

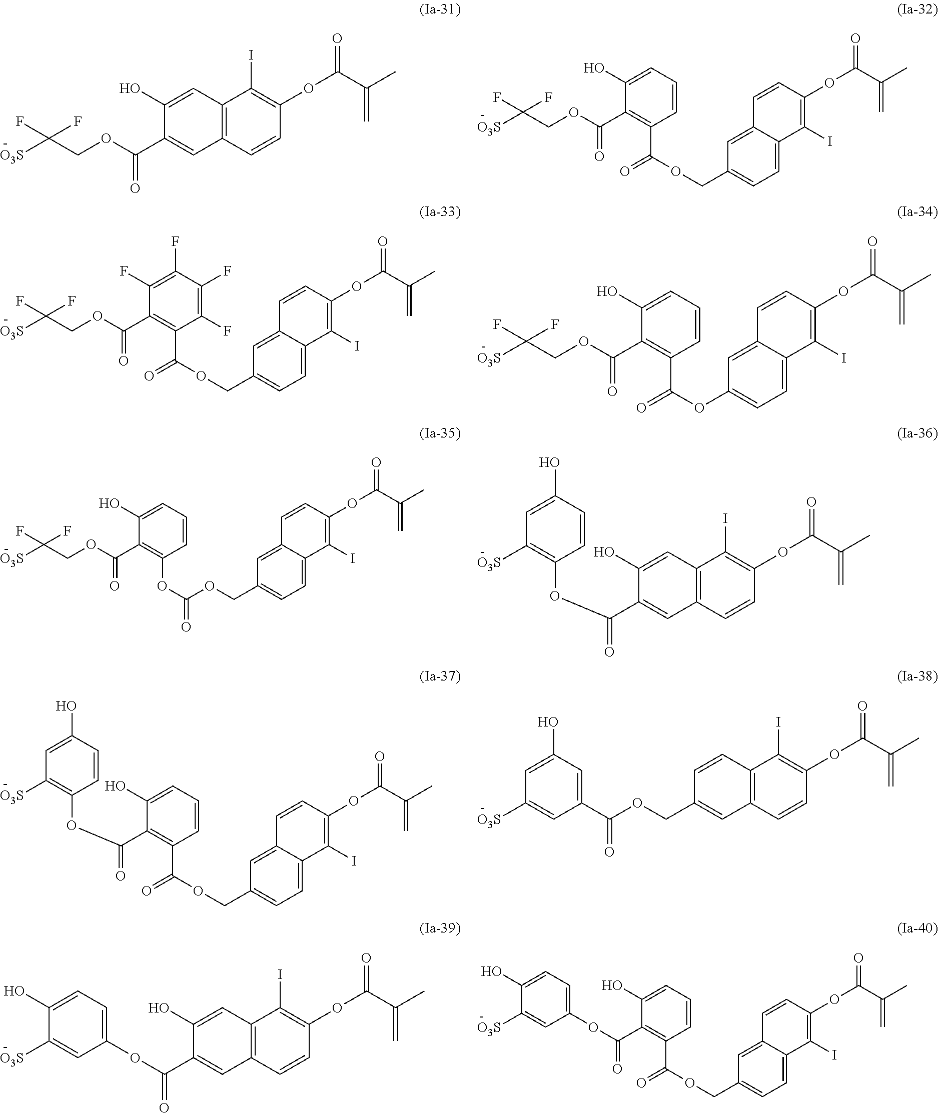

Examples of the organic cation as for Z⁺ include an organic onium cation, an organic sulfonium cation, an organic iodonium cation, an organic ammonium cation, a benzothiazolium cation and an organic phosphonium cation. Of these, an organic sulfonium cation and an organic iodonium cation are preferable, and an arylsulfonium cation is more preferable. Specific examples thereof include a cation represented by any one of formula (b2-1) to formula (b2-5) (hereinafter sometimes referred to as “cation (b2-1)” or the like according to the number of formula):

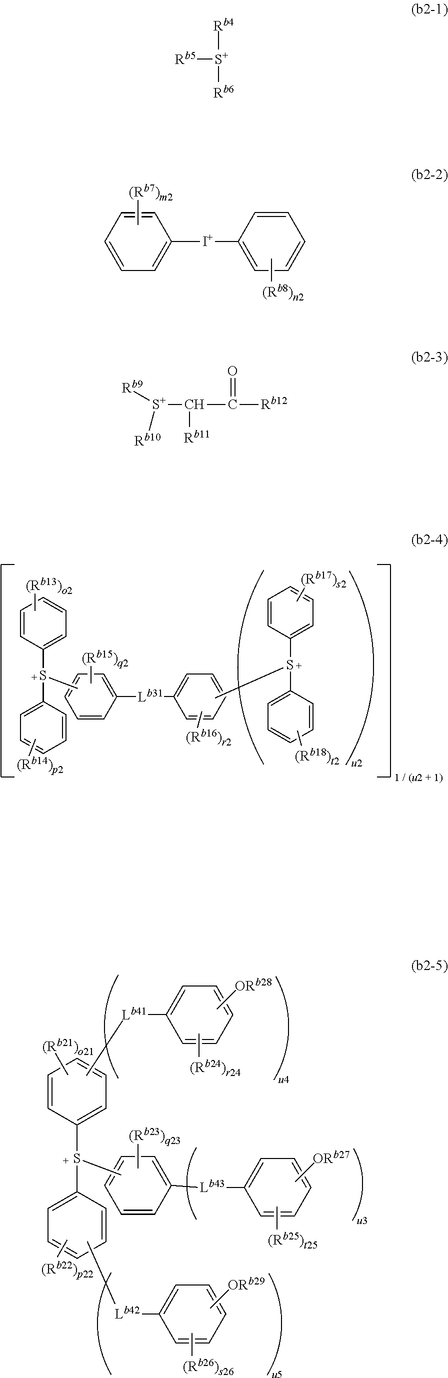

wherein, in formula (b2-1) to formula (b2-5),
R^{b}4 to R^{b}6 each independently represent a chain hydrocarbon group having 1 to 30 carbon atoms, an alicyclic hydrocarbon group having 3 to 36 carbon atoms or an aromatic hydrocarbon group having 6 to 36 carbon atoms, a hydrogen atom included in the chain hydrocarbon group may be substituted with a hydroxy group, an alkoxy group having 1 to 12 carbon atoms, an alicyclic hydrocarbon group having 3 to 12 carbon atoms or an aromatic hydrocarbon group having 6 to 18 carbon atoms, a hydrogen atom included in the alicyclic hydrocarbon group may be substituted with a halogen atom, an aliphatic hydrocarbon group having 1 to 18 carbon atoms, an alkylcarbonyl group having 2 to 4 carbon atoms or a glycidyloxy group, and a hydrogen atom included in the aromatic hydrocarbon group may be substituted with a halogen atom, a hydroxy group, an aliphatic hydrocarbon group having 1 to 18 carbon atoms, an alkyl fluoride group having 1 to 12 carbon atoms or an alkoxy group having 1 to 12 carbon atoms,
R^{b4} and R^{b5} may be bonded to each other to form a ring together with sulfur atoms to which R^{b4} and R^{b5} are bonded, and —CH₂— included in the ring may be replaced by —O—, —S— or —CO—,
R^{b7} and R^{b8} each independently represent a halogen atom, a hydroxy group, an aliphatic hydrocarbon group having 1 to 12 carbon atoms, an alkyl fluoride group having 1 to 12 carbon atoms or an alkoxy group having 1 to 12 carbon atoms,
m2 and n2 each independently represent an integer of 0 to 5,
when m2 is 2 or more, a plurality of R^{b7} may be the same or different, and when n2 is 2 or more, a plurality of R^{b8} may be the same or different,
R^{b9} and R^{b10} each independently represent a chain hydrocarbon group having 1 to 36 carbon atoms or an alicyclic hydrocarbon group having 3 to 36 carbon atoms,
R^{b9} and R^{b10} may be bonded to each other to form a ring together with sulfur atoms to which R^{b9} and R^{b10} are bonded, and —CH₂— included in the ring may be replaced by —O—, —S— or —CO—,
R^{b11} represents a hydrogen atom, a chain hydrocarbon group having 1 to 36 carbon atoms, an alicyclic hydrocarbon group having 3 to 36 carbon atoms or an aromatic hydrocarbon group having 6 to 18 carbon atoms,
R^{b12} represents a chain hydrocarbon group having 1 to 12 carbon atoms, an alicyclic hydrocarbon group having 3 to 18 carbon atoms or an aromatic hydrocarbon group having 6 to 18 carbon atoms, a hydrogen atom included in the chain hydrocarbon group may be substituted with an aromatic hydrocarbon group having 6 to 18 carbon atoms, a hydrogen atom included in the aromatic hydrocarbon group may be substituted with an alkoxy group having 1 to 12 carbon atoms or an alkylcarbonyloxy group having 1 to 12 carbon atoms,
R^{b11} and R^{b12} may be bonded to each other to form a ring, including —CH—CO— to which R^{b11} and R^{b12} are bonded, and —CH₂— included in the ring may be replaced by —O—, —S— or —CO—,
R^{b13} to R^{b18} and R^{b}21 to R^{b2} each independently represent a halogen atom, a hydroxy group, an aliphatic hydrocarbon group having 1 to 12 carbon atoms, an alkyl fluoride group having 1 to 12 carbon atoms or an alkoxy group having 1 to 12 carbon atoms,
R^{b13} and R^{b14}, and R^{b21} and R^{b22} may be bonded to each other to form a ring having a sulfur atom together with the benzene ring to which R^{b13} and R^{b14} are bonded, and —CH₂— included in the ring may be replaced by —O—, —S— or —CO—,
R^{b27} to R^{b29} each independently represent a hydrogen atom or an acid-labile group,
L^{b31} and L^{b41} to L^{b43} represent a sulfur atom or an oxygen atom,
o2, p2, s2 and t2 each independently represent an integer of 0 to 5,
q2 and r2 each independently represent an integer of 0 to 4,
u2 represents 0 or 1,
when o2 is 2 or more, a plurality of R^{b13} are the same or different, when p2 is 2 or more, a plurality of R^{b14} are the same or different, when q2 is 2 or more, a plurality of R^{b15} are the same or different, when r2 is 2 or more, a plurality of R^{b16} are the same or different, when s2 is 2 or more, a plurality of R^{b17} are the same or different, and when t2 is 2 or more, a plurality of R^{b18} are the same or different,
u3 represents an integer of 1 to 3, and satisfies: 1≤u3+q≤5,
u4 and u5 each independently represent an integer of 0 to 3, and satisfy: 0≤u4+o21≤5 and 0≤u5+p22≤5, and
when u3, u4 and u5 are each 2 or more, groups in parentheses are each the same or different.

When u2 is 0, any one of o2, p2, q2 and r2 is preferably 1 or more and at least one of R^{b13} to R^{b16} is preferably a halogen atom, and when u2 is 1, any one of o2, p2, s2, t2, q2 and r2 is preferably 1 or more and at least one of R^{b13} to R^{b18} is preferably a halogen atom.

Further, when u2 is 0, r2 is preferably 1 or more, and more preferably 1. When u2 is 0 and r2 is 1 or more, R^{b16} is preferably a halogen atom.

The aliphatic hydrocarbon group represents a chain hydrocarbon group and an alicyclic hydrocarbon group.

Examples of the chain hydrocarbon group include alkyl groups such as a methyl group, an ethyl group, a propyl group, an isopropyl group, a butyl group, a sec-butyl group, a tert-butyl group, a pentyl group, a hexyl group, an octyl group and a 2-ethylhexyl group.

Particularly, the chain hydrocarbon group of R^{b9} to R^{b12} preferably has 1 to 12 carbon atoms.

The alicyclic hydrocarbon group may be either monocyclic or polycyclic, and examples of the monocyclic alicyclic hydrocarbon group include cycloalkyl groups such as a cyclopropyl group, a cyclobutyl group, a cyclopentyl group, a cyclohexyl group, a cycloheptyl group, a cyclooctyl group and a cyclodecyl group. Examples of the polycyclic alicyclic hydrocarbon group include a decahydronaphthyl group, an adamantyl group, a norbornyl group and the following groups.

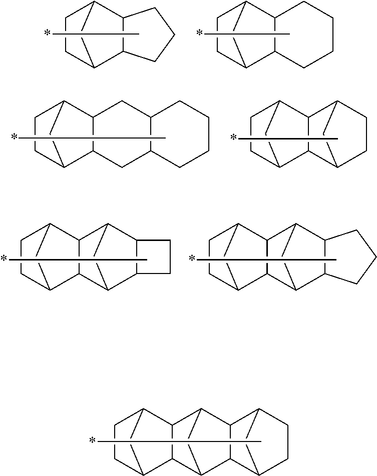

Particularly, the alicyclic hydrocarbon group of R^{b9} to R^{b12} preferably has 3 to 18 carbon atoms, and more preferably 4 to 12 carbon atoms.

Examples of the alicyclic hydrocarbon group in which a hydrogen atom is substituted with an aliphatic hydrocarbon group include a methylcyclohexyl group, a dimethylcyclohexyl group, a 2-methyladamantan-2-yl group, a 2-ethyladamantan-2-yl group, a 2-isopropyladamantan-2-yl group, a methylnorbornyl group, an isobornyl group and the like. In the alicyclic hydrocarbon group in which a hydrogen atom is substituted with an aliphatic hydrocarbon group, the total number of carbon atoms of the alicyclic hydrocarbon group and the aliphatic hydrocarbon group is preferably 20 or less.

The alkyl fluoride group represents an alkyl group having 1 to 12 carbon atoms which has a fluorine atom, and examples thereof include a fluoromethyl group, a difluoromethyl group, a trifluoromethyl group, a perfluorobutyl group and the like. The number of carbon atoms of the alkyl fluoride group is preferably 1 to 9, more preferably 1 to 6, still more preferably 1 to 4.

Examples of the aromatic hydrocarbon group include aryl groups such as a phenyl group, a biphenyl group, a naphthyl group and a phenanthryl group. The aromatic hydrocarbon group may have a chain hydrocarbon group or an alicyclic hydrocarbon group, and examples thereof include aromatic hydrocarbon groups having a chain hydrocarbon group (a tolyl group, a xylyl group, a cumenyl group, a mesityl group, a p-ethylphenyl group, a p-tert-butylphenyl group, a 2,6-diethylphenyl group, a 2-methyl-6-ethylphenyl group, etc.) and aromatic hydrocarbon groups having an alicyclic hydrocarbon group (a p-cyclohexylphenyl group, a p-adamantylphenyl group, etc.) and the like.

When the aromatic hydrocarbon group has a chain hydrocarbon group or an alicyclic hydrocarbon group, a chain hydrocarbon group having 1 to 18 carbon atoms and an alicyclic hydrocarbon group having 3 to 18 carbon atoms are preferable.

Examples of the aromatic hydrocarbon group in which a hydrogen atom is substituted with an alkoxy group include a p-methoxyphenyl group and the like.

Examples of the chain hydrocarbon group in which a hydrogen atom is substituted with an aromatic hydrocarbon group include aralkyl groups such as a benzyl group, a phenethyl group, a phenylpropyl group, a trityl group, a naphthylmethyl group and a naphthylethyl group.

Examples of the alkoxy group include a methoxy group, an ethoxy group, a propoxy group, a butoxy group, a pentyloxy group, a hexyloxy group, a heptyloxy group, an octyloxy group, a decyloxy group and a dodecyloxy group.

Examples of the alkylcarbonyl group include an acetyl group, a propionyl group and a butyryl group.

Examples of the halogen atom include a fluorine atom, a chlorine atom, a bromine atom and an iodine atom.

Examples of the alkylcarbonyloxy group include a methylcarbonyloxy group, an ethylcarbonyloxy group, a propylcarbonyloxy group, an isopropylcarbonyloxy group, a butylcarbonyloxy group, a sec-butylcarbonyloxy group, a tert-butylcarbonyloxy group, a pentylcarbonyloxy group, a hexylcarbonyloxy group, an octylcarbonyloxy group and a 2-ethylhexylcarbonyloxy group.

The ring formed by bonding R^{b4} and R^{b5} each other, together with sulfur atoms to which R^{b4} and R^{b5} are bonded, may be a monocyclic, polycyclic, aromatic, nonaromatic, saturated or unsaturated ring. This ring includes a ring having 3 to 18 carbon atoms and is preferably a ring having 4 to 18 carbon atoms. The ring having a sulfur atom includes a 3-membered to 12-membered ring and is preferably a 3-membered to 7-membered ring and includes, for example, the following rings and the like. * represents a bonding site.

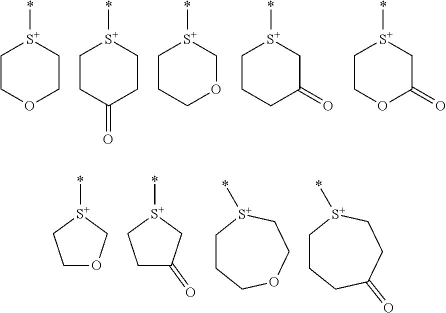

The ring formed by combining R^{b9} and R^{b10} together may be a monocyclic, polycyclic, aromatic, nonaromatic, saturated or unsaturated ring. This ring includes a 3-membered to 12-membered ring and is preferably a 3-membered to 7-membered ring. The ring includes, for example, a thiolan-1-ium ring (tetrahydrothiophenium ring), a thian-1-ium ring, a 1,4-oxathian-4-ium ring and the like.

The ring formed by combining R^{b11} and R^{b12} together may be a monocyclic, polycyclic, aromatic, nonaromatic, saturated or unsaturated ring. This ring includes a 3-membered to 12-membered ring and is preferably a 3-membered to 7-membered ring. Examples thereof include an oxacycloheptane ring, an oxocyclohexane ring, an oxonorbornane ring, an oxoadamantane ring and the like.

Of cation (b2-1) to cation (b2-5), a cation (b2-1) is preferable.

Examples of the cation (b2-1) include the following cations.

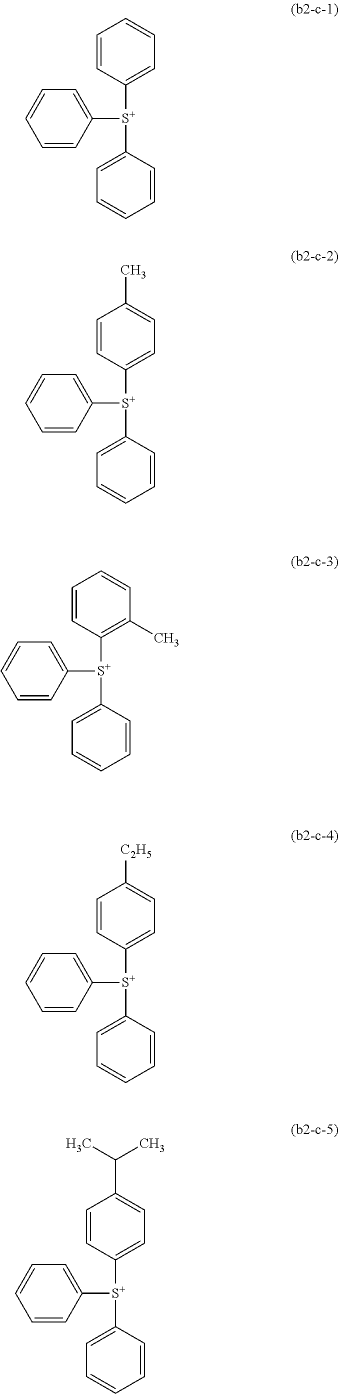 [SMILES: c1ccc([S+](c2ccccc2)c2ccccc2)cc1 ; Cc1ccc([S+](c2ccccc2)c2ccccc2)cc1 ; Cc1ccccc1[S+](c1ccccc1)c1ccccc1 ; CCc1ccc([S+](c2ccccc2)c2ccccc2)cc1 ; CC(C)c1ccc([S+](c2ccccc2)c2ccccc2)cc1]

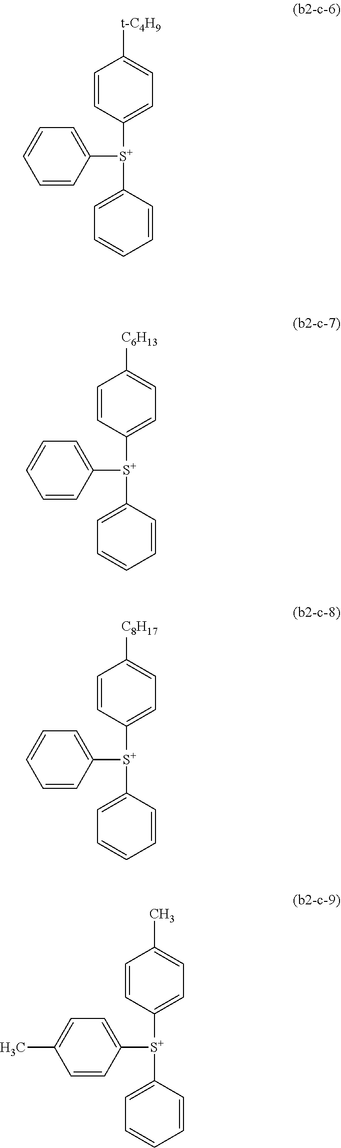

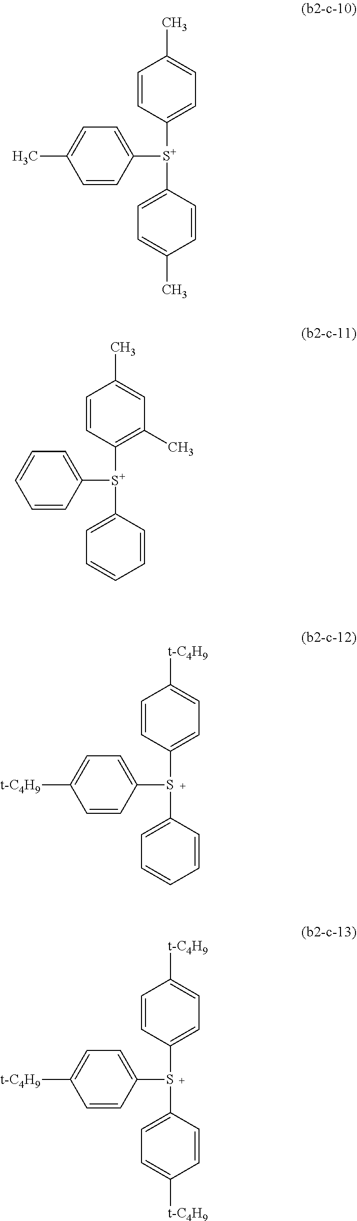

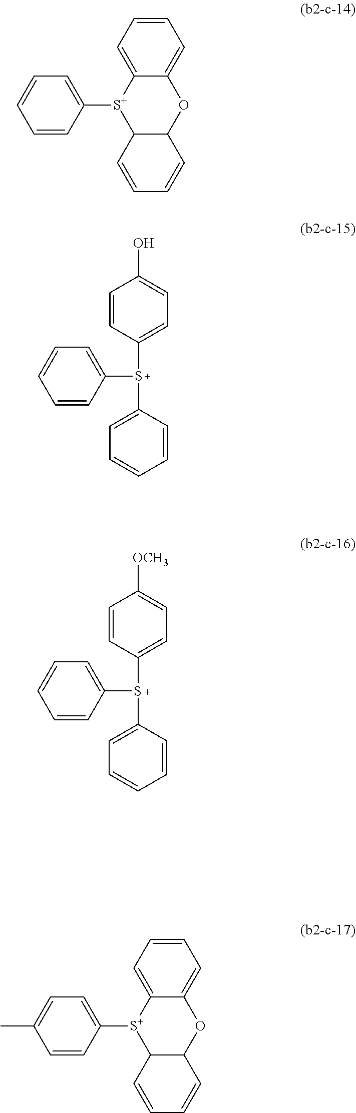

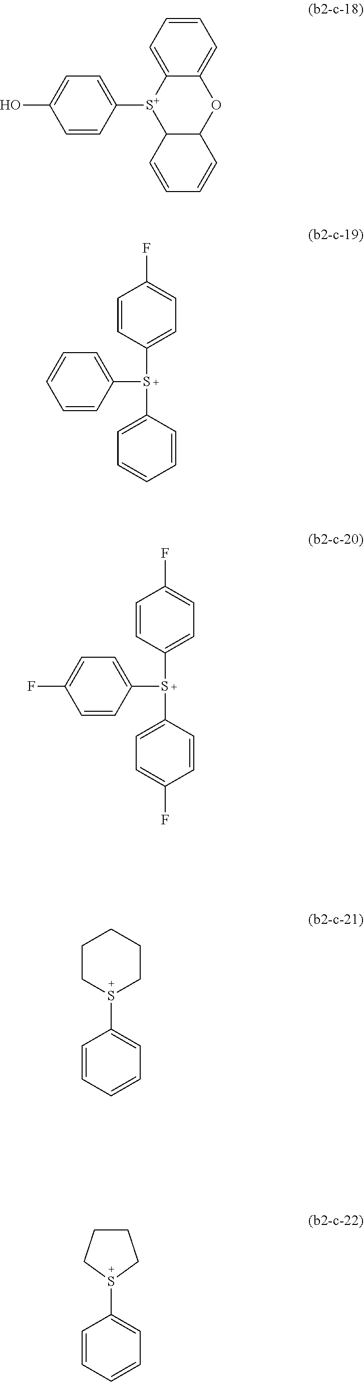

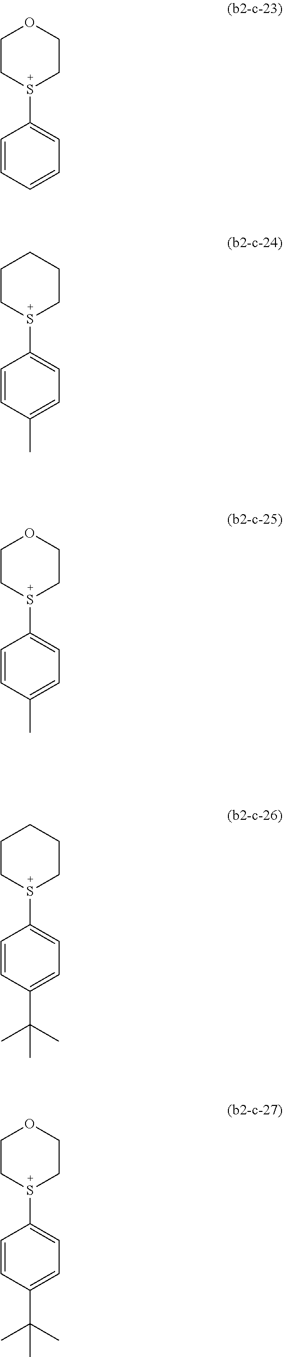

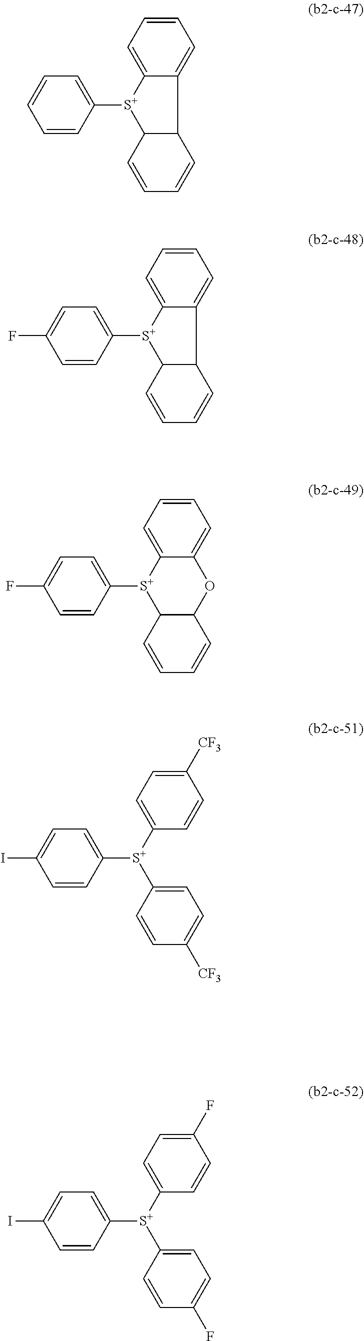 [SMILES: C1=CC2c3ccccc3[S+](c3ccccc3)C2C=C1 ; Fc1ccc([S+]2c3ccccc3C3C=CC=CC32)cc1 ; Fc1ccc([S+]2c3ccccc3OC3C=CC=CC32)cc1 ; FC(F)(F)c1ccc([S+](c2ccc(I)cc2)c2ccc(C(F)(F)F)cc2)cc1 ; Fc1ccc([S+](c2ccc(F)cc2)c2ccc(I)cc2)cc1]

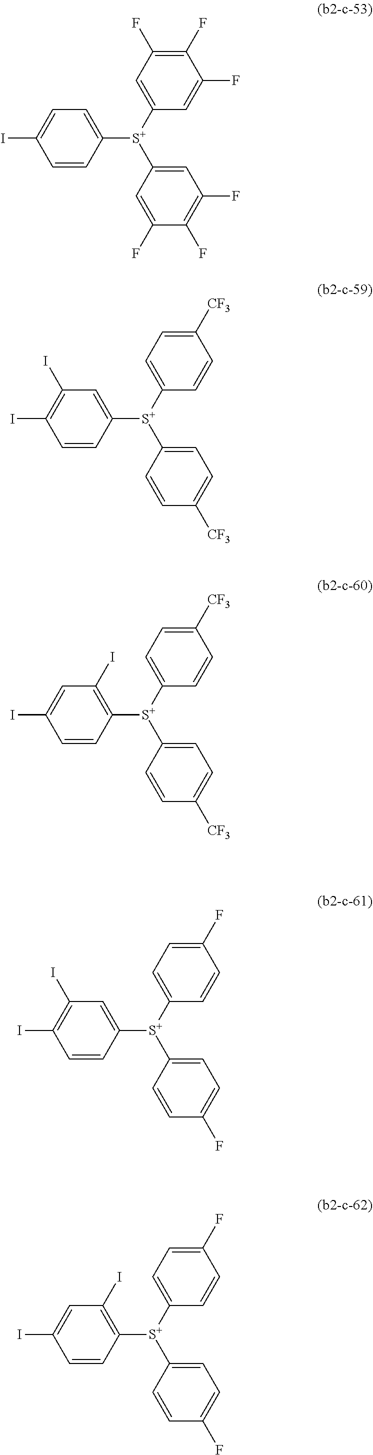 [SMILES: Fc1cc([S+](c2ccc(I)cc2)c2cc(F)c(F)c(F)c2)cc(F)c1F ; FC(F)(F)c1ccc([S+](c2ccc(C(F)(F)F)cc2)c2ccc(I)c(I)c2)cc1 ; FC(F)(F)c1ccc([S+](c2ccc(C(F)(F)F)cc2)c2ccc(I)cc2I)cc1 ; Fc1ccc([S+](c2ccc(F)cc2)c2ccc(I)c(I)c2)cc1 ; Fc1ccc([S+](c2ccc(F)cc2)c2ccc(I)cc2I)cc1]

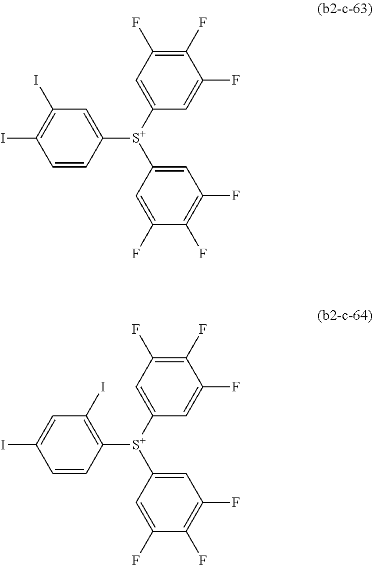 [SMILES: Fc1cc([S+](c2cc(F)c(F)c(F)c2)c2ccc(I)c(I)c2)cc(F)c1F ; Fc1cc([S+](c2cc(F)c(F)c(F)c2)c2ccc(I)cc2I)cc(F)c1F]

Examples of the cation (b2-2) include the following cations.

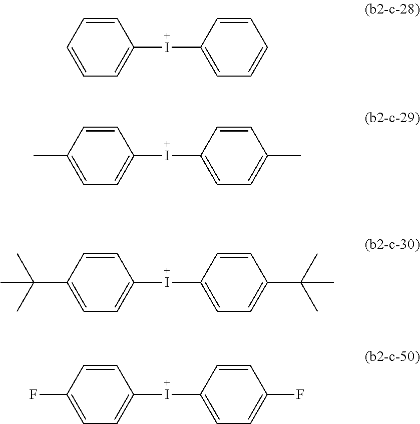

Examples of the cation (b2-3) include the following cations.

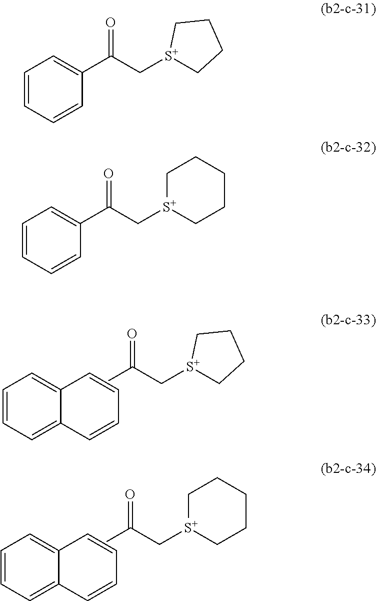

Examples of the cation (b2-4) include the following cations.

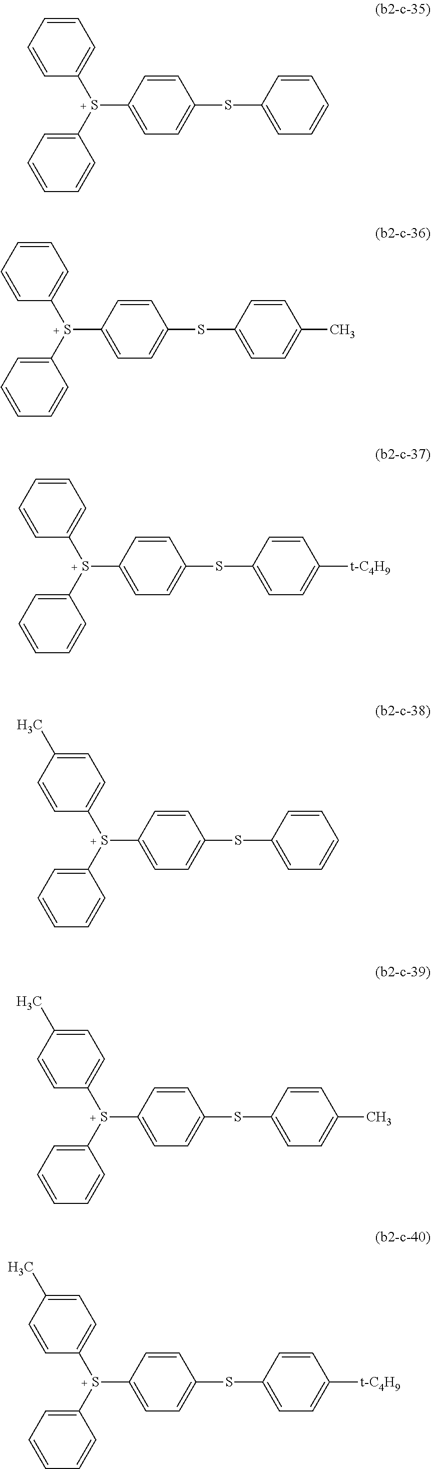

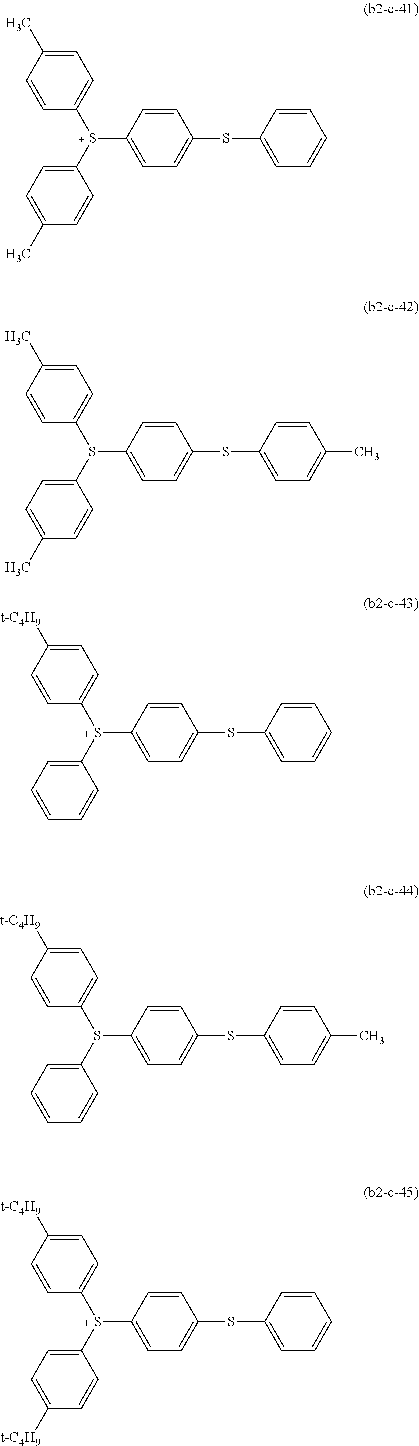

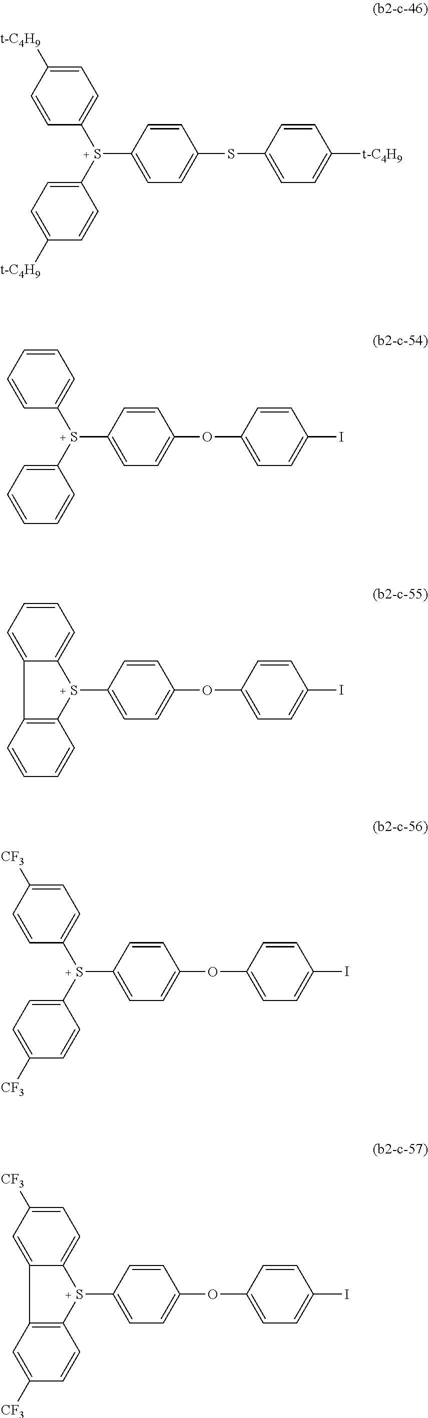

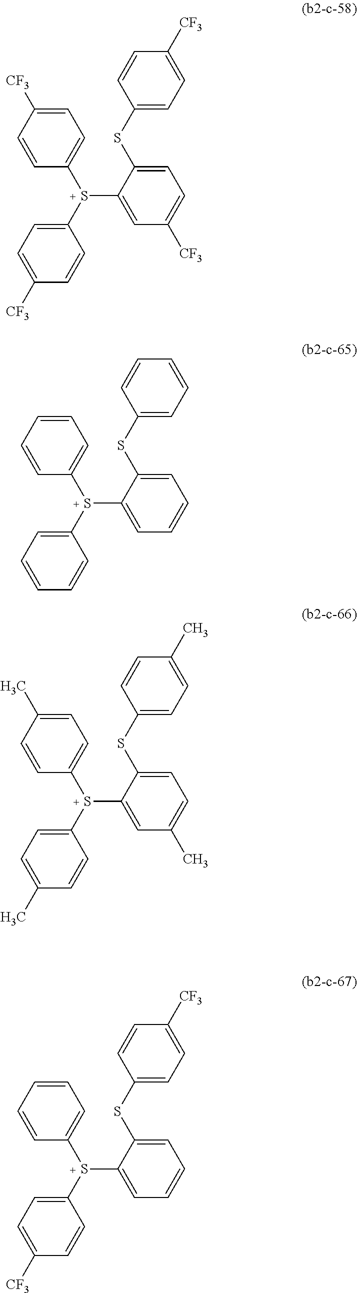

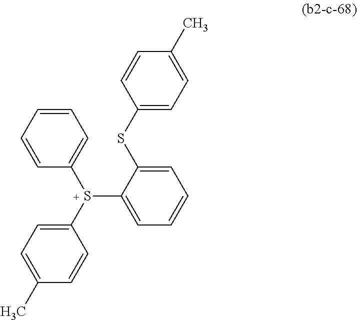

Examples of the cation (b2-5) include the following cations.

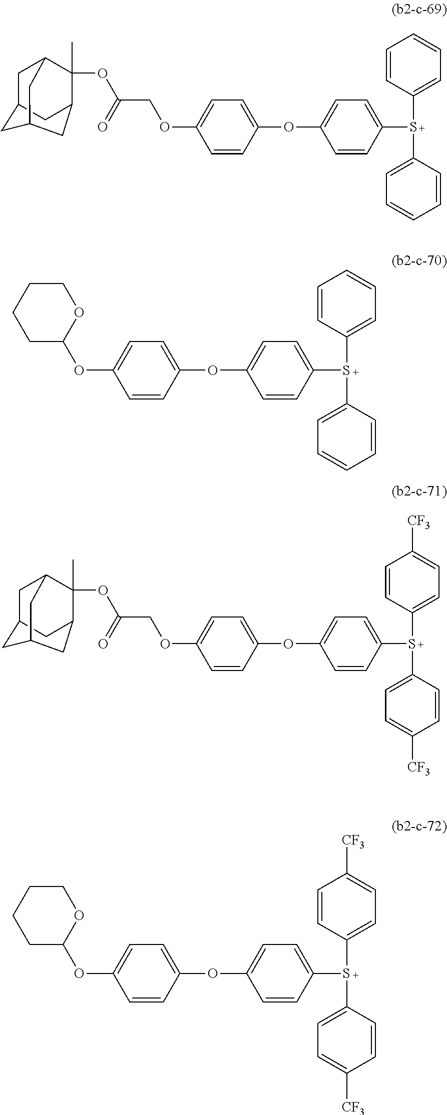

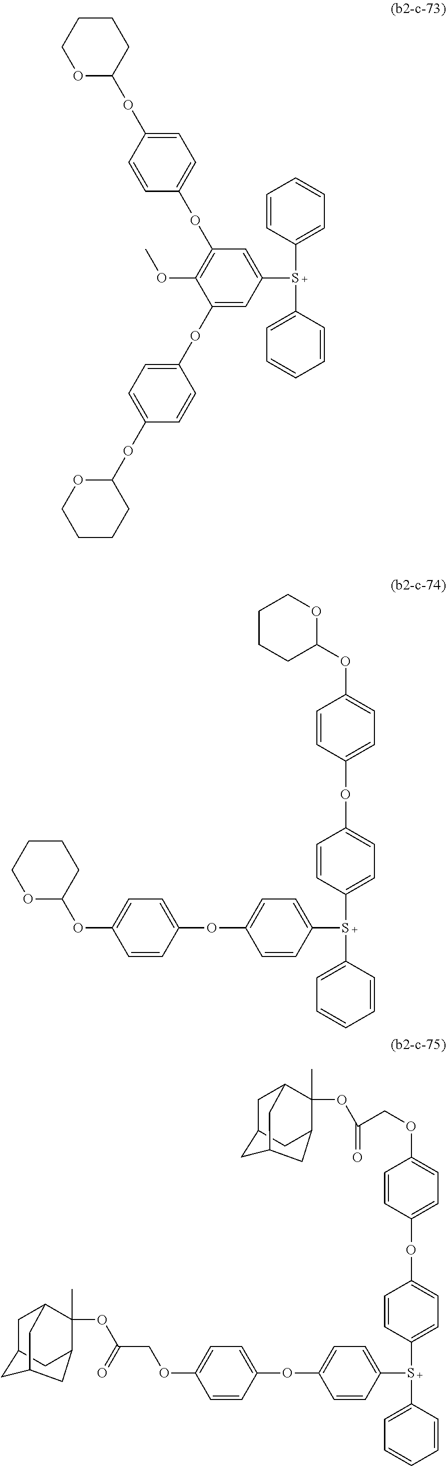

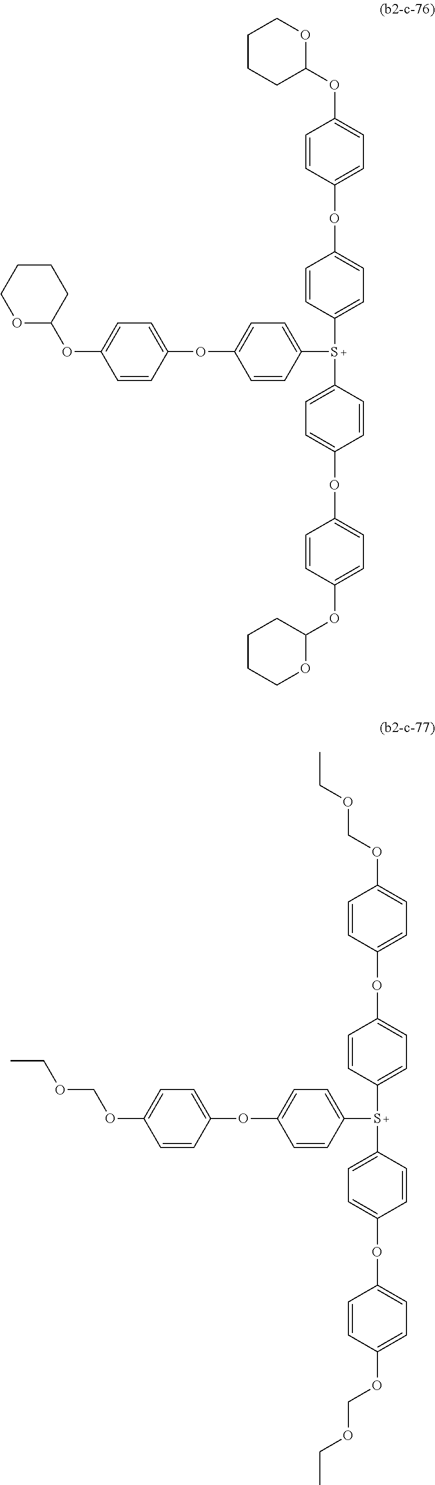

Specific examples of the salt (I) include salts obtained by optionally combining the above-mentioned cations and anions. Specific examples of the salt (I) are shown in the following table.

In the following table, the respective symbols represent symbols imparted to structures showing the above-mentioned anions and cations, and “to” represents that the salt (I) and the anion (I) correspond, respectively. For example, the salt (I-1) indicates a salt composed of an anion represented by formula (Ia-1) and a cation represented by formula (b2-c-1), the salt (I-2) indicates a salt composed of an anion represented by formula (Ia-2) and a cation represented by formula (b2-c-1), and the salt (I-31) indicates a salt composed of an anion represented by formula (Ia-1) and a cation represented by formula (b2-c-1)

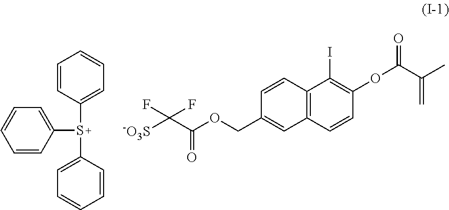

TABLE 1

| Salt (I) | Anion (I) | Cation (I) |
|---|---|---|
| (I-1) to (I-30) | (Ia-1) to (Ia-30) | (b2-c-1) |
| (I-31) to (I-60) | (Ia-1) to (Ia-30) | (b2-c-10) |
| (I-61) to (I-90) | (Ia-1) to (Ia-30) | (b2-c-13) |
| (I-91) to (I-120) | (Ia-1) to (Ia-30) | (b2-c-14) |
| (I-121) to (I-150) | (Ia-1) to (Ia-30) | (b2-c-18) |
| (I-151) to (I-180) | (Ia-1) to (Ia-30) | (b2-c-19) |
| (I-181) to (I-210) | (Ia-1) to (Ia-30) | (b2-c-20) |
| (I-211) to (I-240) | (Ia-1) to (Ia-30) | (b2-c-27) |
| (I-241) to (I-270) | (Ia-1) to (Ia-30) | (b2-c-30) |
| (I-271) to (I-300) | (Ia-1) to (Ia-30) | (b2-c-31) |
| (I-301) to (I-330) | (Ia-1) to (Ia-30) | (b2-c-50) |
| (I-331) to (I-360) | (Ia-1) to (Ia-30) | (b2-c-51) |
| (I-361) to (I-390) | (Ia-1) to (Ia-30) | (b2-c-54) |
| (I-391) to (I-420) | (Ia-1) to (Ia-30) | (b2-c-55) |
| (I-421) to (I-450) | (Ia-1) to (Ia-30) | (b2-c-56) |
| (I-451) to (I-480) | (Ia-1) to (Ia-30) | (b2-c-57) |
| (I-481) to (I-510) | (Ia-1) to (Ia-30) | (b2-c-58) |
| (I-511) to (I-540) | (Ia-1) to (Ia-30) | (b2-c-59) |
| (I-541) to (I-570) | (Ia-1) to (Ia-30) | (b2-c-60) |
| (I-571) to (I-600) | (Ia-1) to (Ia-30) | (b2-c-61) |
| (I-601) to (I-630) | (Ia-1) to (Ia-30) | (b2-c-62) |
| (I-631) to (I-660) | (Ia-1) to (Ia-30) | (b2-c-63) |
| (I-661) to (I-690) | (Ia-1) to (Ia-30) | (b2-c-64) |
| (I-691) to (I-720) | (Ia-1) to (Ia-30) | (b2-c-65) |
| (I-721) to (I-750) | (Ia-1) to (Ia-30) | (b2-c-66) |
| (I-751) to (I-780) | (Ia-1) to (Ia-30) | (b2-c-67) |
| (I-781) to (I-810) | (Ia-1) to (Ia-30) | (b2-c-68) |
| (I-811) to (I-840) | (Ia-1) to (Ia-30) | (b2-c-69) |
| (I-841) to (I-870) | (Ia-1) to (Ia-30) | (b2-c-70) |
| (I-871) to (I-900) | (Ia-1) to (Ia-30) | (b2-c-71) |
| (I-901) to (I-930) | (Ia-1) to (Ia-30) | (b2-c-72) |
| (I-931) to (I-960) | (Ia-1) to (Ia-30) | (b2-c-73) |
| (I-961) to (I-990) | (Ia-1) to (Ia-30) | (b2-c-74) |
| (I-991) to (I-1020) | (Ia-1) to (Ia-30) | (b2-c-75) |
| (I-1021) to (I-1050) | (Ia-1) to (Ia-30) | (b2-c-76) |
| (I-1051) to (I-1080) | (Ia-1) to (Ia-30) | (b2-c-77) |
| (I-1081) to (I-1085) | (Ia-31) to (Ia-35) | (b2-c-1) |
| (I-1086) to (I-1090) | (Ia-31) to (Ia-35) | (b2-c-10) |
| (I-1091) to (I-1095) | (Ia-31) to (Ia-35) | (b2-c-13) |
| (I-1096) to (I-1100) | (Ia-31) to (Ia-35) | (b2-c-14) |
| (I-1101) to (I-1105) | (Ia-31) to (Ia-35) | (b2-c-18) |
| (I-1106) to (I-1110) | (Ia-31) to (Ia-35) | (b2-c-19) |
| (I-1111) to (I-1115) | (Ia-31) to (Ia-35) | (b2-c-20) |
| (I-1116) to (I-1120) | (Ia-31) to (Ia-35) | (b2-c-27) |
| (I-1121) to (I-1125) | (Ia-31) to (Ia-35) | (b2-c-30) |
| (I-1126) to (I-1130) | (Ia-31) to (Ia-35) | (b2-c-31) |
| (I-1131) to (I-1135) | (Ia-31) to (Ia-35) | (b2-c-50) |
| (I-1136) to (I-1140) | (Ia-31) to (Ia-35) | (b2-c-51) |
| (I-1141) to (I-1145) | (Ia-31) to (Ia-35) | (b2-c-54) |
| (I-1146) to (I-1150) | (Ia-31) to (Ia-35) | (b2-c-55) |
| (I-1151) to (I-1155) | (Ia-31) to (Ia-35) | (b2-c-56) |
| (I-1156) to (I-1160) | (Ia-31) to (Ia-35) | (b2-c-57) |
| (I-1161) to (I-1165) | (Ia-31) to (Ia-35) | (b2-c-58) |
| (I-1166) to (I-1170) | (Ia-31) to (Ia-35) | (b2-c-59) |
| (I-1171) to (I-1175) | (Ia-31) to (Ia-35) | (b2-c-60) |
| (I-1176) to (I-1180) | (Ia-31) to (Ia-35) | (b2-c-61) |
| (I-1181) to (I-1185) | (Ia-31) to (Ia-35) | (b2-c-62) |
| (I-1186) to (I-1190) | (Ia-31) to (Ia-35) | (b2-c-63) |
| (I-1191) to (I-1195) | (Ia-31) to (Ia-35) | (b2-c-64) |
| (I-1196) to (I-1200) | (Ia-31) to (Ia-35) | (b2-c-65) |
| (I-1201) to (I-1205) | (Ia-31) to (Ia-35) | (b2-c-66) |
| (I-1206) to (I-1210) | (Ia-31) to (Ia-35) | (b2-c-67) |
| (I-1211) to (I-1215) | (Ia-31) to (Ia-35) | (b2-c-68) |
| (I-1216) to (I-1220) | (Ia-31) to (Ia-35) | (b2-c-69) |
| (I-1221) to (I-1225) | (Ia-31) to (Ia-35) | (b2-c-70) |
| (I-1226) to (I-1230) | (Ia-31) to (Ia-35) | (b2-c-71) |
| (I-1231) to (I-1235) | (Ia-31) to (Ia-35) | (b2-c-72) |
| (I-1236) to (I-1240) | (Ia-31) to (Ia-35) | (b2-c-73) |
| (I-1241) to (I-1245) | (Ia-31) to (Ia-35) | (b2-c-74) |
| (I-1246) to (I-1250) | (Ia-31) to (Ia-35) | (b2-c-75) |
| (I-1251) to (I-1255) | (Ia-31) to (Ia-35) | (b2-c-76) |
| (I-1256) to (I-1260) | (Ia-31) to (Ia-35) | (b2-c-77) |
| (I-1261) to (I-1265) | (Ia-36) to (Ia-40) | (b2-c-1) |
| (I-1266) to (I-1270) | (Ia-36) to (Ia-40) | (b2-c-10) |
| (I-1271) to (I-1275) | (Ia-36) to (Ia-40) | (b2-c-13) |
| (I-1276) to (I-1280) | (Ia-36) to (Ia-40) | (b2-c-14) |
| (I-1281) to (I-1285) | (Ia-36) to (Ia-40) | (b2-c-18) |
| (I-1286) to (I-1290) | (Ia-36) to (Ia-40) | (b2-c-19) |
| (I-1291) to (I-1295) | (Ia-36) to (Ia-40) | (b2-c-20) |
| (I-1296) to (I-1300) | (Ia-36) to (Ia-40) | (b2-c-27) |
| (I-1301) to (I-1305) | (Ia-36) to (Ia-40) | (b2-c-30) |
| (I-1306) to (I-1310) | (Ia-36) to (Ia-40) | (b2-c-31) |
| (I-1311) to (I-1315) | (Ia-36) to (Ia-40) | (b2-c-50) |
| (I-1316) to (I-1320) | (Ia-36) to (Ia-40) | (b2-c-51) |
| (I-1321) to (I-1325) | (Ia-36) to (Ia-40) | (b2-c-54) |
| (I-1326) to (I-1330) | (Ia-36) to (Ia-40) | (b2-c-55) |
| (I-1331) to (I-1335) | (Ia-36) to (Ia-40) | (b2-c-56) |
| (I-1336) to (I-1340) | (Ia-36) to (Ia-40) | (b2-c-57) |
| (I-1341) to (I-1345) | (Ia-36) to (Ia-40) | (b2-c-58) |
| (I-1346) to (I-1350) | (Ia-36) to (Ia-40) | (b2-c-59) |
| (I-1351) to (I-1355) | (Ia-36) to (Ia-40) | (b2-c-60) |
| (I-1356) to (I-1360) | (Ia-36) to (Ia-40) | (b2-c-61) |
| (I-1361) to (I-1365) | (Ia-36) to (Ia-40) | (b2-c-62) |
| (I-1366) to (I-1370) | (Ia-36) to (Ia-40) | (b2-c-63) |
| (I-1371) to (I-1375) | (Ia-36) to (Ia-40) | (b2-c-64) |
| (I-1376) to (I-1380) | (Ia-36) to (Ia-40) | (b2-c-65) |
| (I-1381) to (I-1385) | (Ia-36) to (Ia-40) | (b2-c-66) |
| (I-1386) to (I-1390) | (Ia-36) to (Ia-40) | (b2-c-67) |
| (I-1391) to (I-1395) | (Ia-36) to (Ia-40) | (b2-c-68) |
| (I-1396) to (I-1400) | (Ia-36) to (Ia-40) | (b2-c-69) |
| (I-1401) to (I-1405) | (Ia-36) to (Ia-40) | (b2-c-70) |
| (I-1406) to (I-1410) | (Ia-36) to (Ia-40) | (b2-c-71) |
| (I-1411) to (I-1415) | (Ia-36) to (Ia-40) | (b2-c-72) |
| (I-1416) to (I-1420) | (Ia-36) to (Ia-40) | (b2-c-73) |
| (I-1421) to (I-1425) | (Ia-36) to (Ia-40) | (b2-c-74) |
| (I-1426) to (I-1430) | (Ia-36) to (Ia-40) | (b2-c-75) |
| (I-1431) to (I-1435) | (Ia-36) to (Ia-40) | (b2-c-76) |
| (I-1436) to (I-1440) | (Ia-36) to (Ia-40) | (b2-c-77) |

Of these, salt (I) is preferably a salt obtained by combining an anion represented by any one of formula (Ia-1) to formula (Ia-6), formula (Ia-li) to formula (Ia-16), formula (Ia-20) to formula (Ia-24) and formula (Ia-31) to formula (Ia-35) with a cation represented by any one of formula (b2-c-1), formula (b2-c-10), formula (b2-c-13), formula (b2-c-14), formula (b2-c-18) to formula (b2-c-20), formula (b2-c-27), formula (b2-c-30), formula (b2-c-31), formula (b2-c-50), formula (b2-c-51) and formula (b2-c-54) to formula (b2-c-77).

### <Method for Producing Salt (I)>

The salt (I) can be produced by reacting a salt represented by formula (I-a) with a compound represented by formula (I-b) in the presence of a catalyst in a solvent:

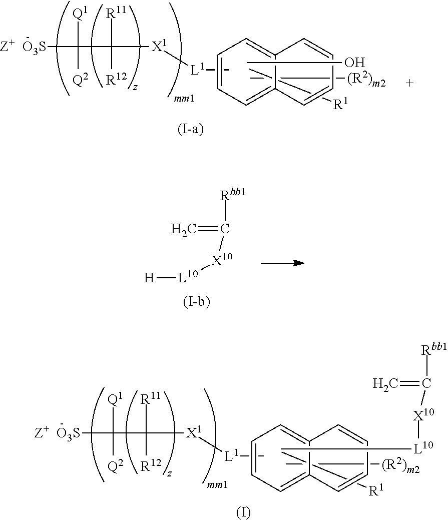

wherein all symbols are the same as defined above.

Examples of the catalyst include carbonyldiimidazole, potassium hydroxide and the like.

Examples of the solvent include chloroform, monochlorobenzene, dimethylformamide, acetonitrile, ethyl acetate, water and the like.

The reaction temperature is usually 15° C. to 80° C., and the reaction time is usually 0.5 to 24 hours.

Examples of the compound represented by formula (I-b) include compounds represented by the following formulas and the like and can be easily produced by a known production method.

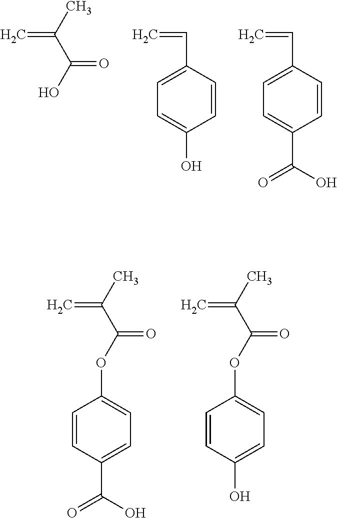

It is possible to produce a salt in which mm1 is 1 and X⁰ is *—CO—O— (* represents a bonding site to C(R¹¹) (R¹²) or C(Q¹) (Q²)) in a salt (I-a) (salt represented by formula (I1-a)), for example, by reacting a salt represented by formula (I1-c) with carbonyldiimidazole in a solvent, followed by a reaction with a compound represented by formula (I1-d):

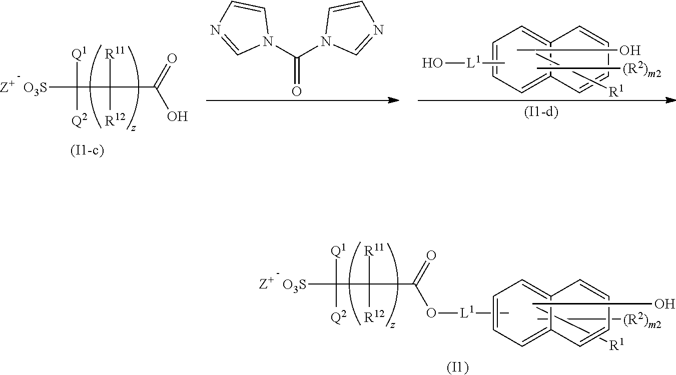

wherein all symbols are the same as defined above, respectively.

Examples of the solvent in this reaction include chloroform, acetonitrile and the like.

The reaction temperature is usually 5° C. to 80° C., and reaction time is usually 0.5 hour to 24 hours.

Examples of the salt represented by formula (I1-c) include salts represented by the following formulas and the like and can be produced by the method mentioned in JP 2008-127367 A.

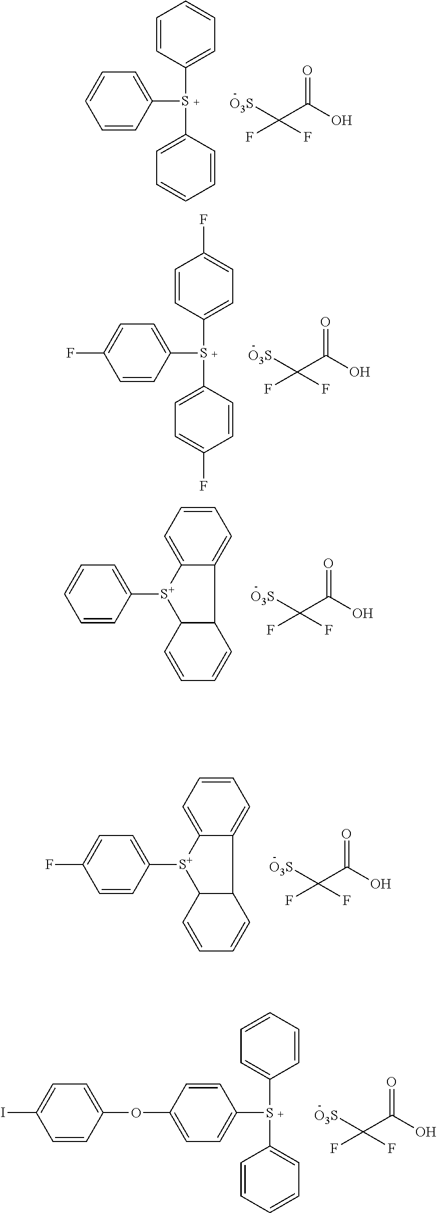

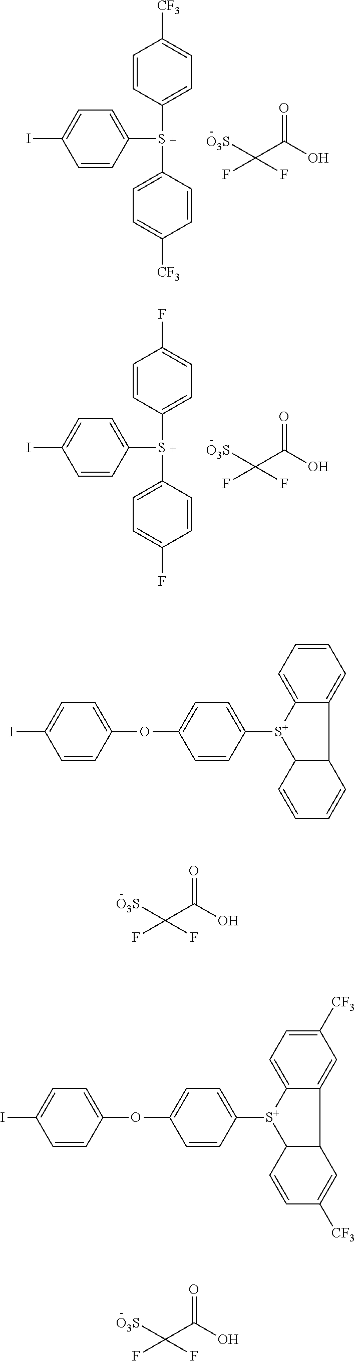

Examples of the compound represented by formula (I1-d) include compounds represented by the following formulas and the like and are easily available on the market, and can also be easily produced by a known production method.

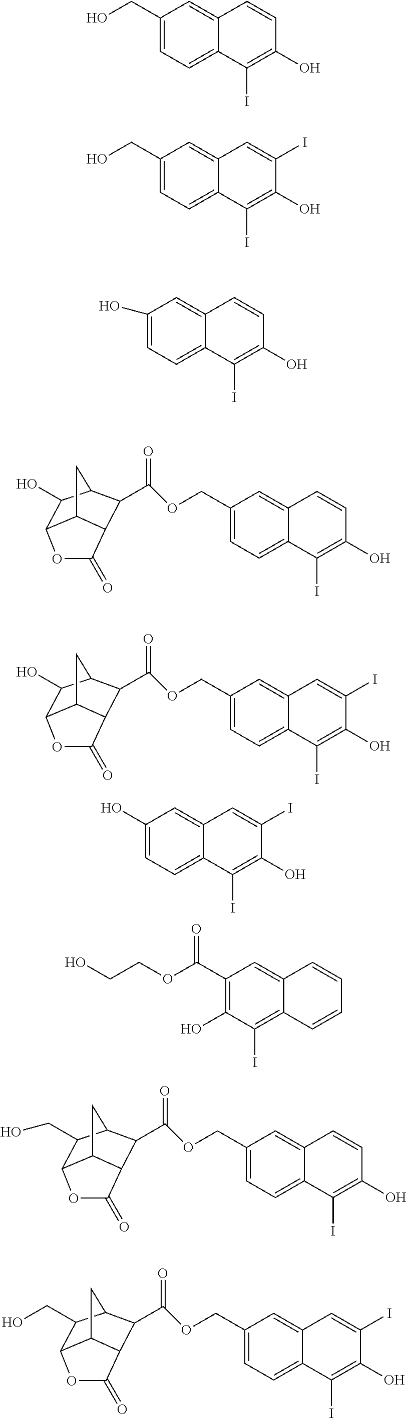

It is possible to produce a salt in which mm1 is 1 and X⁰ is *—O—CO—O— in a salt (I-a) (salt represented by formula (I2-a)), for example, by reacting a salt represented by formula (I2-c) with carbonyldiimidazole in a solvent, followed by a reaction with a compound represented by formula (I1-d).

It is also possible to produce a salt represented by formula (I2-a), for example, by reacting a compound represented by formula (I1-d) with carbonyldiimidazole in a solvent, followed by a reaction with a salt represented by formula (I2-c):

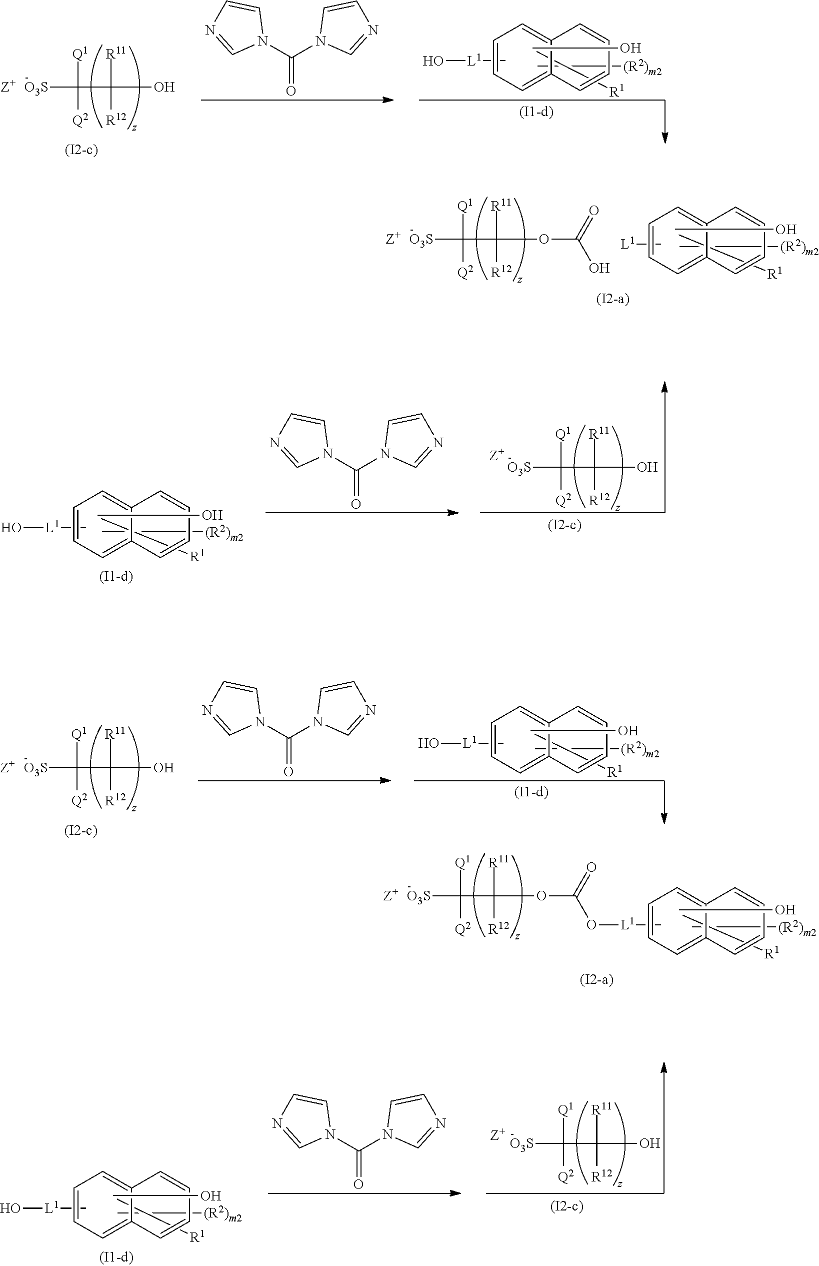

wherein all symbols are the same as defined above, respectively.

Examples of the solvent in this reaction include chloroform, acetonitrile and the like.

The reaction temperature is usually 5° C. to 80° C., and the reaction time is usually 0.5 hour to 24 hours.

Examples of the salt represented by formula (I2-c) include salts represented by the following formulas and the like and can be produced by the method mentioned in JP 2012-193170 A.

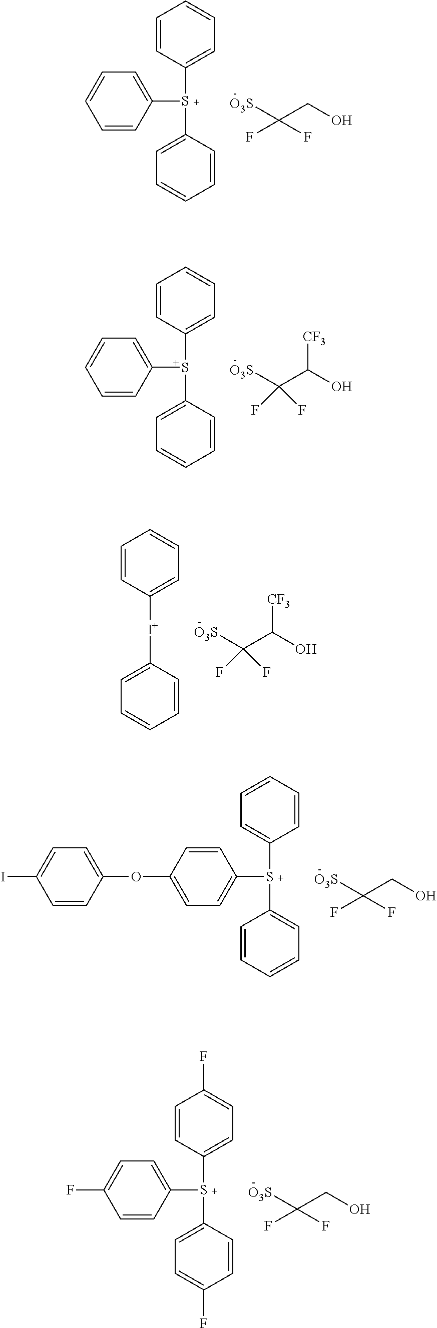

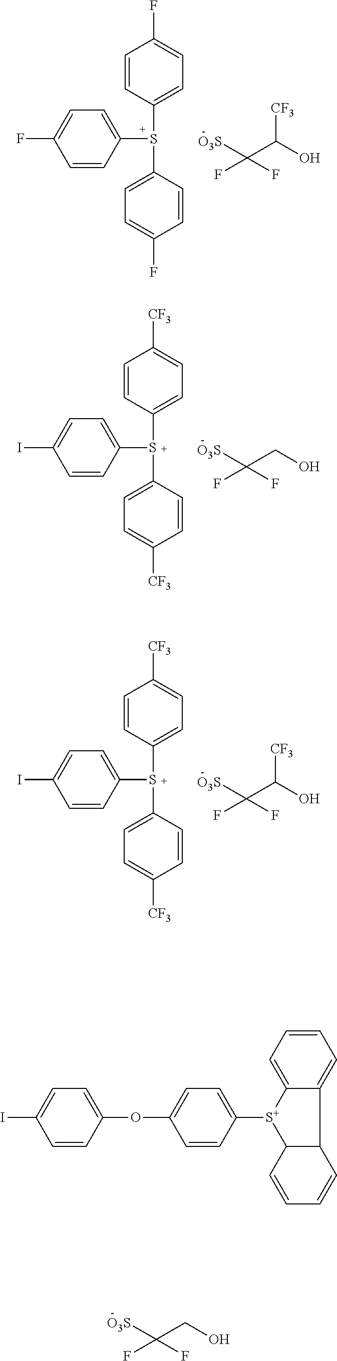

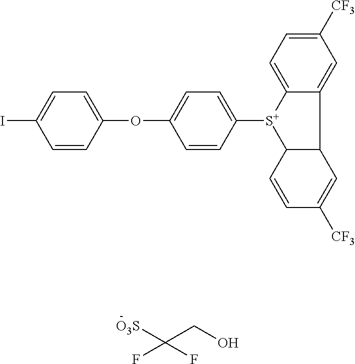

It is possible to produce a salt in which mm1 is 1 and X⁰ is *—O—CO— in a salt (I-a) (salt represented by formula (I3-a)), for example, by reacting a compound represented by formula (I3-d) with carbonyldiimidazole in a solvent, followed by reaction with a salt represented by formula (I2-c) and further a treatment with an acid as necessary:

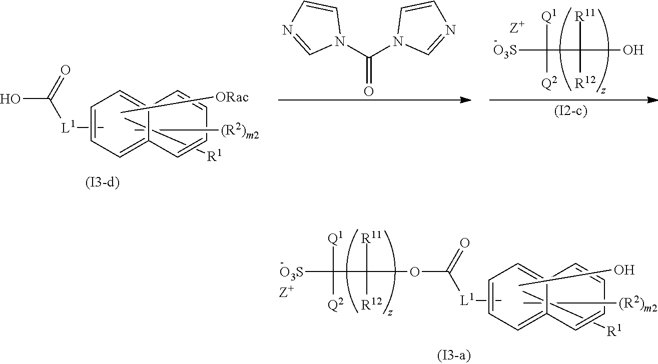

wherein all symbols are the same as defined above, respectively, and R^{ac} represents a hydrogen atom or an acid-labile group.

Examples of the solvent in this reaction include chloroform, acetonitrile and the like.

Examples of the salt include hydrochloric acid, sulfuric acid, p-toluenesulfonic acid, trifluoromethanesulfonic acid and the like.

The reaction temperature is usually 5° C. to 80° C., and the reaction time is usually 0.5 hour to 24 hours.

Examples of the compound represented by formula (I3-d) include salts represented by the following formulas and the like and are easily available on the market, and can also be easily produced by a known production method.

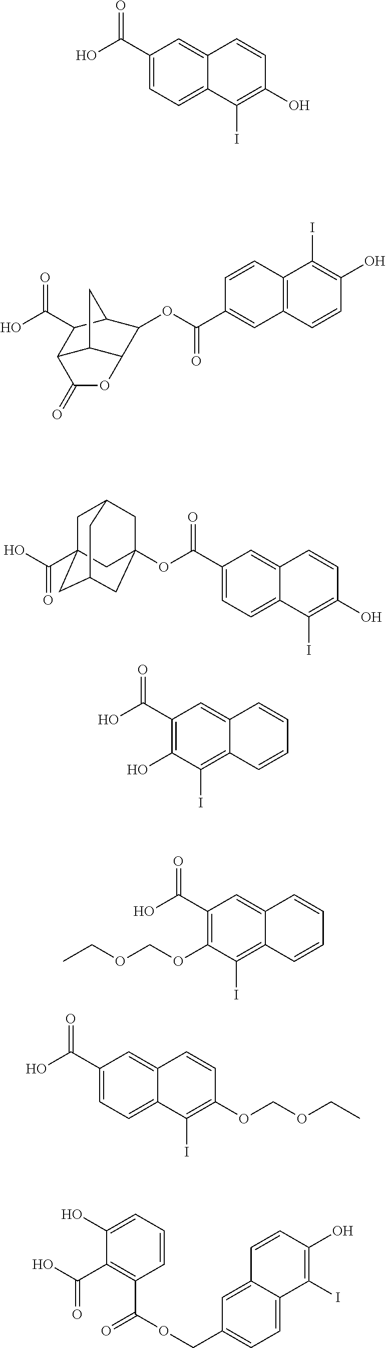

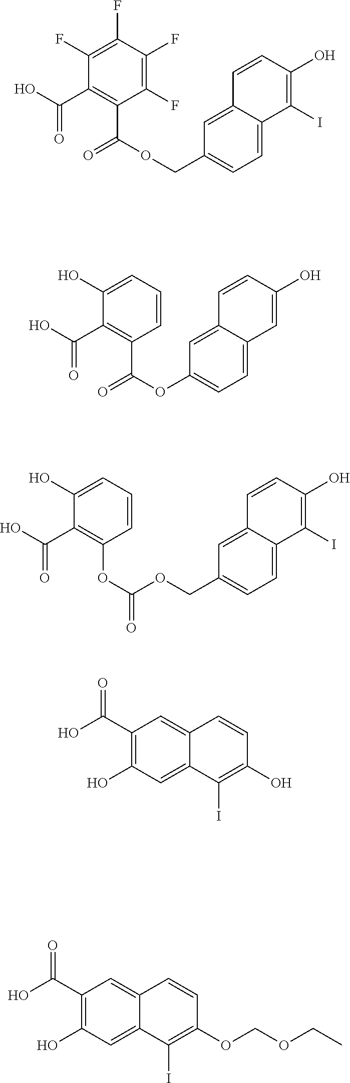 [SMILES: O=C(O)c1c(F)c(F)c(F)c(F)c1C(=O)OCc1ccc2c(I)c(O)ccc2c1 ; O=C(Oc1ccc2cc(O)ccc2c1)c1cccc(O)c1C(=O)O ; O=C(OCc1ccc2c(I)c(O)ccc2c1)Oc1cccc(O)c1C(=O)O ; O=C(O)c1cc2ccc(O)c(I)c2cc1O ; CCOCOc1ccc2cc(C(=O)O)c(O)cc2c1I]

It is possible to produce a salt in which mm1 is 1 and X⁰ is *—O— in a salt (I-a) (salt represented by formula (I4-a)), for example, by reacting a salt represented by formula (I2-c) with a compound represented by formula (I1-d) in the presence of a base in a solvent:

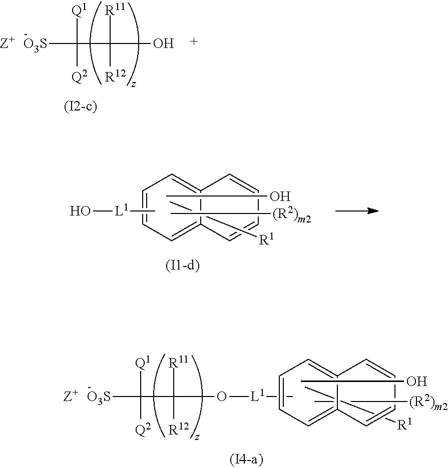

wherein all symbols are the same as defined above, respectively.

Examples of the base in this reaction include potassium hydroxide and the like.

Examples of the solvent in this reaction include acetonitrile and the like.

The reaction temperature is usually 5° C. to 80° C., and the reaction time is usually 0.5 hour to 24 hours.

It is possible to produce a salt in which L¹ is a group obtained by combining an alkanediyl group having 1 to 4 carbon atoms with an alicyclic hydrocarbon group having 3 to 18 carbon atoms in a salt (I-a) (in which —CH₂— included in the alkanediyl group may be replaced by —O— or —CO—, and —CH₂— included in the alicyclic hydrocarbon group may be replaced by —O—, —S—, —SO₂— or —CO—), for example, by replacing a salt represented by formula (I1-c) or a salt represented by formula (I2-c) by salts represented by the following formulas when synthesizing salts represented by formula (I1-a) to formula (I4-a).

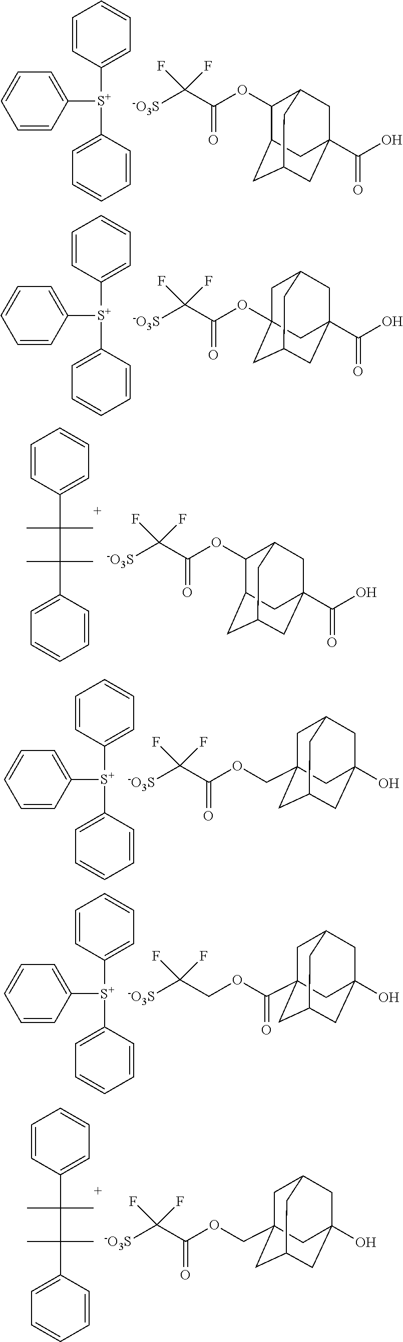

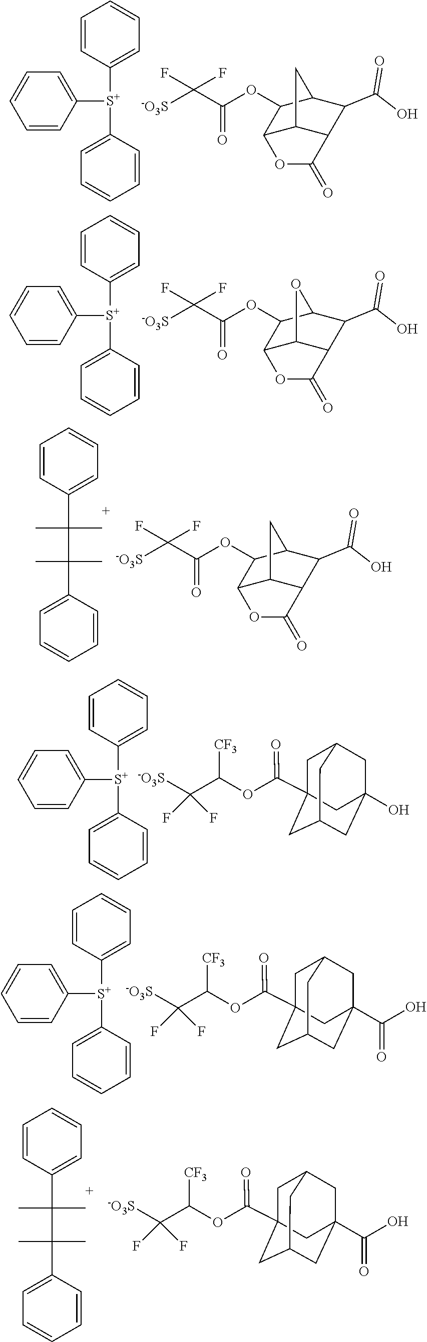

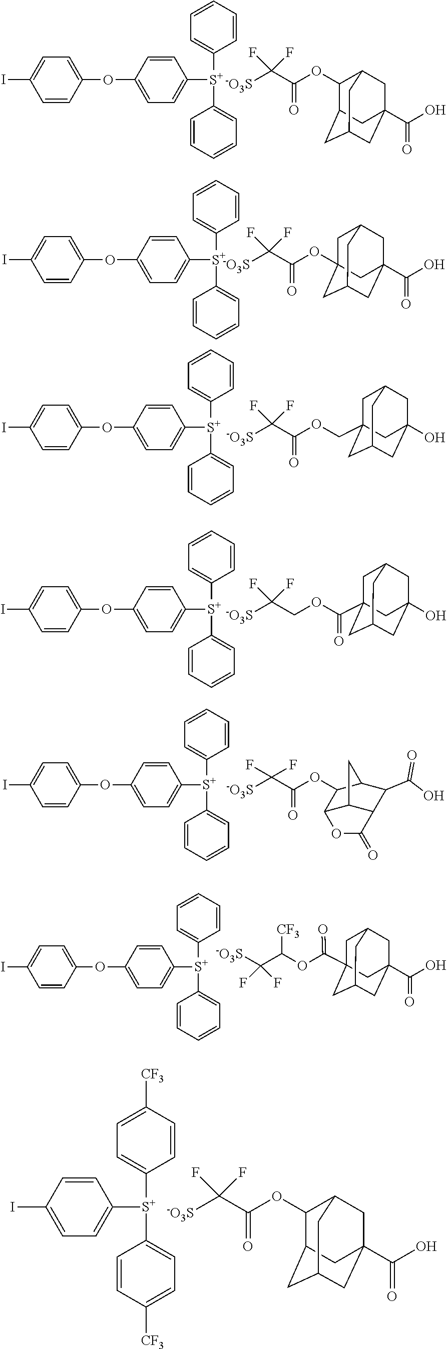

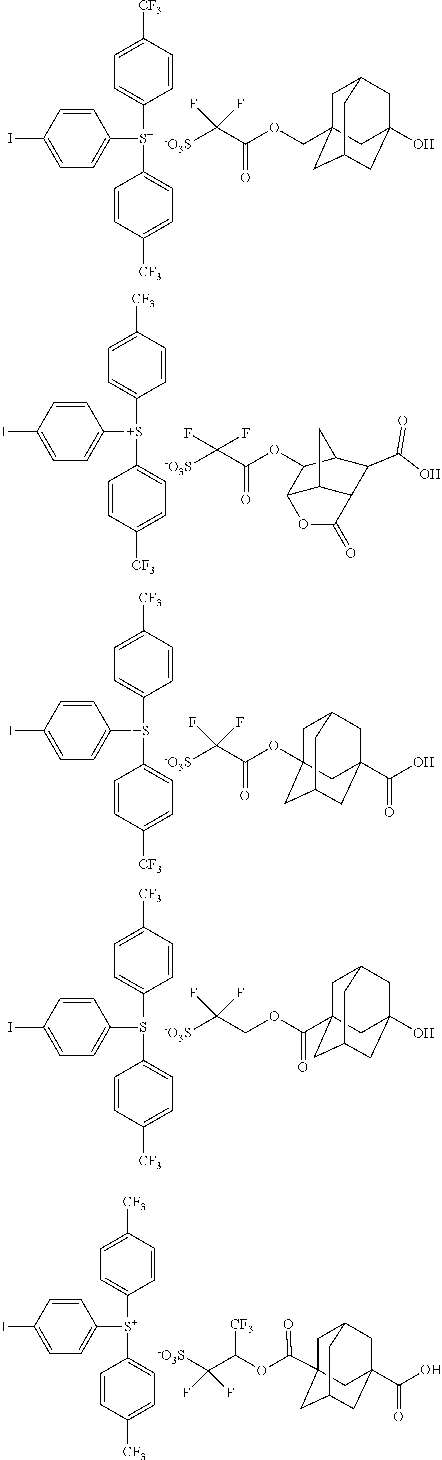

It is possible to produce a salt in which mm1 is 0 in a salt (I-a) (salt represented by formula (I5-a)), for example, by reacting a salt represented by formula (I5-c) with a compound represented by formula (I5-d) in the presence of a catalyst in a solvent, followed by a treatment with an acid as necessary:

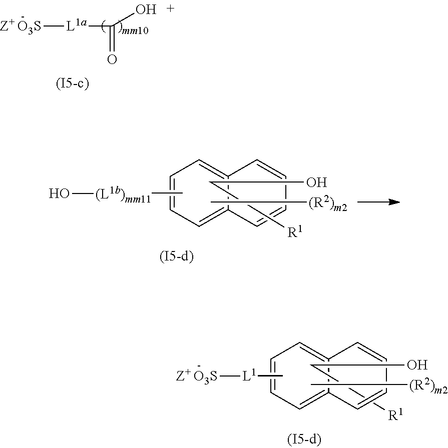

wherein L^{1a} represents a group obtained by removing —(CO)ₘₘ₁₀—O— (L^{1b})ₘₘ₁₁- from L¹, L^{1b} represents a single bond or a hydrocarbon group having 1 to 20 carbon atoms which may have a substituent (—CH₂— included in the hydrocarbon group may be replaced by —O—, —S—, —SO₂— or —CO—), all symbols other than L^{1a} and L^{1b} are the same as defined above, and mm10 and mm11 represent 0 or 1.

Examples of the solvent in this reaction include chloroform, acetonitrile and the like.

Examples of the catalyst include carbonyldiimidazole and the like.

The reaction temperature is usually 5° C. to 80° C., and reaction times is usually 0.5 hour to 24 hours.

Examples of the salt represented by formula (I5-c) include compounds represented by the following formulas and the like and are easily available on the market.

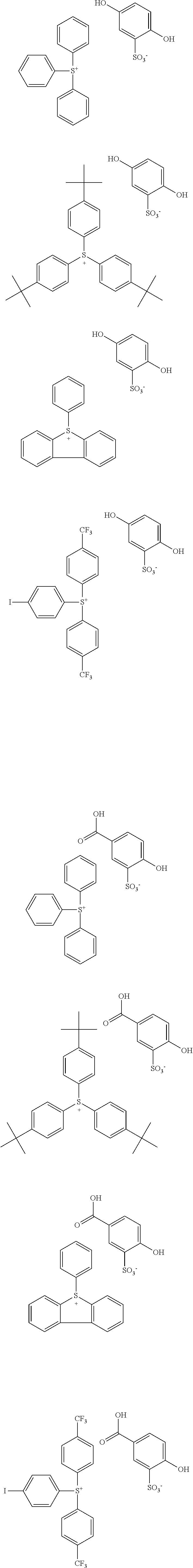

Examples of the compound represented by formula (I5-d) include salts represented by the following formulas and the like and are easily available on the market.

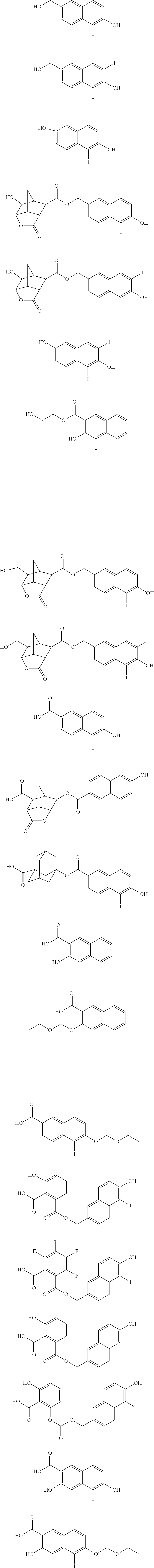

[Structural Unit Derived from Salt Represented by Formula (I)]

The structural unit derived from a salt represented by formula (I) indicates a state where a double bond of CH₂═C—R^{bb1} included in the salt (I) is cleaved, and examples thereof include a structural unit represented by the following formula (IP) (hereinafter sometimes referred to as “structural unit (IP)):

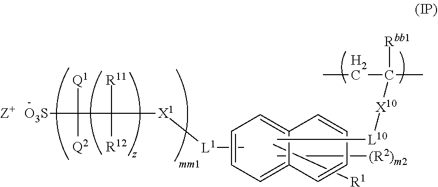

wherein all symbols are the same as defined above, respectively.

The structural unit (IP) functions as an acid generator similarly to the salt (I), and also functions as a structural unit constituting a compound or a resin.

[Resin Including Structural Unit (IP) Derived from Salt Represented by Formula (I)]

The resin of the present invention is a resin including a structural unit derived from a salt represented by formula (I) (hereinafter sometimes referred to as “resin (Ap)”).

The resin (Ap) may be either a homopolymer including one structural unit (IP), or a copolymer including two or more structural units (IP).

The resin (Ap) may include a structural unit other than the structural unit (IP). As mentioned below, examples of the structural unit other than the structural unit (IP) include a structural unit having an acid-labile group (hereinafter sometimes referred to as “structural unit (a1)”), a structural unit having no acid-labile group (hereinafter sometimes referred to as “structural unit (s)”), other structural units (hereinafter sometimes referred to as “structural unit (t)”) and structural units known in the relevant field. Here, “acid-labile group” means a group having a leaving group which is eliminated by contact with an acid, thus converting a constitutional unit into a constitutional unit having a hydrophilic group (e.g., a hydroxy group or a carboxy group).

The content of the structural unit (IP) is usually 0.1 to 100 mol %, preferably 0.5 to 50 mol %, more preferably 0.8 to 30 mol %, and still more preferably 1 to 10 mol %, based on the total amount of the resin (Ap).

Particularly, when used for the resist composition, as mentioned below, it is preferable that the resin (Ap) may further include, in addition to the structural unit (IP), a structural unit (a1).

When used for the resist composition, as mentioned below, the resin (Ap) may be used in combination with a resin including a structural unit (a1) (hereinafter sometimes referred to as “resin (A)”), whether or not the resin includes the structural unit (a1). Hereinafter the resin (Ap) and/or the resin (A) may be sometimes referred to as “resin (A) or the like”.

The resin (A) further includes a structural unit other than the structural unit (a1) similarly to the resin (Ap).

<Structural Unit (a1)>

The structural unit (a1) is derived from a monomer having an acid-labile group (hereinafter sometimes referred to as “monomer (a1)”).

The acid-labile group contained in the resin (A) or the like is preferably a group represented by formula (1) (hereinafter also referred to as group (1)) and/or a group represented by formula (2) (hereinafter also referred to as group (2)):

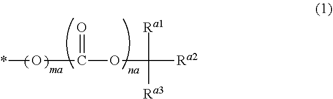

wherein, in formula (1), R^{a1}, R^{a2} and R^{a3} each independently represent an alkyl group having 1 to 8 carbon atoms, an alkenyl group having 2 to 8 carbon atoms, an alicyclic hydrocarbon group having 3 to 20 carbon atoms, an aromatic hydrocarbon group having 6 to 18 carbon atoms, or groups obtained by combining these groups, or R^{a1} and R^{a2} are bonded to each other to form an alicyclic hydrocarbon group having 3 to 20 carbon atoms together with carbon atoms to which R^{a1} and R^{a2} are bonded, and the alkyl group, the alkenyl group, the alicyclic hydrocarbon group and the aromatic hydrocarbon group may have a halogen atom,
ma and na each independently represent 0 or 1, and at least one of ma and na represents 1, and
* represents a bonding site:

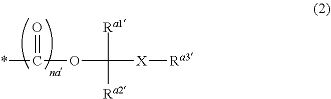

wherein, in formula (2), R^{a1}′ and R^{a2}′ each independently represent a hydrogen atom or a hydrocarbon group having 1 to 12 carbon atoms, R^{a3}′ represents a hydrocarbon group having 1 to 20 carbon atoms, or R^{a2}′ and R^{a3}′ are bonded to each other to form a heterocyclic group having 3 to 20 carbon atoms together with carbon atoms and X to which R^{a2}′ and R^{a3}′ are bonded, —CH₂— included in the hydrocarbon group and the heterocyclic group may be replaced by —O— or —S—, and the hydrocarbon group may have a halogen atom,
X represents an oxygen atom or a sulfur atom,
na′ represents 0 or 1, and
* represents a bonding site.

Examples of the alkyl group in R^{a1}, R^{a2} and R^{a3} include a methyl group, an ethyl group, a propyl group, a butyl group, a pentyl group, a hexyl group, a heptyl group, an octyl group and the like.

Examples of the alkenyl group in R^{a1}, R^{a2} and R^{a3} include an ethenyl group, a propenyl group, an isopropenyl group, a butenyl group, an isobutenyl group, a tert-butenyl group, a pentenyl group, a hexenyl group, a heptenyl group, an octenyl group, an isooctenyl group and a nonenyl group.

The alicyclic hydrocarbon group in R^{a1}, R^{a2} and R^{a3} may be either monocyclic or polycyclic. Examples of the monocyclic alicyclic hydrocarbon group include cycloalkyl groups such as a cyclopentyl group, a cyclohexyl group, a cycloheptyl group and a cyclooctyl group. Examples of the polycyclic alicyclic hydrocarbon group include a decahydronaphthyl group, an adamantyl group, a norbornyl group and the following groups (* represents a bonding site). The number of carbon atoms of the alicyclic hydrocarbon group of R^{a1}, R^{a2} and R^{a3} is preferably 3 to 16.

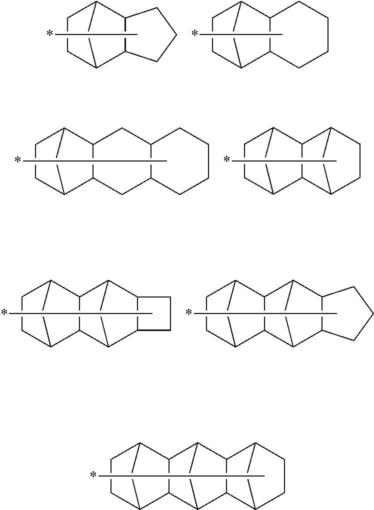

Examples of the aromatic hydrocarbon group in R^{a1}, R^{a2} and R^{a3} include aryl groups such as a phenyl group, a naphthyl group, an anthryl group, a biphenyl group and a phenanthryl group.

Examples of the combined group include groups obtained by combining the above-mentioned alkyl group and alicyclic hydrocarbon group (e.g., alkylcycloalkyl groups or cycloalkylalkyl groups, such as a methylcyclohexyl group, a dimethylcyclohexyl group, a methylnorbornyl group, a cyclohexylmethyl group, an adamantylmethyl group, an adamantyldimethyl group and a norbornylethyl group), aromatic hydrocarbon groups having an aralkyl group such as a benzyl group, or an alkyl group (a p-methylphenyl group, a p-tert-butylphenyl group, a tolyl group, a xylyl group, a cumenyl group, a mesityl group, a 2,6-diethylphenyl group, a 2-methyl-6-ethylphenyl group, etc.), aromatic hydrocarbon groups having an alicyclic hydrocarbon group (a p-cyclohexylphenyl group, a p-adamantylphenyl group, etc.), aryl-cycloalkyl groups such as a phenylcyclohexyl group, and the like.

Preferably, ma is 0 and na is 1.

When R^{a1} and R^{a2} are bonded to each other to form an alicyclic hydrocarbon group, examples of —C(R^{a1}) (R^{a2}) (R^{a3}) include the following groups. The alicyclic hydrocarbon group preferably has 3 to 12 carbon atoms. * represents a bond to —O—.

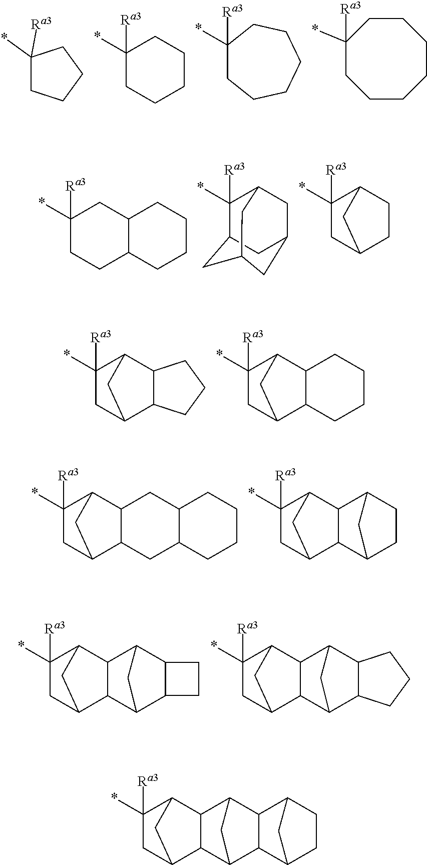

Examples of the hydrocarbon group in R^{a1}′, R^{a2}′ and R^{a3}′ include an alkyl group, an alicyclic hydrocarbon group, an aromatic hydrocarbon group, and groups formed by combining these groups.

Examples of the alkyl group, the alicyclic hydrocarbon group, the aromatic hydrocarbon group, and groups formed by combining these groups include those which are the same as mentioned in R^{a1}, R^{a2} and R^{a3}.

When R^{a2}′ and R^{a3}′ are bonded to each other to form a heterocyclic group together with carbon atoms and X to which R^{a2}′ and R^{a3}′ are bonded, examples of —C(R^{a1}′) (R^{a2}′)—X—R^{a3}′ include the following rings. * represents a bonding site.

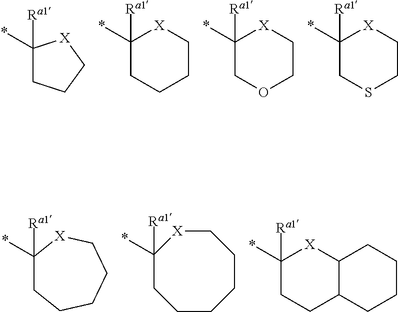

At least one of R^{a1}′ and R^{a2}′ is preferably a hydrogen atom.

na′ is preferably 0.

Examples of the halogen atom which may be possessed by R^{a1}, R^{a2}, R^{a3}, R^{a1}′, R^{a2}′ and R^{a3}′ include a fluorine atom, a chlorine atom, a bromine atom or an iodine atom.

Examples of the group (1) include the following groups.

A group wherein, in formula (1), R^{a1}, R^{a2} and R^{a3} are alkyl groups, ma=0 and na=1. The group is preferably a tert-butoxycarbonyl group.

A group wherein, in formula (1), R^{a1} and R^{a2} are bonded to each other to form an adamantyl group together with carbon atoms to which R^{a1} and R^{a2} are bonded, R^{a3} is an alkyl group, ma=0 and na=1.

A group wherein, in formula (1), R^{a1} and R^{a2} are each independently an alkyl group, R^{a3} is an adamantyl group, ma=0 and na=1.

Specific examples of the group (1) include the following groups. * represents a bond.

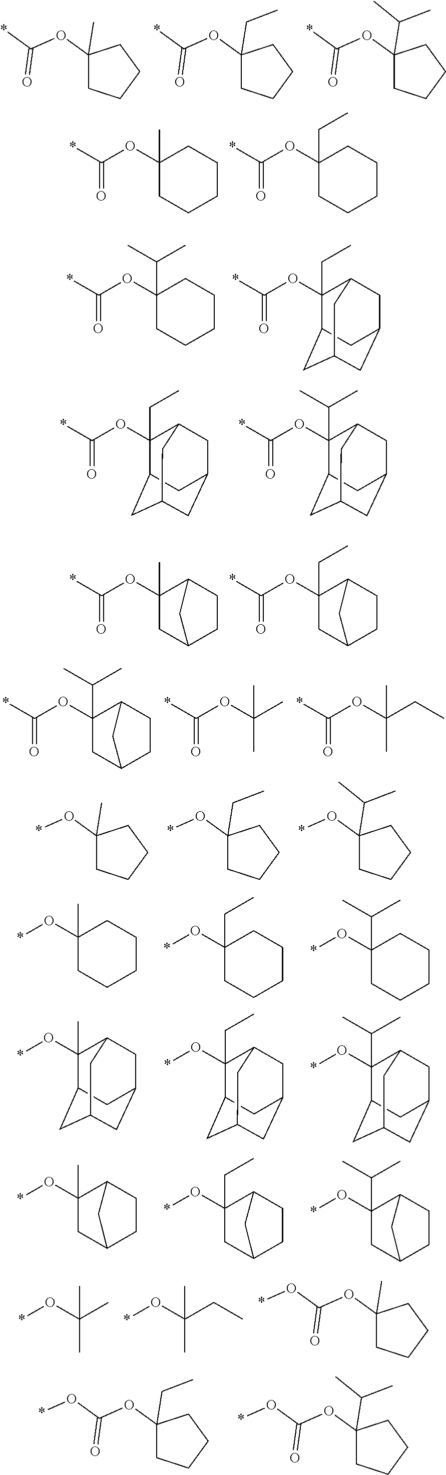

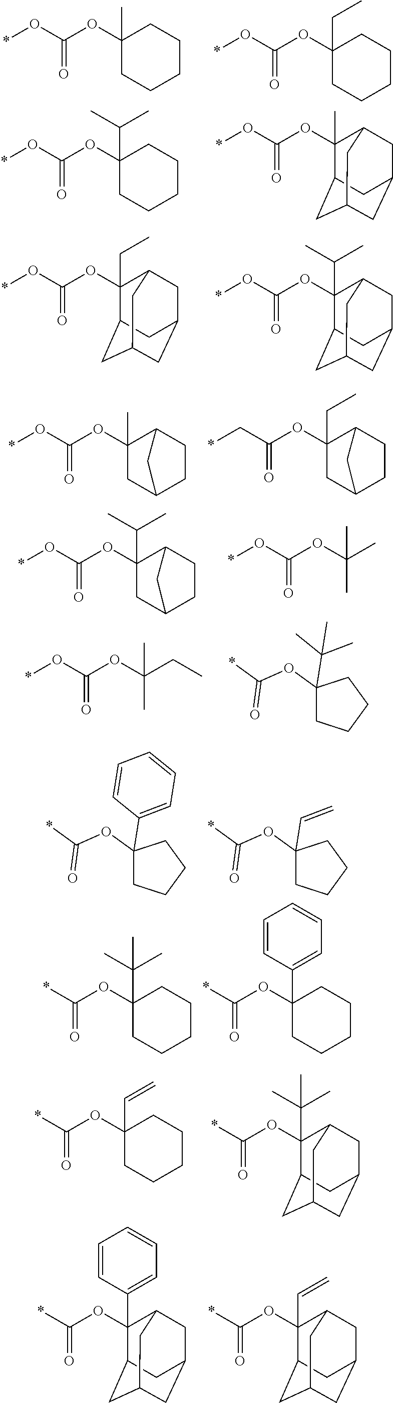

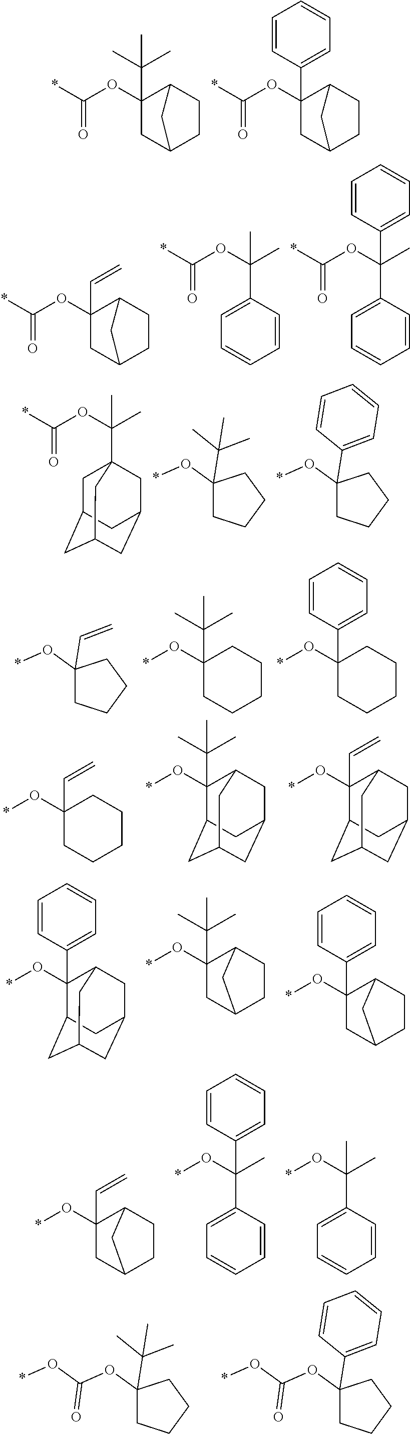

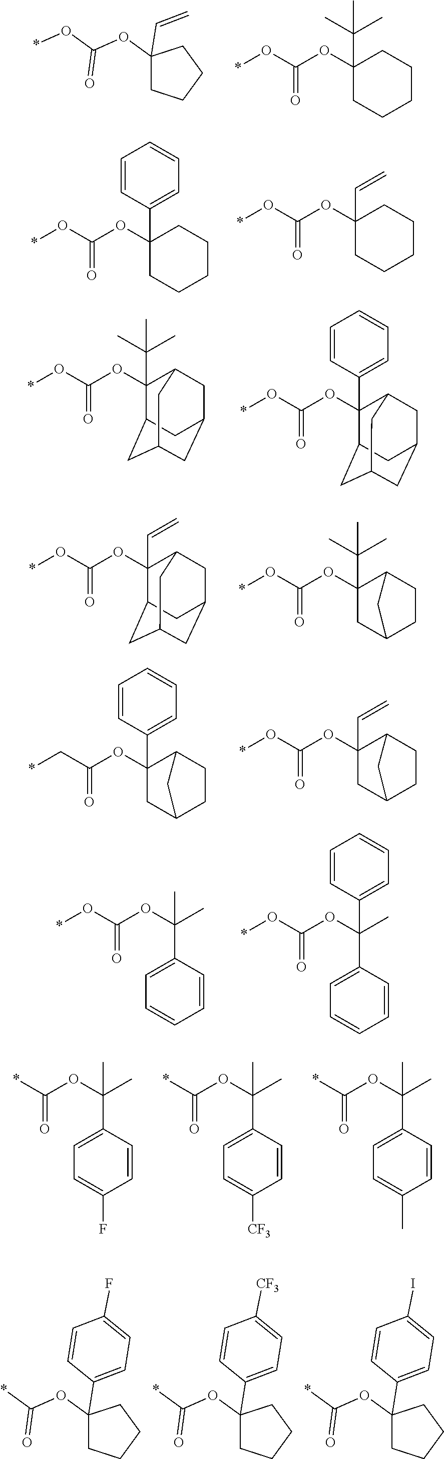

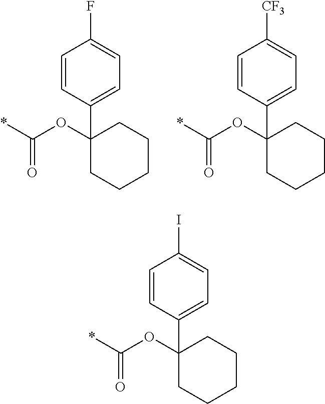

Specific examples of the group (2) include the following groups. * represents a bond.

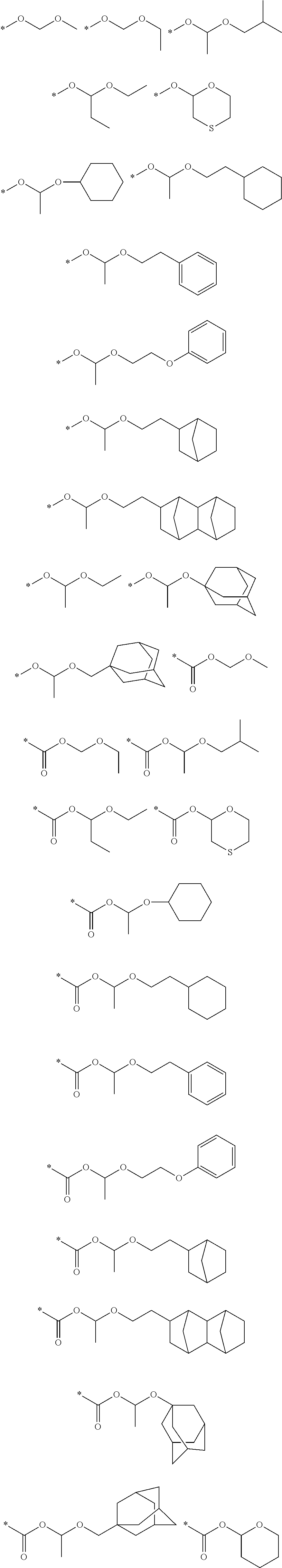

The monomer (a1) is preferably a monomer having an acid-labile group and an ethylenic unsaturated bond, and more preferably a (meth)acrylic monomer having an acid-labile group.

Of the (meth)acrylic monomers having an acid-labile group, those having an alicyclic hydrocarbon group having 5 to 20 carbon atoms are preferably exemplified. When using a resin (A) including a structural unit derived from a monomer (a1) having a bulky structure such as an alicyclic hydrocarbon group or the like in a resist composition, it is possible to improve the resolution of a resist pattern.

The structural unit derived from a (meth)acrylic monomer having a group (1) includes a structural unit represented by formula (a1-0) (hereinafter sometimes referred to as structural unit (a1-0)), a structural unit represented by formula (a1-1) (hereinafter sometimes referred to as structural unit (a1-1)) or a structural unit represented by formula (a1-2) (hereinafter sometimes referred to as structural unit (a1-2)). Preferably, the structural unit is at least one structural unit selected from the group consisting of structural unit (a1-0), structural unit (a1-1) and structural unit (a1-2), and more preferably at least one structural units selected from the group consisting of structural unit (a1-1) and structural unit (a1-2). These structural units may be used alone, or two or more structural units may be used in combination:

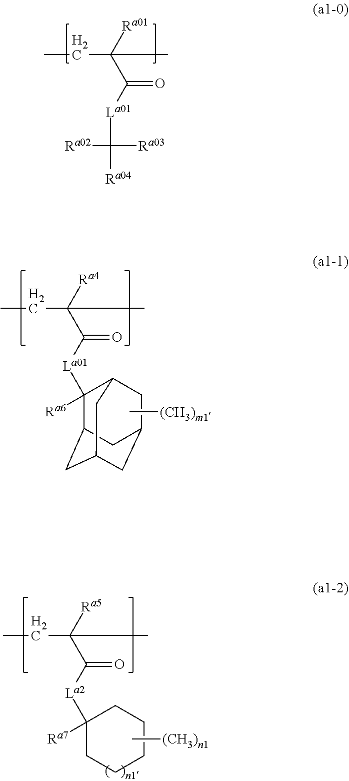

wherein, in formula (a1-0), formula (a1-1) and formula (a1-2),
L^{a01}, L^{a1} and L^{a2} each independently represent —O— or *—O—(CH₂)ₖ₁—CO—O—, k1 represents an integer of 1 to 7, and * represents a bonding site to —CO—,
R^{a01}, R^{a4} and R^{a5} each independently represent a hydrogen atom, a halogen atom or an alkyl group having 1 to 6 carbon atoms which may have a halogen atom,
R^{a02}, R^{a03} and R^{a04} each independently represent an alkyl group having 1 to 8 carbon atoms, an alicyclic hydrocarbon group having 3 to 18 carbon atoms, an aromatic hydrocarbon group having 6 to 18 carbon atoms, or groups obtained by combining these groups, and the alkyl group, the alicyclic hydrocarbon group and the aromatic hydrocarbon group may have a halogen atom,
R^{a6} and R^{a7} each independently represent an alkyl group having 1 to 8 carbon atoms, an alkenyl group having 2 to 8 carbon atoms, an alicyclic hydrocarbon group having 3 to 18 carbon atoms, an aromatic hydrocarbon group having 6 to 18 carbon atoms, or groups obtained by combining these groups, and the alkyl group, the alicyclic hydrocarbon group and the aromatic hydrocarbon group may have a halogen atom,
m1′ represents an integer of 0 to 14,
n1 represents an integer of 0 to 10, and
n1′ represents an integer of 0 to 3.

Examples of the halogen atom include a fluorine atom, a chlorine atom, a bromine atom or an iodine atom.

Examples of the alkyl group as for R^{a01}, R^{a4} and R^{a5} include a methyl group, an ethyl group, a propyl group, a butyl group, a pentyl group or a hexyl group.

R^{a01}, R^{a4} and R^{a5} are preferably a hydrogen atom or a methyl group, and more preferably a methyl group.

L^{a01}, L^{a1} and L^{a2} are preferably an oxygen atom or *—O—(CH₂)ₖ₀₁—CO—O— (in which k01 is preferably an integer of 1 to 4, and more preferably 1), and more preferably an oxygen atom.

Examples of the alkyl group, the alkenyl group, the alicyclic hydrocarbon group, the aromatic hydrocarbon group, and groups obtained by combining these groups in R^{a02}, R^{a03}, R^{a04}, R^{a6} and R^{a7} include the same groups as mentioned as for R^{a1}, R^{a2} and R^{a3} of formula (1).

The alkyl group in R^{a02}, R^{a03} and R^{a04} is preferably an alkyl group having 1 to 6 carbon atoms, more preferably a methyl group or an ethyl group, and still more preferably a methyl group.

The alkyl group in R^{a6} and R^{a7} is preferably an alkyl group having 1 to 6 carbon atoms, more preferably a methyl group, an ethyl group, an isopropyl group or a t-butyl group, and still more preferably an ethyl group, an isopropyl group or a t-butyl group.

The alkenyl group in R^{a6} and R^{a7} is preferably an alkenyl group having 2 to 6 carbon atoms, and more preferably an ethenyl group, a propenyl group, an isopropenyl group or a butenyl group.

The number of carbon atoms of the alicyclic hydrocarbon group as for R^{a02}, R^{a03}, R^{a04}, R^{a6} and R^{a7} is preferably 5 to 12, and more preferably 5 to 10.

The number of carbon atoms of the aromatic hydrocarbon group of R^{a02}, R^{a03}, R^{a04}, R^{a6} and R^{a7} is preferably 6 to 12, and more preferably 6 to 10.

The total number of carbon atoms of the groups obtained by combining the alkyl group with the alicyclic hydrocarbon group is preferably 18 or less.

The total number of carbon atoms of the groups obtained by combining the alkyl group with the aromatic hydrocarbon group is preferably 18 or less.

R^{a02} and R^{a03} are preferably an alkyl group having 1 to 6 carbon atoms or an aromatic hydrocarbon group having 6 to 12 carbon atoms, and more preferably a methyl group, an ethyl group, a phenyl group or a naphthyl group.

R^{a04} is preferably an alkyl group having 1 to 6 carbon atoms or an alicyclic hydrocarbon group having 5 to 12 carbon atoms, and more preferably a methyl group, an ethyl group, a cyclohexyl group or an adamantyl group.

R^{a6} and R^{a7} are preferably an alkyl group having 1 to 6 carbon atoms, an alkenyl group having 2 to 6 carbon atoms or an aromatic hydrocarbon group having 6 to 12 carbon atoms, more preferably a methyl group, an ethyl group, an isopropyl group, a t-butyl group, an ethenyl group, a phenyl group or a naphthyl group, and still more preferably an ethyl group, an isopropyl group, a t-butyl group, an ethenyl group or a phenyl group.

m1′ is preferably an integer of 0 to 3, and more preferably 0 or 1.

n1 is preferably an integer of 0 to 3, and more preferably 0 or 1.

n1′ is preferably 0 or 1.

The structural unit (a1-0) includes, for example, a structural unit represented by any one of formula (a1-0-1) to formula (a1-0-21) and a structural unit in which a methyl group corresponding to R^{a01} in the structural unit (a1-0) is substituted with a hydrogen atom, a halogen atom, a haloalkyl group (alkyl group having a halogen atom) or other alkyl groups, and is preferably a structural unit represented by any one of formula (a1-0-1) to formula (a1-0-10), formula (a1-0-13), formula (a1-0-14) and formula (a1-0-19) to formula (a1-0-21).

[chemical structure]

The structural unit (a1-1) includes, for example, structural units derived from the monomers mentioned in JP 2010-204646 A. Of these structural units, a structural unit represented by any one of formula (a1-1-1) to formula (a1-1-7) and a structural unit in which a methyl group corresponding to R in the structural unit (a1-1) is substituted with a hydrogen atom, a halogen atom, a haloalkyl group or other alkyl groups are preferable, and a structural unit represented by any one of formula (a1-1-1) to formula (a1-1-4) is more preferable.

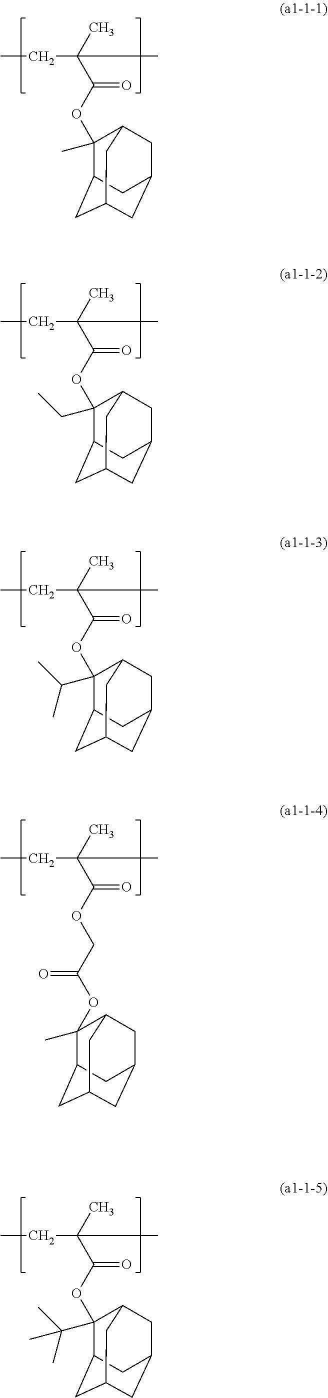

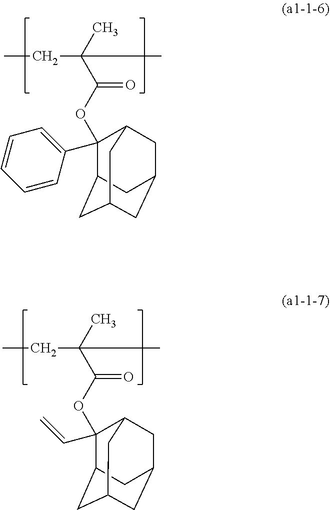

Examples of the structural unit (a1-2) include a structural unit represented by any one of formula (a1-2-1) to formula (a1-2-20), and a structural unit in which a methyl group corresponding to R^{a5} in the structural unit (a1-2) is substituted with a hydrogen atom, a halogen atom, a haloalkyl group or other alkyl groups, and a structure unit represented by any one of formula (a1-2-2), formula (a1-2-5), formula (a1-2-6) and formula (a1-2-10) to formula (a1-2-20) is preferable.

[chemical structure]

When the resin (A) or the like includes a structural unit (a1-0) and/or a structural unit (a1-1) and/or a structural unit (a1-2), the total content of them is 10 mol % or more, preferably 15 mol % or more, more preferably 20 mol % or more, still more preferably 25 mol % or more, yet more preferably 30 mol % or more, further preferably 40 mol % or more, and still further preferably 50 mol % or more, based on all structural units of the resin (A) or the like. The total content is also 95 mol % or less, preferably 90 mol % or less, more preferably 85 mol % or less, still more preferably 70 mol % or less, and yet more preferably 65 mol % or less. Specifically, the total content is 10 to 95 mol %, preferably 15 to 90 mol %, more preferably 20 to 85 mol %, still more preferably 25 to 70 mol %, and yet more preferably 30 to 70 mol %.

When the resin (A) or the like includes a structural unit (a1-0), the content is 5 mol % or more, preferably 10 mol % or more, more preferably 15 mol % or more, still more preferably 20 mol % or more, yet more preferably 25 mol % or more, further preferably 30 mol % or more, and still further preferably 35 mol % or more, based on all structural units of the resin (A) or the like. The content is also 80 mol % or less, preferably 75 mol % or less, more preferably 70 mol % or less, still more preferably 65 mol % or less, and yet more preferably 60 mol % or less. Specifically, the content is 5 to 80 mol %, preferably 5 to 75 mol %, and more preferably 10 to 70 mol %.

When the resin (A) or the like includes a structural unit (a1-1) and/or a structural unit (a1-2), the total content of them is 10 mol % or more, preferably 15 mol % or more, more preferably 20 mol % or more, still more preferably 25 mol % or more, yet more preferably 30 mol % or more, further preferably 40 mol % or more, and still further preferably 50 mol % or more, based on all structural units of the resin (A) or the like. The total content is also 95 mol % or less, preferably 90 mol % or less, more preferably 85 mol % or less, still more preferably 80 mol % or less, yet more preferably 75 mol % or less, further preferably 70 mol % or less, and still further preferably 65 mol % or less. Specifically, the total content is 10 to 95 mol %, preferably 15 to 85 mol %, more preferably 15 to 80 mol %, still more preferably 20 to 80 mol %, and yet more preferably 20 to 75 mol %.

In the structural unit (a1), examples of the structural unit having a group (2) include a structural unit represented by formula (a1-4) (hereinafter sometimes referred to as “structural unit (a1-4)”):

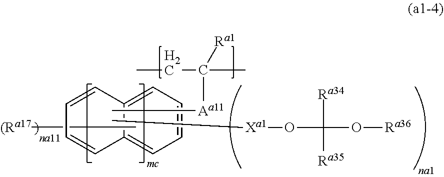

wherein, in formula (a1-4), all symbols are the same as defined above.

Examples of the halogen atom in R^{a1} and R^{a17} include a fluorine atom, a chlorine atom and a bromine atom.

Examples of the alkyl group having 1 to 6 carbon atoms which may have a halogen atom in R^{a1} and R^{a17} include a trifluoromethyl group, a difluoromethyl group, a methyl group, a perfluoroethyl group, a 2,2,2-trifluoroethyl group, a 1,1,2,2-tetrafluoroethyl group, an ethyl group, a perfluoropropyl group, a 2,2,3,3,3-pentafluoropropyl group, a propyl group, a perfluorobutyl group, a 1,1,2,2,3,3,4,4-octafluorobutyl group, a butyl group, a perfluoropentyl group, a 2,2,3,3,4,4,5,5,5-nonafluoropentyl group, a pentyl group, a hexyl group, a perfluorohexyl group, a chloromethyl group, a bromomethyl group and an iodomethyl group. The number of carbon atoms of the alkyl group is preferably 1 to 4, more preferably 1 to 3, still more preferably a methyl group or an ethyl group, and yet more preferably a methyl group.

R^{a1} is preferably a hydrogen atom or an alkyl group having 1 to 4 carbon atoms, more preferably a hydrogen atom, a methyl group or an ethyl group, and still more preferably a hydrogen atom or a methyl group.

Examples of the alkoxy group in R^{a17} include a methoxy group, an ethoxy group, a propoxy group, an isopropoxy group, a butoxy group, a sec-butoxy group, a tert-butoxy group, a pentyloxy group and a hexyloxy group. The alkoxy group is preferably an alkoxy group having 1 to 4 carbon atoms, more preferably a methoxy group or an ethoxy group, and still more preferably a methoxy group.

Examples of the alkoxyalkyl group in R^{a17} include a methoxymethyl group, an ethoxyethyl group, a propoxymethyl group, an isopropoxymethyl group, a butoxymethyl group, a sec-butoxymethyl group and a tert-butoxymethyl group. The alkoxyalkyl group is preferably an alkoxyalkyl group having 2 to 8 carbon atoms, more preferably a methoxymethyl group or an ethoxyethyl group, and still more preferably a methoxymethyl group.

Examples of the alkoxyalkoxy group in R^{a17} include a methoxymethoxy group, a methoxyethoxy group, an ethoxymethoxy group, an ethoxyethoxy group, a propoxymethoxy group, an isopropoxymethoxy group, a butoxymethoxy group, a sec-butoxymethoxy group and a tert-butoxymethoxy group. The alkoxyalkoxy group is preferably an alkoxyalkoxy group having 2 to 8 carbon atoms, and more preferably a methoxyethoxy group or an ethoxyethoxy group.

Examples of the alkylcarbonyl group in R^{a17} include an acetyl group, a propionyl group and a butyryl group. The alkylcarbonyl group is preferably an alkylcarbonyl group having 2 to 3 carbon atoms, and more preferably an acetyl group.

Examples of the alkylcarbonyloxy group in R^{a17} include an acetyloxy group, a propionyloxy group and a butyryloxy group. The alkylcarbonyloxy group is preferably an alkylcarbonyloxy group having 2 to 3 carbon atoms, and more preferably an acetyloxy group.

R^{a17} is preferably a halogen atom, a hydroxy group, an alkyl group having 1 to 4 carbon atoms, an alkoxy group having 1 to 4 carbon atoms or an alkoxyalkoxy group having 2 to 8 carbon atoms, more preferably a fluorine atom, an iodine atom, a hydroxy group, a methyl group, a methoxy group, an ethoxy group, an ethoxyethoxy group or an ethoxymethoxy group, and still more preferably a fluorine atom, an iodine atom, a hydroxy group, a methyl group, a methoxy group or an ethoxyethoxy group.

na12 is preferably 0, 1 or 2, more preferably 0 or 1, and still more preferably 0.

Examples of the alkanediyl group in A^{a11} include branched alkanediyl groups such as a methylene group, an ethylene group, a propane-1,3-diyl group, a propane-1,2-diyl group, a butane-1,4-diyl group, a pentane-1,5-diyl group, a hexane-1,6-diyl group, a butane-1,3-diyl group, a 2-methylpropane-1,3-diyl group, a 2-methylpropane-1,2-diyl group, a pentane-1,4-diyl group, a 2-methylbutane-1,4-diyl group, a heptane-1,6-diyl group, an octane-1,8-diyl group, a nonane-1,9-diyl group and a decane-1,10-diyl group. The number of carbon atoms of the alkanediyl group is preferably 1 to 6, more preferably 1 to 4, still more preferably 1 to 3, and yet more preferably 1 or 2. Of these, A^{a11} is preferably a methylene group or an ethylene group.

Examples of the alkyl group as for R^{a18} include a methyl group, an ethyl group, an n-propyl group, an isopropyl group, an n-butyl group and a tert-butyl group.

When —CH₂— included in the alkanediyl group as for A^{a11} is replaced by —O—, —CO— or —NR^{a18}, the number of carbon atoms before replacement is taken as the number of carbon atoms of the hydrocarbon group.

Examples of the group in which —CH₂— in the alkanediyl group as for A^{a11} is replaced by —O— or —CO— include a hydroxy group (a group in which —CH₂— included in the methyl group is replaced by —O—), a carboxy group (a group in which —CH₂—CH₂— included in the ethyl group is replaced by —O—CO—), a carbonyl group (a group in which —CH₂— included in the methylene group is replaced by —CO—), an oxy group (a group in which —CH₂ included in the methylene group is replaced by —O—), an amino group (a group in which —CH₂— included in the alkanediyl group is replaced by —NR^{a28}—), an alkoxy group (a group in which —CH₂— at any position included in the alkyl group is replaced by —O—), an alkoxycarbonyl group (a group in which —CH₂—CH₂— at any position included in the alkyl group is replaced by —O—CO—), an alkylcarbonyl group (a group in which —CH₂— at any position included in the alkyl group is replaced by —CO—), an alkylamino group (a group in which —CH₂— at any position included in the alkyl group is replaced by —NR^{a18}—), a peptide group (a group in which —CH₂—CH₂— at any position included in the ethylene group is replaced by —CO—NR^{a18}—), an alkanediyloxy group (a group in which —CH₂— at any position included in the alkanediyl group is replaced by —O—), an alkanediyloxycarbonyl group (a group in which —CH₂—CH₂— at any position included in the alkanediyl group is replaced by —O—CO—), an alkanediylcarbonyl group (a group in which —CH₂— at any position included in the alkanediyl group is replaced by —CO—), an alkanediylcarbonyloxy group (a group in which —CH₂—CH₂— at any position included in the alkanediyl group is replaced by —CO—O—), an alkanediylsulfonyl group (a group in which —CH₂— at any position included in the alkanediyl group is replaced by —SO₂—), an alkanediylthio group (a group in which —CH₂— at any position included in the alkanediyl group is replaced by —S—), an alkanediylamino group (a group in which —CH₂— at any position included in the alkanediyl group is replaced by —NR^{a18}—) and the like. Examples of these replaced groups include those which are the same as mentioned herein.

Examples of the alkylamino group include alkylamino groups having 1 to 11 carbon atoms, for example, a methylamino group, an ethylamino group, a propylamino group, a butylamino group, a pentylamino group, a hexylamino group and an octylamino group.

Examples of the alkanediylamino group include alkanediylthio groups having 1 to 11 carbon atoms, for example, a methyleneamino group, an ethyleneimino group, a propyleneamino group and the like. The number of carbon atoms of the alkanediylamino group is preferably 1 to 8, more preferably 1 to 6, still more preferably 1 to 4, and yet more preferably 1 to 3.

Examples of the group in which —CH₂— included in the alkanediyl group as for A^{a11} is replaced by —O—, —CO— or —NR^{a28} include, for example, *—O—, *—CO—O—, *—O—CO—, *—CO—O-A^{a12}-CO— O—, *—O—CO-A^{a12}-O—, *—O-A^{a12}-CO—O—, *—CO—O-A^{a12}-O—CO— and *—O— CO-A^{a12}-O—CO—. Of these, *—CO—O—, *—CO—O-A^{a12}-CO—O— or *—O— A^{a12}˜CO—O—, *—CO—NR^{a18}— are preferable. Here, A^{a12} represents an alkanediyl group having 1 to 8 carbon atoms, * represents a bonding site to carbon atoms to which R^{a1} is bonded. Examples of the alkanediyl group as for A^{a12} include the same alkanediyl group as for A^{a11} as long as the upper limit of the number of carbon atoms permits.

A^{a11} is preferably a single bond, *—CO—O— or *—CO—O— A^{a12}-CO—O—, more preferably a single bond, *—CO—O— or *—CO—O—CH₂—CO—O—, and still more preferably a single bond or *—CO— O—.

Examples of the hydrocarbon group in R^{a34}, R^{a35} and R^{a36} include an alkyl group, an alicyclic hydrocarbon group, an aromatic hydrocarbon group, and groups obtained by combining these groups.

Examples of the alkyl group include a methyl group, an ethyl group, a propyl group, a butyl group, a pentyl group, a hexyl group, a heptyl group, an octyl group and the like.

The alicyclic hydrocarbon group may be either monocyclic or polycyclic. Examples of the monocyclic alicyclic hydrocarbon group include cycloalkyl groups such as a cyclopentyl group, a cyclohexyl group, a cycloheptyl group and a cyclooctyl group. Examples of the polycyclic alicyclic hydrocarbon group include a decahydronaphthyl group, an adamantyl group, a norbornyl group, and the following groups (* represents a bonding site).

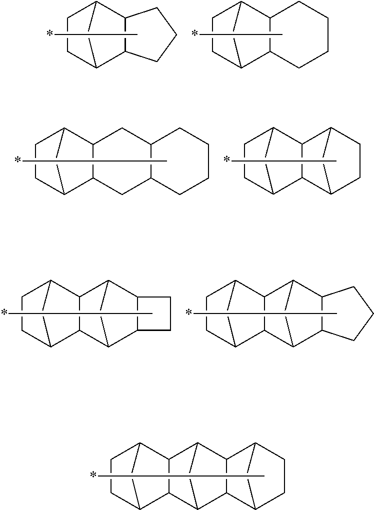

Examples of the aromatic hydrocarbon group include aryl groups such as a phenyl group, a naphthyl group, an anthryl group, a biphenyl group and a phenanthryl group.

Examples of the combined group include groups obtained by combining the above-mentioned alkyl group and alicyclic hydrocarbon group (e.g., alkylcycloalkyl groups or cycloalkylalkyl groups, such as a methylcyclohexyl group, a dimethylcyclohexyl group, a methylnorbornyl group, a cyclohexylmethyl group, an adamantylmethyl group, an adamantyldimethyl group and a norbornylethyl group), aralkyl groups such as a benzyl group, aromatic hydrocarbon groups having an alkyl group (a p-methylphenyl group, a p-tert-butylphenyl group, a tolyl group, a xylyl group, a cumenyl group, a mesityl group, a 2,6-diethylphenyl group, a 2-methyl-6-ethylphenyl group, etc.), aromatic hydrocarbon groups having an alicyclic hydrocarbon group (a p-cyclohexylphenyl group, a p-adamantylphenyl group, etc.), aryl-cycloalkyl groups such as a phenylcyclohexyl group and the like. Particularly, examples of R^{a36} include an alkyl group having 1 to 18 carbon atoms, an alicyclic hydrocarbon group having 3 to 18 carbon atoms, an aromatic hydrocarbon group having 6 to 18 carbon atoms, or groups formed by combining these groups.

Examples of the divalent hydrocarbon group having 2 to 20 carbon atoms formed by bonding R^{a35} and R^{a36} from each other together with —C—O— to which R^{a35} and R^{a36} are added include the following groups. * represents a bonding site, and one is a bonding site to R^{a34}.

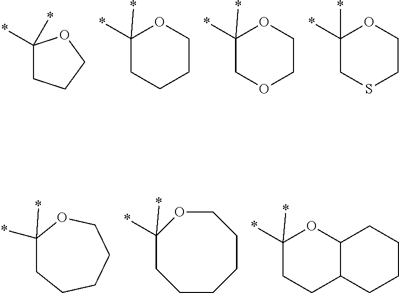

R^{a34} is preferably a hydrogen atom.

R^{a35} is preferably a hydrogen atom, an alkyl group having 1 to 12 carbon atoms or an alicyclic hydrocarbon group having 3 to 12 carbon atoms, and more preferably a methyl group or an ethyl group.

The hydrocarbon group of R^{a36} is preferably an alkyl group having 1 to 18 carbon atoms, an alicyclic hydrocarbon group having 3 to 18 carbon atoms, an aromatic hydrocarbon group having 6 to 18 carbon atoms, or groups formed by combining these groups, and more preferably an alkyl group having 1 to 18 carbon atoms, an alicyclic hydrocarbon group having 3 to 18 carbon atoms or an aralkyl group having 7 to 18 carbon atoms. The alkyl group and the alicyclic hydrocarbon group in R^{a36} are preferably unsubstituted. The aromatic hydrocarbon group in R^{a36} is preferably an aromatic ring having an aryloxy group having 6 to 10 carbon atoms.

—OC(R^{a34}) (R^{a35})—O—R^{a36} in the structural unit (a1-4) is eliminated by contacting with an acid (e.g., p-toluenesulfonic acid) to form a hydroxy group or a carboxy group.

—X^{a1}—OC(R^{a34}) (R^{a35})—O—R^{a36} is preferably bonded at the o-position or the p-position, and more preferably at the p-position of the benzene ring, with respect to a bonding site of A^{a11}. In the case of the naphthalene ring, it may be bonded at any of the 2-position to the 8-position with respect to the 1-position which is the bonding site of A^{a11}, and preferably bonded at the 3-position or the 4-position.

The structural unit (a1-4) includes, for example, structural units derived from the monomers mentioned in JP 2010-204646 A. The structural unit preferably includes structural units represented by formula (a1-4-1) to formula (a1-4-42) and a structural unit in which a hydrogen atom corresponding to R^{a1} in the structural unit (a1-4) is substituted with a halogen atom, a haloalkyl group or an alkyl group, and more preferably structural units represented by formula (a1-4-1) to formula (a1-4-5), formula (a1-4-10), formula (a1-4-13) and formula (a1-4-14).

[chemical structure]

[chemical structure]

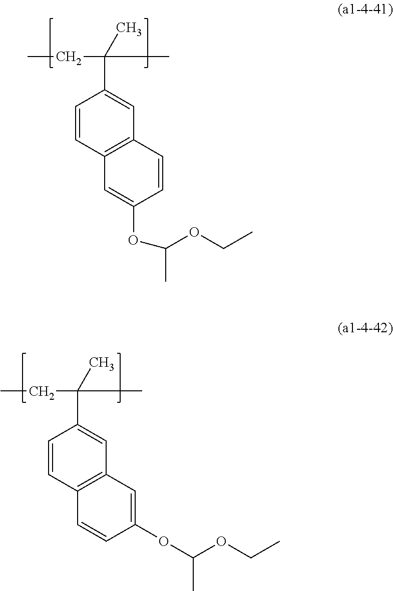

When the resin (A) includes a structural unit (a1-4), the content is 10 mol % or more, preferably 15 mol % or more, still more preferably 20 mol % or more, yet more preferably 25 mol % or more, further preferably 30 mol % or more, still further preferably 40 mol % or more, and yet further preferably 50 mol % or more, based on the total of all structural units of the resin (A). The content is also 95 mol % or less, preferably 90 mol % or less, more preferably 85 mol % or less, still more preferably 80 mol % or less, further preferably 75 mol % or less, still further preferably 70 mol % or less, and yet further preferably 65 mol % or less. Specifically, the content is preferably 10 to 95 mol %, more preferably 15 to 90 mol %, still more preferably 20 to 85 mol %, yet more preferably 20 to 70 mol %, and particularly preferably 20 to 60 mol %.

When the resin (A) or the like includes the structural unit (a1-4), the content is 10 mol % or more, preferably 15 mol % or more, more preferably 20 mol % or more, still more preferably 25 mol % or more, yet more preferably 30 mol % or more, further preferably 40 mol % or more, and still further preferably 50 mol % or more based on the total of all structural units of the resin (A) or the like. The content is also 95 mol % or less, preferably 90 mol % or less, more preferably 85 mol % or less, still more preferably 80 mol % or less, yet more preferably 75 mol % or less, further preferably 70 mol % or less, and still further preferably 65 mol % or less. Specifically, the content is 10 to 95 mol %, preferably 15 to 90 mol %, more preferably 20 to 85 mol %, still more preferably 20 to 70 mol %, and yet more preferably 20 to 65 mol %.

The structural unit derived from a (meth)acrylic monomer having a group (2) also includes a structural unit represented by formula (a1-5) (hereinafter sometimes referred to as “structural unit (a1-5)”):

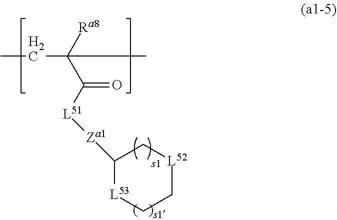

wherein, in formula (a1-5),
R^{aB} represents an alkyl group having 1 to 6 carbon atoms which may have a halogen atom, a hydrogen atom or a halogen atom,
Z^{a1} represents a single bond or *—(CH₂)ₕ₃—CO-L⁵⁴-, h3 represents an integer of 1 to 4, and * represents a bonding site to L⁵¹,
L⁵¹, L⁵², L⁵³ and L⁵⁴ each independently represent —O— or —S—,
s1 represents an integer of 1 to 3, and
s1′ represents an integer of 0 to 3.

The halogen atom includes a fluorine atom and a chlorine atom and is preferably a fluorine atom.

Examples of the alkyl group having 1 to 6 carbon atoms which may have a halogen atom include a methyl group, an ethyl group, a propyl group, a butyl group, a pentyl group, a hexyl group, a heptyl group, an octyl group, a fluoromethyl group and a trifluoromethyl group.

In formula (a1-5), R^{aB} is preferably a hydrogen atom, a methyl group or a trifluoromethyl group,
L⁵¹ is preferably an oxygen atom,
one of L⁵² and L⁵³ is preferably —O— and the other one is preferably —S—,
s1 is preferably 1,
s1′ is preferably an integer of 0 to 2, and
Z^{a1} is preferably a single bond or *—CH₂—CO—O—.

The structural unit (a1-5) includes, for example, structural units derived from the monomers mentioned in JP 2010-61117 A. Of these structural units, structural units represented by formula (a1-5-1) to formula (a1-5-4) are preferable, and structural units represented by formula (a1-5-1) or formula (a1-5-2) are more preferable.

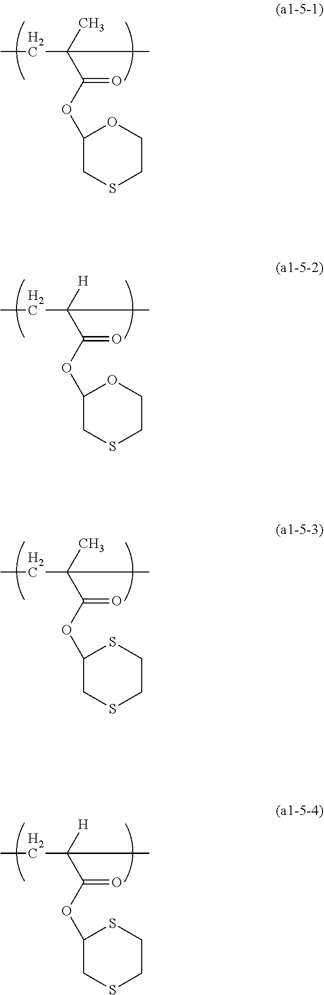

When the resin (A) or the like includes the structural unit (a1-5), the content is 1 mol % or more, preferably 2 mol % or more, more preferably 3 mol % or more, still more preferably 5 mol % or more, yet more preferably 10 mol % or more, further preferably 20 mol % or more, and still further preferably 25 mol % or more, based on all structural units of the resin (A) or the like. The content is also 80 mol % or less, preferably 70 mol % or less, more preferably 60 mol % or less, still more preferably 50 mol % or less, yet more preferably 45 mol % or less, further preferably 40 mol % or less, and still further preferably 30 mol % or less. Specifically, the content is preferably 1 to 50 mol %, more preferably 3 to 45 mol %, still more preferably 5 to 40 mol %, and yet more preferably 5 to 30 mol %.

Examples of the structural unit having a group (1) in the structural unit (a1) include a structural unit represented by formula (a1-6) (hereinafter sometimes referred to as “structural unit (a1-6)”):

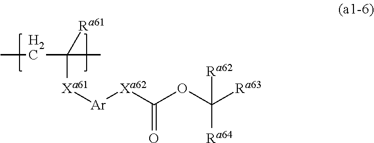

wherein, in formula (a1-6),
R^{a61} represents a hydrogen atom, a halogen atom, or an alkyl group having 1 to 6 carbon atoms which may have a halogen atom,
R^{a62}, R^{a63} and R^{a64} each independently represent an alkyl group having 1 to 6 carbon atoms or a cyclic hydrocarbon group having 3 to 18 carbon atoms which may have a substituent, or R^{a62} and R^{a63} may be bonded to each other to form a ring having 3 to 20 carbon atoms together with carbon atoms to which R^{a62} and R^{a63} are bonded,
X^{a61} represents a single bond, —CO—O—* or —CO—NR⁵—*, * represents a bonding site to —Ar, and R⁵ represents a hydrogen atom or an alkyl group having 1 to 4 carbon atoms,
X^{a62} represents a single bond, *—O-L^{a61}- or *—CO—O— L^{a62}-, * represents a bonding site to —Ar, and L^{a61} and L^{a62} each independently represent an alkanediyl group having 1 to 4 carbon atoms, and
Ar represents an aromatic hydrocarbon group having 6 to 20 carbon atoms which may have a substituent.

Examples the halogen atom in R^{a61} include a fluorine atom, a chlorine atom and a bromine atom. Of these, a fluorine atom is preferable.

Examples of the alkyl group having 1 to 6 carbon atoms which may have a halogen atom in R^{a61} include a methyl group, an ethyl group, a propyl group, a butyl group, a pentyl group, a hexyl group, a fluoromethyl group, a difluoromethyl group, a trifluoromethyl group, a perfluoroethyl group, a 2,2,2-trifluoroethyl group, a 1,1,2,2-tetrafluoroethyl group, a perfluoropropyl group, a 2,2,3,3,3-pentafluoropropyl group, a perfluorobutyl group, a 1,1,2,2,3,3,4,4-octafluorobutyl group, a perfluoropentyl group, a 2,2,3,3,4,4,5,5,5-nonafluoropentyl group and a perfluorohexyl group.

R^{a61} is preferably a hydrogen atom, a methyl group or a trifluoromethyl group.

Examples of the alkyl group in R^{a62}, R^{a63} and R^{a64} include a methyl group, an ethyl group, a propyl group, a butyl group, a pentyl group, a hexyl group and the like. The alkyl group is preferably an alkyl group having 1 to 4 carbon atoms, more preferably an alkyl group having 1 to 3 carbon atoms, and still more preferably a methyl group or an ethyl group.

Examples of the cyclic hydrocarbon group in R^{a62}, R^{a63} and R^{a64} include an alicyclic hydrocarbon group and an aromatic hydrocarbon group.

The alicyclic hydrocarbon group in R^{a62}, R^{a63} and R^{a64} may be either monocyclic or polycyclic. Examples of the monocyclic alicyclic hydrocarbon group include a cyclopentyl group, a cyclohexyl group, a cycloheptyl group, a cyclooctyl group and the like. Examples of the polycyclic alicyclic hydrocarbon group include a decahydronaphthyl group, an adamantyl group, a norbornyl group and the like. The number of carbon atoms of the alicyclic hydrocarbon group is preferably 3 to 16, and more preferably 3 to 12.

Examples of the aromatic hydrocarbon group include a phenylene group, a naphthylene group and the like.

Examples of the substituent which may be possessed by the cyclic hydrocarbon group include a halogen atom, a hydroxy group, an alkyl group having 1 to 6 carbon atoms, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group or a methacryloyloxy group.

Examples of the ring formed by bonding R^{a62} and R^{a63} to each other include an adamantane ring, a cyclopentane ring or a cyclohexane ring. Specifically, when R^{a62} and R^{a63} are bonded to each other to form a ring, examples of —C(R^{a62}) (R^{a63}) (R^{a64}) include the following groups. * represents a bonding site to the oxygen atom. The number of carbon atoms of the ring is preferably 3 to 16, and more preferably 3 to 12.

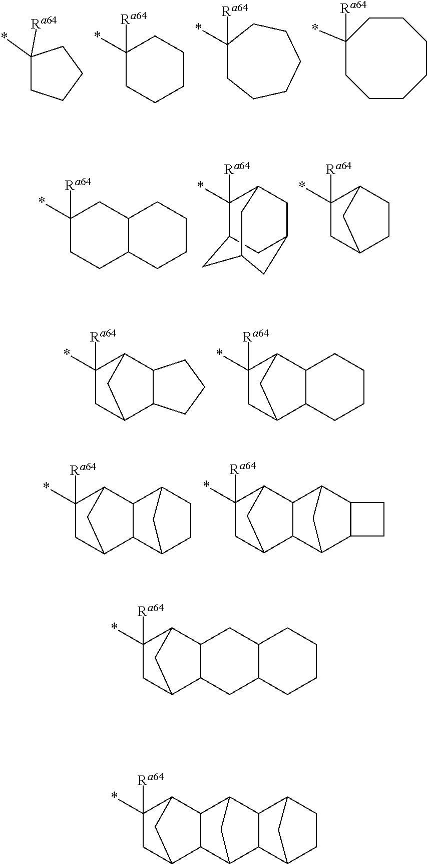

Examples of the alkanediyl group having 1 to 4 carbon atoms in L^{a61} and L^{a62} include a methylene group, an ethylene group, a propane-1,3-diyl group, a propane-1,2-diyl group, a butane-1,4-diyl group, a butane-1,3-diyl group, a 2-methylpropane-1,3-diyl group, a 2-methylpropane-1,2-diyl group and the like.

Preferably, L^{a61} and L^{a62} each independently is a methylene group or an ethylene group.

X^{a61} is preferably a single bond or —CO—O—*, and more preferably a single bond.

X^{a62} is preferably a single bond or *—O-L^{a61}-, and more preferably a single bond.

Examples of the aromatic hydrocarbon group having 6 to 20 carbon atoms as for Ar include a phenylene group, a naphthylene group, an anthrylene group, a biphenylene group and a phenanthrylene group.

Examples of the substituent which may be possessed by the aromatic hydrocarbon group include a halogen atom, a hydroxy group, an alkyl group having 1 to 6 carbon atoms, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group or a methacryloyloxy group.

Examples of the halogen atom include a fluorine atom, a chlorine atom and a bromine atom.

Examples of the alkyl group include a methyl group, an ethyl group, a propyl group, an isopropyl group, a butyl group, a sec-butyl group, a tert-butyl group, a pentyl group and a hexyl group. The alkyl group is preferably an alkyl group having 1 to 4 carbon atoms, more preferably a methyl group or an ethyl group, and still more preferably a methyl group.

Examples of the alkoxy group include a methoxy group, an ethoxy group, a propoxy group, an isopropoxy group, a butoxy group, a sec-butoxy group, a tert-butoxy group, a pentyloxy group and a hexyloxy group. The alkoxy group is preferably an alkoxy group having 1 to 4 carbon atoms, more preferably a methoxy group or an ethoxy group, and still more preferably a methoxy group.

Examples of the alkoxyalkyl group include a methoxymethyl group, an ethoxyethyl group, a propoxymethyl group, an isopropoxymethyl group, a butoxymethyl group, a sec-butoxymethyl group and a tert-butoxymethyl group. The alkoxyalkyl group is preferably an alkoxyalkyl group having 2 to 8 carbon atoms, more preferably a methoxymethyl group or an ethoxyethyl group, and still more preferably a methoxymethyl group.

Examples of the alkoxyalkoxy group include a methoxymethoxy group, a methoxyethoxy group, an ethoxymethoxy group, an ethoxyethoxy group, a propoxymethoxy group, an isopropoxymethoxy group, a butoxymethoxy group, a sec-butoxymethoxy group and a tert-butoxymethoxy group. The alkoxyalkoxy group is preferably an alkoxyalkoxy group having 2 to 8 carbon atoms, and more preferably a methoxyethoxy group or an ethoxyethoxy group.

Examples of the alkylcarbonyl group include an acetyl group, a propionyl group and a butyryl group. The alkylcarbonyl group is preferably an alkylcarbonyl group having 2 to 3 carbon atoms, and more preferably an acetyl group.

Examples of the alkylcarbonyloxy group include an acetyloxy group, a propionyloxy group and a butyryloxy group. The alkylcarbonyloxy group is preferably an alkylcarbonyloxy group having 2 to 3 carbon atoms, and more preferably an acetyloxy group.

The substituent is preferably a halogen atom, a hydroxy group, an alkyl group having 1 to 4 carbon atoms, an alkoxy group having 1 to 4 carbon atoms or an alkoxyalkoxy group having 2 to 8 carbon atoms, more preferably a fluorine atom, an iodine atom, a hydroxy group, a methyl group, a methoxy group, an ethoxy group, an ethoxyethoxy group or an ethoxymethoxy group, and still more preferably a fluorine atom, an iodine atom, a hydroxy group, a methyl group, a methoxy group or an ethoxyethoxy group.

Ar is preferably a phenylene group which may have a substituent, and more preferably a phenylene group which may have a hydroxy group.

Examples of the structural unit (a1-6) include structural units represented respectively by formula (a1-6-1) to formula (a1-6-43), and structural units represented respectively by formula (a1-6-1) to formula (a1-6-9) are preferable, a structural unit represented respectively by formula (a1-6-1), formula (a1-6-2), formula (a1-6-4), formula (a1-6-5), formula (a1-6-7) or formula (a1-6-8) is more preferable, and a structural unit represented respectively by formula (a1-6-1) or formula (a1-6-2) is still more preferable.

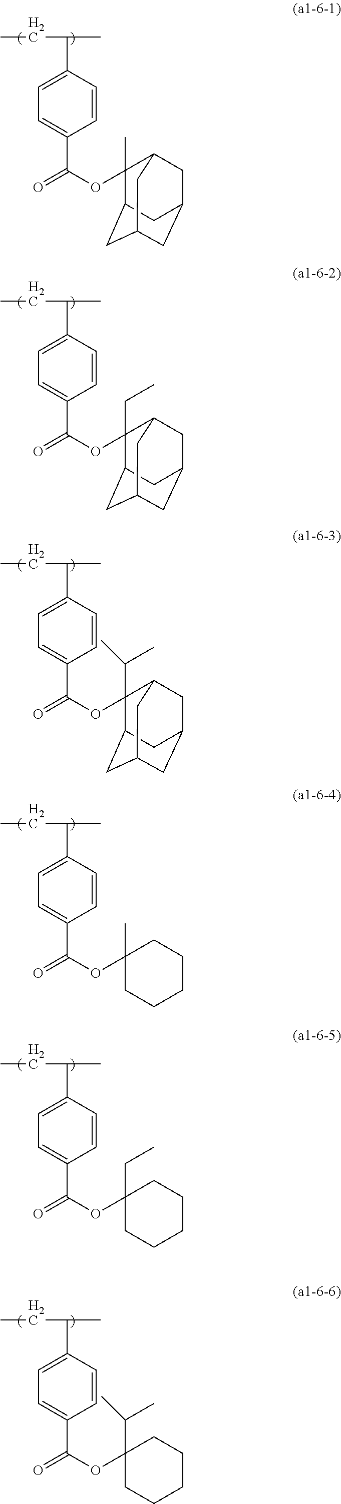

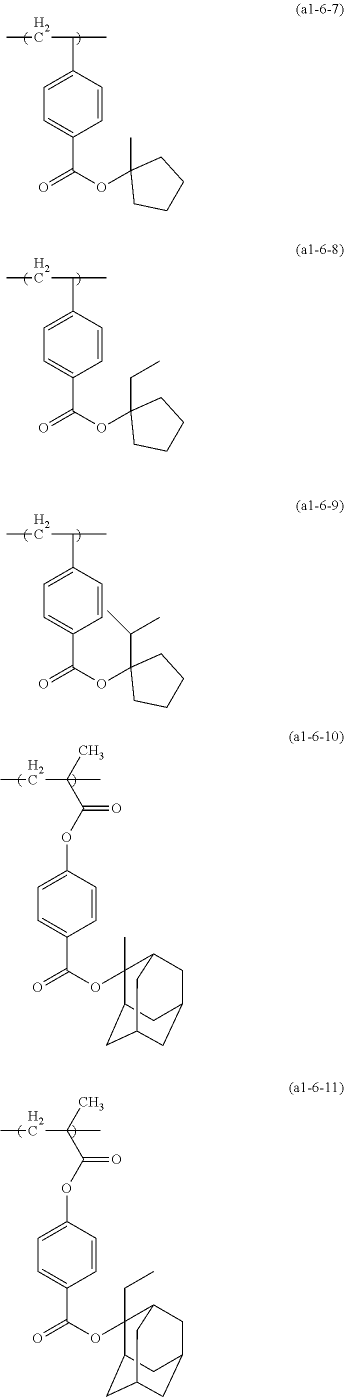

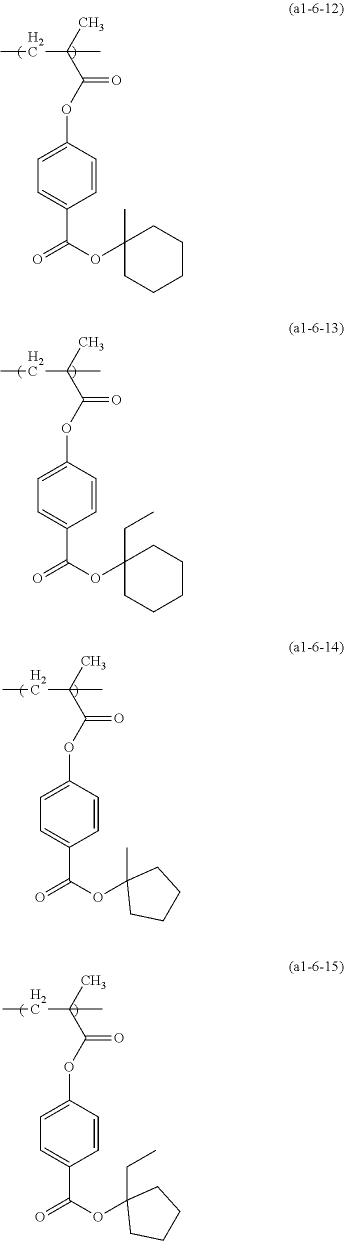

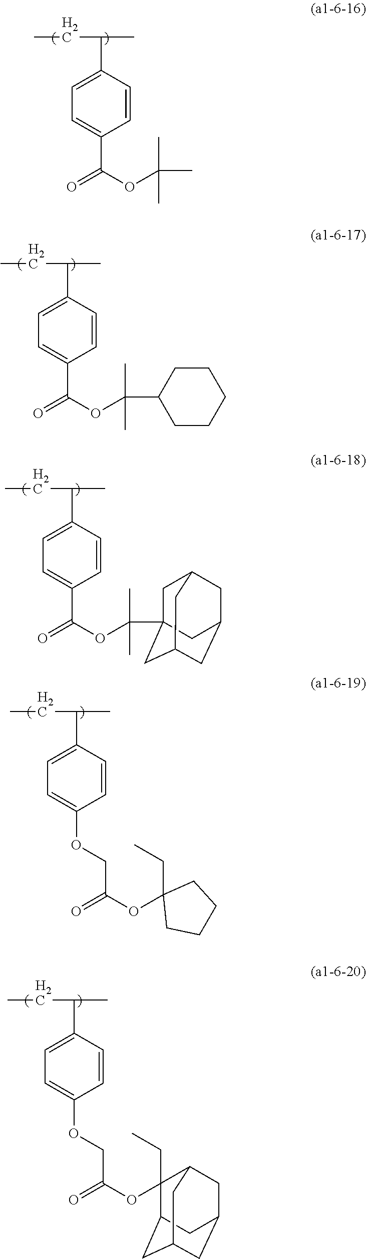

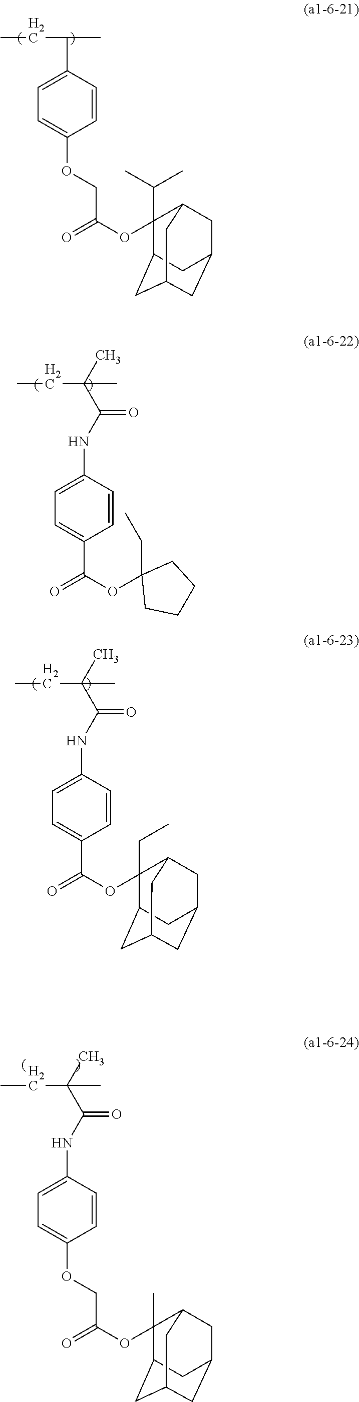

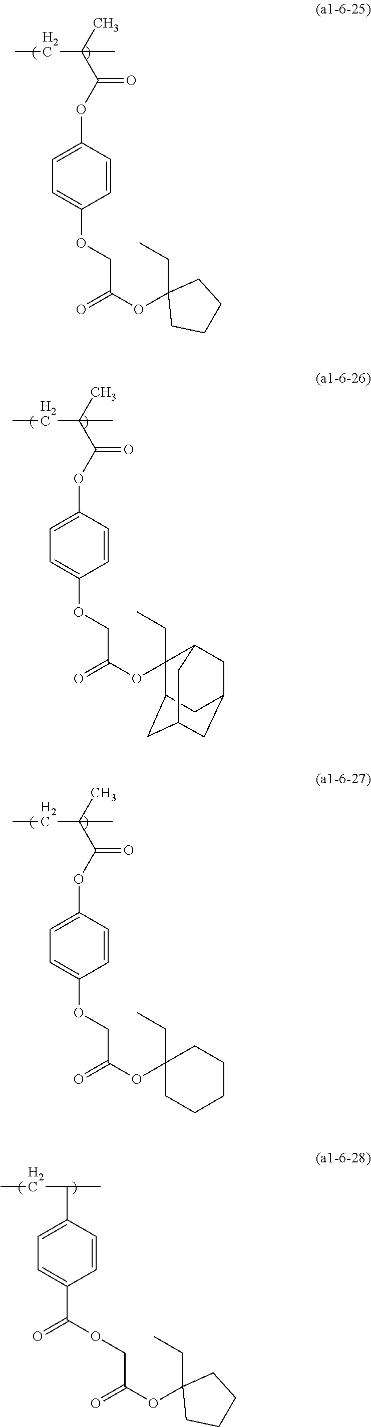

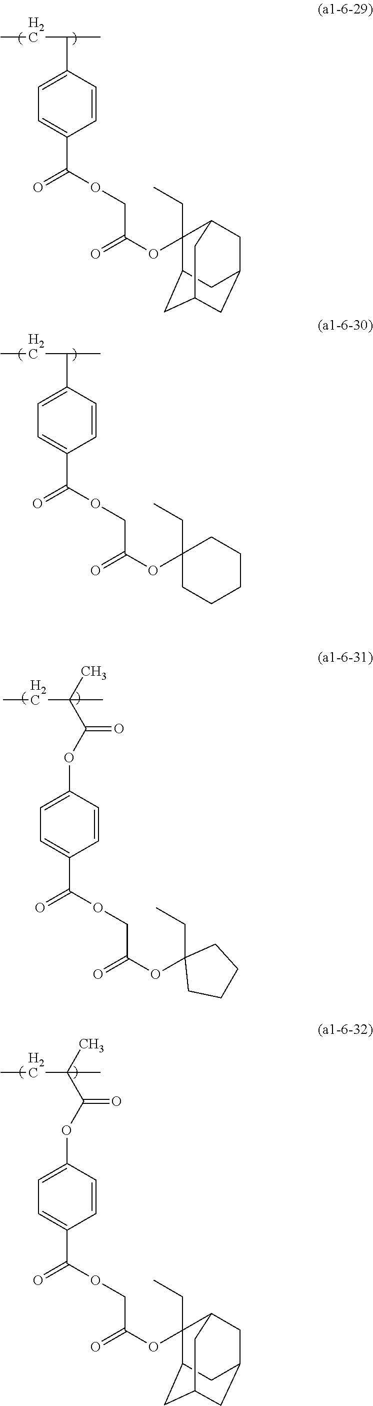

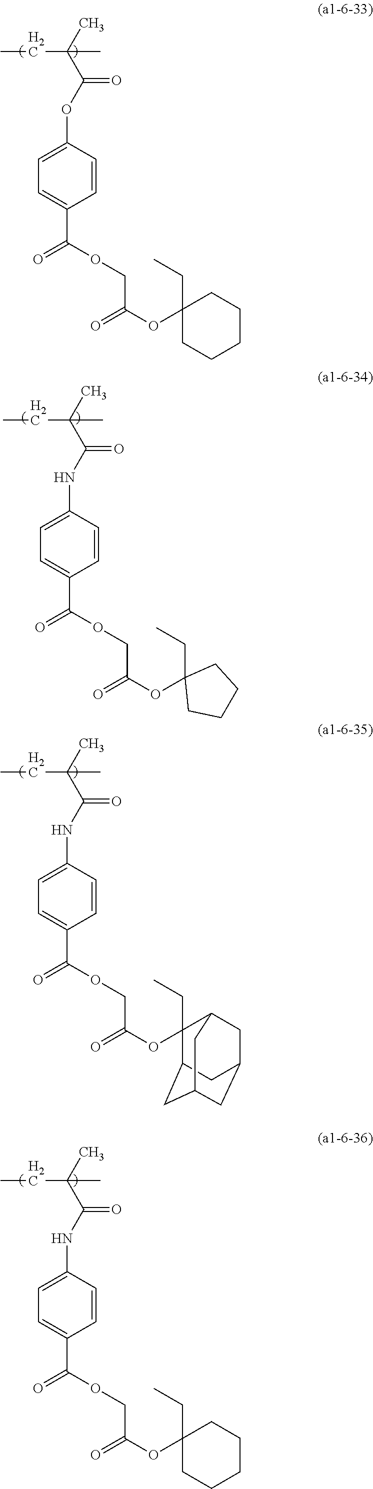

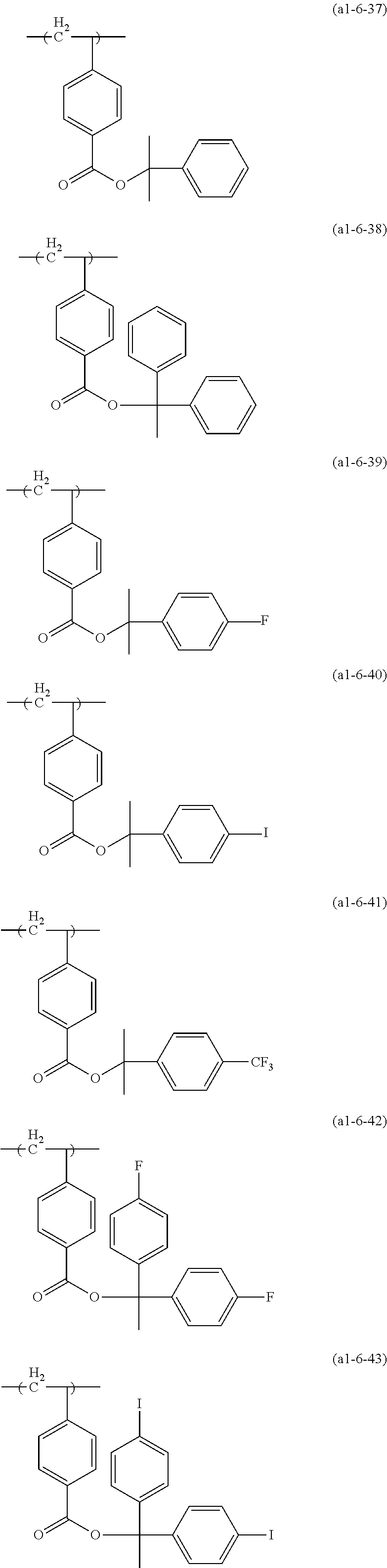

It is also possible to exemplify, as the structural unit (a1-6), structural units in which the hydrogen atom corresponding to R^{a61} is substituted with a methyl group, a halogen atom or a haloalkyl group in structural units represented respectively by formula (a1-6-1) to formula (a1-6-9), formula (a1-6-16) to formula (a1-6-21), and formula (a1-6-28) to formula (a1-6-30), and formula (a1-6-37) to formula (a1-6-43), and structural units in which the methyl group corresponding to R^{a61} is substituted with a hydrogen atom, a halogen atom or a haloalkyl group in structural units represented respectively by formula (a1-6-10) to formula (a1-6-15), formula (a1-6-22) to formula (a1-6-27) and formula (a1-6-31) to formula (a1-6-36).

When the resin (A) includes a structural unit (a1-6), the content is 3 mol % or more, preferably 5 mol % or more, more preferably 7 mol % or more, still more preferably 10 mol % or more, yet more preferably 20 mol % or more, further preferably 30 mol % or more, and still further preferably 40 mol % or more, based on all structural units of the resin (A). The content is also 80 mol % or less, preferably 75 mol % or less, more preferably 70 mol % or less, and still more preferably 65 mol % or less. Specifically, the content is preferably 3 to 80 mol %, more preferably 5 to 75 mol %, still more preferably 7 to 70 mol %, further preferably 7 to 65 mol %, and still further preferably 10 to 65 mol %.

Examples of the structural unit (a1) also include the following structural units.

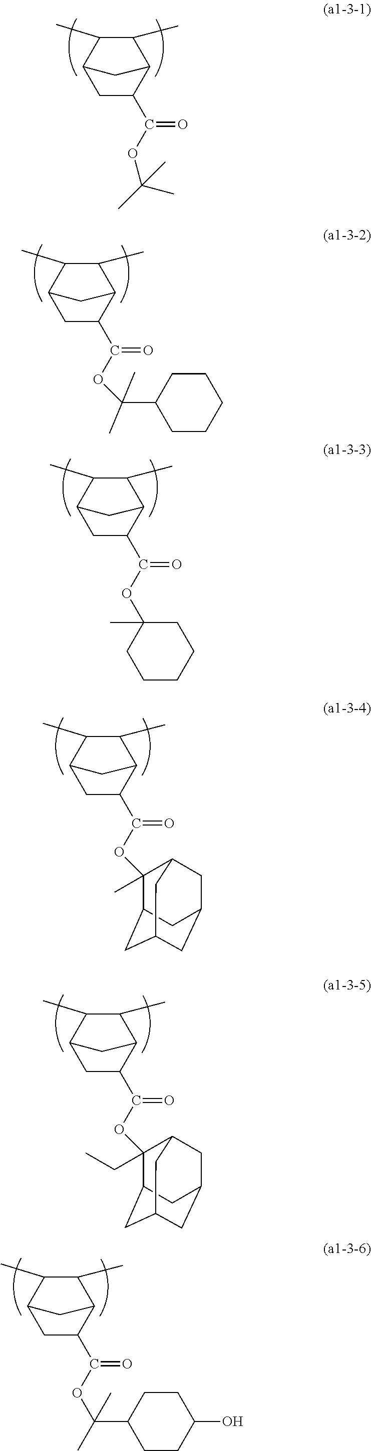

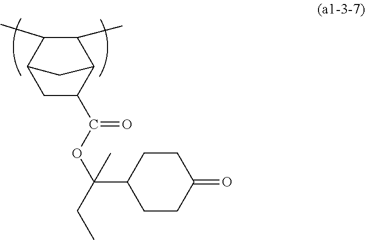

When the resin (A) or the like includes structural units represented by formulas (a1-3-1) to (a1-3-7), the content is 10 mol % or more, preferably 15 mol % or more, more preferably 20 mol % or more, still more preferably 25 mol % or more, yet more preferably 30 mol % or more, further preferably 40 mol % or more, and still further preferably 50 mol % or more, based on all structural units of the resin (A) or the like. The content is also 95 mol % or less, preferably 90 mol % or less, more preferably 85 mol % or less, still more preferably 80 mol % or less, yet more preferably 75 mol % or less, further preferably 70 mol % or less, and still further preferably 60 mol % or less. Specifically, the content is preferably 10 to 95 mol %, more preferably 15 to 90 mol %, still more preferably 20 to 85 mol %, yet more preferably 20 to 70 mol %, and particularly preferably 20 to 60 mol %.

<Structural Unit (s)>

The structural unit (s) is derived from a monomer having no acid-labile group (hereinafter sometimes referred to as “monomer (s)”). It is possible to use, as the monomer from which the structural unit (s) is derived, a monomer having no acid-labile group known in the resist field.

The structural unit (s) preferably has a hydroxy group, a carboxy group or a lactone ring. When a resin including a structural unit having a hydroxy group or a carboxy group and having no acid-labile group (hereinafter sometimes referred to as “structural unit (a2)”) and/or a structural unit having a lactone ring and having no acid-labile group (hereinafter sometimes referred to as “structural unit (a3)”) is used in the resist composition of the present invention, it is possible to improve the resolution of a resist pattern and the adhesion to a substrate. Examples of the structural unit (s) include, in addition to the above-mentioned structural unit, a structural unit having a non-leaving hydrocarbon group (hereinafter sometimes referred to as structural unit (a5)), a structural unit having a sultone structure (hereinafter sometimes referred to as structural unit (a6)), or a structural unit which is decomposed upon exposure to radiation to generate an acid (hereinafter sometimes referred to as structural unit (a7)).

<Structural Unit (a2)>

The structural unit (a2) is a structural unit represented by formula (a2) and has an alcoholic hydroxy group, a phenolic hydroxy group or a phenolic carboxy group:

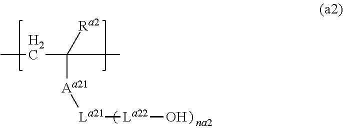

wherein, in formula (a2),
R^{a2} represents a hydrogen atom, a halogen atom or an alkyl group having 1 to 6 carbon atoms which may have a halogen atom,
A^{a21} represents a single bond or an alkanediyl group having 1 to 12 carbon atoms, and —CH₂— included in the alkanediyl group may be replaced by —O—, —CO— or —NR^{a28}—,
R^{a28} represents a hydrogen atom or an alkyl group having 1 to 4 carbon atoms,
L^{a21} represents a single bond or a hydrocarbon group having 1 to 28 carbon atoms, the hydrocarbon group may have a substituent, and —CH₂— included in the hydrocarbon group may be replaced by —O—, —CO—, —S— or —SO₂—,
L^{a22} represents a single bond or a chain hydrocarbon group having 1 to 12 carbon atoms which may have a fluorine atom, and —CH₂— included in the chain hydrocarbon group may be replaced by —O— or —CO—, and
na2 represents an integer of 1 to 5, and when na2 is 2 or more, a plurality of L^{a22} may be the same or different from each other.

Examples of the halogen atom in R^{a2} include a fluorine atom, a chlorine atom and a bromine atom.

Examples of the alkyl group having 1 to 6 carbon atoms which may have a halogen atom in R^{a2} include a trifluoromethyl group, a difluoromethyl group, a methyl group, a perfluoroethyl group, a 2,2,2-trifluoroethyl group, a 1,1,2,2-tetrafluoroethyl group, an ethyl group, a perfluoropropyl group, a 2,2,3,3,3-pentafluoropropyl group, a propyl group, a perfluorobutyl group, a 1,1,2,2,3,3,4,4-octafluorobutyl group, a butyl group, a perfluoropentyl group, a 2,2,3,3,4,4,5,5,5-nonafluoropentyl group, a pentyl group, a hexyl group, a perfluorohexyl group, a chloromethyl group, a bromomethyl group, an iodomethyl group and the like. The number of carbon atoms of the alkyl group is preferably 1 to 4, more preferably 1 to 3, and still more preferably 1 or 2.

R^{a2} is preferably a hydrogen atom or an alkyl group having 1 to 4 carbon atoms, more preferably a hydrogen atom, a methyl group or an ethyl group, and still more preferably a hydrogen atom or a methyl group.

Examples of the alkanediyl group as for A^{a21} include linear alkanediyl groups such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group and a dodecane-1,12-diyl group; and branched alkanediyl groups such as a butane-1,3-diyl group, a 2-methylpropane-1,3-diyl group, a 2-methylpropane-1,2-diyl group, a pentane-1,4-diyl group, a 2-methylbutane-1,4-diyl group, a heptane-1,6-diyl group, an octane-1,7-diyl group, a nonane-1,8-diyl group and a decane-1,9-diyl group.

The number of carbon atoms of the alkanediyl group is preferably 1 to 10, more preferably 1 to 8, still more preferably 1 to 6, yet more preferably 1 to 4, further preferably 1 to 3, and still further preferably 1 or 2. A^{a21} is also preferably a methylene group or an ethylene group.

Examples of the alkyl group as for R^{a28} include a methyl group, an ethyl group, an n-propyl group, an isopropyl group, an n-butyl group and a tert-butyl group.

When —CH₂— included in the alkanediyl group as for A^{a21} is replaced by —O—, —CO— or —NR^{a28}—, the number of carbon atoms before replacement is taken as the number of carbon atoms of the alkanediyl group.

Examples of the group in which —CH₂— included in the alkanediyl group as for A^{a21} is replaced by —O—, —CO— or —NR^{a28} include a hydroxy group (a group in which —CH₂— included in the methyl group is replaced by —O—), a carboxy group (a group in which —CH₂—CH₂— included in the ethyl group is replaced by —O—CO—), a carbonyl group (a group in which —CH₂— included in the methylene group is replaced by —CO—), an oxy group (a group in which —CH₂— included in the methylene group is replaced by —O—), an amino group (a group in which —CH₂— included in the methyl group is replaced by —NR^{a28}—), an alkoxy group (a group in which —CH₂— at any position included in the alkyl group is replaced by —O—), an alkoxycarbonyl group (in which —CH₂—CH₂— at any position included in the alkyl group is replaced by —O—CO—), an alkylcarbonyl group (a group in which —CH₂— at any position included in the alkyl group is replaced by —CO—), an alkylamino group (a group in which —CH₂— at any position included in the alkyl group is replaced by —NR^{a28}—), a peptide group (a group in which —CH₂—CH₂— included in the ethylene group is replaced by —CO— NR^{a28}—), an alkanediylamino group (a group in which —CH₂— at any position included in the alkanediyl group is replaced by —NR^{a28}—) and the like. Examples of these replaced groups include those which are the same as mentioned herein.

Examples of the alkylamino group include alkylamino groups having 1 to 11 carbon atoms, for example, a methylamino group, an ethylamino group, a propylamino group, a butylamino group, a pentylamino group, a hexylamino group, an octylamino group and the like. The number of carbon atoms of the alkylamino group is preferably 1 to 8, more preferably 1 to 6, still more preferably 1 to 4, and yet more preferably 1 to 3.

Examples of the alkanediylamino group include alkanediylamino groups having 1 to 11 carbon atoms, for example a methyleneamino group, an ethyleneimino group, a propyleneamino group and the like. The number of carbon atoms of the alkanediylamino group is preferably 1 to 8, more preferably 1 to 6, still more preferably 1 to 4, and yet more preferably 1 to 3.

Of these, examples of the group in which —CH₂— included in the alkanediyl group as for A^{a21} is replaced by —O—, —CO— or —NR^{a28}— include *—O—, *—CO—O—, *—O—CO—, *—CO—O— A^{a22}-CO—O—, *—O—CO-A^{a22}-O—, *—O-A^{a22}-CO—O—, *—CO—O-A^{a22}-O—CO—, *—O—CO-A^{a22}-O—CO— and *—CO—NR^{a28}—. Of these, *—CO—O—, *—CO—O— A^{a22}-CO—O—, or *—O-A^{a22}-CO—O— and *—CO—NR^{a28}— are preferable. Here, A^{a22} represents an alkanediyl group having 1 to 8 carbon atoms, and * represents a bonding site to carbon atoms to which R^{a2} is bonded. Examples of the alkanediyl group as for A^{a22} include the same alkanediyl group as for A^{a21} as long as the upper limit of the number of carbon atoms permit.

A^{a21} is preferably a single bond, *—CO—O— or *—CO—O— A^{a22}-CO—O—, more preferably a single bond, *—CO—O— or *—CO—O—CH₂—CO—O—, and still more preferably a single bond or *—CO— O—.

The hydrocarbon group in L^{a2} is an (na2+1)-valent hydrocarbon group, and examples thereof include cyclic hydrocarbon groups such as a linear or branched chain hydrocarbon group, a monocyclic or polycyclic (including a spiro ring, a fused ring or a bridged ring) alicyclic hydrocarbon group and an aromatic hydrocarbon group, and the hydrocarbon group may be groups obtained by combining two or more of these groups (e.g., a hydrocarbon group formed from an alicyclic hydrocarbon group or an aromatic hydrocarbon group and a chain hydrocarbon group).

Examples of the chain hydrocarbon group as for L^{a2} include di- to hexa-valent chain hydrocarbon groups such as an alkanediyl group, an alkanetriyl group, an alkanetetrayl group, an alkanepentayl group and an alkanehexayl group.

Examples of the alkanediyl group include the same alkanediyl groups as for A^{a21}.

Examples of the alkanetriyl group include a methanetriyl group, an ethanetriyl group, a propanetriyl group, a butanetriyl group, a pentanetriyl group, a hexanetriyl group, a heptanetriyl group, an octanetriyl group, a nonanetriyl group, a decanetriyl group, an undecanetriyl group, a dodecanetriyl group, a tridecanetriyl group, a tetradecanetriyl group, a pentadecanetriyl group, a hexadecanetriyl group and a heptadecanetriyl group.

Examples of the alkanetetrayl group include a methanetetrayl group, an ethanetetrayl group, a propanetetrayl group, a butanetetrayl group, a pentanetetrayl group, a hexanetetrayl group, a heptanetetrayl group, an octanetetrayl group, a nonanetetrayl group, a decanetetrayl group, an undecanetetrayl group, a dodecanetetrayl group, a tridecanetetrayl group, a tetradecanetetrayl group, a pentadecanetetrayl group, a hexadecanetetrayl group and a heptadecanetetrayl group.

Examples thereof also include groups in which one or more hydrogen atoms of the above-mentioned groups are substituted with a bonding site.

The number of carbon atoms of the chain hydrocarbon group is preferably 1 to 18, more preferably 1 to 12, still more preferably 1 to 10, yet more preferably 1 to 9, further preferably 1 to 8, still further preferably 1 to 6, yet further preferably 1 to 5, and particularly preferably 1 to 4.

Examples of the monocyclic and polycyclic alicyclic hydrocarbon group in L^{a21} include the following alicyclic hydrocarbon groups and the like. The bonding site can be any position.

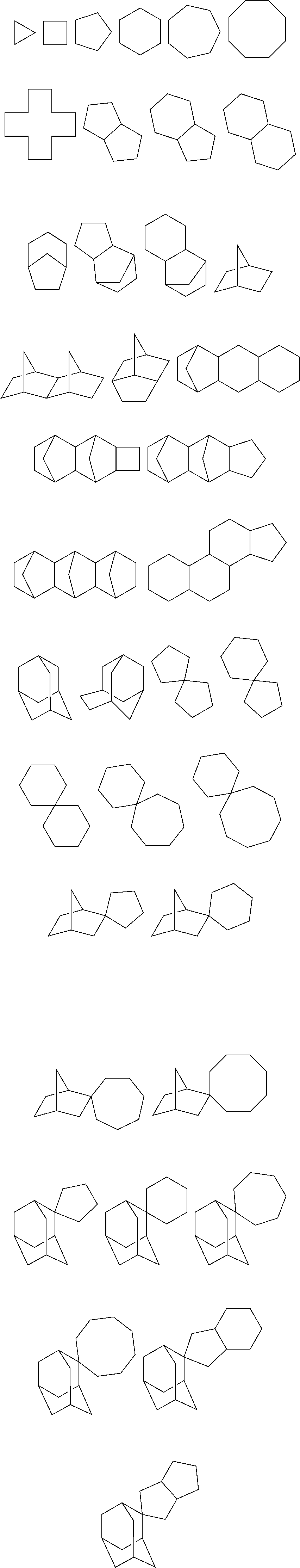

Examples thereof include di- to hexa-valent alicyclic hydrocarbon groups such as a cycloalkanediyl group, a cycloalkanetriyl group, a cycloalkanetetrayl group, a cycloalkanepentayl group and a cycloalkanehexayl group.

Specific examples thereof include monocyclic alicyclic hydrocarbon groups such as a cyclobutane-1,3-diyl group, a cyclopentane-1,3-diyl group, a cyclohexane-1,4-diyl group, a cyclooctane-1,5-diyl group, a cyclopropanetriyl group, a cyclobutanetriyl group, a cyclopentanetriyl group, a cyclohexanetriyl group, a cycloheptanetriyl group, a cyclooctanetriyl group, a cyclodecanetriyl group, a cyclopropanetetrayl group, a cyclobutanetetrayl group, a cyclopentanetetrayl group, a cyclohexanetetrayl group, a cycloheptanetetrayl group, a cyclooctanetetrayl group and a cyclodecanetetrayl group, and

polycyclic alicyclic hydrocarbon groups such as a norbornane-1,4-diyl group, a norbornane-2,5-diyl group, a adamantane-1,5-diyl group, an adamantane-2,6-diyl group, a decahydronaphthalenediyl group, a bicyclo[3.3.0]octanediyl group, a norbornanetriyl group, an adamantanetriyl group, a decahydronaphthalenetriyl group, a bicyclo[3.3.0]octanetriyl group, a norbornanetetrayl group, an adamantanetetrayl group, a decahydronaphthalenetetrayl group and a bicyclo[3.3.0]octanetetrayl group.

Examples thereof also include groups in which one or more hydrogen atoms of the above-mentioned groups are substituted with a bonding site.

The number of carbon atoms of the alicyclic hydrocarbon group is preferably 3 to 20, more preferably 3 to 18, still more preferably 3 to 16, and yet more preferably 3 to 12.

Examples of the aromatic hydrocarbon group in L^{a21} include di- to hexa-valent aromatic hydrocarbon groups such as an arylene group, an arenetriyl group, an arenetetrayl group, an arenepentayl group and an arenehexayl group.

Specific examples thereof include aromatic hydrocarbon groups such as a phenylene group, a naphthylene group, an anthrylene group, a biphenylene group, a phenanthrylene group, a benzenetriyl group, a naphthalenetriyl group, a anthracenetriyl group, a biphenylenetriyl group, a phenanthrenetriyl group, a benzenetetrayl group, a naphthalenetetrayl group, an anthracenetetrayl group, a biphenylenetetrayl group and a phenanthrenetetrayl group.

Examples thereof also include groups in which one or more hydrogen atoms of the above-mentioned groups are substituted with a bonding site.

The number of carbon atoms of the aromatic hydrocarbon group is preferably 6 to 20, more preferably 6 to 18, still more preferably 6 to 14, and yet more preferably 6 to 10.

Examples of the group obtained by combining two or more groups include a group obtained by combining an alicyclic hydrocarbon group with a chain hydrocarbon group, a group obtained by combining an aromatic hydrocarbon group with a chain hydrocarbon group, a group obtained by combining an alicyclic hydrocarbon group with an aromatic hydrocarbon group, and a group obtained by combining an alicyclic hydrocarbon group, a chain hydrocarbon group and an aromatic hydrocarbon group. In combination, two or more of alicyclic hydrocarbon groups, aromatic hydrocarbon groups and chain hydrocarbon groups may be respectively combined. Any group may be bonded to A^{a21} and L^{a22}.

Examples of the group in which —CH₂— included in the hydrocarbon group is replaced by —O—, —S—, —SO₂— or —CO— include a hydroxy group (a group in which —CH₂— included in methyl group is replaced by —O—), a carboxy group (a group in which —CH₂—CH₂— included in ethyl group is replaced by —O— CO—), a thiol group (a group in which —CH₂— included in methyl group is replaced by —S—), an alkoxy group (a group in which —CH₂— at any position included in alkyl group is replaced by —O—), an alkylthio group (a group in which —CH₂— at any position included in alkyl group is replaced by —S—), an alkoxycarbonyl group (a group in which —CH₂—CH₂— at any position included in the alkyl group is replaced by —O—CO—), an alkylsulfonyl group (a group in which —CH₂— at any position included in the alkyl group is replaced by —SO₂—), an alkylcarbonyl group (a group in which —CH₂— at any position included in the alkyl group is replaced by —CO—), an alkylcarbonyloxy group (a group in which —CH₂—CH₂— at any position included in the alkyl group is replaced by —CO—O—), an oxy group (a group in which —CH₂— included in the methylene group is replaced by —O—), a carbonyl group (a group in which —CH₂— included in the methylene group is replaced by —CO—), a thio group (a group in which —CH₂— included in methylene group is replaced by —S—), a sulfonyl group (a group in which —CH₂— included in the methylene group is replaced by —SO₂—), an alkanediyloxy group (a group in which —CH₂— at any position included in the alkanediyl group is replaced by —O—), an alkanediyloxycarbonyl group (a group in which —CH₂—CH₂— at any position included in the alkanediyl group is replaced by —O—CO—), an alkanediylcarbonyl group (a group in which —CH₂— at any position included in the alkanediyl group is replaced by —CO—), an alkanediylcarbonyloxy group (a group in which —CH₂—CH₂— at any position included in the alkanediyl group is replaced by —CO—O—), an alkanediylsulfonyl group (a group in which —CH₂— at any position included in the alkanediyl group is replaced by —SO₂—), an alkanediylthio group (a group in which —CH₂— at any position included in the alkanediyl group is replaced by —S—), a cycloalkoxy group, a cycloalkylalkoxy group, an alkoxycarbonyloxy group, an aromatic hydrocarbon group-carbonyloxy group, an aromatic hydrocarbon group-carbonyl group, an aromatic hydrocarbon group-oxy group, and groups obtained by combining two or more of these group. Examples thereof also include groups in which one or more hydrogen atoms of the above-mentioned groups are substituted with a bonding site. Examples of these replaced groups include the same groups as mentioned herein.

Examples of the group in which —CH₂— included in the alicyclic hydrocarbon group is replaced by —O—, —S—, —CO— or —SO₂— include the same groups as mentioned herein.

Examples of the group in which —CH₂— included in the combined group is replaced by —O—, —S—, —CO— or —SO₂— also include the following groups. The bonding site can be any position.

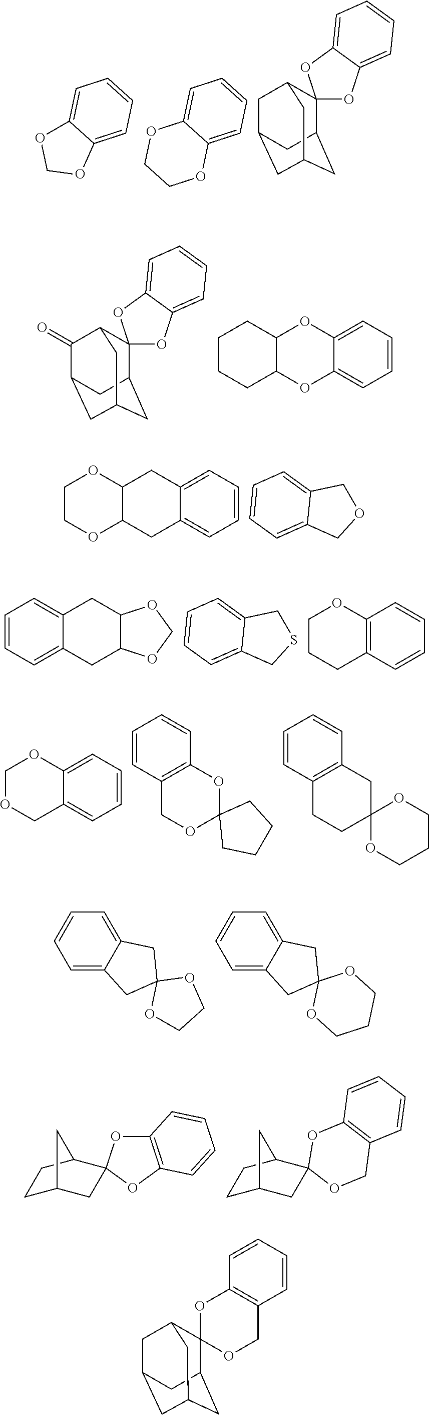

The hydrocarbon group in L^{a21} may have one or a plurality of substituents. Examples of the substituent include a halogen atom, a haloalkyl group having 1 to 12 carbon atoms, an alkyl group having 1 to 16 carbon atoms (—CH₂— included in the alkyl group may be replaced by —O— or —CO—), an acryloyloxy group or a methacryloyloxy group.

Examples of the halogen atom include a fluorine atom, a chlorine atom, a bromine atom, an iodine atom and the like.

The hydrocarbon group in L^{a21} can substantially have a substituent such as a haloalkyl group by having a halogen atom as the substituent. Examples of the haloalkyl group include an alkyl fluoride group, an alkyl chloride group, an alkyl bromide group, an alkyl iodide group, for example, a chloromethyl group, a bromomethyl group, an iodomethyl group, a fluoromethyl group, a difluoromethyl group, a trifluoromethyl group, a perfluoroethyl group, a perfluoropropyl group, a perfluorobutyl group and the like. The number of carbon atoms of the haloalkyl group is preferably 1 to 8, more preferably 1 to 6, still more preferably 1 to 4, and yet more preferably 1 to 3.

The hydrocarbon group as for L^{a21} can substantially have a substituent such as an alkyl group by including a branched structure in L^{a2}. Examples of the alkyl group include a methyl group, an ethyl group, an n-propyl group, an isopropyl group, an n-butyl group, a sec-butyl group, a tert-butyl group, a pentyl group, a hexyl group, a heptyl group, a 2-ethylhexyl group, an octyl group, a nonyl group, a decyl group, an undecyl group and a dodecyl group. The number of carbon atoms of the alkyl group is preferably 1 to 12, more preferably 1 to 10, still more preferably 1 to 8, yet more preferably 1 to 6, further preferably 1 to 4, and still further preferably 1 to 3.

L^{a21} can substantially have a substituent such as a hydroxy group, a carboxy group, an alkoxy group, an alkoxycarbonyl group, an alkylcarbonyl group, an alkylcarbonyloxy group, an alkoxyalkyl group, an alkoxyalkoxy group, an acryloyloxy group or a methacryloyloxy group, by the group in which —CH₂— included in the hydrocarbon group as for L^{a21} is replaced by —O— or —CO—. The number of carbon atoms of the alkoxy group is preferably 1 to 12, more preferably 1 to 6, still more preferably 1 to 4, and yet more preferably 1 to 3. The number of carbon atoms of the alkoxycarbonyl group, the alkylcarbonyl group and the alkylcarbonyloxy group is preferably 2 to 12, more preferably 2 to 6, still more preferably 2 to 4, and yet more preferably 2 to 3. The number of carbon atoms of the alkoxyalkyl group and the alkoxyalkoxy group is preferably 2 to 12, more preferably 2 to 8, still more preferably 2 to 6, and yet more preferably 2 to 4.

Examples of the above group include those which are the same as mentioned herein.

The substituent which may be possessed by the hydrocarbon group in L^{a21} is preferably a halogen atom, a haloalkyl group having 1 to 8 carbon atoms or an alkyl group having 1 to 12 carbon atoms (—CH₂— included in the alkyl group may be replaced by —O— or —CO—), more preferably a halogen atom, a haloalkyl group having 1 to 6 carbon atoms or an alkyl group having 1 to 10 carbon atoms (—CH₂— included in the alkyl group may be replaced by —O— or —CO—), still more preferably a halogen atom, a haloalkyl group having 1 to 4 carbon atoms, an alkyl group having 1 to 4 carbon atoms, a hydroxy group, alkoxy group having 1 to 4 carbon atoms or an alkoxyalkoxy group having 2 to 8 carbon atoms, yet more preferably a fluorine atom, an iodine atom, a trifluoromethyl group, a methyl group, a hydroxy group, a methoxy group, an ethoxy group, an ethoxyethoxy group or an ethoxymethoxy group, and still preferably a fluorine atom, an iodine atom, a trifluoromethyl group, a methyl group, a hydroxy group, a methoxy group or an ethoxyethoxy group.

When the alkanediyl group as for L^{a21} is replaced by —O— or —CO—, for example, it is also preferably *-L^{a23}-X^{a21}-(L^{a23} represents an alkanediyl group having 1 to 8 carbon atoms, X^{a21} represents —O—, —O—CO—, —CO—O— or —O—CO—O—, and * represents a bonding site to A^{a21}).

L^{a21} is preferably a single bond, a chain hydrocarbon group having 1 to 12 carbon atoms which may have a substituent (in which —CH₂— included in the chain hydrocarbon group may be replaced by —O— or —CO—), a cyclic hydrocarbon group having 3 to 20 carbon atoms which may have a substituent (in which —CH₂— included in the cyclic hydrocarbon group may be replaced by —O—, —S—, —CO— or —SO₂—), or groups obtained by combining a chain hydrocarbon group having 1 to 8 carbon atoms which may have a substituent with a cyclic hydrocarbon group having 3 to 20 carbon atoms which may have a substituent (in which —CH₂— included in the chain hydrocarbon group may be replaced by —O— or —CO—, and —CH₂— included in the cyclic hydrocarbon group may be replaced by —O—, —S—, —SO₂— or —CO—), and more preferably a chain hydrocarbon group having 1 to 10 carbon atoms which may have a substituent (in which —CH₂— included in the chain hydrocarbon group may be replaced by —O— or —CO—), a cyclic hydrocarbon group having 3 to 18 carbon atoms which may have a substituent (in which —CH₂— included in the cyclic hydrocarbon group may be replaced by —O—, —S—, —CO— or —SO₂—), or groups obtained by combining a chain hydrocarbon group having 1 to 6 carbon atoms which may have a substituent with a cyclic hydrocarbon group having 3 to 18 carbon atoms which may have a substituent (in which —CH₂— included in the chain hydrocarbon group may be replaced by —O— or —CO—, and —CH₂— included in the cyclic hydrocarbon group may be replaced by —O—, —S—, —SO₂— or —CO—).

Examples of the chain hydrocarbon group having 1 to 12 carbon atoms as for L^{a22} include linear alkanediyl groups such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group and a dodecane-1,12-diyl group; and branched alkanediyl groups such as an ethane-1,1-diyl group, a propane-1,1-diyl group, a propane-1,2-diyl group, a propane-2,2-diyl group, a 1-methylpropane-1,3-diyl group, a 2-methylpropane-1,3-diyl group, a 2-methylpropane-1,2-diyl group, a 1-dimethylpropane-1,3-diyl group, a pentane-2,4-diyl group, a pentane-1,4-diyl group and a 2-methylbutane-1,4-diyl group. The number of carbon atoms of the chain hydrocarbon group is preferably 1 to 10, more preferably 1 to 8, still more preferably 1 to 6, and yet more preferably 1 to 4.

Examples of the group in which —CH₂— included in the chain hydrocarbon group is replaced by —O— or —CO— include the same groups as mentioned herein.

The number of fluorine atoms possessed by L^{a22} may be either 1, or 2 or more.

na2 is preferably an integer of 1 to 4, and more preferably an integer of 1 to 3.

Examples of the structural unit (a2) when L^{a21} is a single bond or a chain hydrocarbon group include the following structural units. Of the following structural units, the structural unit in which the methyl group corresponding to R^{a2} is substituted with a hydrogen atom or the like is preferable structural unit of the structural unit (a2), similarly to the following structural units.

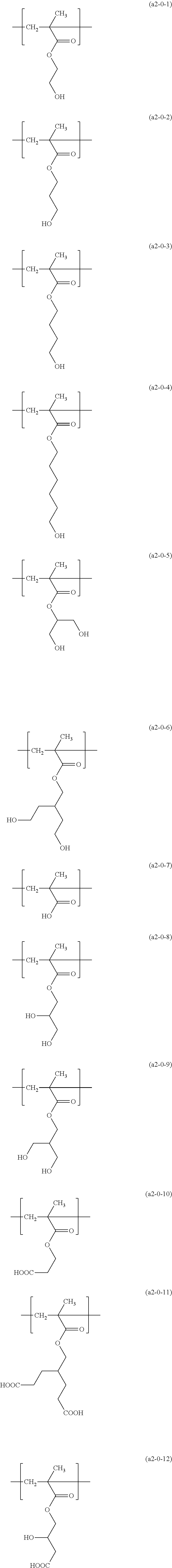

When a resist pattern is produced from the resist composition of the present invention, in the case of using, as an exposure source, high energy rays such as KrF excimer laser (248 nm), electron beam or extreme ultraviolet light (EUV), a structural unit (a2) having a phenolic hydroxy group is preferably used as the structural unit (a2), and the below-mentioned structural unit (a2-A) is more preferably used. When using ArF excimer laser (193 nm) or the like, a structural unit (a2) having an alcoholic hydroxy group is preferably used as the structural unit (a2), and more preferably a structural unit (a2-1) mentioned below. The structural unit (a2) may be included alone, or two or more structural units may be included.

In the structural unit (a2), examples of the structural unit having a phenolic hydroxy group include a structural unit represented by formula (a2-A) (hereinafter sometimes referred to as “structural unit (a2-A)”):

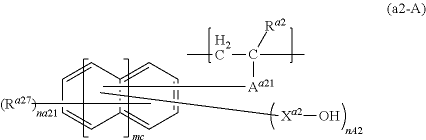

wherein, in formula (a2-A), all symbols are the same as defined above.

Examples of R^{a2} and A^{a21} include the same groups as mentioned in formula (a2), respectively.

Examples of the halogen atom as for R^{a27} include a fluorine atom, a chlorine atom, an iodine atom and a bromine atom.

Examples of the alkyl group having 1 to 6 carbon atoms which may have a halogen atom as for R^{a27} include a trifluoromethyl group, a difluoromethyl group, a methyl group, a perfluoroethyl group, a 2,2,2-trifluoroethyl group, a 1,1,2,2-tetrafluoroethyl group, an ethyl group, a perfluoropropyl group, a 2,2,3,3,3-pentafluoropropyl group, a propyl group, a perfluorobutyl group, a 1,1,2,2,3,3,4,4-octafluorobutyl group, a butyl group, a perfluoropentyl group, a 2,2,3,3,4,4,5,5,5-nonafluoropentyl group, a pentyl group, a hexyl group, a perfluorohexyl group, a chloromethyl group, a bromomethyl group and an iodomethyl group. The number of carbon atoms of the alkyl group is preferably 1 to 4, more preferably 1 to 3, still more preferably a methyl group or an ethyl group, and yet more preferably methyl group.

Examples of the alkoxy group as for R^{a27} include a methoxy group, an ethoxy group, a propoxy group, an isopropoxy group, a butoxy group, a sec-butoxy group and a tert-butoxy group. The number of carbon atoms of the alkoxy group is preferably 1 to 4, more preferably 1 to 3, still more preferably a methoxy group or an ethoxy group, and yet more preferably a methoxy group.

Examples of the alkoxyalkyl group as for R^{a27} include a methoxymethyl group, an ethoxyethyl group, a propoxymethyl group, an isopropoxymethyl group, a butoxymethyl group, a sec-butoxymethyl group and a tert-butoxymethyl group. The alkoxyalkyl group is preferably an alkoxyalkyl group having 2 to 8 carbon atoms, more preferably a methoxymethyl group or an ethoxyethyl group, and still more preferably a methoxymethyl group.

Examples of the alkoxyalkoxy group as for R^{a27} include a methoxymethoxy group, a methoxyethoxy group, an ethoxymethoxy group, an ethoxyethoxy group, a propoxymethoxy group, an isopropoxymethoxy group, a butoxymethoxy group, a sec-butoxymethoxy group and a tert-butoxymethoxy group. The alkoxyalkoxy group is preferably an alkoxyalkoxy group having 2 to 8 carbon atoms, and more preferably a methoxyethoxy group or an ethoxyethoxy group.

Examples of the alkylcarbonyl group as for R^{a27} include an acetyl group, a propionyl group and a butyryl group. The alkylcarbonyl group is preferably an alkylcarbonyl group having 2 to 3 carbon atoms, and more preferably an acetyl group.

Examples of the alkylcarbonyloxy group as for R^{a27} include an acetyloxy group, a propionyloxy group and a butyryloxy group. The alkylcarbonyloxy group is preferably an alkylcarbonyloxy group having 2 to 3 carbon atoms, and more preferably an acetyloxy group.

R^{a27} is preferably a halogen atom, a hydroxy group, an alkyl group having 1 to 4 carbon atoms, an alkoxy group having 1 to 4 carbon atoms or an alkoxyalkoxy group having 2 to 8 carbon atoms, more preferably a fluorine atom, an iodine atom, a hydroxy group, a methyl group, a methoxy group, an ethoxy group, an ethoxyethoxy group or an ethoxymethoxy group, and still more preferably a fluorine atom, an iodine atom, a hydroxy group, a methyl group, a methoxy group or an ethoxyethoxy group.

nA2 may be 1, 2, 3 or 4 and is preferably 1, 2 or 3, and more preferably 1 or 2.

na22 is preferably 0, 1 or 2, and more preferably 0 or 1.

At least one hydroxy group is preferably bonded at the meta-position or the para-position, and more preferably at the meta-position of the benzene ring, with respect to the bonding site of A^{a21}. When two hydroxy groups are bonded to the benzene ring, each hydroxy group is preferably bonded at the meta-position and the para-position. In the case of the naphthalene ring, it may be bonded at any of the 2-position to the 8-position with respect to the 1-position which is the bonding site of A^{a21}, and preferably bonded at the 3-position or the 4-position.

Examples of the structural unit (a2-A) include structural units derived from the monomers mentioned in JP 2010-204634 A and JP 2012-12577 A.

Examples of the structural unit (a2-A) include structural units represented by formula (a2-2-1) to formula (a2-2-32) and formula (a2-3-1) to formula (a2-3-24) and a structural unit in which a methyl group corresponding to R^{a2} in the structural unit (a2-A) is substituted with a hydrogen atom, a halogen atom, a haloalkyl group or other alkyl groups in these structural units.

[chemical structure]

[chemical structure]

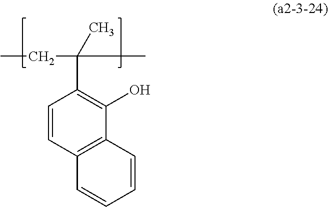

When the structural unit (a2-A) is included in the resin (A) or the like, the content of the structural unit (a2-A) is preferably 5 mol % or more, more preferably 10 mol % or more, still more preferably 15 mol or more, and yet more preferably 20 mol % or more, based on all structural units. The content is also preferably 80 mol % or less, more preferably 70 mol % or less, still more preferably 65 mol % or less, yet more preferably 60 mol % or less, further preferably 50 mol % or less, still further preferably 45 mol % or less, and yet further preferably 40 mol % or less. Specifically, the content is preferably 5 to 80 mol %, more preferably 10 to 70 mol %, still more preferably 15 to 65 mol %, and yet more preferably 20 to 65 mol %.

The structural unit (a2-A) can be included in the resin (A) by polymerizing, for example, with a structural unit (a1-4) and treating with an acid such as p-toluenesulfonic acid. The structural unit (a2-A) can also be included in the resin (A) by polymerizing with acetoxystyrene and treating with an alkali such as tetramethylammonium hydroxide.

Example of the structural unit (a2), when L^{a21} is a cyclic hydrocarbon group, structural units having an alcoholic hydroxyl group or a carboxyl group include the structural unit represented by formula (a2-B) (hereinafter sometimes referred to as “structural unit (a2-B)”), the structural unit represented by formula (a2-C) (hereinafter sometimes referred to as “structural unit (a2-C)”), and the structural unit represented by formula (a2-D) (hereinafter sometimes referred to as “structural unit (a2-D)”).

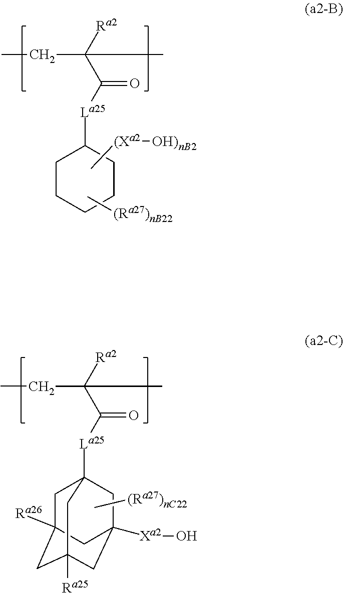

[In formulas (a2-B) and (a2-C),

R^{a2} has the same meaning as in formula (a2).

R^{a27} has the same meaning as in formula (a2-A).

L^{a25} represents —O— or *—O—(CH₂)ₖ₂—CO—O—, where k2 represents an integer from 1 to 7. The * indicates the binding site with —CO—.

X^{a2} represents a single bond or —CO—.

R^{a25} and R^{a26} each independently represent a hydrogen atom, a methyl group, or a hydroxyl group.

nB2 represents an integer from 1 to 5. When nB2 is 2 or more, multiple X^{a2} may be identical or different.

nB22 represents an integer from 0 to 8. When nB22 is 2 or more, multiple Ra27 may be identical or different. nC22 represents an integer from 0 to 10. When nC22 is 2 or more, multiple Ra27 may be identical or different.]

L^{a25} is preferably —O— or —O—(CH₂)_{f1}—CO—O— (where f1 represents an integer from 1 to 4), and more preferably —O—.

R^{a2} is preferably a methyl group.

X^{a2} is preferably a single bond.

R^{a25} is preferably a hydrogen atom.

R^{a26} is preferably a hydrogen atom or a hydroxyl group.

R^{a27} is preferably a halogen atom, a hydroxyl group, a carboxyl group, an alkyl group having 1 to 4 carbon atoms which may have a halogen atom, an alkoxy group having 1 to 4 carbon atoms, or an alkoxyalkoxy group having 2 to 8 carbon atoms, more preferably a fluorine atom, an iodine atom, a hydroxyl group, a methyl group, a methoxy group, an ethoxy group, an ethoxyethoxy group, or an ethoxymethoxy group, and even more preferably a fluorine atom, an iodine atom, a hydroxyl group, a methyl group, a methoxy group, or an ethoxyethoxy group.

nB2 is preferably 1, 2, 3, or 4, more preferably 1, 2, or 3, and even more preferably 1 or 2.

nB22 is preferably an integer from 0 to 3, more preferably 0, 1, or 2, and even more preferably 0 or 1. nC22 is preferably an integer from 0 to 6, more preferably an integer from 0 to 3, and even more preferably 0 or 1.

As structural units (a2-B) and (a2-C), for example, structural units derived from the monomers described in JP 2010-204646 A, the following structural units, and structural units in which a methyl group or a hydrogen atom corresponding to Ra2 in the following structural units is replaced with a hydrogen atom, a halogen atom, a haloalkyl group, or another alkyl group can be mentioned. Among them, structural units represented by any of formulas (a2-B-1) to (a2-B-5) and formulas (a2-C-1) to (a2-C-9) are preferred.

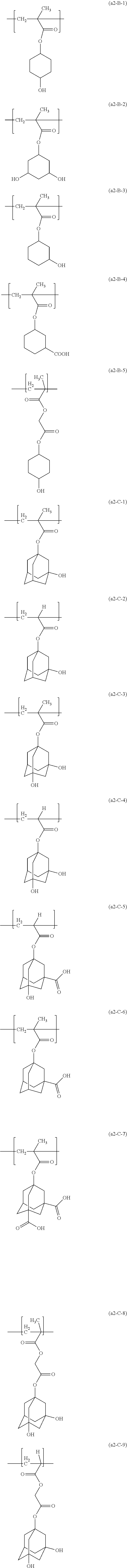

When the resin (A) or the like contains the structural unit (a2-B) or the structural unit (a2-C), the content thereof is 1 mol % or more relative to the total structural units of the resin (A) or the like, and preferably 2 mol % or more. Additionally, it is 45 mol % or less, preferably 40 mol % or less, more preferably 35 mol % or less, even more preferably 20 mol % or less, and most preferably 10 mol % or less. Specifically, it is 1 to 45 mol %, preferably 1 to 40 mol %, more preferably 1 to 35 mol %, even more preferably 1 to 20 mol %, and most preferably 1 to 10 mol %.

As the structural unit (a2-D), it is more preferable that it is a structural unit represented by the following formula (a2-D1) (hereinafter sometimes referred to as ‘structural unit (a2-D1)’).

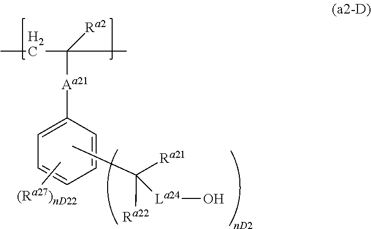

[In formula (a2-D),

R^{a2} and A^{a21} have the same meanings as in formula (a2).

R^{a27} has the same meaning as in formula (a2-A).

R^{a21} and R^{a22} each independently represent a fluoroalkyl group having 1 to 4 carbon atoms.

L^{a24} represents a single bond or an alkylene group having 1 to 3 carbon atoms, which may be substituted with a fluorine atom.

nD2 represents an integer from 1 to 5. When nD2 is 2 or more, multiple groups within parentheses may be identical or different.

nD22 represents an integer from 0 to 4. When nD22 is 2 or more, multiple Ra27 groups may be identical or different. However, 1 nD2+nD22 5.]

As the fluoroalkyl groups for R^{a21} and R^{a22}, examples include trifluoromethyl group, 2,2,2-trifluoroethyl group, 3,3,3-trifluoropropyl group, and 4,4,4-trifluorobutyl group. Trifluoromethyl group is preferred for R^{a21} and R^{a22}.

As the alkylene groups for L^{a24}, examples include methylene group, ethan-1,1-diyl group, propan-1,1-diyl group, and propan-2,2-diyl group.

L^{a24} is preferably a single bond or a methylene group.

R^{a27} is preferably a halogen atom, a hydroxyl group, a carboxyl group, an alkyl group having 1 to 4 carbon atoms which may have a halogen atom, an alkoxy group having 1 to 4 carbon atoms, or an alkoxyalkoxy group having 2 to 8 carbon atoms, more preferably a fluorine atom, an iodine atom, a hydroxyl group, a methyl group, a methoxy group, an ethoxy group, an ethoxyethoxy group, or an ethoxymethoxy group, and even more preferably a fluorine atom, an iodine atom, a hydroxyl group, a methyl group, a methoxy group, or an ethoxyethoxy group.

nD2 is preferably 1, 2, 3, or 4, more preferably 1, 2, or 3, even more preferably 1 or 2, and most preferably 1. Additionally, it is preferred that nD2 is 1 and the group within parentheses is bonded at the para position.

nD22 is preferably an integer from 0 to 3, more preferably an integer from 0 to 2, even more preferably 0 or 1, and most preferably 0.

As the structural unit (a2-D), it is more preferable that it is a structural unit represented by the following formula (a2-D1) (hereinafter sometimes referred to as ‘structural unit (a2-D1)’).

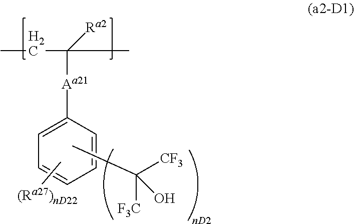

[In formula (a2-D1),

R^{a2}, A^{a21}, R^{a27}, nD2, and nD22 each have the same meanings as in formula (a2-D).]

In formula (a2-D1), R^{a2} is preferably a hydrogen atom or a methyl group.

A^{a21} is preferably a single bond.

As the structural unit (a2-D), the following structural units can be mentioned.

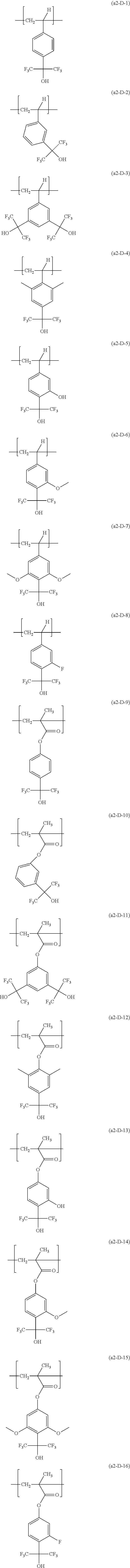

In the structural units represented by formulas (a2-D-1) to (a2-D-8), structural units in which the hydrogen atom corresponding to Ra2 is replaced by a methyl group or the like, and in the structural units represented by formulas (a2-D-9) to (a2-D-16), structural units in which the methyl group corresponding to Ra2 is replaced by a hydrogen atom or the like, can also be cited as specific examples of the structural unit (a2-D). Among them, the structural units represented by formulas (a2-D-1) to (a2-D-8) are preferred, the structural units represented by formulas (a2-D-1) to (a2-D-4) are more preferred, and the structural unit represented by formula (a2-D-1) is even more preferred.

When the resin (A) or the like contains the structural unit (a2-D), the content thereof is preferably 3 mol % or more relative to the total structural units of the resin (A) or the like, more preferably 5 mol % or more, and even more preferably 10 mol % or more. Additionally, it is preferably 80 mol % or less, more preferably 75 mol % or less, even more preferably 70 mol % or less, and most preferably 65 mol % or less. Specifically, it is preferably 3 to 80 mol %, more preferably 5 to 75 mol %, even more preferably 10 to 70 mol %, and most preferably 10 to 65 mol %.

<Structural Unit (a3)>

The lactone ring possessed by the structural unit (a3) may be a monocyclic ring such as a β-propiolactone ring, a γ-butyrolactone ring or a δ-valerolactone ring, or a fused ring of a monocyclic lactone ring and the other ring. Preferably, a γ-butyrolactone ring, an adamantanelactone ring or a bridged ring including a γ-butyrolactone ring structure (e.g., a structural unit represented by the following formula (a3-2)) is exemplified.

The structural unit (a3) is preferably a structural unit represented by formula (a3-1), formula (a3-2), formula (a3-3) or formula (a3-4). These structural units may be included alone, or two or more structural units may be included:

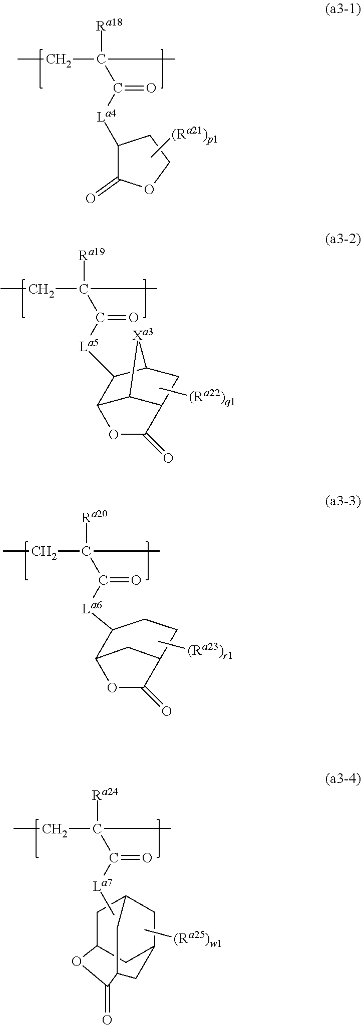

wherein, in formula (a3-1), formula (a3-2), formula (a3-3) and formula (a3-4),
L^{a4}, L^{a5} and L^{a6} each independently represent —O— or a group represented by *—O—(CH₂)ₖ₃—CO—O— (k3 represents an integer of 1 to 7),
L^{a7} represents —O—, *—O-L^{aB}-O—, *—O-L^{a8}-CO—O—, *—O— L^{a8}-CO—O-L^{a9}-CO—O— or *—O-L^{a8}-O—CO-L^{a9}_0,
L^{a8} and L^{a9} each independently represent an alkanediyl group having 1 to 6 carbon atoms,
* represents a bonding site to a carbonyl group,
R^{a18}, R^{a19}, R^{a20} and R^{a24} each independently represent a hydrogen atom, a halogen atom, or an alkyl group having 1 to 6 carbon atoms which may have a halogen atom,
X^{a3} represents —CH₂— or an oxygen atom,
R^{a21} represents an aliphatic hydrocarbon group having 1 to 4 carbon atoms,
R^{a22}, R^{a23} and R^{a25} each independently represent a carboxy group, a cyano group or an aliphatic hydrocarbon group having 1 to 4 carbon atoms,
p1 represents an integer of 0 to 5,
q1 represents an integer of 0 to 3,
r1 represents an integer of 0 to 3,
w1 represents an integer of 0 to 8, and
when p1, q1, r1 and/or w1 are 2 or more, a plurality of R^{a2}, R^{a22}, R^{a23} and/or R^{a25} may be the same or different from each other.

Examples of the aliphatic hydrocarbon group in R^{a2}, R^{a22}, R^{a23} and R^{a25} include alkyl groups such as a methyl group, an ethyl group, a propyl group, an isopropyl group, a butyl group, a sec-butyl group and a tert-butyl group.

Examples of the halogen atom in R^{a18}, R^{a19}, R^{a20} and R^{a24} include a fluorine atom, a chlorine atom, a bromine atom and an iodine atom.

Examples of the alkyl group in R^{a18}, R^{a19}, R^{a20} and R^{a24} include a methyl group, an ethyl group, a propyl group, an isopropyl group, a butyl group, a sec-butyl group, a tert-butyl group, a pentyl group and a hexyl group, and the alkyl group is preferably an alkyl group having 1 to 4 carbon atoms, and more preferably a methyl group or an ethyl group.

Examples of the alkyl group having a halogen atom in R^{a18}, R^{a19}, R^{a20} and R^{a24} include a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group, a perfluorohexyl group, a trichloromethyl group, a tribromomethyl group, a triiodomethyl group and the like.

Examples of the alkanediyl group in L^{as} and L^{a9} include a methylene group, an ethylene group, a propane-1,3-diyl group, a propane-1,2-diyl group, a butane-1,4-diyl group, a pentane-1,5-diyl group, a hexane-1,6-diyl group, a butane-1,3-diyl group, a 2-methylpropane-1,3-diyl group, a 2-methylpropane-1,2-diyl group, a pentane-1,4-diyl group and a 2-methylbutane-1,4-diyl group.

In formula (a3-1) to formula (a3-3), preferably, L^{a4} to L^{a6} are each independently —O— or a group in which k3 is an integer of 1 to 4 in *—O—(CH₂)ₖ₃—CO—O—, more preferably —O— and *—O—CH₂—CO—O—, and still more preferably an oxygen atom,
R^{a1s} to R^{a21} are preferably a methyl group,
preferably, R^{a22} and R^{a23} are each independently a carboxy group, a cyano group or a methyl group, and
preferably, p1, q1 and r1 are each independently an integer of 0 to 2, and more preferably 0 or 1.

In formula (a3-4), R^{a24} is preferably a hydrogen atom or an alkyl group having 1 to 4 carbon atoms, more preferably a hydrogen atom, a methyl group or an ethyl group, and still more preferably a hydrogen atom or a methyl group,
R^{a25} is preferably a carboxy group, a cyano group or a methyl group,
L^{a7} is preferably —O— or *—O-L^{aB}-CO—O—, and more preferably —O—, —O—CH₂—CO—O— or —O—C₂H₄—CO—O—, and
w1 is preferably an integer of 0 to 2, and more preferably 0 or 1.

Particularly, formula (a3-4) is preferably formula (a3-4)′:

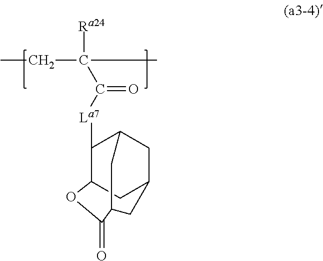

wherein R^{a24} and L^{a7} are the same as defined above.

Examples of the structural unit (a3) include structural units derived from the monomers mentioned in JP 2010-204646 A, the monomers mentioned in JP 2000-122294 A and the monomers mentioned in JP 2012-41274 A. The structural unit (a3) is preferably a structural unit represented by any one of formula (a3-1-1), formula (a3-1-2), formula (a3-2-1), formula (a3-2-2), formula (a3-3-1), formula (a3-3-2) and formula (a3-4-1) to formula (a3-4-12), and structural units in which methyl groups corresponding to R^{a18}, R^{a19}, R^{a20} and R^{a24} in formula (a3-1) to formula (a3-4) are substituted with hydrogen atoms in the above structural units.

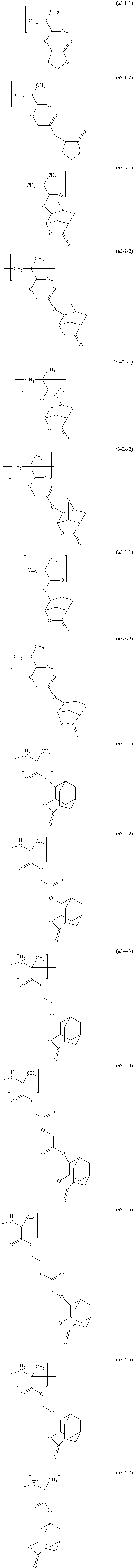

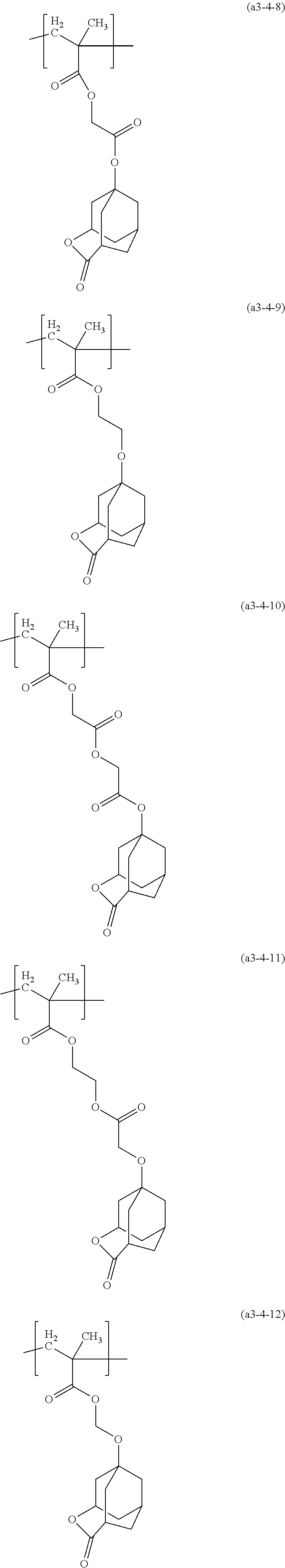

When the resin (A) or the like includes the structural unit (a3), the total content is 1 mol % or more, preferably 3 mol % or more, more preferably 5 mol % or more, and still more preferably 10 mol %, based on all structural units of the resin (A). The content is also 70 mol % or less, preferably 65 mol % or less, more preferably 60 mol % or less, still more preferably 50 mol % or less, yet more preferably 40 mol % or less, further preferably 30 mol % or less, still further preferably 25 mol % or less, and yet further preferably 20 mol % or less. Specifically, the content is 1 to 70 mol %, preferably 3 to 70 mol %, more preferably 3 to 65 mol %, still more preferably 5 to 65 mol %, yet more preferably 5 to 60 mol %, and further preferably 10 to 60 mol %.

Each content of the structural unit (a3-1), the structural unit (a3-2), the structural unit (a3-3) or the structural unit (a3-4) is preferably 1 mol % or more, more preferably 3 mol % or more, still more preferably 5 mol % or more, and yet more preferably 10 mol % or more, based on all structural units of the resin (A) or the like. The content is also preferably 60 mol % or less, more preferably 55 mol % or less, still more preferably 50 mol % or less, further preferably 40 mol % or less, still further preferably 30 mol % or less, yet further preferably 25 mol % or less, and yet still further preferably 20 mol % or less. Specifically, the content is preferably 1 to 60 mol %, more preferably 3 to 50 mol %, still more preferably 5 to 50 mol %, and yet more preferably 10 to 50 mol %.

<Structural Unit (a4)>

The structural unit represented by formula (a4) is shown below:

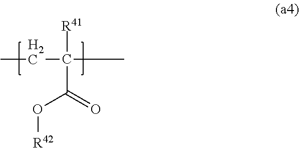

wherein, in formula (a4),
R⁴¹ represents a hydrogen atom or a methyl group, and
R⁴² represents a saturated hydrocarbon group having 1 to 24 carbon atoms which has a halogen atom, and —CH₂— included in the saturated hydrocarbon group may be replaced by —O— or —CO—.

Examples of the chain saturated hydrocarbon group represented by R⁴² include a chain saturated hydrocarbon group and a monocyclic or polycyclic alicyclic saturated hydrocarbon group, and groups formed by combining these groups.

Examples of the chain saturated hydrocarbon group include a methyl group, an ethyl group, a propyl group, a butyl group, a pentyl group, a hexyl group, a heptyl group, an octyl group, a decyl group, a dodecyl group, a pentadecyl group, a hexadecyl group, a heptadecyl group and an octadecyl group.

Examples of the monocyclic or polycyclic alicyclic saturated hydrocarbon group include cycloalkyl groups such as a cyclopentyl group, a cyclohexyl group, a cycloheptyl group and a cyclooctyl group; and polycyclic alicyclic saturated hydrocarbon groups such as a decahydronaphthyl group, an adamantyl group, a norbornyl group, and the following groups (* represents a bonding site).

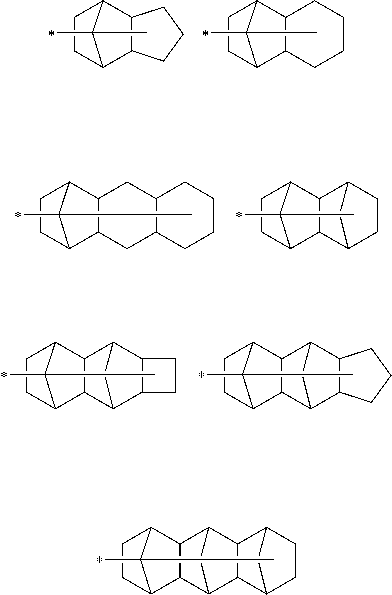

Examples of the groups formed by combination include groups formed by combining one or more alkyl groups or one or more alkanediyl groups with one or more alicyclic saturated hydrocarbon groups, for example, an -alkanediyl group-alicyclic saturated hydrocarbon group, an -alicyclic saturated hydrocarbon group-alkyl group, an -alkanediyl group-alicyclic saturated hydrocarbon group-alkyl group and the like.

Examples of the alkanediyl group include linear alkanediyl groups such as a methylene group, an ethylene group, a propane-1,3-diyl group, a butane-1,4-diyl group, a pentane-1,5-diyl group and a hexane-1,6-diyl group; and branched alkanediyl groups such as a propane-1,2-diyl group, a butane-1,3-diyl group, a 2-methylpropane-1,2-diyl group, a 1-methylbutane-1,4-diyl group and a 2-methylbutane-1,4-diyl group. The end of the branched alkanediyl group may be a methyl group.

Examples of the halogen atom possessed by the saturated hydrocarbon group included in R⁴² include a fluorine atom, a chlorine atom, a bromine atom or an iodine atom.

Examples of the group in which —CH₂— of the saturated hydrocarbon group included in R⁴² is replaced by —O— or —CO— include a hydroxy group (a group in which —CH₂— included in the methyl group is replaced by —O—), a carboxy group (a group in which —CH₂—CH₂— included in the ethyl group is replaced by —O—CO—), a carbonyl group (a group in which —CH₂— included in the methylene group is replaced by —CO—), an oxy group (a group in which —CH₂— included in the methylene group is replaced by —O—), an alkoxy group (a group in which —CH₂— at any position included in the alkyl group is replaced by —O—), an alkoxycarbonyl group (a group in which —CH₂—CH₂— at any position included in the alkyl group is replaced by —O— CO—), an alkylcarbonyl group (a group in which —CH₂— at any position included in the alkyl group is replaced by —CO—), an alkylcarbonyloxy group (a group in which —CH₂—CH₂— at any position included in the alkyl group is replaced by —CO—O—), an alkanediyloxy group (a group in which —CH₂— at any position included in the alkanediyl group is replaced by —O—), an alkanediyloxycarbonyl group (a group in which —CH₂—CH₂— at any position included in the alkanediyl group is replaced by —O—CO—), an alkanediylcarbonyl group (a group in which —CH₂— at any position included in the alkanediyl group is replaced by —CO—), an alkanediylcarbonyloxy group (a group in which —CH₂—CH₂— at any position included in the alkanediyl group is replaced by —CO—O—), a cycloalkoxy group, a cycloalkylalkoxy group, groups obtained by combining two or more of these groups and the like.

The structural unit (a4) is preferably a structural unit in which R⁴² is a saturated hydrocarbon group having a fluorine atom, and an example of the structural unit (a4) in which R⁴² is a saturated hydrocarbon group having a fluorine atom includes a structural unit represented by formula (a4-1) (hereinafter sometimes referred to as structural unit (a4-1)), a structural unit represented by formula (a4-2) (hereinafter sometimes referred to as structural unit (a4-2)) and a structural unit represented by formula (a4-3) (hereinafter sometimes referred to as structural unit (a4-3)).

The structural unit represented by formula (a4-1) is a structural unit represented by the following:

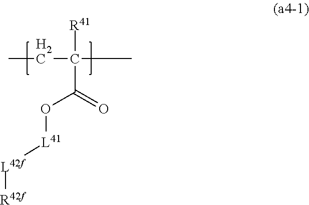

wherein, in formula (a4-1),
R⁴¹ represents a hydrogen atom or a methyl group,
L⁴¹ represents a single bond or an alkanediyl group having 1 to 4 carbon atoms,
L^{42f} represents an alkanediyl group having 1 to 8 carbon atoms which has a fluorine atom, or a cycloalkanediyl group having 3 to 12 carbon atoms which has a fluorine atom, and
R^{42f} represents a hydrogen atom or a fluorine atom.

Examples of the alkanediyl group in L⁴¹ include linear alkanediyl groups such as a methylene group, an ethylene group, a propane-1,3-diyl group and a butane-1,4-diyl group; and branched alkanediyl groups such as an ethane-1,1-diyl group, a propane-1,2-diyl group, a butane-1,3-diyl group, a 2-methylpropane-1,3-diyl group and a 2-methylpropane-1,2-diyl group. The end of the branched alkanediyl group may be a methyl group.

Examples of the alkanediyl group having a fluorine atom in L^{42f} include linear alkanediyl groups having a fluorine atom, such as a methylene group, an ethylene group having a fluorine atom, a propane-1,3-diyl group having a fluorine atom, a butane-1,4-diyl group having a fluorine atom, a pentane-1,5-diyl group having a fluorine atom, a hexane-1,6-diyl group having a fluorine atom, a heptane-1,7-diyl group having a fluorine atom and an octane-1,8-diyl group having a fluorine atom; and branched alkanediyl group having a fluorine atom, such as an ethane-1,1-diyl group having a fluorine atom, a propane-1,1-diyl group having a fluorine atom, a propane-1,2-diyl group having a fluorine atom, a propane-2,2-diyl group having a fluorine atom, a pentane-2,4-diyl group having a fluorine atom, a 2-methylpropane-1,3-diyl group having a fluorine atom, a 2-methylpropane-1,2-diyl group having a fluorine atom, a pentane-1,4-diyl group having a fluorine atom and a 2-methylbutane-1,4-diyl group having a fluorine atom. The end of the branched alkanediyl group may be a methyl group which may have a fluorine atom.

Examples of cycloalkanediyl group having a fluorine atom in L^{42f} include monocyclic cycloalkanediyl groups such as a cyclobutane-1,3-diyl group having a fluorine atom, a cyclopentane-1,3-diyl group having a fluorine atom, a cyclohexane-1,4-diyl group having a fluorine atom, a cyclohexene-3,6-diyl group having a fluorine atom and a cyclooctane-1,5-diyl group having a fluorine atom, and

polycyclic cycloalkanediyl groups having a fluorine atom, such as a norbornane-1,4-diyl group having a fluorine atom, a norbornane-2,5-diyl group having a fluorine atom, a 5-norbornene-2,3-diyl group having a fluorine atom, an adamantane-1,5-diyl group having a fluorine atom, and an adamantane-2,6-diyl group having a fluorine atom.

The number of fluorine atoms of the alkanediyl group and the cycloalkanediyl group each having a fluorine atom in L^{42f} may be 1 or more, and preferably 2 or more.

The alkanediyl group and the cycloalkanediyl group each having a fluorine atom in L^{42f} are preferably a perfluoroalkanediyl group and a perfluorocycloalkanediyl group, respectively.

Examples of the perfluoroalkanediyl group in L^{42f} include a difluoromethylene group, a perfluoroethylene group, a perfluoroethylfluoromethylene group, a perfluoropropane-1,3-diyl group, a perfluoropropane-1,2-diyl group, a perfluoropropane-2,2-diyl group, a perfluorobutane-1,4-diyl group, a perfluorobutane-2,2-diyl group, a perfluorobutane-1,2-diyl group, a perfluoropentane-1,5-diyl group, a perfluoropentane-2,2-diyl group, a perfluoropentane-3,3-diyl group, a perfluorohexane-1,6-diyl group, a perfluorohexane-2,2-diyl group, a perfluorohexane-3,3-diyl group, a perfluoroheptane-1,7-diyl group, a perfluoroheptane-2,2-diyl group, a perfluoroheptane-3,4-diyl group, a perfluoroheptane-4,4-diyl group, a perfluorooctane-1,8-diyl group, a perfluorooctane-2,2-diyl group, a perfluorooctane-3,3-diyl group, a perfluorooctane-4,4-diyl group and the like.

Examples of the perfluorocycloalkanediyl group in L^{42f} include a perfluorocyclohexanediyl group, a perfluorocyclopentanediyl group, a perfluorocycloheptanediyl group, a perfluoroadamantanediyl group and the like.

L⁴¹ is preferably a single bond, a methylene group or an ethylene group, and more preferably a single bond or a methylene group.

L^{42f} is preferably a perfluoroalkanediyl group having 1 to 6 carbon atoms, and more preferably a perfluoroalkanediyl group having 1 to 3 carbon atoms.

Examples of the structural unit (a4-1) include the following structural units, and structural units in which a methyl group corresponding to R⁴¹ in the structural unit (a4-1) in the following structural units is substituted with a hydrogen atom.

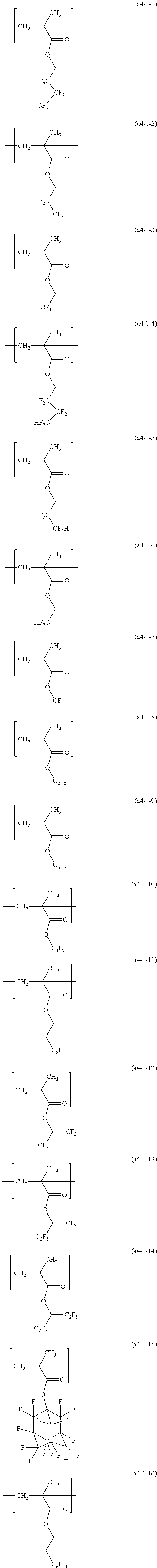

Examples of the structural unit represented by formula (a4-2) include the following structural unit:

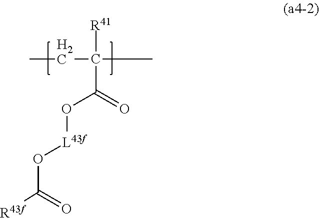

wherein, in formula (a4-2),
R⁴¹ represents a hydrogen atom or a methyl group,
L^{43f} represents a saturated hydrocarbon group having 1 to 20 carbon atoms which may have a fluorine atom, and —CH₂— included in the saturated hydrocarbon group may be replaced by —O— or —CO—, and
R^{43f} represents a saturated hydrocarbon group having 1 to 20 carbon atoms which may have a fluorine atom, and —CH₂— included in the saturated hydrocarbon group may be replaced by —O— or —CO—,
in which at least one of L^{43f} and R^{43f} has a fluorine atom, and the upper limit of the total number of carbon atoms of L^{43f} and R^{43f} is 21.

Examples of the saturated hydrocarbon group in L^{43f} and R^{43f} include a chain saturated hydrocarbon group and a monocyclic or polycyclic alicyclic saturated hydrocarbon group, and groups formed by combining these groups. Examples of the saturated hydrocarbon group in L^{43f} and R^{43f} include groups which are the same as the saturated hydrocarbon groups mentioned as the saturated hydrocarbon group as for R⁴² in formula (a4) as long as the upper limit of the total number of carbon atoms of L^{43f} and R^{43f} permit.

The saturated hydrocarbon group as for L^{43f} is preferably an alkanediyl group having 1 to 6 carbon atoms or a group represented by formula (L^{43f}-1):

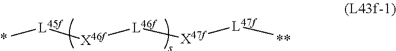

wherein, in formula (L^{43f}-1),
s represents 0 or 1,
L^{45f} and L^{46f} each independently represent a divalent saturated hydrocarbon group having 1 to 5 carbon atoms which may have a substituent,
L^{47f} represents a single bond or a divalent saturated hydrocarbon group having 1 to 5 carbon atoms which may have a substituent,
X^{46f} and X^{47f} each independently represent —O—, —CO—, —CO—O— or —O—CO—,
in which the total number of carbon atoms of L^{45f}, L^{46f}, L^{47f}, X^{46f} and X^{47f} is 7 or less, and
* and ** are bonding sites, and ** is a bonding site to —O—CO—R⁴ 3 f.

Examples of the divalent saturated hydrocarbon group represented by L^{45f}, L^{46f} and L^{47f} in the group represented by formula (L43f-1) include a linear or branched alkanediyl group. Specific examples thereof include a methylene group, an ethylene group, a propane-1,3-diyl group, a propane-1,2-diyl group, a butane-1,4-diyl group, a 1-methylpropane-1,3-diyl group, a 2-methylpropane-1,3-diyl group, a 2-methylpropane-1,2-diyl group and the like.

s is preferably 0.

Examples of the group represented by formula (L43f-1) include the following groups. In the following, * and ** each represent a bonding site, and ** is a bonding site to —O—CO—R^{43f}.

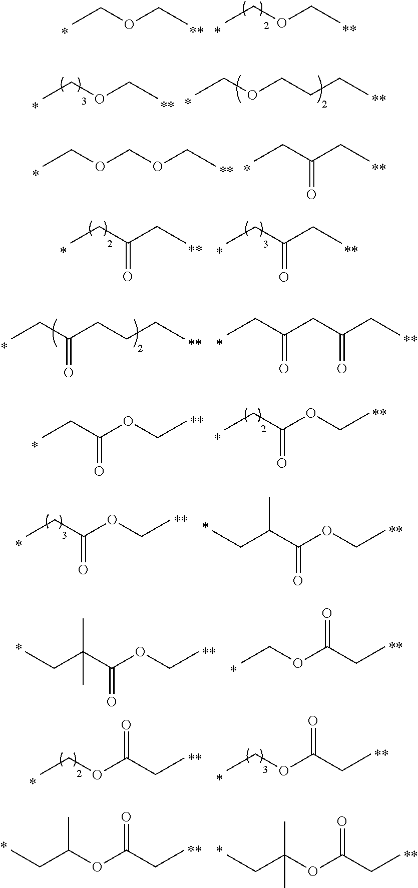

R^{43f} is preferably a saturated hydrocarbon group having a fluorine atom.

In this case, examples of the structural unit represented by formula (a4-2) include a structural unit represented by formula (a4-2A) or formula (a4-2B):

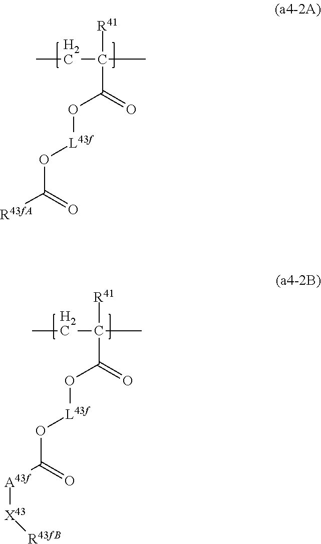

wherein, in formula (a4-2A) and formula (a4-2B),
R⁴¹ and L^{43f} are the same as defined in formula (a4-2),
R^{43fA} represents a saturated hydrocarbon group having 1 to 20 carbon atoms which has a fluorine atom,
A^{43f} represents a divalent saturated hydrocarbon group having 1 to 17 carbon atoms which may have a fluorine atom, and —CH₂— included in the saturated hydrocarbon group may be replaced by —O— or —CO—,
X⁴³ represents **—O—CO— or **—CO—O—, and ** represents a bonding site to A⁴³f, and
R^{43fB} represents a saturated hydrocarbon group having 1 to 17 carbon atoms which may have a fluorine atom,
in which, in formula (a4-2A), the total number of carbon atoms of L^{43f} and A^{43f} is 21 or less, and in formula (a4-2B), the total number of carbon atoms of L^{43f}, A^{43f}, X⁴³ and R^{43fB} is 21 or less, and at least one of A^{43f} and R^{43fB} has at least one fluorine atom.

Examples of the saturated hydrocarbon group as for R^{43fA} include the same saturated hydrocarbon groups as mentioned as for R⁴² as long as the upper limit of the number of carbon atoms permit.

R^{43fA} is preferably an alkyl group having 1 to 13 carbon atoms which has a fluorine atom, a cycloalkyl group having 3 to 12 carbon atoms which has a fluorine atom, or group obtained by combining these groups, and more preferably *—(CF₂)_{n43f}—R^{42′} (* represents a bonding site to a carbonyl group, n43f represents an integer of 1 to 6, and R^{42′} represents a hydrogen atom or a fluorine atom) or a perfluoro cycloalkyl group having 3 to 12 carbon atoms.

Examples of the saturated hydrocarbon group as for A^{43f} and R^{43fB} include the same saturated hydrocarbon groups as that mentioned as the saturated hydrocarbon group as for R⁴² in formula (a4) as long as the upper limit of the number of carbon atoms permit.

A^{43f} is preferably a divalent chain hydrocarbon group which may have a fluorine atom, a divalent alicyclic hydrocarbon group, or groups obtained by combining these groups, more preferably a divalent chain hydrocarbon group having a fluorine atom, and still more preferably an alkanediyl fluoride group having 1 to 6 carbon atoms.

The chain saturated hydrocarbon group which may have a fluorine atom as for R^{43fB} is preferably an alkyl fluoride group such as a trifluoromethyl group, a difluoromethyl group, a methyl group, a perfluoroethyl group, a 2,2,2-trifluoroethyl group, a 1,1,2,2-tetrafluoroethyl group, an ethyl group, a perfluoropropyl group, a 2,2,3,3,3-pentafluoropropyl group, a propyl group, a perfluorobutyl group, a 1,1,2,2,3,3,4,4-octafluorobutyl group, a butyl group, a perfluoropentyl group, a 2,2,3,3,4,4,5,5,5-nonafluoropentyl group, a pentyl group, a hexyl group, a perfluorohexyl group, a heptyl group, a perfluoro heptyl group, an octyl group and a perfluorooctyl group; a cyclopropylmethyl group, a cyclopropyl group, a cyclobutylmethyl group, a cyclopentyl group, a cyclohexyl group, a perfluoro cyclohexyl group, an adamantyl group, an adamantylmethyl group, an adamantyldimethyl group, a norbornyl group, a norbornylmethyl group, a perfluoroadamantyl group or a perfluoroadamantylmethyl group.

In formula (a4-2B), an example of the structure that the group represented by *-A^{f43}-X⁴³—R^{f43B} can take includes the following structures (* is a bonding site to a carbonyl group).

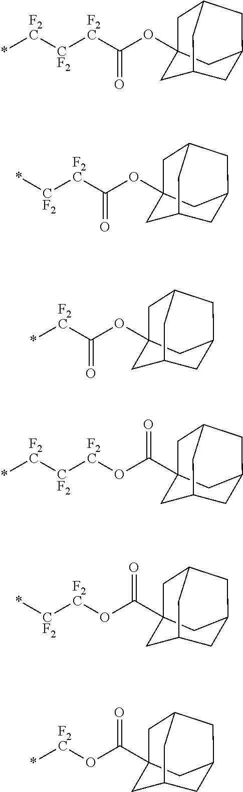 [SMILES: *C(F)(F)C(F)(F)C(F)(F)C(=O)OC12CC3CC(CC(C3)C1)C2 ; *C(F)(F)C(F)(F)C(=O)OC12CC3CC(CC(C3)C1)C2 ; *C(F)(F)C(=O)OC12CC3CC(CC(C3)C1)C2 ; *C(F)(F)C(F)(F)C(F)(F)OC(=O)C12CC3CC(CC(C3)C1)C2 ; *C(F)(F)C(F)(F)OC(=O)C12CC3CC(CC(C3)C1)C2 ; *C(F)(F)OC(=O)C12CC3CC(CC(C3)C1)C2]

Examples of the structural unit represented by formula (a4-2A) include the following structural units, and structural units in which the methyl group corresponding to R⁴¹ in the structural unit represented by formula (a4-2A) in the following structural units is substituted with a hydrogen atom.

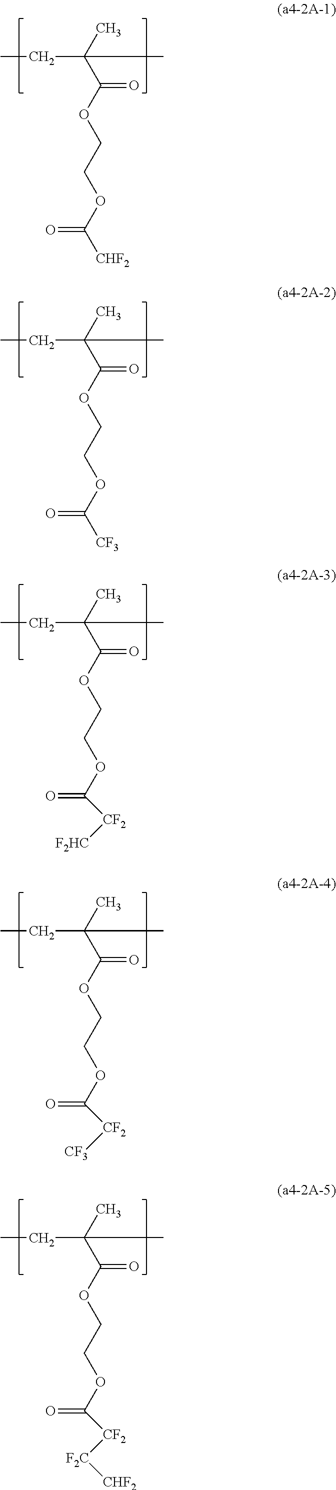

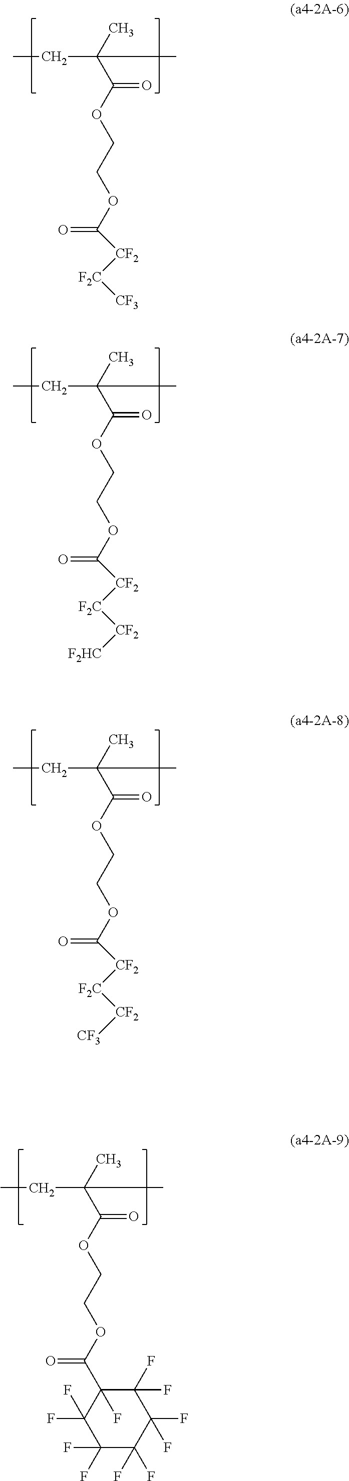

Examples of the structural unit represented by formula (a4-2B) include the following structural units, and structural units in which the methyl group corresponding to R⁴¹ in the structural unit represented by formula (a4-2B) in the following structural units is substituted with a hydrogen atom.

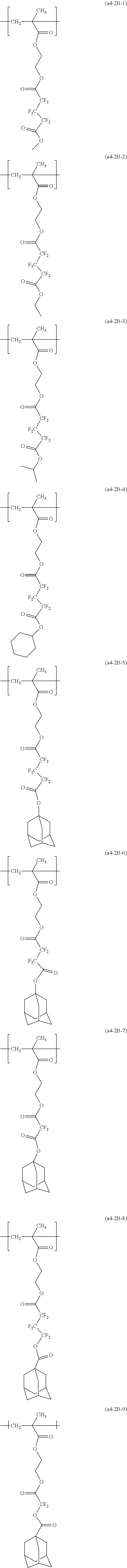

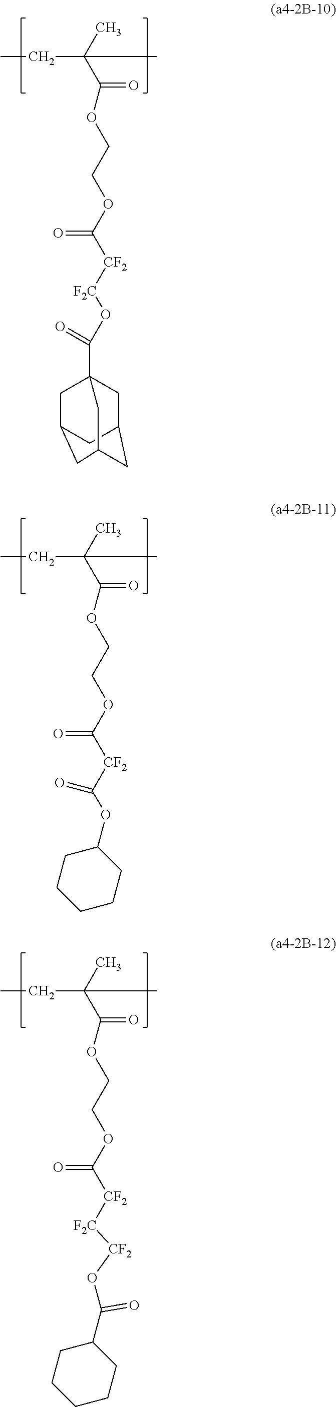

The structural unit (a4) also includes a structural unit represented by formula (a4-3):

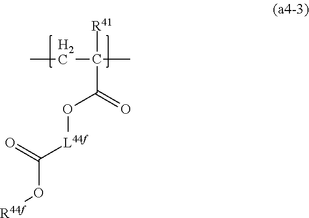

wherein, in formula (a4-3),
R⁴¹ represents a hydrogen atom or a methyl group,
L^{44f} each independently represent a saturated hydrocarbon group having 1 to 20 carbon atoms which may have a fluorine atom, and —CH₂— included in the saturated hydrocarbon group may be replaced by —O— or —CO—, and
R^{44f} each independently represent a saturated hydrocarbon group having 1 to 20 carbon atoms which may have a fluorine atom, and —CH₂— included in the saturated hydrocarbon group may be replaced by —O— or —CO—,
in which at least one of L^{44f} and R^{44f} has a fluorine atom, and the upper limit of the total number of carbon atoms of L^{44f} and R^{44f} is 21, respectively.

Examples of the saturated hydrocarbon group in L⁴⁴f and R^{44f} include a chain saturated hydrocarbon group and a monocyclic or polycyclic alicyclic saturated hydrocarbon group, and groups formed by combining these groups. Examples of the saturated hydrocarbon group in L^{44f} and R^{44f} include groups which are the same as saturated hydrocarbon groups as mentioned as for R⁴² in formula (a4) as long as the upper limit of the number of carbon atoms of L^{43f} and R^{43f} permit.

L^{44f} is more preferably an alkanediyl group having 1 to 13 carbon atoms (—CH₂— included in the alkanediyl group may be replaced by —O— or —CO—), and more preferably a group represented by —(CH₂)₁—, —(CH₂)ⱼ₂—O—(CH₂)ⱼ₃— or —(CH₂)ⱼ₄—CO— O—(CH₂)ⱼ₅— (j1 to j5 each independently represent an integer of 1 to 6 represents), and still more preferably an alkanediyl group having 1 to 4 carbon atoms (one —CH₂— included in the alkanediyl group may be replaced by —O—, and —CH₂—CH₂— included in the alkanediyl group may be replaced by —CO—O— or —O—CO—).

R^{44f} is preferably a saturated hydrocarbon group having 1 to 10 carbon atoms which has a fluorine atom, more preferably an alkyl group having 1 to 10 carbon atoms which has a fluorine atom or an alicyclic saturated hydrocarbon group having 1 to 10 carbon atoms which has a fluorine atom, still more preferably an alkyl group having 1 to 10 carbon atoms which has a fluorine atom, and yet more preferably an alkyl group having 1 to 6 carbon atoms which has a fluorine atom.

Examples of the structural unit represented by formula (a4-3) include the following structural units, and structural units in which the methyl group corresponding to R⁴¹ in the structural unit (a4-3) is substituted with a hydrogen atom in the structural units represented by the following formulas.

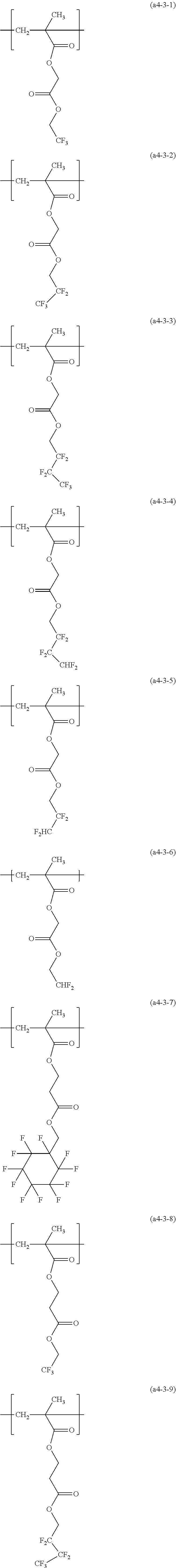

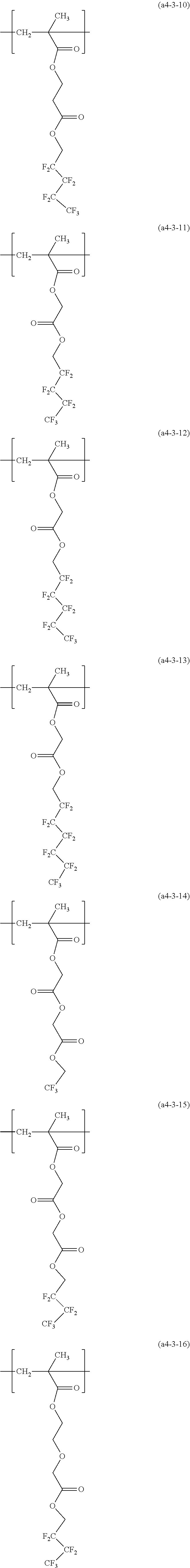

When the resin (A) or the like includes the structural unit (a4), the content is preferably 1 to 20 mol %, more preferably 2 to 15 mol %, and still more preferably 3 to 10 mol %, based on all structural units of the resin (A) or the like.

<Structural Unit (a5)>

Examples of a non-leaving hydrocarbon group possessed by the structural unit (a5) include groups having a linear, branched or cyclic hydrocarbon group. Of these, the structural unit (a5) is preferably a group having an alicyclic hydrocarbon group.

The structural unit (a5) includes, for example, a structural unit represented by formula (a5-1):

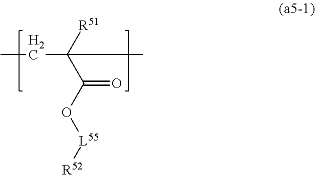

wherein, in formula (a5-1),
R⁵¹ represents a hydrogen atom or a methyl group,
R⁵² represents an alicyclic hydrocarbon group having 3 to 18 carbon atoms, and a hydrogen atom included in the alicyclic hydrocarbon group may be substituted with an aliphatic hydrocarbon group having 1 to 8 carbon atoms, and
L⁵⁵ represents a single bond or a divalent saturated hydrocarbon group having 1 to 18 carbon atoms, and —CH₂— included in the saturated hydrocarbon group may be replaced by —O— or —CO—.

The alicyclic hydrocarbon group in R⁵² may be either monocyclic or polycyclic. The monocyclic alicyclic hydrocarbon group includes, for example, a cyclopropyl group, a cyclobutyl group, a cyclopentyl group and a cyclohexyl group. The polycyclic alicyclic hydrocarbon group includes, for example, an adamantyl group and a norbornyl group.

The aliphatic hydrocarbon group having 1 to 8 carbon atoms includes, for example, alkyl groups such as a methyl group, an ethyl group, a propyl group, an isopropyl group, a butyl group, a sec-butyl group, a tert-butyl group, a pentyl group, a hexyl group, an octyl group and a 2-ethylhexyl group.

Examples of the alicyclic hydrocarbon group having a substituent includes a 3-methyladamantyl group and the like.

R⁵² is preferably an unsubstituted alicyclic hydrocarbon group having 3 to 18 carbon atoms, and more preferably an adamantyl group, a norbornyl group or a cyclohexyl group.

Examples of the divalent saturated hydrocarbon group in L⁵⁵ include a divalent chain saturated hydrocarbon group and a divalent alicyclic saturated hydrocarbon group, and a divalent chain saturated hydrocarbon group is preferable.

The divalent chain saturated hydrocarbon group includes, for example, alkanediyl groups such as a methylene group, an ethylene group, a propanediol group, a butanediyl group and a pentanediyl group.

The divalent alicyclic saturated hydrocarbon group may be either monocyclic or polycyclic. Examples of the monocyclic alicyclic saturated hydrocarbon group include cycloalkanediyl groups such as a cyclopentanediyl group and a cyclohexanediyl group. Examples of the polycyclic divalent alicyclic saturated hydrocarbon group include an adamantanediyl group and a norbornanediyl group.

The group in which —CH₂— included in the divalent saturated hydrocarbon group represented by L⁵⁵ is replaced by —O— or —CO— includes, for example, groups represented by formula (L1-1) to formula (L¹-4). In the following formulas, * and ** each represent a bonding site, and * represents a bonding site to an oxygen atom:

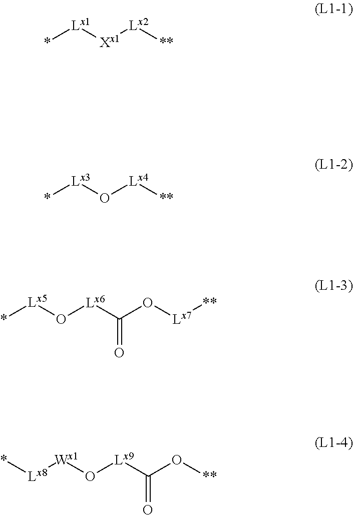

wherein, in formula (L1-1),
X^{x1} represents *—O—CO— or *—CO—O— (* represents a bonding site to L^{x1}),
L^{x1} represents a divalent aliphatic saturated hydrocarbon group having 1 to 16 carbon atoms,
L^{x2} represents a single bond or a divalent aliphatic saturated hydrocarbon group having 1 to 15 carbon atoms, and the total number of carbon atoms of L^{x1} and L^{x2} is 16 or less:
   wherein, in formula (L1-2),
L^{x3} represents a divalent aliphatic saturated hydrocarbon group having 1 to 17 carbon atoms,
L^{x4} represents a single bond or a divalent aliphatic saturated hydrocarbon group having 1 to 16 carbon atoms, and
the total number of carbon atoms of L^{x3} and L^{x4} is 17 or less:
   wherein, in formula (L1-3),
L^{x5} represents a divalent aliphatic saturated hydrocarbon group having 1 to 15 carbon atoms,
L^{x6} and L^{x7} each independently represent a single bond or a divalent aliphatic saturated hydrocarbon group having 1 to 14 carbon atoms, and
the total number of carbon atoms of L^{x5}, L^{x6} and L^{x7} is 15 or less:
   wherein, in formula (L1-4),
L^{x8} and L^{x9} represent a single bond or a divalent aliphatic saturated hydrocarbon group having 1 to 12 carbon atoms, and
W^{x1} represents a divalent alicyclic saturated hydrocarbon group having 3 to 15 carbon atoms,
in which the total number of carbon atoms of L^{x8}, L^{x9} and W^{x1} is 15 or less.

L^{x1} is preferably a divalent aliphatic saturated hydrocarbon group having 1 to 8 carbon atoms, and more preferably a methylene group or an ethylene group.

L^{x2} is preferably a single bond or a divalent aliphatic saturated hydrocarbon group having 1 to 8 carbon atoms, and more preferably a single bond.

L^{x3} is preferably a divalent aliphatic saturated hydrocarbon group having 1 to 8 carbon atoms.

L^{x4} is preferably a single bond or a divalent aliphatic saturated hydrocarbon group having 1 to 8 carbon atoms.

L^{x5} is preferably a divalent aliphatic saturated hydrocarbon group having 1 to 8 carbon atoms, and more preferably a methylene group or an ethylene group.

L^{x6} is preferably a single bond or a divalent aliphatic saturated hydrocarbon group having 1 to 8 carbon atoms, and more preferably a methylene group or an ethylene group.

L^{x7} is preferably a single bond or a divalent aliphatic saturated hydrocarbon group having 1 to 8 carbon atoms.

L^{x8} is preferably a single bond or a divalent aliphatic saturated hydrocarbon group having 1 to 8 carbon atoms, and more preferably a single bond or a methylene group.

L^{x9} is preferably a single bond or a divalent aliphatic saturated hydrocarbon group having 1 to 8 carbon atoms, and more preferably a single bond or a methylene group.

W^{x1} is preferably a divalent alicyclic saturated hydrocarbon group having 3 to 10 carbon atoms, and more preferably a cyclohexanediyl group or an adamantanediyl group.

The group represented by formula (L1-1) includes, for example, the following divalent groups.

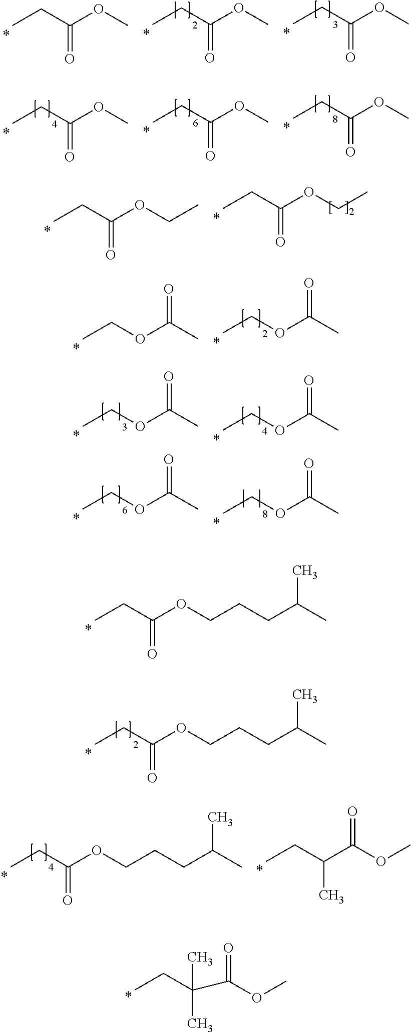

The group represented by formula (L1-2) includes, for example, the following divalent groups.

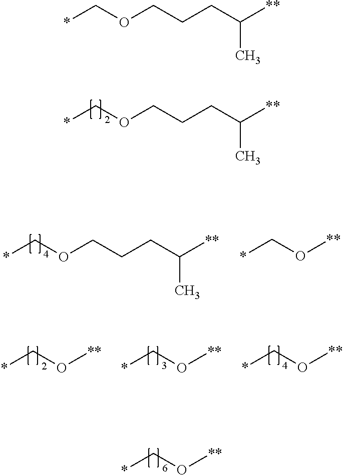

The group represented by formula (L1-3) includes, for example, the following divalent groups.

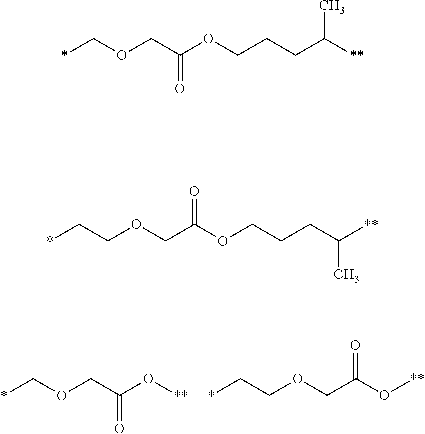

The group represented by formula (L1-4) includes, for example, the following divalent groups.

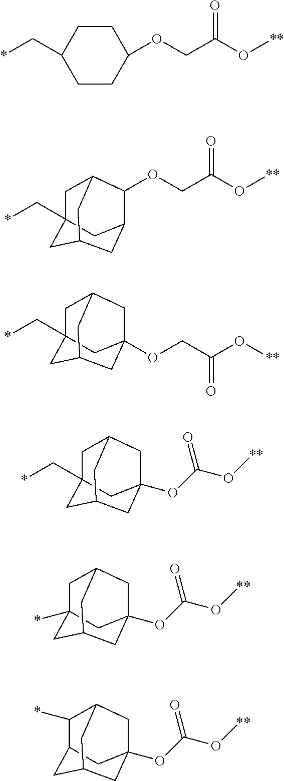

L⁵⁵ is preferably a single bond or a group represented by formula (L1-1).

Examples of the structural unit (a5-1) include the following structural units and structural units in which a methyl group corresponding to R⁵¹ in the structural unit (a5-1) in the following structural units is substituted with a hydrogen atom.

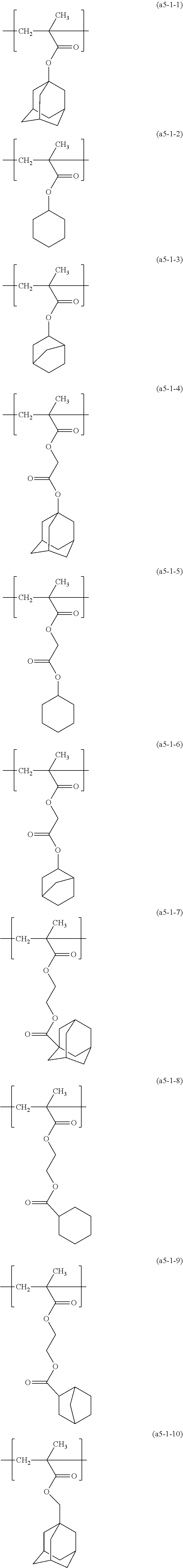

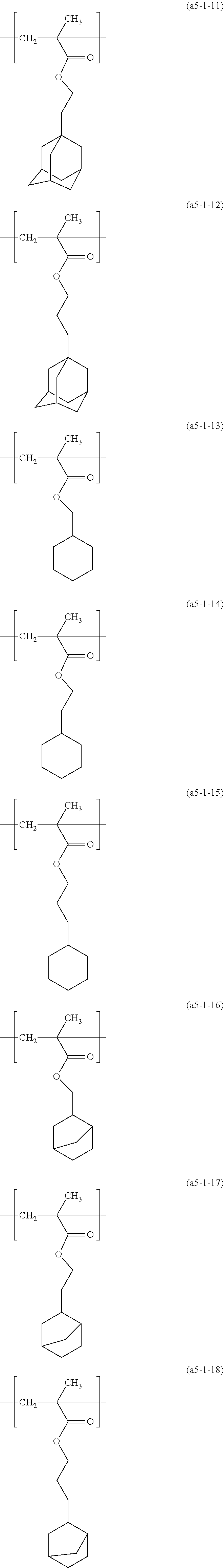

When the resin (A) or the like includes the structural unit (a5), the content is preferably 1 to 30 mol %, more preferably 2 to 20 mol %, and still more preferably 3 to 15 mol %, based on all structural units of the resin (A) or the like.

<Structural Unit (a6)>

The structural unit (a6) is a structural unit having an —SO₂— group, and it is preferable to have an —SO₂— group in a side chain.

The structural unit having an —SO₂— group may have a linear structure having an —SO₂— group, a branched structure having an —SO₂— group, or a cyclic structure (monocyclic and polycyclic structure) having an —SO₂— group. The structural unit is preferably a structural unit which has a cyclic structure having an —SO₂— group, and more preferably a structural unit which has a cyclic structure (sultone ring) having —SO₂—O—.

Examples of the sultone ring include rings represented by the following formula (T1-1), formula (T1-2), formula (T1-3) and formula (T1-4). The bonding site can be any position. The sultone ring may be monocyclic, and is preferably polycyclic. The polycyclic sultone ring means a bridged ring which has —SO₂—O— as an atomic group constituting the ring, and examples thereof include rings represented by formula (T1-1) and formula (T1-2). The sultone ring may have, as the atomic group constituting the ring, a heteroatom, in addition to —SO₂—O—, like the ring represented by formula (T1-2). Examples of the heteroatom include an oxygen atom, a sulfur atom or a nitrogen atom, and an oxygen atom is preferable.

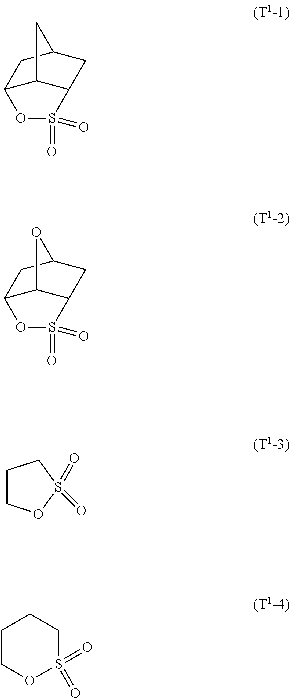 [SMILES: O=S1(=O)OC2CC3CC2C1C3 ; O=S1(=O)OC2CC3CC1C2O3 ; O=S1(=O)CCCO1 ; O=S1(=O)CCCCO1]

The sultone ring may have a substituent, and examples of the substituent include an alkyl group having 1 to 12 carbon atoms which may have a halogen atom or a hydroxy group, a halogen atom, a hydroxy group, a cyano group, an alkoxy group having 1 to 12 carbon atoms, an aryl group having 6 to 12 carbon atoms, an aralkyl group having 7 to 12 carbon atoms, a glycidyloxy group, an alkoxycarbonyl group having 2 to 12 carbon atoms and an alkylcarbonyl group having 2 to 4 carbon atoms.

Examples of the halogen atom include a fluorine atom, a chlorine atom, a bromine atom and an iodine atom.

Examples of the alkyl group include a methyl group, an ethyl group, a propyl group, a butyl group, a pentyl group, a hexyl group, an octyl group and a decyl group, and the alkyl group is preferably an alkyl group having 1 to 6 carbon atoms, and more preferably a methyl group.

Examples of the alkyl group having a halogen atom include a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group, a perfluorohexyl group, a trichloromethyl group, a tribromomethyl group and a triiodomethyl group, and a trifluoromethyl group is preferable.

Examples of the alkyl group having a hydroxy group include hydroxyalkyl groups such as a hydroxymethyl group and a 2-hydroxyethyl group.

Examples of the alkoxy group include a methoxy group, an ethoxy group, a propoxy group, a butoxy group, a pentyloxy group, a hexyloxy group, a heptyloxy group, an octyloxy group, a decyloxy group and a dodecyloxy group.

Examples of the aryl group include a phenyl group, a naphthyl group, an anthryl group, a p-methylphenyl group, a p-tert-butylphenyl group, a p-adamantylphenyl group, a tolyl group, a xylyl group, a cumyl group, a mesityl group, a biphenyl group, a phenanthryl group, a 2,6-diethylphenyl group and a 2-methyl-6-ethylphenyl group.

Examples of the aralkyl group include a benzyl group, a phenethyl group, a phenylpropyl group, a naphthylmethyl group and a naphthylethyl group.

Examples of the alkoxycarbonyl group include groups in which an alkoxy group is bonded with a carbonyl group, such as a methoxycarbonyl group or an ethoxycarbonyl group, and preferably include an alkoxycarbonyl group having 6 or less carbon atoms and more preferably include a methoxycarbonyl group.

Examples of the alkylcarbonyl group include an acetyl group, a propionyl group and a butyryl group.

From the viewpoint that it is easy to produce a monomer from which the structural unit (a6) is derived, a sultone ring having no substituent is preferable.

The sultone ring is preferably a ring represented by the following formula (T1′):

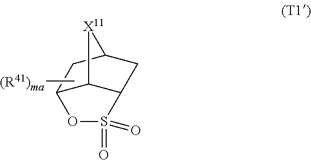

wherein, in formula (T1′),
X¹¹ represents an oxygen atom, a sulfur atom or a methylene group,
R⁴¹ represents an alkyl group having 1 to 12 carbon atoms which may have a halogen atom or a hydroxy group, a halogen atom, a hydroxy group, a cyano group, an alkoxy group having 1 to 12 carbon atoms, an aryl group having 6 to 12 carbon atoms, an aralkyl group having 7 to 12 carbon atoms, a glycidyloxy group, an alkoxycarbonyl group having 2 to 12 carbon atoms, or an alkylcarbonyl group having 2 to 4 carbon atoms,
ma represents an integer of 0 to 9, and when ma is 2 or more, a plurality of R⁴¹ may be the same or different, and
the bonding site may be any position.

X¹¹ is preferably an oxygen atom or a methylene group, and more preferably a methylene group.

and more preferably a methylene group.

Examples of R⁴¹ include those which are the same as the substituent of the sultone ring, and an alkyl group having 1 to 12 carbon atoms which may have a halogen atom or a hydroxy group is preferable.

ma is preferably 0 or 1, and more preferably 0.

Examples of the ring represented by formula (T1′) include the following rings. The bonding site may be any position. Particularly, the bonding site is preferably the 1-position or the 3-position.

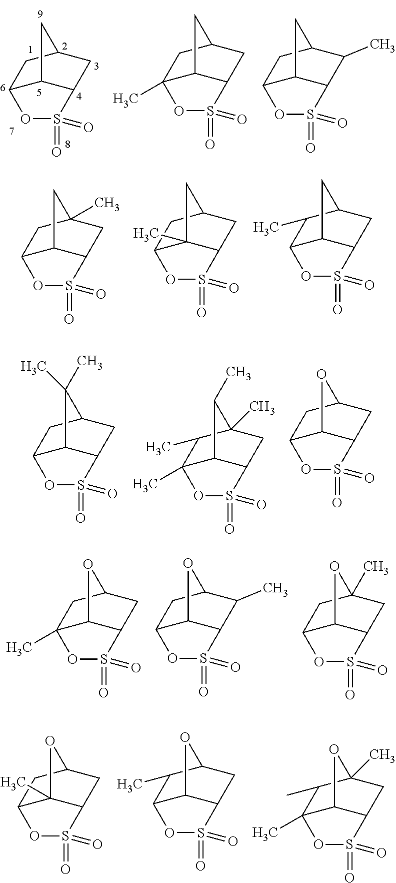

It is preferable that the structural unit having an —SO₂— group further has a group derived from a polymnerizable group. Examples of the polymerizable group include a vinyl group, an acryloyl group, a methacryloyl group, an acryloyloxy group, a methacryloyloxy group, an acryloylamino group, a methacryloylamino group, an acryloylthio group, a methacryloylthio group and the like.

Particularly, the monomer from which the structural unit (a6) is derived is preferably a monomer having an ethylenically unsaturated bond, and more preferably a (meth)acrylic monomer.

The structural unit (a6) is preferably a structural unit represented by formula (a6-0):

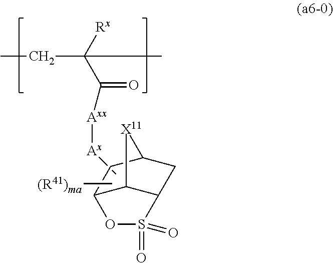

wherein, in formula (a6-0),
R^{x} represents an alkyl group having 1 to 6 carbon atoms which may have a halogen atom, a hydrogen atom or a halogen atom,
A^{xx} represents an oxygen atom, —N(R^{c})— or a sulfur atom,
A^{x} represents a divalent saturated hydrocarbon group having 1 to 18 carbon atoms, and —CH₂— included in the saturated hydrocarbon group may be replaced by —O—, —CO— or —N(R^{d})—,
X¹¹ represents an oxygen atom, a sulfur atom or a methylene group,
R⁴¹ represents an alkyl group having 1 to 12 carbon atoms which may have a halogen atom or a hydroxy group, a halogen atom, a hydroxy group, a cyano group, an alkoxy group having 1 to 12 carbon atoms, an aryl group having 6 to 12 carbon atoms, an aralkyl group having 7 to 12 carbon atoms, a glycidyloxy group, an alkoxycarbonyl group having 2 to 12 carbon atoms, or an alkylcarbonyl group having 2 to 4 carbon atoms,
ma represents an integer of 0 to 9, and when ma is 2 or more, a plurality of R⁴¹ may be the same or different, and
R^{c} and R^{d} each independently represent a hydrogen atom or an alkyl group having 1 to 6 carbon atoms.

Examples of the halogen atom as for R^{x} include a fluorine atom, a chlorine atom, a bromine atom and an iodine atom.

Examples of the alkyl group as for R^{x} include a methyl group, an ethyl group, an n-propyl group, an isopropyl group, an n-butyl group, a sec-butyl group, a tert-butyl group, an n-pentyl group and an n-hexyl group, and an alkyl group having 1 to 4 carbon atoms is preferable, and a methyl group or an ethyl group is more preferable.

Examples of the alkyl group having a halogen atom as for R^{x} include a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group, a perfluorohexyl group, a trichloromethyl group, a tribromomethyl group and a triiodomethyl group.

R^{x} is preferably a hydrogen atom or an alkyl group having 1 to 4 carbon atoms, more preferably a hydrogen atom, a methyl group or an ethyl group, and still more preferably a hydrogen atom or a methyl group.

Examples of the divalent saturated hydrocarbon group as for A^{x} include a linear alkanediyl group, a branched alkanediyl group and a monocyclic or polycyclic divalent alicyclic saturated hydrocarbon group, and the divalent saturated hydrocarbon group may be those obtained by combining two or more of these groups.

Specific examples thereof include linear alkanediyl groups such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, a heptadecane-1,17-diyl group, an ethane-1,1-diyl group, a propane-1,1-diyl group and a propane-2,2-diyl group;
branched alkanediyl groups such as a butane-1,3-diyl group, a 2-methylpropane-1,3-diyl group, a 2-methylpropane-1,2-diyl group, a pentane-1,4-diyl group and a 2-methylbutane-1,4-diyl group;
monocyclic divalent alicyclic saturated hydrocarbon groups which are cycloalkanediyl groups such as a cyclobutane-1,3-diyl group, a cyclopentane-1,3-diyl group, a cyclohexane-1,4-diyl group and a cyclooctane-1,5-diyl group; and
polycyclic divalent alicyclic saturated hydrocarbon groups such as a norbornane-1,4-diyl group, a norbornane-2,5-diyl group, an adamantane-1,5-diyl group and an adamantane-2,6-diyl group.

The bonding site to the sultone ring as for A^{x} can be any position and is preferably the 1-position.

Examples of the structural unit (a6-0) include the following structural units.

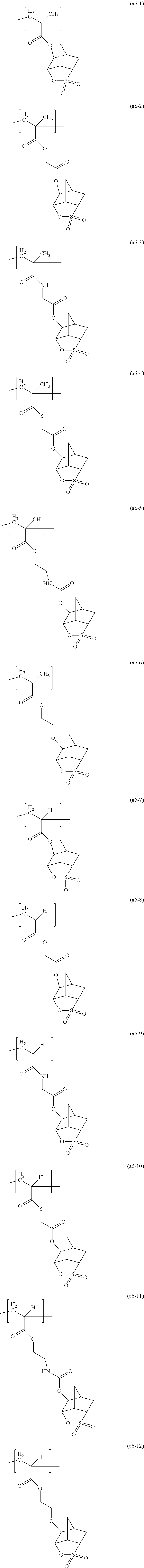

Of these, structural units represented by formula (a6-1), formula (a6-2), formula (a6-6), formula (a6-7), formula (a6-8) and formula (a6-12) are preferable, and structural units represented by formula (a6-1), formula (a6-2), formula (a6-7) and (a6-8) are more preferable.

When the resin (A) or the like includes the structural unit (a6), the content is preferably 1 to 50 mol %, more preferably 2 to 40 mol %, and still more preferably 3 to 30 mol %, based on all structural units of the resin (A) or the like.

<Structural Unit (a7)>

The resin (A) or the like may further include a structural unit other than the structural unit (I) which is decomposed upon exposure to radiation to generate an acid (hereinafter sometimes referred to as “structural unit (a7)”). Specific examples of the structural unit (a7) include the structural units mentioned in JP 2016-79235 A, and a structural unit having a sulfonate group or a carboxylate group and an organic cation in a side chain or a structural unit having a sulfonio group and an organic anion in a side chain are preferable.

The structural unit having a sulfonate group or a carboxylate group and an organic cation in a side chain is preferably a structural unit represented by formula (a7-A):

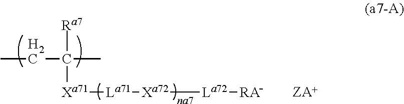

wherein, in formula (a7-A),
R^{a7} represents a hydrogen atom, a halogen atom, or an alkyl group having 1 to 6 carbon atoms which may have a halogen atom,
X^{a71} represents a single bond, *—O—**, *—CO—O—**, *—O—CO—O—**, *—CO—NR^{a71}—**, *—NR^{a71}—CO—O—**, *—O—CO—NR^{a71}—** or *-Ax-Ph-Ay-**,
R^{a71} represents a hydrogen atom or an alkyl group having 1 to 6 carbon atoms,
Ph represents a phenylene group which may have a substituent,
Ax and Ay each independently represent one bond species selected from the group consisting of a single bond, an ether bond, a thioether bond, an ester bond, an amide bond and a carbonic acid ester bond,
* and ** represent a bonding site, and * represent a bonding site to carbon atoms to which R^{a7} is bonded,
L^{a71} and L^{a72} each independently represent a hydrocarbon group having 1 to 24 carbon atoms which may have a substituent, and —CH₂— included in the hydrocarbon group may be replaced by —O—, —S—, —CO— or —SO₂—,
X^{a72} represents ***—CO—O—, ***—O—CO—, ***—O—CO—O—, ***—O—, and *** represents a bonding site to L^{a71},
na7 represents an integer of 0 to 2, and when na7 is 2, a plurality of groups in parentheses may be the same or different from each other,
RA⁻ represents a sulfonate anion or a carboxyl anion, and
ZA⁺ represents an organic cation.

Examples of the alkyl group having 1 to 6 carbon atoms in R^{a7} and Ra7¹ include a methyl group, an ethyl group, an n-propyl group, an isopropyl group, an n-butyl group, a sec-butyl group, a tert-butyl group, a pentyl group and a hexyl group.

Examples of the halogen atom in R^{a7} include a fluorine atom, a chlorine atom, a bromine atom and an iodine atom.

Examples of the alkyl group having 1 to 6 carbon atoms which has a halogen atom in R^{a7} include a trifluoromethyl group, a difluoromethyl group, a perfluoroethyl group, a 2,2,2-trifluoroethyl group, a 1,1,2,2-tetrafluoroethyl group, a perfluoropropyl group, a 2,2,3,3,3-pentafluoropropyl group, a perfluorobutyl group, a 1,1,2,2,3,3,4,4-octafluorobutyl group, a perfluoropentyl group, a 2,2,3,3,4,4,5,5,5-nonafluoropentyl group, a perfluorohexyl group, a trichloromethyl group, a dichloromethyl group, a triiodomethyl group, a diiodo methyl group, a tribromomethyl group, a dibromo methyl group and the like.

R^{a7} is preferably a hydrogen atom or an alkyl group having 1 to 4 carbon atoms, more preferably a hydrogen atom or an alkyl group having 1 to 3 carbon atoms, still more preferably a hydrogen atom, a methyl group or an ethyl group, and yet more preferably a hydrogen atom or a methyl group.

When X^{a71} is a group represented by *-Ax-Ph-Ay-**, preferred is a linking group represented by the following formula (X¹⁰)

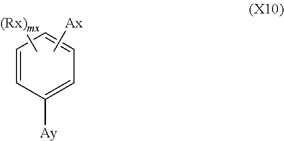

wherein, in formula (X¹⁰),
A^{x} represents bond species bonded to carbon atoms to which R^{a7} is bonded, and represents one bond species selected from the group consisting of a single bond, an ether bond, a thioether bond, an ester bond, a carbonic acid ester bond and an amide bond,
Ay represents bond species to which L^{a71} is bonded, and represents one bond species selected from the group consisting of a single bond, an ether bond, a thioether bond, an ester bond, a carbonic acid ester bond and an amide bond,
Rx represents a halogen atom, a hydroxy group, an alkyl fluoride group having 1 to 6 carbon atoms, an alkyl group having 1 to 18 carbon atoms, or an alkoxy group having 1 to 6 carbon atoms, and
mx represents an integer of 0 to 4, and when mx is an integer of 2 or more, a plurality of Rx may be the same or different from each other.

When one of Ax and Ay is a single bond, the other is preferably one selected from the group consisting of an ether bond, a thioether bond, an ester bond, a carbonic acid ester bond and an amide bond.

When either Ax or Ay is an amide bond, a bond represented by —CO—NR^{a71}— is preferable.

The bonding site of Ay in the phenylene group is preferably the m-position or the p-position, and more preferably the p-position, with respect to the bonding site of Ax.

Particularly, Rx is preferably a fluorine atom, an iodine atom, a trifluoromethyl group, a methyl group or an ethyl group,

mx is preferably 0, 1 or 2.

Examples of X^{a71} include a single bond and groups represented by the following formula (X¹⁰-1) to formula (X¹⁰-10). * represents a bonding site to carbon atoms to which —R^{a7} is bonded. ** represents a bonding site to L^{a71}. X²⁰ represents —O— or —NR^{a71}—.

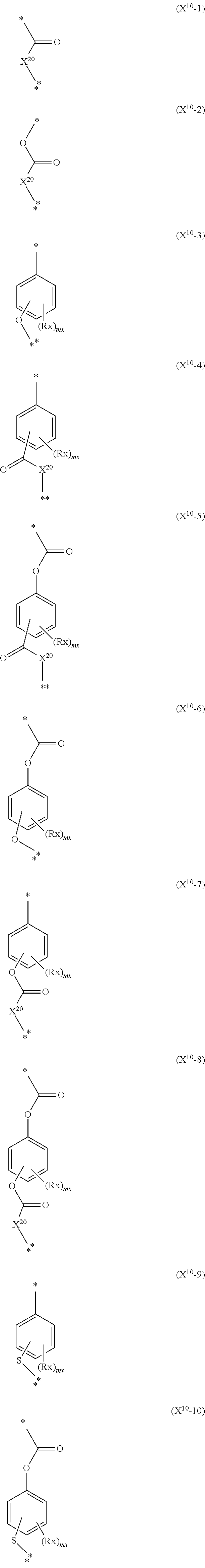

Specific examples of the groups represented by formula (X¹⁰-1) to formula (X¹⁰-10) include the following groups.

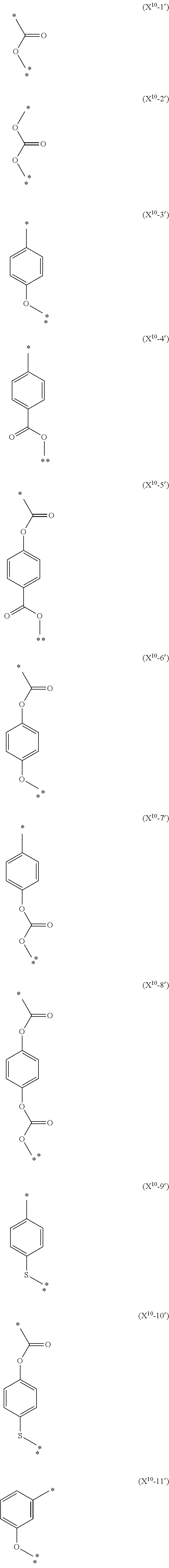

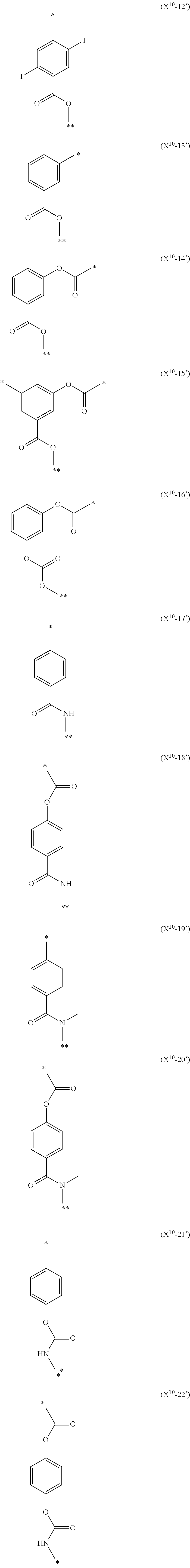

Particularly, X^{a71} is preferably a single bond and a group represented by any one of formula (X¹⁰-1′) and formula (X¹⁰-3′) to formula (X¹⁰-9′), more preferably a single bond or a group represented by any one of formula (X¹⁰-1′), formula (X¹⁰-4′), formula (X¹⁰-5′), formula (X¹⁰-6′) and formula (X¹⁰-9′), and still more preferably a single bond, a group represented by formula (X¹⁰-1′), a group represented by formula (X¹⁰-5′) or a group represented by formula (X¹⁰-6′) or formula (X¹⁰-9′).

Examples of the hydrocarbon group as for L^{a71} include a chain hydrocarbon group, an alicyclic hydrocarbon group, an aromatic hydrocarbon group, and groups which are obtained by removing one hydrogen atom from a group formed by combining two or more of these groups, and are bonded to X^{a71} and X^{a72}. Examples of the hydrocarbon group as for L^{a72} include a chain hydrocarbon group, an alicyclic hydrocarbon group, an aromatic hydrocarbon group, and groups which are obtained by removing one hydrogen atom from a group formed by combining two or more of these groups, and at bonded to X^{a71} and RA⁻.

Examples of the chain hydrocarbon group as for L^{a71} and L^{a72} include groups obtained by removing one hydrogen atom of an alkyl group or an alkenyl group. The alkyl group may be either linear or branched, and specific examples thereof include a methyl group, an ethyl group, an n-propyl group, an iso-propyl group, an n-butyl group, a sec-butyl group, an iso-butyl group, a tert-butyl group, a pentyl group, a hexyl group, a heptyl group, an octyl group, a nonyl group, a decyl group, an undecyl group, a dodecyl group, a tridecyl group, a tetradecyl group, a pentanedecyl group, a heptadecyl group and the like. Examples of the alkenyl group include an ethenyl group, a propenyl group, an isopropenyl group, a butenyl group, an isobutenyl group, a tert-butenyl group, a pentenyl group, a hexenyl group, a heptenyl group, an octenyl group, an isooctenyl group, a nonenyl group and the like.

The number of carbon atoms of the chain hydrocarbon group is preferably 1 to 20, more preferably 1 to 18, still more preferably 1 to 10 carbon atoms.

Examples of the alicyclic hydrocarbon group as for L^{a71} and L^{a72} include groups obtained by removing one hydrogen atom of a monocyclic or polycyclic cycloalkyl group. Examples of the monocyclic cycloalkyl group include a cyclobutyl group, a cycloheptyl group, a cyclohexyl group, a cyclopentyl group, a cyclooctyl group and the like.

Examples of the polycyclic cycloalkyl group as for L^{a71} and L^{a72} include a cycloalkyl group having a crosslinked structure, a cycloalkyl group in which two or more rings are fused, or a cycloalkyl group in which two rings are bonded by spiro bonding. Examples of the cycloalkyl group having a crosslinked structure include a norbornyl group, an adamantyl group and the like. Examples of the cycloalkyl group in which two or more rings are fused include a bicyclo[4,4,0]decane group, a steroid group (steroid skeleton) and the like. Examples of the cycloalkyl group in which two rings are bonded by spiro bonding include a spirocyclic cycloalkyl group in which one cycloalkyl group selected from the group consisting of a cyclopentyl group, a cyclohexyl group, a norbornyl group and an adamantyl group, and a cycloalkyl group having 5 to 8 carbon atoms are bonded by spiro bonding, and the like. A double bond may be formed between two carbon atoms included in the alicyclic hydrocarbon group. More specifically, alicyclic hydrocarbon groups represented by the following formulas are exemplified.

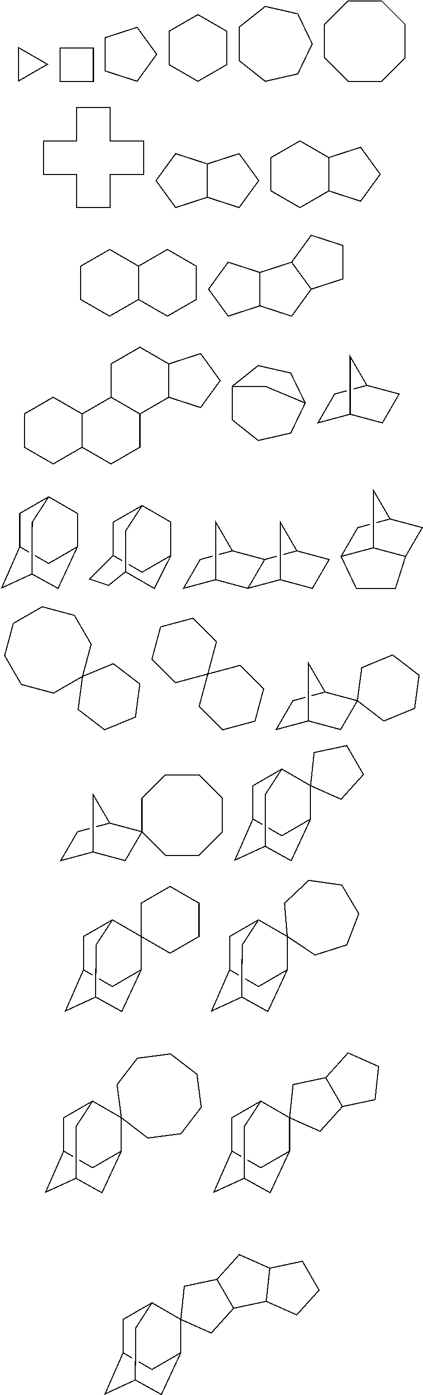

When the alicyclic hydrocarbon group is a monocyclic cycloalkyl group, the number of carbon atoms of the alicyclic hydrocarbon group is preferably 3 to 18, more preferably 3 to 12, and still more preferably 3 to 8. When the alicyclic hydrocarbon group is a polycyclic cycloalkyl group, the number of carbon atoms of the alicyclic hydrocarbon group is preferably 6 to 18, and more preferably 7 to 12.

Examples of the aromatic hydrocarbon group L^{a7l} and L^{a72} include groups obtained by removing one hydrogen atom of an aryl group. Examples of the aryl group include a phenyl group, a naphthyl group, an anthryl group, a biphenyl group, a fluorenyl group and the like. The number of carbon atoms of the aromatic hydrocarbon group is preferably 5 to 14, more preferably 6 to 14, and still more preferably 6 to 10.

When —CH₂— included in the hydrocarbon group as for L^{a71} and L^{a72} is replaced by —O—, —CO—, —S— or —SO₂—, the number of carbon atoms before replacement is taken as the total number of the hydrocarbon group.

Of the hydrocarbon groups as for L^{a71} and L^{a72}, examples of the group in which —CH₂— included in the chain hydrocarbon group is replaced by —O—, —CO—, —S— or —SO₂— include a hydroxy group (a group in which —CH₂— included in the methyl group is replaced by —O—), a carboxy group (a group in which —CH₂—CH₂— included in the ethyl group is replaced by —O—CO—), a thiol group (a group in which —CH₂— included in the methyl group is replaced by —S—), a carbonyl group (a group in which —CH₂— included in the methylene group is replaced by —CO—), an oxy group (a group in which —CH₂— included in the methylene group is replaced by —O—), a thio group (a group in which —CH₂— included in the methylene group is replaced by —S—), a sulfonyl group (a group in which —CH₂— included in the methylene group is replaced by —SO₂—), an alkoxy group (a group in which —CH₂— at any position included in the alkyl group is replaced by O—), an alkoxycarbonyl group (a group in which —CH₂—CH₂— at any position included in the alkyl group is replaced by —O—CO—), an alkylcarbonyl group (a group in which —CH₂— at any position included in the alkyl group is replaced by —CO—), an alkylcarbonyloxy group (a group in which —CH₂—CH₂— at any position included in the alkyl group is replaced by —CO—O—), an alkanediyloxy group (a group in which —CH₂— at any position included in the alkanediyl group is replaced by —O—), an alkanediyloxycarbonyl group (a group in which —CH₂—CH₂— at any position included in the alkanediyl group is replaced by —O—CO—), an alkanediylcarbonyl group (a group in which —CH₂— at any position included in the alkanediyl group is replaced by —CO—), an alkanediylcarbonyloxy group (a group in which —CH₂—CH₂— at any position included in the alkanediyl group is replaced by —CO—O—) and the like. Examples of these replaced groups include the same groups as mentioned herein as long as the upper limit of the number of carbon atoms permits.

Of the hydrocarbon groups as for L^{a71} and L^{a72}, examples of the group in which —CH₂— included in the alicyclic hydrocarbon group is replaced by —O—, —CO—, —S— or —SO₂— include groups having a structure such as cyclic ether, cyclic ketone, cyclic ester (lactone), cyclic thioether, cyclic acetal or cyclic sulfonic acid ester (sultone). Specific examples thereof include alicyclic hydrocarbon groups represented by the following formulas. The bonding site of the alicyclic hydrocarbon group represented by the following formulas can be any position.

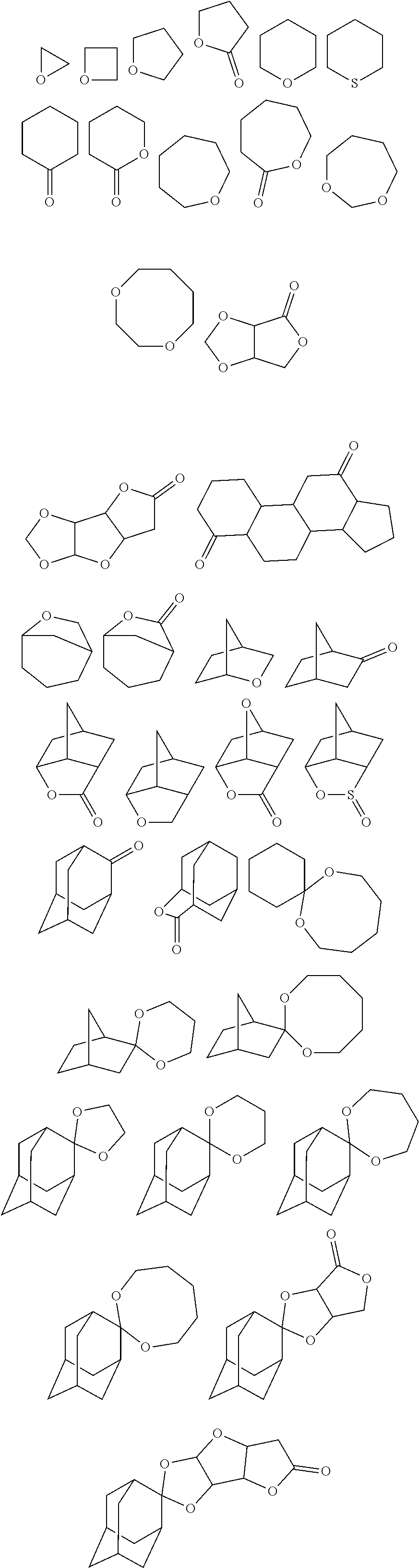

Of the hydrocarbon groups as for L^{a71} and L^{a72}, —CH₂— included in the aromatic hydrocarbon group may be replaced by —O— or —S—, and examples of the group in which —CH₂— is replaced by —O— or —S— include groups derived from a furan ring or a thiophene ring, respectively.

Examples of the groups obtained by combining two or more groups of the chain hydrocarbon group, the alicyclic hydrocarbon group and the aromatic hydrocarbon group as for L^{a71} and L^{a72} include a group obtained by combining the chain hydrocarbon group with the alicyclic hydrocarbon group, a group obtained by combining the chain hydrocarbon group with the aromatic hydrocarbon group, a group obtained by combining the alicyclic hydrocarbon group with the aromatic hydrocarbon group, a group obtained by combining the chain hydrocarbon group, the alicyclic hydrocarbon group and the aromatic hydrocarbon group. The group obtained by combining the alicyclic hydrocarbon group with the aromatic hydrocarbon group may also be a fused ring.

Examples of the substituent which may be possessed by the hydrocarbon group as for L^{a71} and L^{a72} include a halogen atom, a cyano group and a nitro group. Examples of the halogen atom include a fluorine atom, a chlorine atom, a bromine atom, an iodine atom and the like.

When L^{a71} and L^{a72} are groups obtained by combining an alicyclic hydrocarbon group or an aromatic hydrocarbon group with a chain hydrocarbon group, the chain hydrocarbon group may be substantially regarded as a substituent which is possessed by the alicyclic hydrocarbon group or the aromatic hydrocarbon group. By replacing —CH₂— of the chain hydrocarbon group included in the hydrocarbon group as for L^{a71} and L^{a72} by —O—, —CO—, —S— or —SO₂—, the hydrocarbon group as for L^{a71} and L^{a72} can substantially have a substituent such as a hydroxy group, a carboxy group, an alkoxy group, an alkylcarbonyl group, an alkoxycarbonyl group, an alkylcarbonyloxy group, a thiol group or a sulfonyl group.

na7 is preferably 0 or 1.

Examples of ZA⁺ in formula (a7-A) include the same cations as in the salt represented by formula (B1) or the like.

In formula (a7-A), when the hydrocarbon group as for L^{a71} and L^{a72} is a saturated hydrocarbon group, it is possible to include the same groups as mentioned as divalent linking group as for A^{b7} in formula (a7-B) mentioned below.

Examples of the structural unit represented by formula (a7-A) also include a structural unit or the like represented by formula (a7-A1)

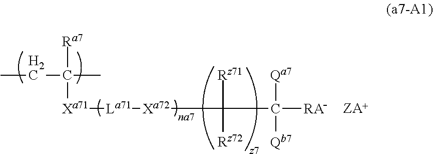

wherein, in formula (a7-A1),
R^{a7}, X^{a71}, L^{a71}, X^{a72}, na7, RA⁻ and ZA⁺ are the same as defined above,
z7 represents an integer of 0 to 6, and
Q^{a7}, Q^{b7}, R^{z71} and R^{z72} each independently represent a hydrogen atom, a fluorine atom, a perfluoroalkyl group having 1 to 6 carbon atoms or an alkyl group having 1 to 6 carbon atoms, and when z7 is 2 or more, a plurality of R^{z71} and R^{z72} may be the same or different from each other.

Examples of the perfluoroalkyl group having 1 to 6 carbon atoms or alkyl group having 1 to 6 carbon atoms in Q^{a7}, Q^{b7}, R^{z71} and R^{z72} include the same groups as mentioned as for R^{a7}.

Examples of the structural unit represented by formula (a7-A) include the following structural units, structural units in which the group corresponding to the methyl group as for R^{a7} is substituted with a hydrogen atom, a halogen atom (e.g., a fluorine atom) or an alkyl group having 1 to 6 carbon atoms which may have a halogen atom (e.g., a trifluoromethyl group, etc.), and structural units mentioned in WO 2012/050015 A. ZA⁺ represents an organic cation.

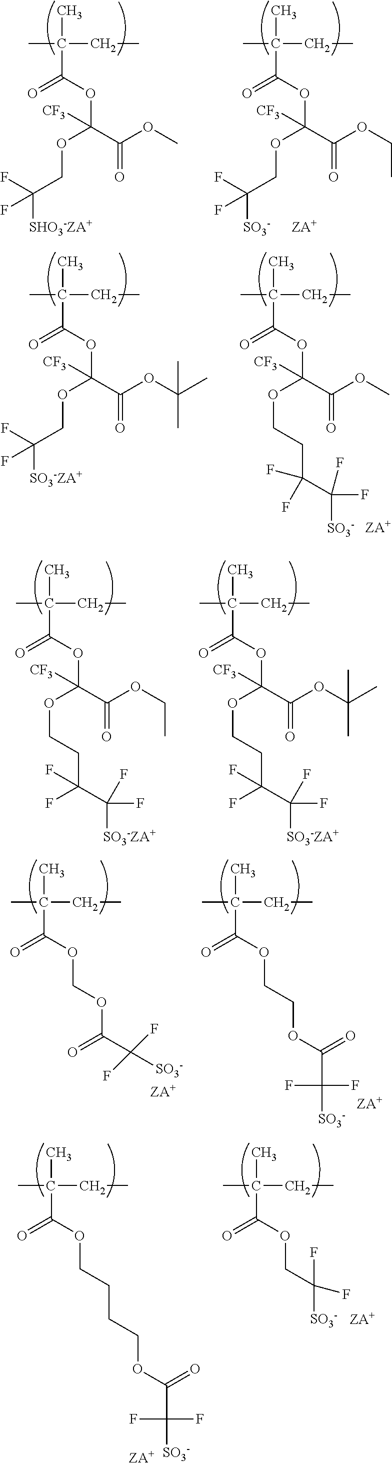

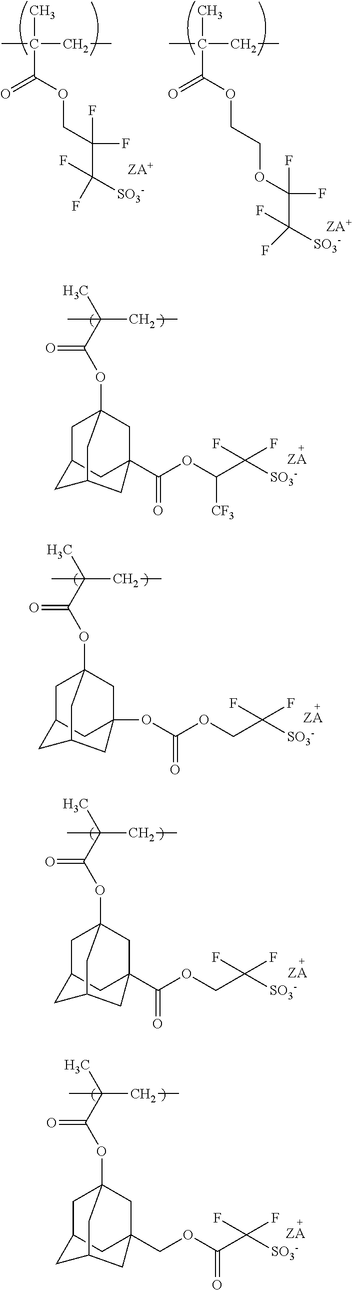

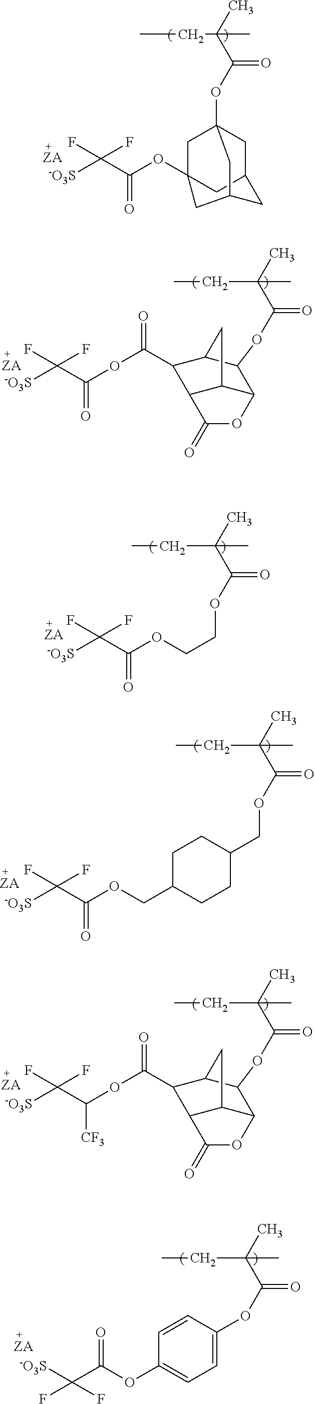

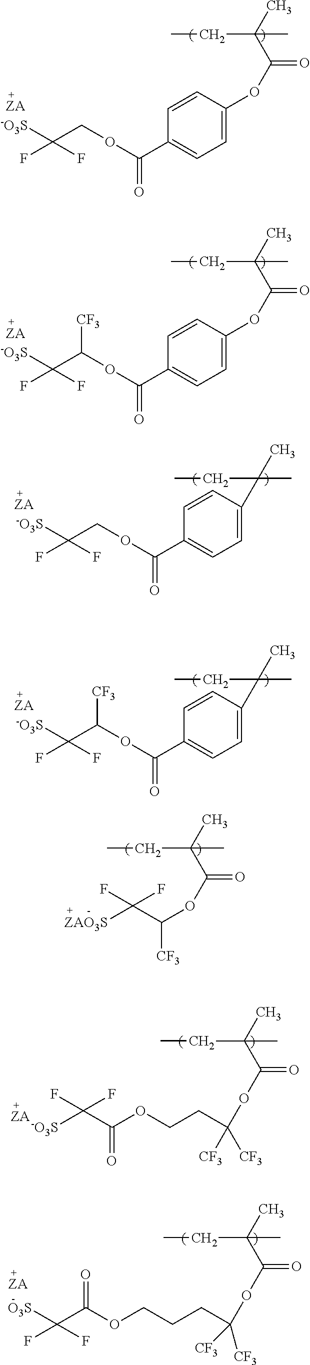

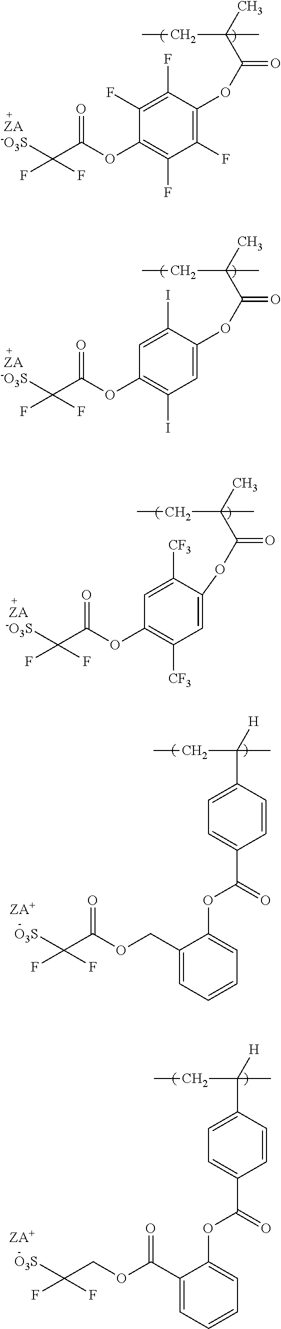

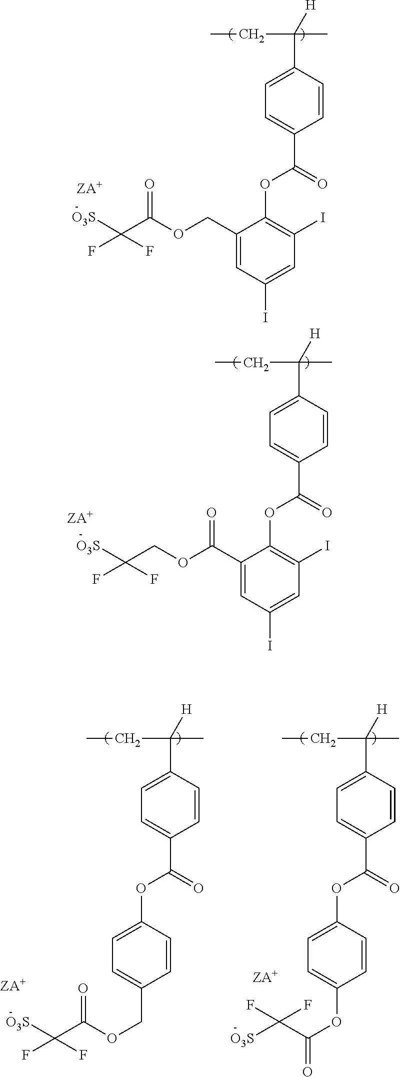

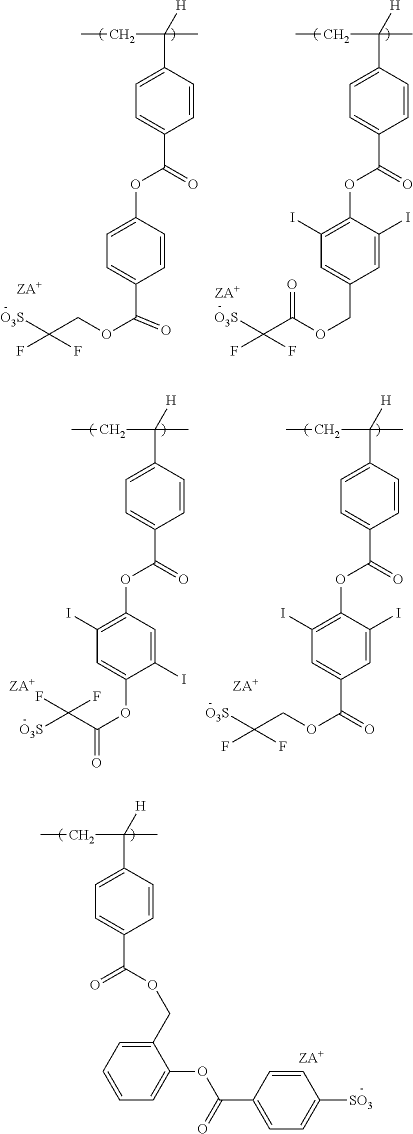

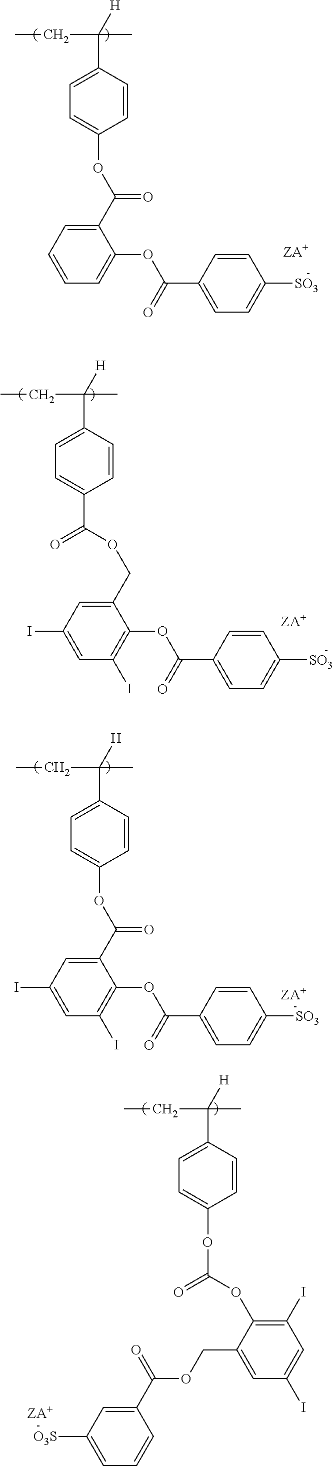

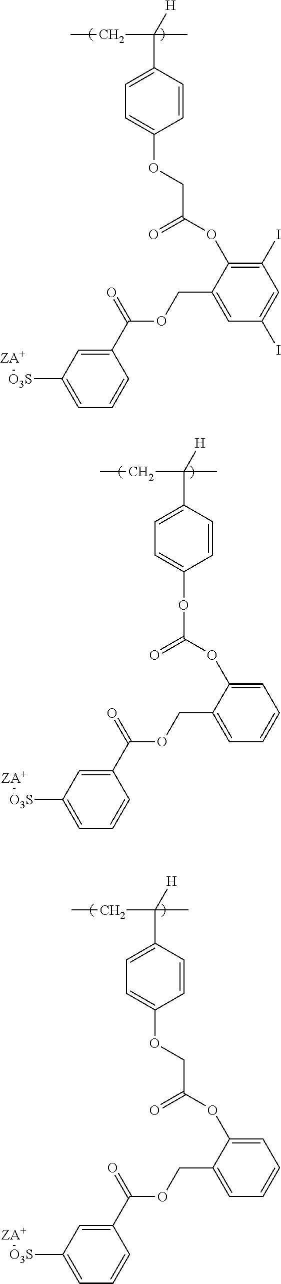

The structural unit having a sulfonio group and an organic anion in the side chain is preferably a structural unit represented by formula (a7-B):

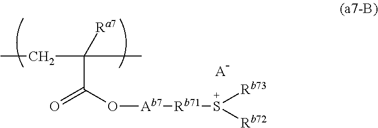

wherein, in formula (a7-B),
R^{a7} represents a hydrogen atom, a halogen atom, or an alkyl group having 1 to 6 carbon atoms which may have a halogen atom,
A^{b7} represents a single bond or a divalent linking group,
R^{b71} represents a divalent aromatic hydrocarbon group having 6 to 18 carbon atoms which may have a substituent,
R^{b72} and R^{b73} each independently represent a hydrocarbon group having 1 to 18 carbon atoms which may have a substituent, R^{b72} and R^{b73} may be bonded to each other to form a ring together with sulfur atoms to which R^{b72} and R^{b73} are bonded, and
A⁻ represents an organic anion.

Examples of the halogen atom and the alkyl group which may have a halogen atom as for R^{a7} include the same halogen atoms and the alkyl groups which may have a halogen atom of formula (a7-A).

Examples of the divalent aromatic hydrocarbon group having 6 to 18 carbon atoms represented by R^{b71} include a phenylene group and a naphthylene group.

Examples of the hydrocarbon group represented by R^{b72} and R^{b73} include an alkyl group, an alicyclic hydrocarbon group, an aromatic hydrocarbon group, and groups formed by combining these groups.

Examples of the alkyl group, the alicyclic hydrocarbon group, the aromatic hydrocarbon group, and the groups formed by combining these groups include the same groups as mentioned above.

Examples of the divalent linking group represented by A^{b7} include a divalent saturated hydrocarbon group having 1 to 18 carbon atoms, and —CH₂— included in the divalent saturated hydrocarbon group may be replaced by —O—, —S— or —CO—.

Examples of the divalent saturated hydrocarbon group include divalent chain saturated hydrocarbon groups such as a linear or branched alkanediyl group, a monocyclic or polycyclic divalent alicyclic saturated hydrocarbon group, or a combination thereof.

Specific examples thereof include linear alkanediyl groups such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group and a dodecane-1,12-diyl group; branched alkanediyl groups such as a butane-1,3-diyl group, a 2-methylpropane-1,3-diyl group, a 2-methylpropane-1,2-diyl group, a pentane-1,4-diyl group and a 2-methylbutane-1,4-diyl group; divalent monocyclic alicyclic saturated hydrocarbon groups which are monocyclic cycloalkanediyl groups, such as a cyclobutane-1,3-diyl group, a cyclopentane-1,3-diyl group, a cyclohexane-1,4-diyl group and a cyclooctane-1,5-diyl group; and divalent polycyclic alicyclic saturated hydrocarbon groups which are polycyclic cycloalkanediyl groups, such as a norbornane-1,4-diyl group, a norbornane-2,5-diyl group, an adamantane-1,5-diyl group and an adamantane-2,6-diyl group.

Those in which —CH₂— included in the saturated hydrocarbon group are replaced by —O—, —S— or —CO— include, for example, the following divalent groups. Before replacing —CH₂— included in the saturated hydrocarbon group by —O—, —S— or —CO—, the number of carbon atoms is 17 or less. In the following formulas, * and ** represent a bonding site, and * represents a bonding site to R^{b71}.

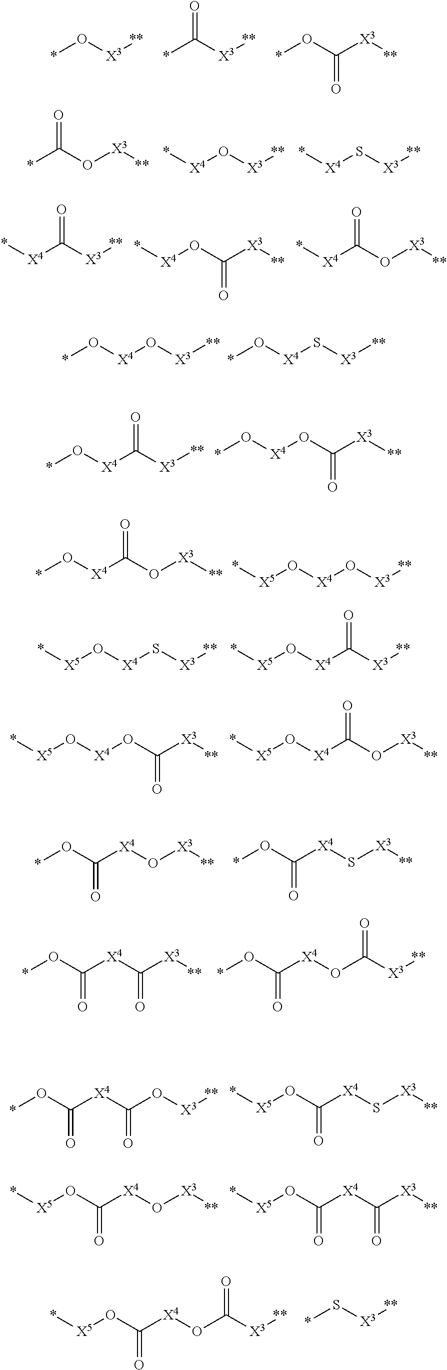

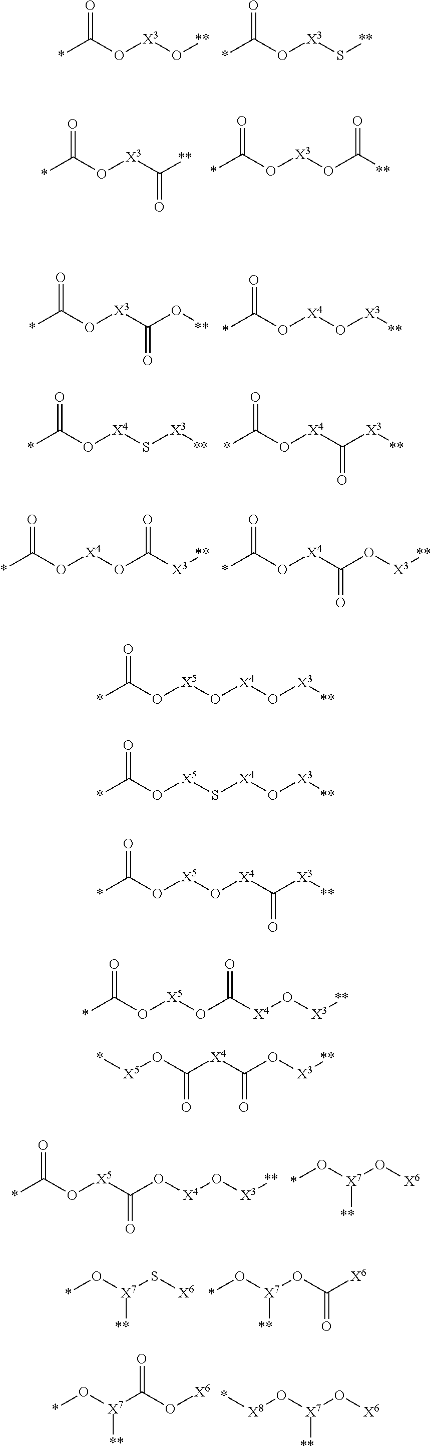

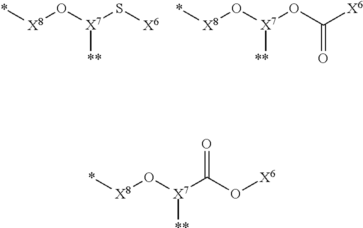

wherein, X³ represents a divalent saturated hydrocarbon group having 1 to 16 carbon atoms,
X⁴ represents a divalent saturated hydrocarbon group having 1 to 15 carbon atoms,
X⁵ represents a divalent saturated hydrocarbon group having 1 to 13 carbon atoms,
X⁶ represents a divalent saturated hydrocarbon group having 1 to 14 carbon atoms,
X⁷ represents a trivalent saturated hydrocarbon group having 1 to 14 carbon atoms, and
X⁸ represents a divalent saturated hydrocarbon group having 1 to 13 carbon atoms.

Examples of the structural unit including a cation in formula (a7-B) include the following structural units and structural units in which a group corresponding to a methyl group of R^{a7} is substituted with a hydrogen atom, a halogen atom (e.g., a fluorine atom, etc.) or an alkyl group having 1 to 6 carbon atoms which may have a halogen atom (e.g., a trifluoromethyl group, etc.) and the like.

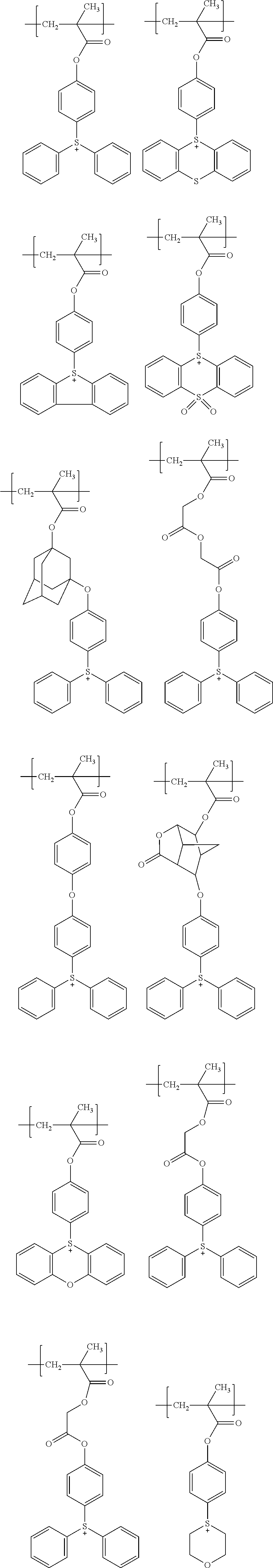

Examples of the organic anion represented by A-include a sulfonic acid anion, a sulfonylimide anion, a sulfonylmethide anion and a carboxylic acid anion. The organic anion represented by A⁻ is preferably a sulfonic acid anion. The sulfonic acid anion is more preferably those which are the same as anions to be mentioned in the acid generator (B). Examples of the sulfonic acid anion, the sulfonylimide anion, the sulfonylmethide anion and the carboxylic acid anion include those which are the same as anions to be mentioned in the acid generator (B).

Examples of the structural unit represented by formula (a7-B) include the followings.

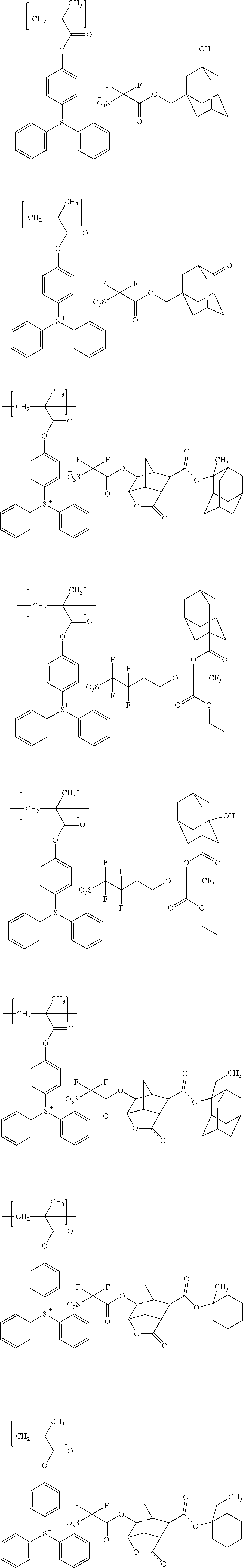

When the resin (A) includes a structural unit (a7), the structural unit (a7) may be included alone, or two or more structural units may be included. The total content of the structural unit (a7) is preferably 1 to 30 mol %, more preferably 1 to 25 mol %, still more preferably 2 to 20 mol % yet more preferably still more preferably 3 to 15 mol %, and further preferably 3 to 10 mol %, based on all structural units of the resin (A1).

The resin (A) or the like is preferably a resin composed of a structural unit (a1) and a structural unit (s), that is, a copolymer of a monomer (a1) and a monomer (s).

The structural unit (a1) is preferably at least one selected from the group consisting of a structural unit (a1-0), a structural unit (a1-1) and a structural unit (a1-2) (preferably the structural unit having a cyclohexyl group and a cyclopentyl group), more preferably at least two, and still more preferably at least two selected from the group consisting of a structural unit (a1-1) and a structural unit (a1-2).

The structural unit (s) is preferably at least one selected from the group consisting of a structural unit (a2) and a structural unit (a3). The structural unit (a2) is preferably a structural unit (a2-1) or a structural unit (a2-A). The structural unit (a3) is preferably at least one selected from the group consisting of a structural unit represented by formula (a3-1), a structural unit represented by formula (a3-2) and a structural unit represented by formula (a3-4).

The respective structural units constituting the resin (A) or the like may be used alone, or two or more structural units may be used in combination. Using a monomer from which these structural units are derived, it is possible to produce by a known polymerization method (e.g., radical polymerization method). The content of the respective structural units included in the resin (A) or the like can be adjusted according to the amount of the monomer used in the polymerization.

The weight-average molecular weight of the resin (Ap) and the resin (A) is preferably 2,000 or more (more preferably 2,500 or more, and still more preferably 3,000 or more), and 50,000 or less (more preferably 30,000 or less, and still more preferably 15,000 or less), and an oligomer having a weight-average molecular weight of less than the above value may be included. The structural unit (IP) may constitute a dimer, a trimer, and a compound having a weight-average molecular weight of less than 2,000. As used herein, the weight-average molecular weight is a value determined by gel permeation chromatography under the analysis conditions mentioned in Examples.

### [Acid Generator]

The acid generator of the present invention is an acid generator including a salt (I) and/or a structural unit (IP). When using the salt (I) as the acid generator in the resist composition, one salt (I) may be included, and two or more salt (I) may also be included. When using the structural unit (IP) as the acid generator in the resist composition, a compound or resin including the structural unit (IP) may be used alone or in combination of two or more thereof. The acid generator of the present invention may include both the salt (I) and the structural unit (IP). The structural unit (IP) can be included as a compound or a resin obtained by polymerizing a plurality thereof. The acid generator of the present invention may further include, in addition to the salt (I) and/or the structural unit (IP), a compound acting as an acid generator known in the resist field (hereinafter sometimes referred to as “compound (B)”) and/or a carboxylic acid generator known in the resist field. The compound (B) may be used alone or in combination of two or more thereof.

### [Resist Composition]

The resist composition of the present invention includes the acid generator of the present invention. The acid generator here may be either a salt (I) or a resin (Ap) including a structural unit (IP). The resist composition of the present invention includes at least one of the carboxylate (I) and the structural unit (IP), and may include both of them. That is, the resist composition of the present invention may include an acid generator including the structural unit (IP) of the present invention or the salt (I) of the present invention. The structural unit (IP) may be in a form of either compound or resin. In other words, the resist composition of the present invention may include, as the acid generator, a resin (Ap) and a salt (I).

It is preferable that the resist composition of the present invention further includes a resin. The resin is preferably a resin including a structural unit (a1) having an acid-labile group. That is, the resist composition preferably includes:
(a) a salt (I) and a resin (A), or
(b) a resin (Ap) including a structural unit (IP) and a structural unit (a1) having an acid-labile group, or
(c) a resin (Ap) including a structural unit (IP) and a resin (A). Of these, the resist composition including (b) mentioned above is preferable. The resist composition of the present invention may also include two or more resins (A) and/or resins (Ap). The (b) and (c) mentioned above may further include a salt (I).

It is preferable that the resist composition of the present invention further includes a compound (B) and a quencher such as a salt generating an acid having an acidity lower than that of an acid generated from the acid generator (hereinafter sometimes referred to as “quencher (C)”), and includes a solvent (hereinafter sometimes referred to as “solvent (E)”). The resist composition of the present invention may further include a resin other than the resins mentioned above.

### <Acid Generator>

In the resist composition of the present invention, the content of the acid generator is preferably 1% by mass or more and 45% by mass or less, more preferably 1% by mass or more and 40% by mass or less, and still more preferably 3% by mass or more and 35% by mass or less, based on the solid content of the resist composition.

The content of the resin (Ap) of the present invention is preferably 80% by mass or more and 99% by mass or less, and more preferably 90% by mass or more and 99% by mass or less, based on the solid content of the resist composition.

In the resist composition of the present invention, when including a compound (B), the total content of the acid generator is preferably 1% by mass or more and 45% by mass or less, more preferably 1% by mass or more and 40% by mass or less, and still more preferably 3% by mass or more and 35% by mass or less, based on the solid content of the resist composition. When including a resin (A) mentioned below, the content of the compound (B) is preferably 1 part by mass or more and 45 parts by mass or less, more preferably 1 part by mass or more and 40 parts by mass or less, and still more preferably 3 parts by mass or more and 40 parts by mass or less, based on 100 parts by mass of the resin (A).

<Resin Other than Resin (A) or the Like>

In the resist composition of the present invention, a resin other than the resin (A) or the like may be used in combination.

Examples of the resin other than the resin (A) include, for example, a resin including a structural unit (a4) or a structural unit (a5) (hereinafter sometimes referred to as resin (X)) and the like.

Examples of the resin (X) particularly include a resin composed only of a structural unit (a4) and a resin composed of a structural unit (a4) and a structural unit (a5) (hereinafter, the resin composed only of a structural unit (a4) and the resin composed of a structural unit (a4) and a structural unit (a5) sometimes referred collectively to as resin (X)) and the like.

Examples of the structural unit which may be further included in the resin (X) include a structural unit (a2), a structural unit (a3) and structural units derived from other known monomers.

When the resin (X) includes a structural unit (a4), the content of the structural unit (a4) in the resin (X) is 20 mol % or more, preferably 30 mol % or more, more preferably 40 mol % or more, and still more preferably 45 mol % or more, based on the total of all structural units of the resin (X). The content is also 100 mol % or less, preferably 80 mol % or less, more preferably 70 mol % or less, still more preferably 60 mol % or less, and yet more preferably 55 mol % or less. Specifically, the content is 20 to 100 mol %, preferably 20 to 80 mol %, more preferably 30 to 70 mol %, still more preferably 40 to 60 mol %, and yet more preferably 45 to 55 mol %. When the resin (X) includes a structural unit (a5), the content of the structural unit (a5) is 20 mol % or more, preferably 30 mol % or more, more preferably 40 mol % or more, and still more preferably 45 mol % or more, based on the total of all structural units of the resin (X). The content is also 100 mol % or less, preferably 80 mol % or less, more preferably 70 mol % or less, still more preferably 60 mol % or less, and yet more preferably 55 mol % or less. Specifically, the content is 20 to 100 mol %, preferably 20 to 80 mol %, more preferably 30 to 70 mol %, still more preferably 40 to 60 mol %, and yet more preferably 45 to 55 mol %. When the resin (X) includes a structural unit (a4) and a structural unit (a5), the total content of the structural unit (a4) and the structural unit (a5) is 40 mol % or more, preferably 60 mol % or more, more preferably 70 mol % or more, and still more preferably 80 mol % or more, based on the total of all structural units of the resin (X). The content is also 100 mol % or less. Specifically, the content is 40 to 100 mol %, preferably 60 to 100 mol %, more preferably 70 to 100 mol %, and still more preferably 80 to 100 mol %.

Particularly, the resin (X) is preferably a resin composed only of a structural unit (a4) and/or a structural unit (a5). In this case, a ratio of structural unit (a4):structural unit (a5) is 0:100 to 100:0, preferably 10:90 to 90:10, and more preferably 30:70 to 70:30 or 40:60 to 60:40.

The respective structural unit constituting the resin (AX) and the resin (X) may be used alone, or two or more structural units may be used in combination. Using a monomer from which these structural units are derived, it is possible to produce by a known polymerization method (e.g. radical polymerization method). The content of the respective structural units included in the resin (X) can be adjusted according to the amount of the monomer used in the polymerization.

The weight-average molecular weight of the resin (X) is preferably 6,000 or more (more preferably 7,000 or more) and 80,000 or less (more preferably 60,000 or less), and an oligomer having a weight-average molecular weight of less than the above range may be included. The measurement means of the weight-average molecular weight of the resin (X) and the resin (X) is the same as in the case of the resin (A) or the like.

When the resist composition of the present invention includes the resin (X), the content is preferably 1 to 60 parts by mass, more preferably 1 to 50 parts by mass, still more preferably 1 to 40 parts by mass, yet more preferably 1 to 30 parts by mass, and particularly preferably 1 to 8 parts by mass, based on 100 parts by mass of the resin (A) or the like.

The total content of the resin (A) and the resin (Ap) in the resist composition is preferably 80% by mass or more and 99% by mass or less, and more preferably 90% by mass or more and 99% by mass or less, based on the solid content of the resist composition. The total content of the resin (A) and the resin (Ap) is preferably 80% by mass or more and 99% by mass or less, and more preferably 90% by mass or more and 99% by mass or less, based on the solid content of the resist composition. When including resins other than the resin (A) or the like, the total content of the resin (A) or the like and resins other than the resin (A) or the like is preferably 80% by mass or more and 99% by mass or less, and more preferably 90% by mass or more and 99% by mass or less, based on the solid content of the resist composition. The solid content of the resist composition and the content of the resin thereto can be measured by a known analysis means such as liquid chromatography or gas chromatography.

### <Compound (B)>

Either a nonionic compound or an ionic compound may be used as the compound (B). Examples of the nonionic compound include sulfonate esters (e.g., 2-nitrobenzyl ester, aromatic sulfonate, oxime sulfonate, N-sulfonyloxyimide, sulfonyloxyketone, diazonaphthoquinone 4-sulfonate), sulfones (e.g., disulfone, ketosulfone, sulfonyldiazomethane) and the like. Typical examples of the ionic compound include onium salts containing an onium cation (e.g., diazonium salt, phosphonium salt, sulfonium salt, iodonium salt). Examples of the anion of the onium salt include sulfonic acid anion, benzenesulfonyl anion, sulfonylimide anion, sulfonylmethide anion and carboxylic acid anion.

Specific examples of the compound (B) include compounds generating an acid upon exposure to radiation mentioned in JP 63-26653 A, JP 55-164824 A, JP 62-69263 A, JP 63-146038 A, JP 63-163452 A, JP 62-153853 A, JP 63-146029 A, U.S. Pat. Nos. 3,779,778, 3,849,137, DE Patent No. 3914407 and EP Patent No. 126,712. Compounds produced by a known method may also be used.

The compound (B) is preferably a salt represented by formula (B1) (hereinafter sometimes referred to as “compound (B1)”, in which the salt (I) is excluded) or a salt represented by formula (B1-A3) (hereinafter sometimes referred to as “acid generator (B1-A3)”):

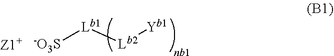

wherein
L^{b1} represents an (nb1+1)-valent hydrocarbon group which may have a substituent, and —CH₂— included in the saturated hydrocarbon group may be replaced by —O—, —S—, —CO— or —SO₂—,
L^{b2} represents a divalent hydrocarbon group having 1 to 24 carbon atoms which may have a substituent, and —CH₂— included in the hydrocarbon group may be replaced by —O—, —S—, —CO— or —SO₂—,
Y^{b1} represents a methyl group which may have a substituent or a cyclic hydrocarbon group having 3 to 24 carbon atoms which may have a substituent, and —CH₂— included in the cyclic hydrocarbon group may be replaced by —O—, —S—, —SO₂— or —CO—,
nb1 represents an integer of 1 to 6, and when nb1 is 2 or more, a plurality of groups in parentheses may be the same or different from each other, and
Z1⁺ represents an organic cation.

In formula (B1), examples of the (nb1+1)-valent hydrocarbon group as for L^{b1} include a chain hydrocarbon group, an alicyclic hydrocarbon group, an aromatic hydrocarbon group, and groups which are obtained by removing nb1 hydrogen atoms from a group obtained by combining these two or more groups, and are bonded to one or more L^{b2}.

Examples of the chain hydrocarbon group include groups obtained by removing nb1 hydrogen atoms of the alkyl group or alkenyl group. The alkyl group may be either linear or branched, and specific examples thereof include a methane group, an ethane group, a propane group, an iso-propyl group, an n-butyl group, a sec-butyl group, an iso-butyl group, a tert-butyl group, a heptyl group, a hexyl group, a heptyl group, an octyl group, a nonyl group, a decane group, an undecane group, a dodecane group, a tridecane group, a tetradecane group, a pentanedecane, a heptadecane group and the like. Examples of the alkenyl group include an ethenyl group, a propenyl group, an isopropenyl group, a butenyl group, an isobutenyl group, a tert-butenyl group, a pentenyl group, a hexenyl group, a heptenyl group, an octenyl group, an isooctenyl group and a nonenyl group.

The number of carbon atoms of the chain hydrocarbon group is preferably 1 to 36, more preferably 1 to 20, and still more preferably 1 to 10.

Examples of the alicyclic hydrocarbon group include groups obtained by removing nb1 hydrogen atoms of the monocyclic or polycyclic cycloalkyl group. Examples of the monocyclic cycloalkyl group include a cyclobutyl group, a cyclopentane group, a cyclohexane group, a cyclopentyl group, a cyclooctyl group and the like.

Examples of the polycyclic cycloalkyl group include a cycloalkyl group having a crosslinked structure, a cycloalkyl group in which two or more rings are fused, or a cycloalkyl group in which two rings are bonded by spiro bonding. Examples of the cycloalkyl group having a crosslinked structure include a norbornane group, an adamantane group and the like. Examples of the cycloalkyl group in which two or more rings are fused include a bicyclo[4,4,0]decane group, a steroid group (steroid skeleton) and the like. Examples of the ring in which two rings are bonded by spiro bonding include a spirocyclic cycloalkyl group in which one cycloalkyl group selected from the group consisting of a cyclopentyl group, a cyclohexyl group, a norbornane group and an adamantyl group, and a cycloalkyl group having 5 to 8 carbon atoms are bonded by spiro bonding, and the like. A double bond may be formed between two carbon atoms included in the alicyclic hydrocarbon group.

More specifically, alicyclic hydrocarbon groups represented by the following formulas are exemplified.

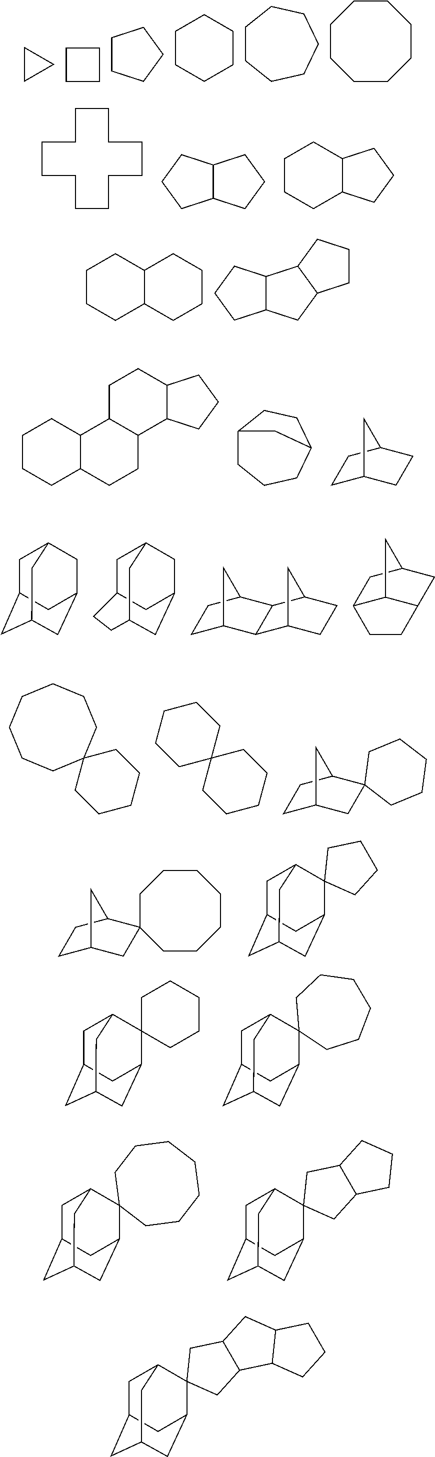

When the alicyclic hydrocarbon group is a monocyclic cycloalkyl group, the number of carbon atoms of the alicyclic hydrocarbon group is preferably 3 to 12, and more preferably 3 to 8. When the alicyclic hydrocarbon group is a polycyclic cycloalkyl group, the number of carbon atoms of the alicyclic hydrocarbon group is preferably 7 to 12.

Examples of the aromatic hydrocarbon group include aryl groups obtained by removing nb1 hydrogen atoms. Examples of the aryl group include a phenyl group, a naphthyl group, an anthryl group, a biphenyl group, a fluorene group and the like.

The number of carbon atoms of the aromatic hydrocarbon group is preferably 5 to 14, and more preferably 5 to 10.

When —CH₂— included in the hydrocarbon group as for L^{b1} is replaced by —O—, —CO—, —S— or —SO₂—, the number of carbon atoms before replacement is taken as the number of carbon atoms of the hydrocarbon group.

Of the hydrocarbon group as for L^{b1}, examples of the group in which —CH₂— included in the chain hydrocarbon group is replaced by —O—, —CO—, —S— or —SO₂— include a hydroxy group (a group in which —CH₂— included in the methyl group is replaced by —O—), a carboxy group (a group in which —CH₂—CH₂— included in the ethyl group is replaced by —O—CO—), a carbonyl group (a group in which —CH₂— included in the methylene group is replaced by —CO—), an oxy group (a group in which —CH₂— included in the methylene group is replaced by —O—), an alkoxy group (a group in which —CH₂— at any position included in the alkyl group is replaced by —O—), an alkoxycarbonyl group (a group in which —CH₂—CH₂— at any position included in alkyl group is replaced by —O—CO—), an alkylcarbonyl group (a group in which —CH₂— at any position included in the alkyl group is replaced by —CO—), an alkylcarbonyloxy group (a group in which —CH₂—CH₂— at any position included in the alkyl group is replaced by —CO—O—), an alkanediyloxy group (a group in which —CH₂— at any position included in the alkanediyl group is replaced by —O—), an alkanediyloxycarbonyl group (a group in which —CH₂—CH₂— at any position included in the alkanediyl group is replaced by —O—CO—), an alkanediylcarbonyl group (a group in which —CH₂— at any position included in the alkanediyl group is replaced by —CO—), an alkanediylcarbonyloxy group (a group in which —CH₂—CH₂— at any position included in the alkanediyl group is replaced by —CO—O—) and the like.

Of the hydrocarbon group as for L^{b1}, examples of the group in which —CH₂— included in the alicyclic hydrocarbon group is replaced by —O—, —CO—, —S— or —SO₂— include groups having a structure such as cyclic ether, cyclic ketone, cyclic ester (lactone), cyclic thioether, cyclic acetal, cyclic sultone and the like. Specific examples thereof include alicyclic hydrocarbon groups represented by the following formulas. The bonding site of the alicyclic hydrocarbon group represented by the following formulas can be any position.

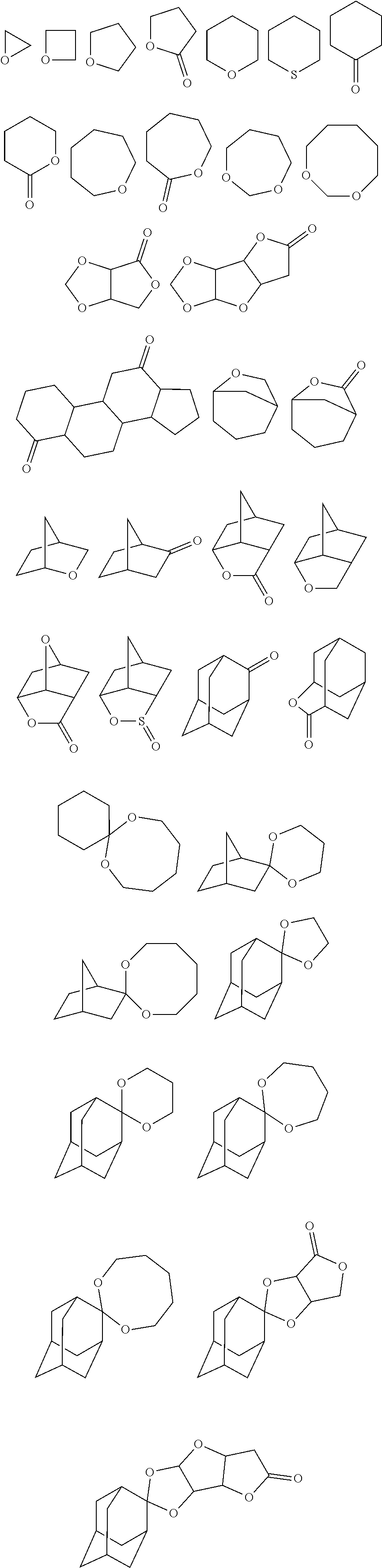

Of the hydrocarbon group as for L^{b1}, —CH₂— included in the aromatic hydrocarbon group may be replaced by —O— or —S—, and examples of the group in which —CH₂— is replaced by —O— or —S— include a furan ring or a thiophene ring.

Examples of the group obtained by combining two or more groups of the chain hydrocarbon group, the alicyclic hydrocarbon group and the aromatic hydrocarbon group include a group obtained by combining the chain hydrocarbon group with the alicyclic hydrocarbon group, a group obtained by combining the chain hydrocarbon group with the aromatic hydrocarbon group, a group obtained by combining the alicyclic hydrocarbon group with the aromatic hydrocarbon group, and a group obtained by combining the chain hydrocarbon group, the alicyclic hydrocarbon group and the aromatic hydrocarbon group, and also include combined groups mentioned above. The group obtained by combining the alicyclic hydrocarbon group with the aromatic hydrocarbon group may also be a fused ring.

Examples of the divalent hydrocarbon group as for L^{b2} include a divalent chain hydrocarbon group, a divalent alicyclic hydrocarbon group, a divalent aromatic hydrocarbon group, and groups obtained by combining these two or more groups, and groups which are obtained by removing one hydrogen atom and are bonded to one or more Y^{b1}.

Examples of the divalent chain hydrocarbon group, the divalent alicyclic hydrocarbon group, the divalent aromatic hydrocarbon group, and the divalent group obtained by combining these two or more groups as for L^{b2} include the chain hydrocarbon group, the alicyclic hydrocarbon group, the aromatic hydrocarbon group, and groups obtained by removing one hydrogen atom from the group formed by combining these two or more groups mentioned as for L^{b1}, respectively, as long as the number of carbon atoms permits.

Examples of the substituent which may be possessed by the hydrocarbon group as for L^{b1} and L^{b2} include a halogen atom, a cyano group and a nitro group. Examples of the halogen atom include a fluorine atom, a chlorine atom, a bromine atom, an iodine atom and the like.

When L^{b1} and L^{b2} are groups obtained by combining an alicyclic hydrocarbon group or an aromatic hydrocarbon group with a chain hydrocarbon group, the chain hydrocarbon group may be substantially regarded as a substituent which is possessed by the alicyclic hydrocarbon group or the aromatic hydrocarbon group. By replacing —CH₂— of the chain hydrocarbon group included in the hydrocarbon group as for L^{b1} and L^{b2} by —O—, —CO—, —S— or —SO₂—, the hydrocarbon group as for L^{b1} and L^{b2} can substantially have a substituent such as a hydroxy group, a carboxyl group, an alkoxy group, an alkylcarbonyl group, an alkoxycarbonyl group, an alkylcarbonyl group, an alkylcarbonyloxy group, a thiol group or a sulfonyl group.

Examples of the cyclic hydrocarbon group as for Y^{b1} include an alicyclic hydrocarbon group, an aromatic hydrocarbon group, and an alicyclic hydrocarbon group and an aromatic hydrocarbon group.

Examples of the alicyclic hydrocarbon group and the aromatic hydrocarbon group as for Y^{b1} include same alicyclic hydrocarbon groups and aromatic hydrocarbon groups as mentioned as for L^{b1}, respectively, and when having no substituent, they may be a monovalent alicyclic hydrocarbon group and an aromatic hydrocarbon group.

Examples of the substituent which may be possessed by the methyl group as for Y^{b1} include a halogen atom, a cyano group, a hydroxy group, a nitro group and the like. Examples of the substituent which may be possessed by the cyclic hydrocarbon group as for Y^{b1} include a halogen atom, a cyano group, a nitro group, or a hydrocarbon group having 1 to 18 carbon atoms which may have a halogen atom, a cyano group or a nitro group (—CH₂— included in the hydrocarbon group may be replaced by —O—, —S—, —CO— or —SO₂—). The number of carbon atoms of the hydrocarbon group which may be possessed by the cyclic hydrocarbon group as for Y^{b1} is not included in the number of carbon atoms of the hydrocarbon group of the cyclic hydrocarbon group as for Y^{b1}.

Examples of the hydrocarbon group having 1 to 18 carbon atoms which may be possessed, as the substituent, by the cyclic hydrocarbon group as for Y^{b1} include a chain hydrocarbon group, an alicyclic hydrocarbon group, an aromatic hydrocarbon group, and groups obtained by combining these groups. Examples of the chain hydrocarbon group, the alicyclic hydrocarbon group, the aromatic hydrocarbon group, and groups obtained by combining these two or more groups include the same groups as mentioned in the chain hydrocarbon group, the alicyclic hydrocarbon group, the aromatic hydrocarbon group, and groups obtained by combining these two or more groups as for L^{b1} as long as the upper limit of the number of carbon atoms permit. Examples of the hydrocarbon group having 1 to 18 carbon atoms which may be possessed, as the substituent, by the cyclic hydrocarbon group as for Y^{b1} include the same groups as mentioned as for the group in which —CH₂— included in the hydrocarbon group as for L^{b1} is replaced by —O—, —S—, —CO— or —SO₂— as long as the upper limit of the number of carbon atoms permit. The hydrocarbon group having 1 to 18 carbon atoms which may be possessed, as the substituent, by the cyclic hydrocarbon group as for Y^{b1} may constitute a protecting group or a leaving group (an acid-labile group or a base-labile group) which is generally used in the relevant field.

The anion included in the salt represented by formula (B1) is preferably an anion represented by the following formula (B1-1) or an anion represented by formula (B1-2):

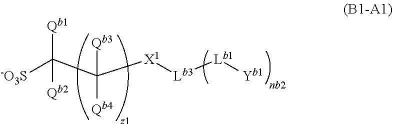

wherein, in formula (B1-1),
L^{b2} and Y^{b1} are the same as defined in formula (B1),
Q^{b1}, Q^{b2}, Q^{b3} and Q^{b4} each independently represent a hydrogen atom, a fluorine atom, a perfluoroalkyl group having 1 to 6 carbon atoms or an alkyl group having 1 to 6 carbon atoms,
z1 represents an integer of 0 to 6,
X¹ represents —O—CO—, —CO—O—, —O—CO—O— or —O—,
L^{b3} represents an (nb2+1) hydrocarbon group having 1 to 24 carbon atoms which may have a substituent, and —CH₂— included in the hydrocarbon group may be replaced by —O—, —S—, —CO— or —SO₂—, and
nb2 represents an integer of 1 to 3.

Examples of the alkyl group as for Q^{b1}, Q^{b2}, Q^{b3} and Q^{b4} include a methyl group, an ethyl group, a propyl group, an isopropyl group, a butyl group, a sec-butyl group, a tert-butyl group, a pentyl group and a hexyl group.

Examples of the perfluoroalkyl group having 1 to 6 carbon atoms as for Q^{b1}, Q^{b2}, Q^{b3} and Q^{b4} include a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group and a perfluorohexyl group.

Q^{b1} and Q^{b2} preferably includes a fluorine atom or a perfluoroalkyl group in at least one of Q^{b1} and Q^{b2}, more preferably a fluorine atom or a perfluoroalkyl group, still more preferably a fluorine atom or a trifluoromethyl group, and yet more preferably both are fluorine atoms.

Preferably, Q^{b3} and Q^{b4} are each independently a hydrogen atom, a fluorine atom or a perfluoroalkyl group having 1 to 3 carbon atoms, Q^{b3} is preferably a hydrogen atom, a fluorine atom or a perfluoroalkyl group having 1 to 3 carbon atoms, and Q^{b4} is preferably a hydrogen atom or a fluorine atom.

z1 is preferably 0 to 3, and more preferably 0, 1 or 2.

X¹ is preferably —O—CO— or —CO—O—.

Examples of the hydrocarbon group in L^{b3} include the same hydrocarbon groups as mentioned as for L^{b1} of formula (B1) as long as the upper limit of the number of carbon atoms permit.

In formula (B1-1), L^{b3} is preferably a chain hydrocarbon group having 1 to 12 carbon atoms which may have a substituent (—CH₂— included in the chain hydrocarbon group may be replaced by —O— or —CO—), an alicyclic hydrocarbon group having 3 to 18 carbon atoms which may have a substituent (—CH₂— included in the alicyclic hydrocarbon group may be replaced by —O—, —S—, —CO— or —SO₂—), an aromatic hydrocarbon group having 6 to 10 carbon atoms which may have a substituent (—CH₂— included in the alicyclic hydrocarbon group may be replaced by —O— or —S—) or groups obtained by combining these two or more groups, and more preferably a chain hydrocarbon group having 1 to 6 carbon atoms, or a group represented by the following formula (Lb3-1). When the chain hydrocarbon group is replaced by —O— or —CO—, the number of —CH₂— included in the chain hydrocarbon group is replaced by —O— or —CO— is preferably 1 to 4, and one —CH₂—CH₂— included in the chain hydrocarbon group is preferably replaced by —O—CO— or —CO—O—, or —CH₂—CH₂—CH₂— included in the chain hydrocarbon group is preferably replaced by —O—CO—O—:

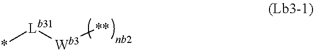

wherein, in formula (Lb3-1),
nb2 is the same as defined in formula (B1-1),
L^{b3}1 represents a single bond or a chain hydrocarbon group having 1 to 12 carbon atoms, —CH₂— included in the chain hydrocarbon group may be replaced by —O— or —CO—, and may have a substituent,
W^{b3} represents an alicyclic hydrocarbon group having 3 to 18 carbon atoms or an aromatic hydrocarbon group having 6 to 10 carbon atoms, —CH₂— included in the alicyclic hydrocarbon group may be replaced by —O—, —S—, —CO— or —SO₂—, —CH₂— included in the aromatic hydrocarbon group may be replaced by —O— or —S—, and the alicyclic hydrocarbon group may have a substituent, and
* and ** represent a bonding site, and * represents a bonding site to X¹.

In formula (Lb3-1), examples of the chain hydrocarbon group as for L^{b3}1 include the same chain hydrocarbon groups as mentioned as the chain hydrocarbon groups as for L^{b1} as long as the upper limit of the number of carbon atoms permit.

In formula (Lb3-1), examples of the alicyclic hydrocarbon group and the aromatic hydrocarbon group as for W^{b3} include alicyclic hydrocarbon groups and aromatic hydrocarbon groups as mentioned as for L^{b1} as long as the upper limit of the number of carbon atoms permit.

In formula (Lb3-1), examples of the substituent which may be possessed by the chain hydrocarbon group as for L^{b3}1 and the substituent which may be possessed by the alicyclic hydrocarbon group and the aromatic hydrocarbon group as for W^{b1} include the same substituents as mentioned as for the substituent which may be possessed by the hydrocarbon group as for L^{b1}.

L^{b3}1 is preferably a single bond or an alkanediyl group having 1 to 6 carbon atoms (—CH₂— included in the alkanediyl group may be replaced by —O— or —CO—).

Particularly, the alicyclic hydrocarbon group and the aromatic hydrocarbon group as for W^{b1} are preferably an alicyclic hydrocarbon group and an aromatic hydrocarbon group mentioned below. In the alicyclic hydrocarbon group and the aromatic hydrocarbon group mentioned below, * and ** represent a bonding site, * represents a bonding site to X¹ or L^{b3}1, ** represents a hydrogen atom, a substituent or a bonding site to L^{b2}, and at least one ** represents a bonding site to L^{b2}. In the alicyclic hydrocarbon group mentioned below, —CH₂— included in the alicyclic hydrocarbon group may be replaced by —O—, —S—, —CO— or —SO₂—. When —CH₂— included in the alicyclic hydrocarbon group is replaced by —O—, —S—, —CO— or —SO₂—, it is preferable to form an ether ring, an ester ring (lactone), a carbonic acid ester ring, a sultone ring or an acetal ring.

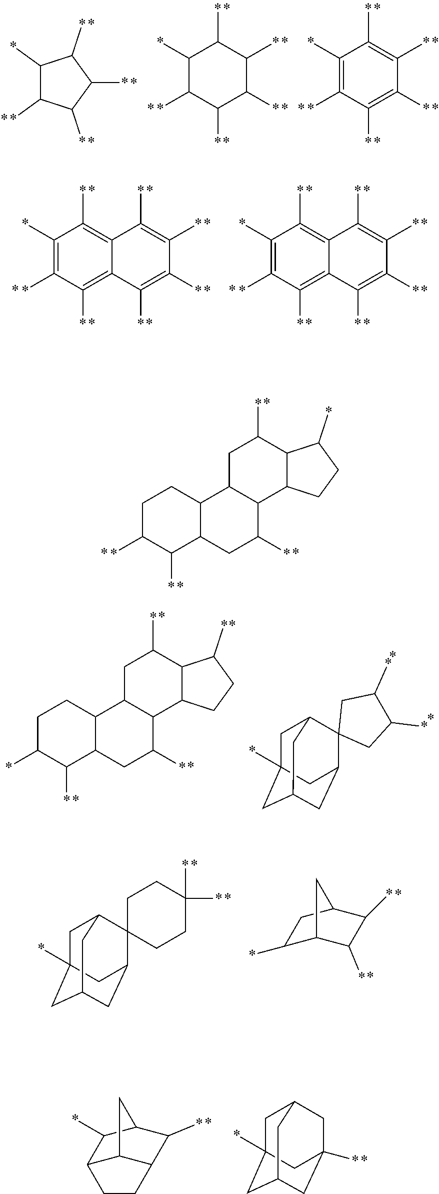

In formula (B1-1), L^{b2} is preferably a single bond or an alkanediyl group having 1 to 12 carbon atoms (—CH₂— included in the alkanediyl group may be replaced by —O— or —CO—), and preferably a single bond, —O—, —O—CO—, —CO—O—, —O—CO—O— or *-Lb21-X²-L^{b22}-** (one of L^{b21} and L^{b22} represents at least an alkanediyl group having 1 to 6 carbon atoms, and the other one represents a single bond or an alkanediyl group having 1 to 6 carbon atoms, X² represents —O—, —CO—O—, —O—CO— or —O—CO—O—, * and ** represent a bonding site, and ** represents a bonding site to Y^{b1}, in which the total number of carbon atoms of L^{b21}, X² and L^{b22} is 12 or less).

In formula (B1-1), L^{b2} is preferably a single bond or an alkanediyl group having 1 to 12 carbon atoms (—CH₂— included in the alkanediyl group may be replaced by —O— or —CO—), and preferably a single bond, —O—, —O—CO—, —CO—O—, —O—CO—O— or *-Lb21-X²-Lb22-** (one of Lb21 and Lb22 represents at least an alkanediyl group having 1 to 6 carbon atoms, and the other one represents a single bond or an alkanediyl group having 1 to 6 carbon atoms, X² represents —O—, —CO—O—, —O—CO— or —O—CO—O—, * and ** represent a bonding site, and ** represents a bonding site to Y^{b1}, in which the total number of carbon atoms of L^{b21}, X² and L^{b22} is 12 or less).

In formula (B1-1), Y^{b1} is preferably an alicyclic hydrocarbon group having 3 to 18 carbon atoms which may have a substituent, or a phenyl group which may have a substituent, and more preferably groups represented by the following formula (Y1) to formula (Y36), specifically. In formula (Y1) to formula (Y36), R^{Yb} represents an alkyl group having 1 to 4 carbon atoms of substituents which may be possessed by the cyclic hydrocarbon group as for Y^{b1}, R^{Yc} represents a hydrogen atom or a substituent which may be possessed by the cyclic hydrocarbon group as for Y^{b1}, and * represents a bonding site to L^{b2}. The alicyclic hydrocarbon group and the aromatic hydrocarbon group represented by the following formulas are not particularly shown in the following formulas, but may have any other substituents.

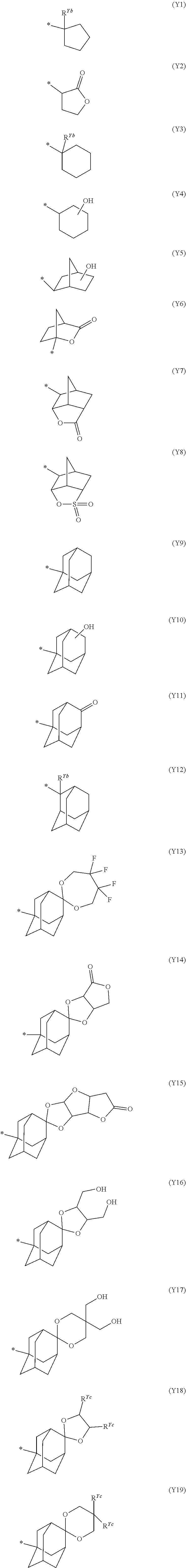

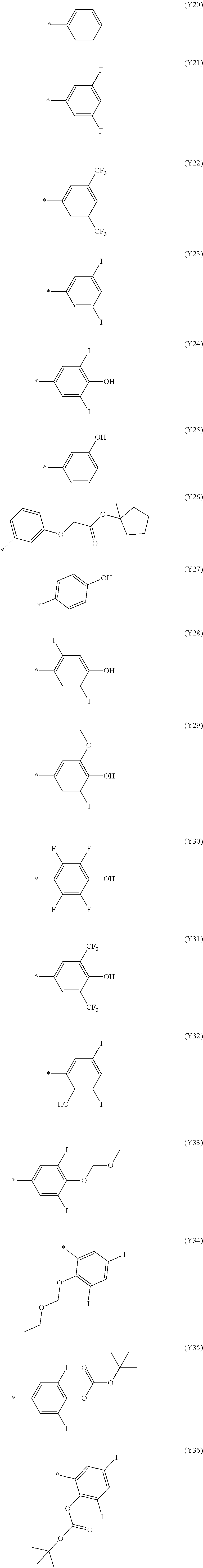

The anion included in the salt represented by formula (B1-1) is preferably an anion represented by formula (B1-A1-1) to formula (B1-A1-85) [hereinafter sometimes referred to as “anion (B1-A1-1)” or the like according to the number of formula number], and more preferably an anion represented by any one of formula (B1-A1-1) to formula (B1-A1-4), formula (B1-A1-9), formula (B1-A1-10), formula (B1-A1-24) to formula (B1-A1-33), formula (B1-A1-36) to formula (B1-A1-40) and formula (B1-A1-47) to formula (B1-A1-85).

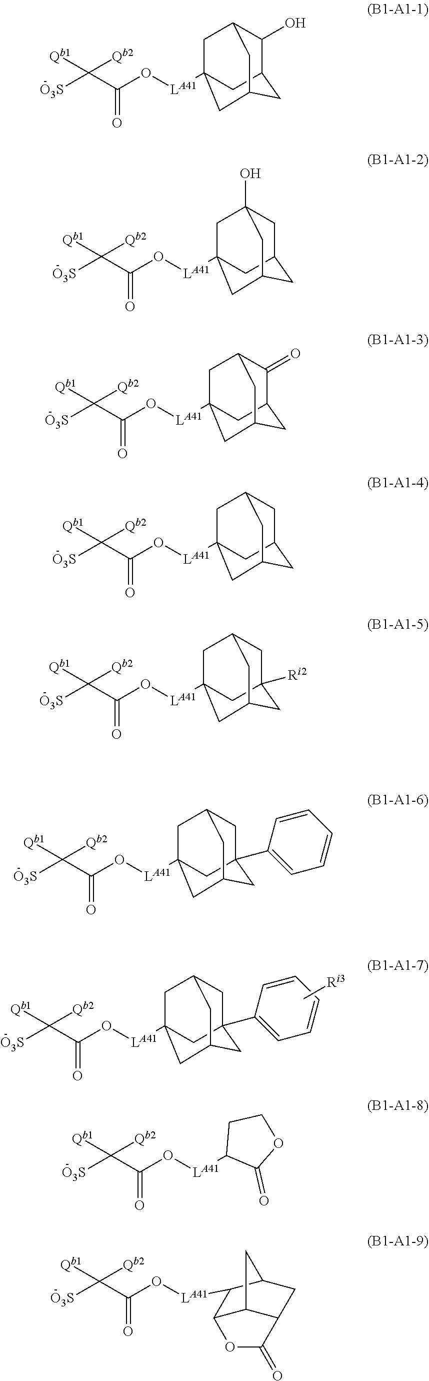

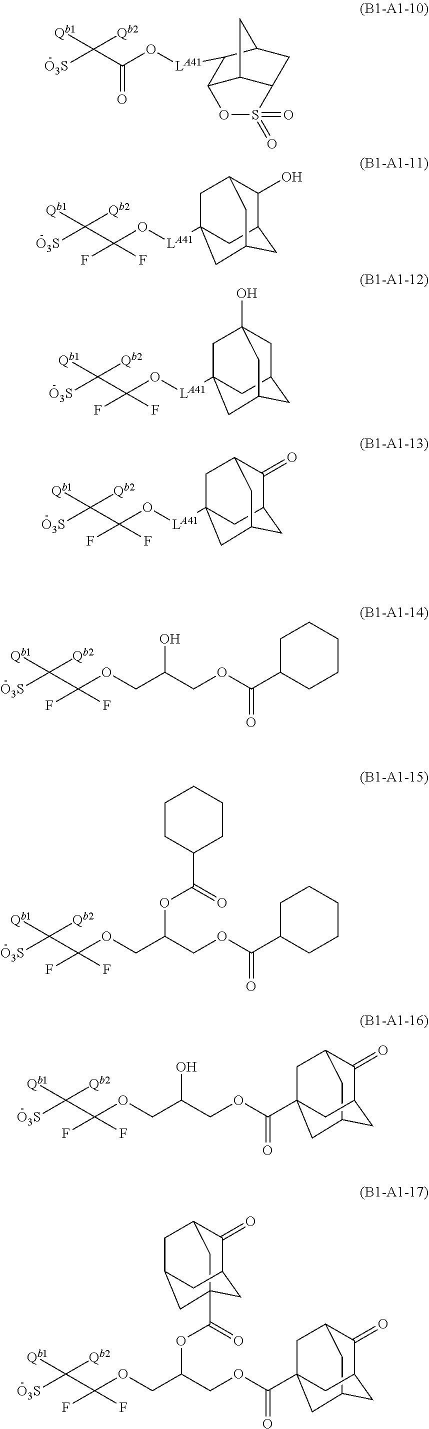

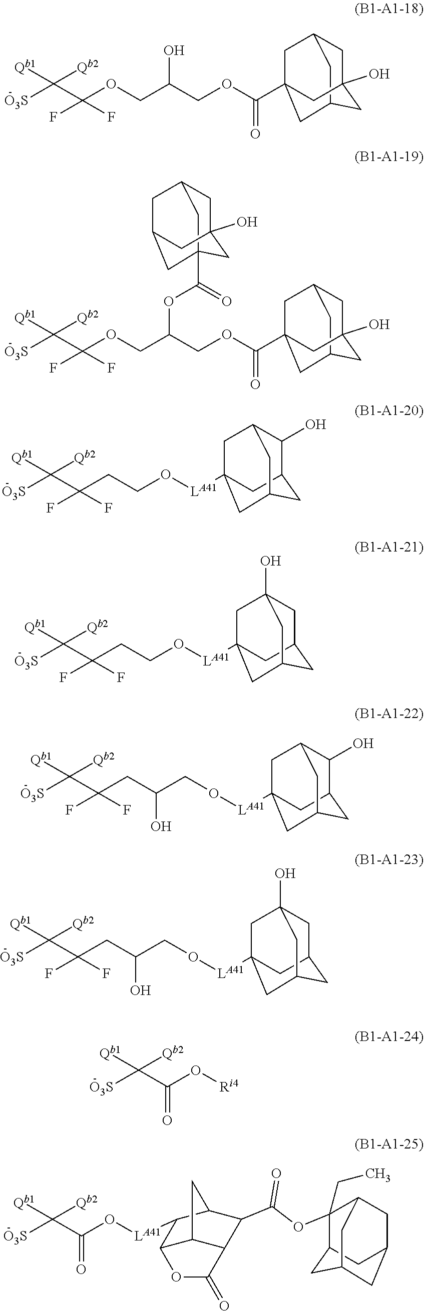

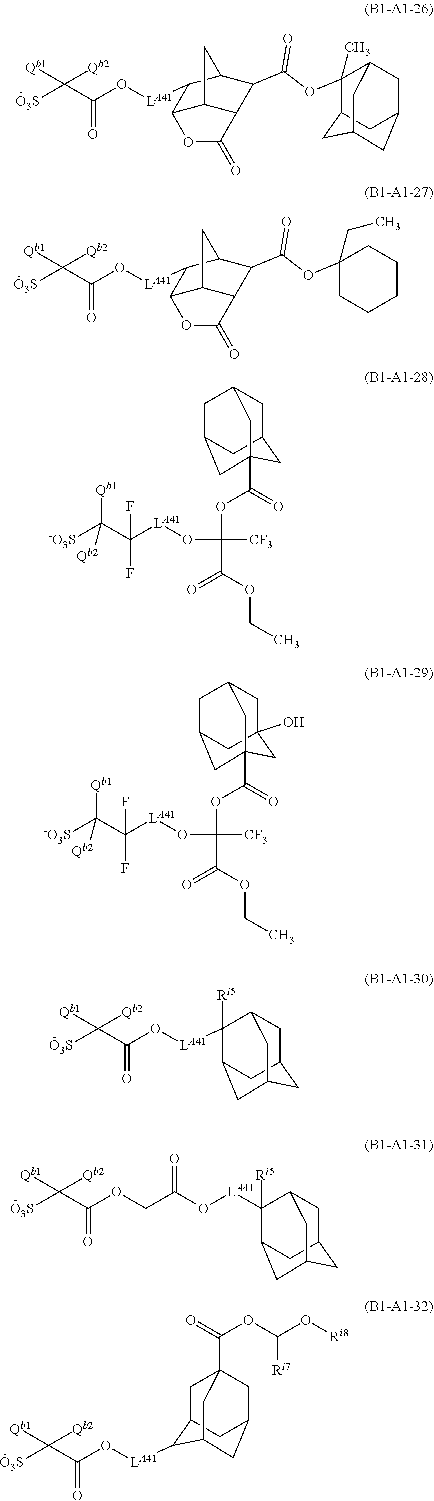

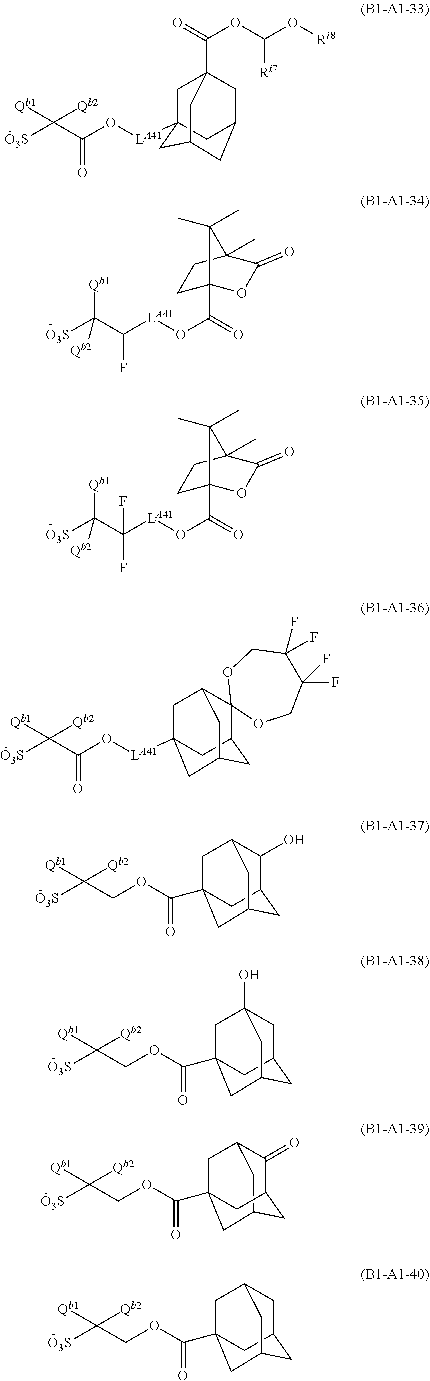

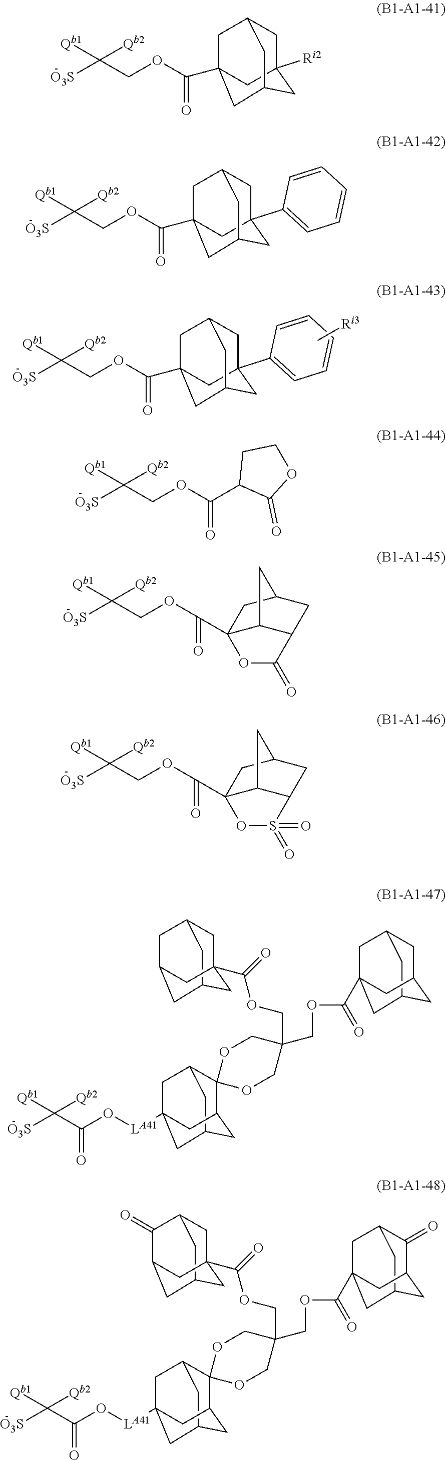

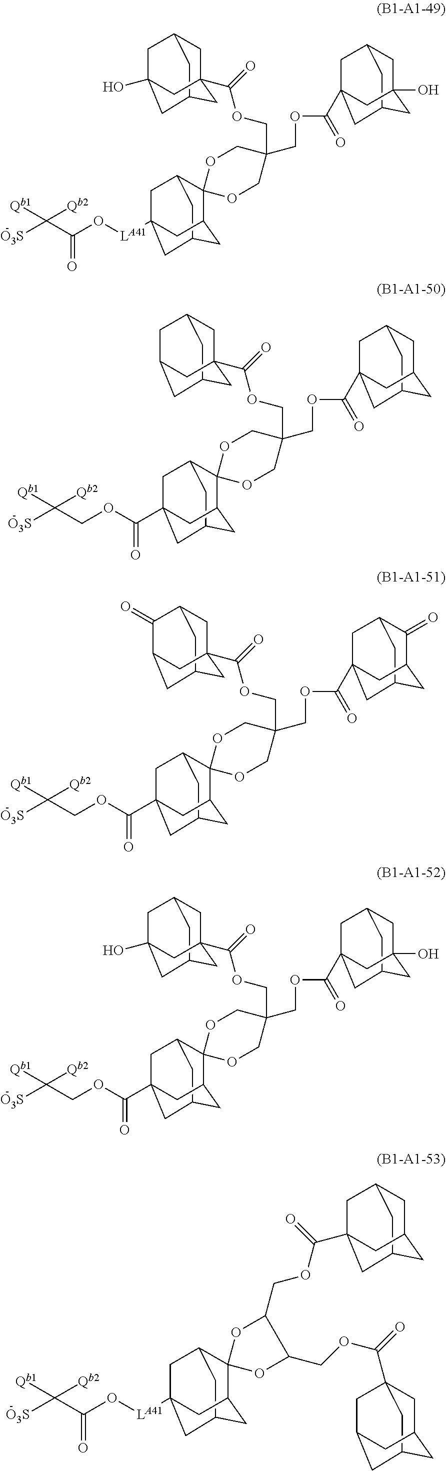

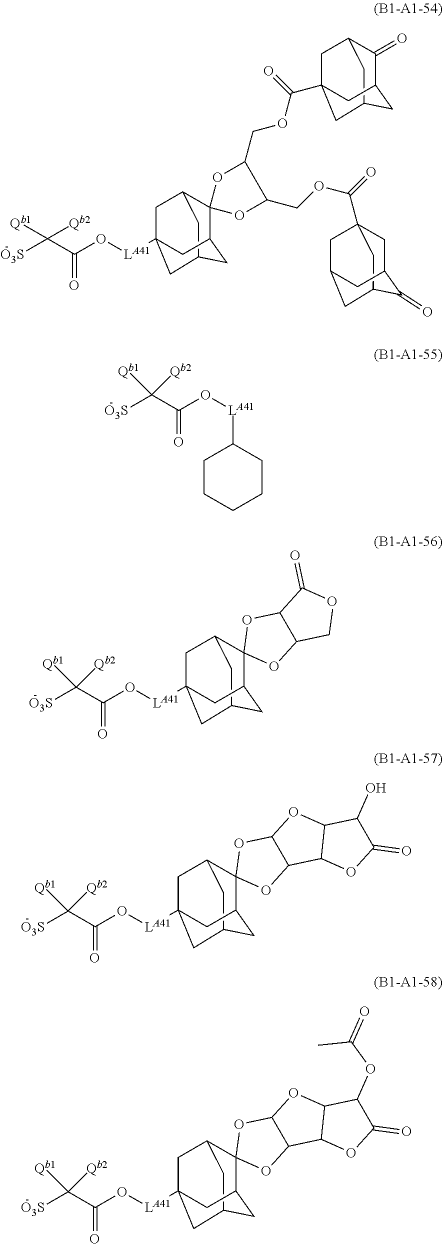

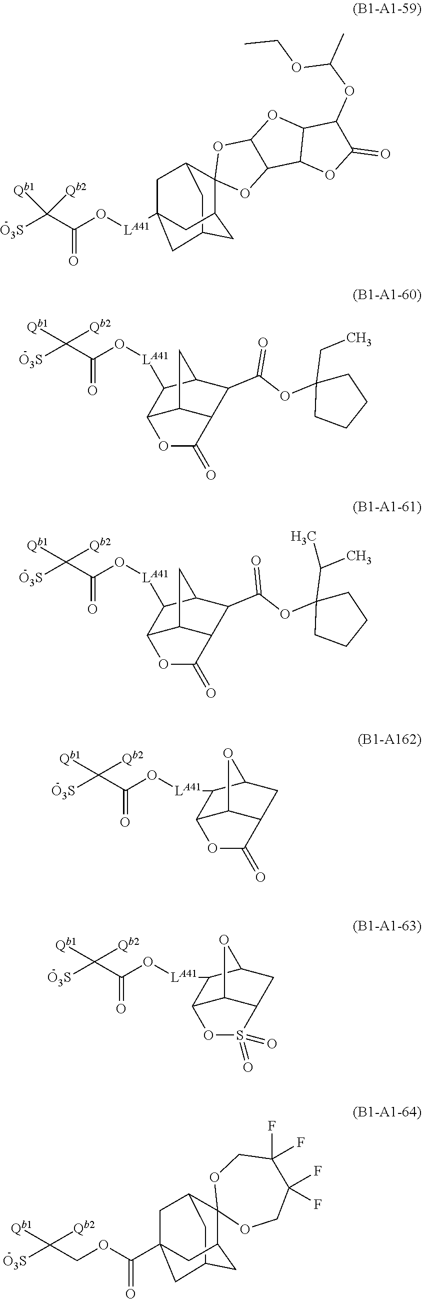

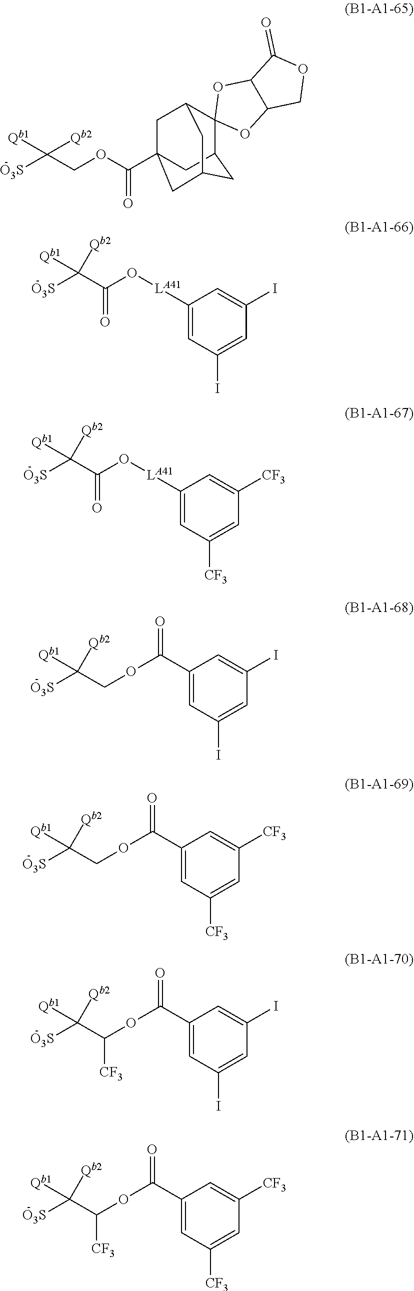

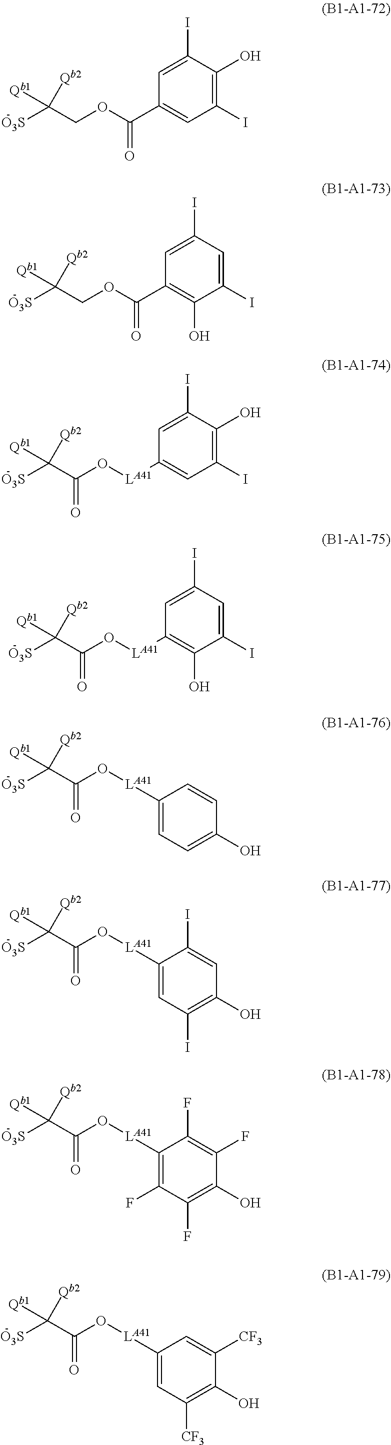

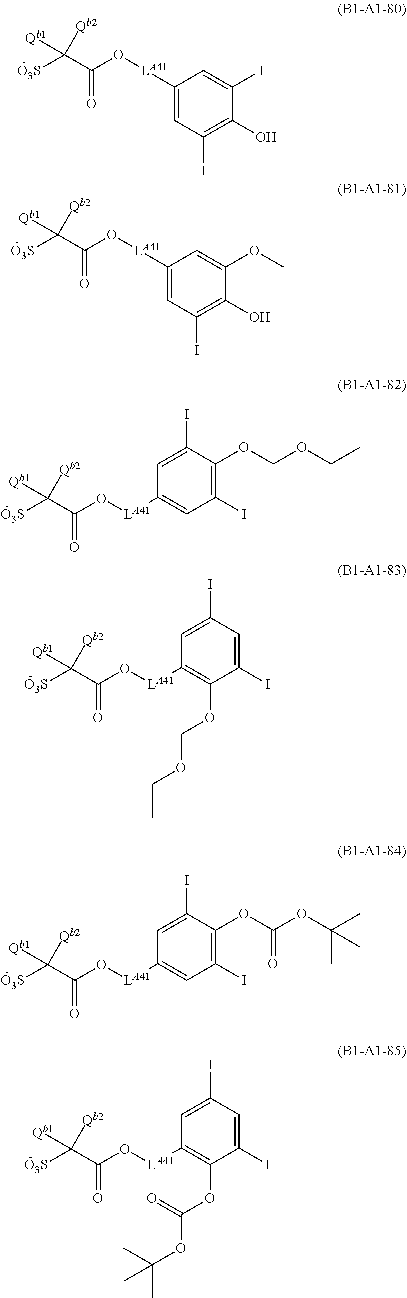

Here, Rⁱ² to Rⁱ⁷ are each independently, for example, an alkyl group having 1 to 4 carbon atoms, and preferably a methyl group or an ethyl group. Rⁱ⁸ is, for example, an aliphatic hydrocarbon group having 1 to 12 carbon atoms, preferably an alkyl group having 1 to 4 carbon atoms, an alicyclic hydrocarbon group having 5 to 12 carbon atoms, or a group formed by combining these groups, and more preferably a methyl group, an ethyl group, a cyclohexyl group or an adamantyl group. L^{A41} is a single bond or an alkanediyl group having 1 to 4 carbon atoms.

Q^{b1} and Q^{b2} are the same as defined above.

Specific examples of the anion in the salt represented by formula (B1-1) include anions mentioned in JP 2010-204646 A.

Examples of preferable anion included in the salt represented by formula (B1-1) include anions represented by formula (B1a-1) to formula (B1a-70).

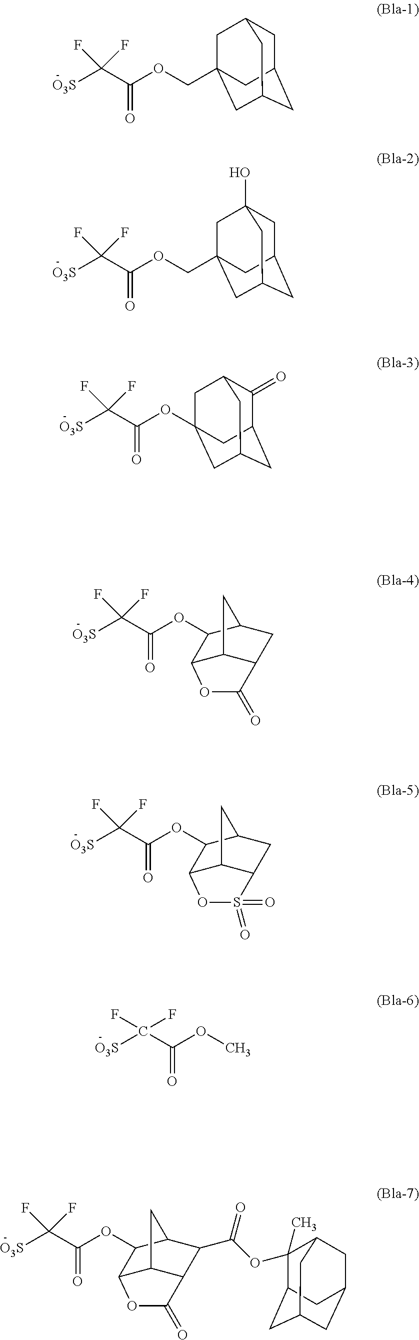

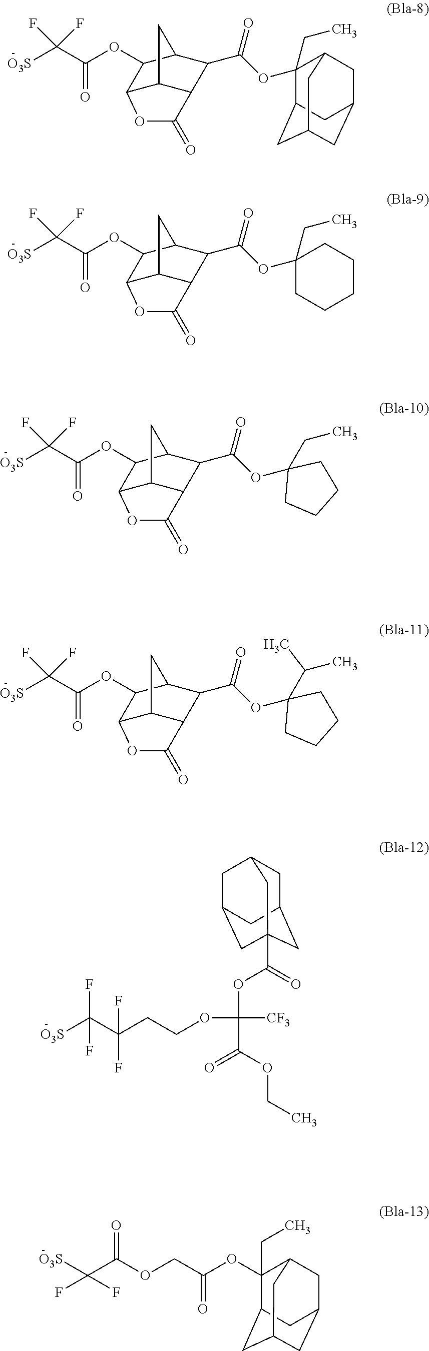

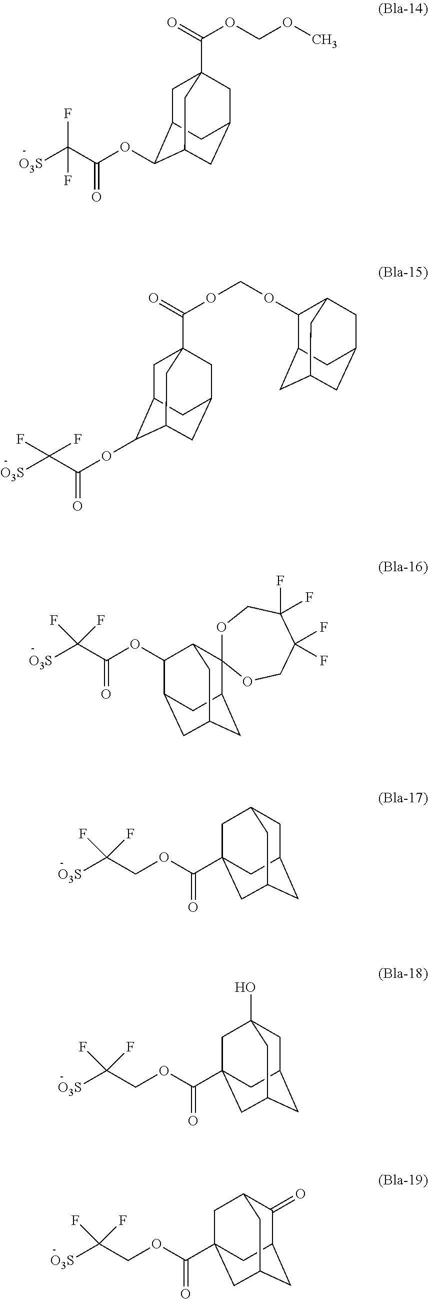

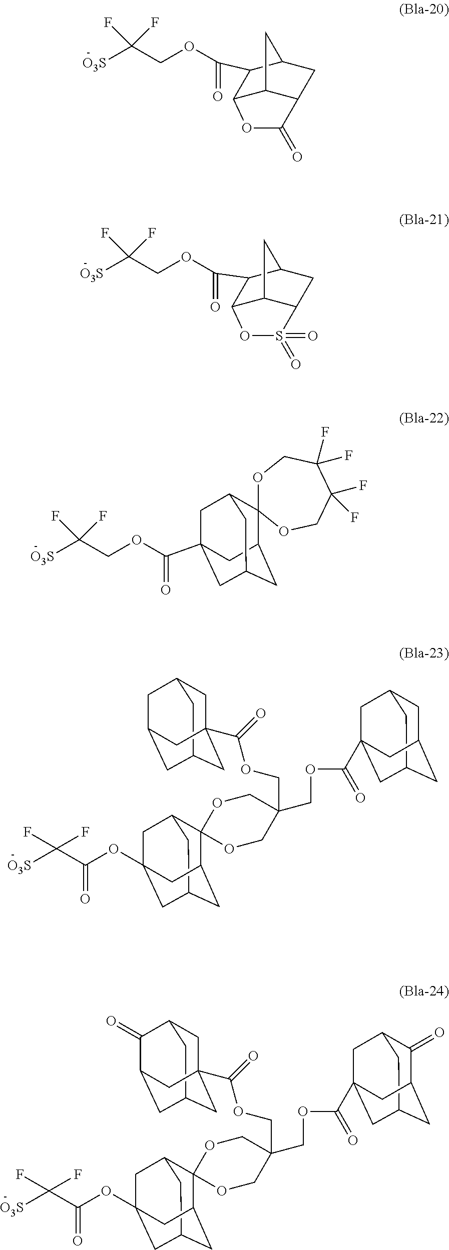

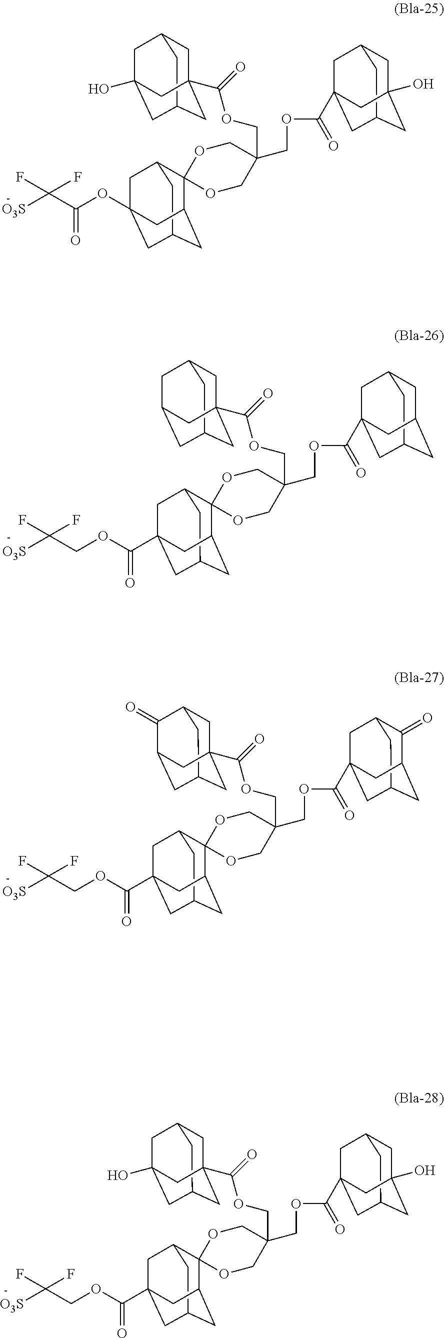

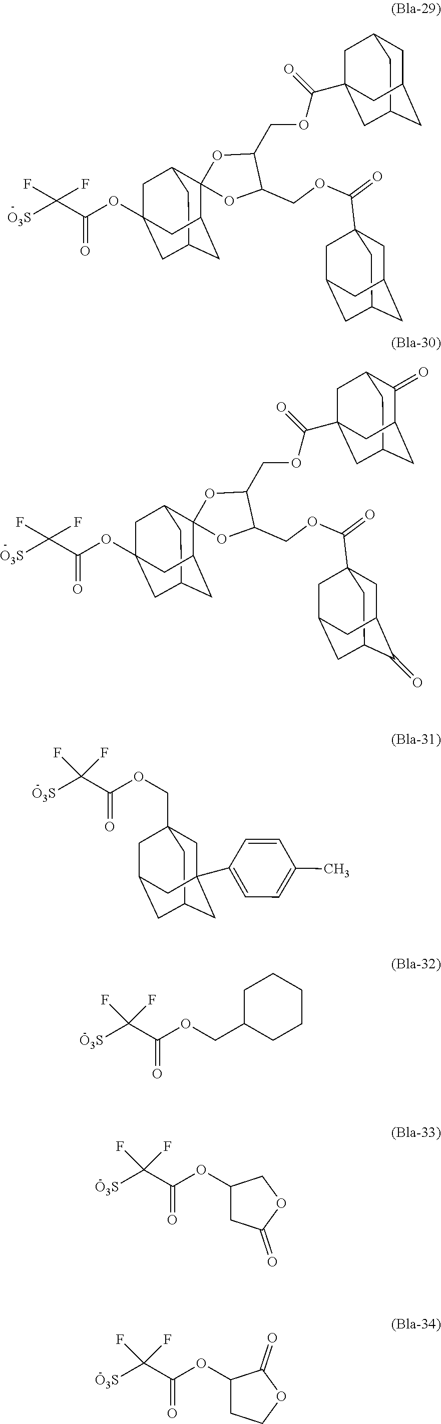

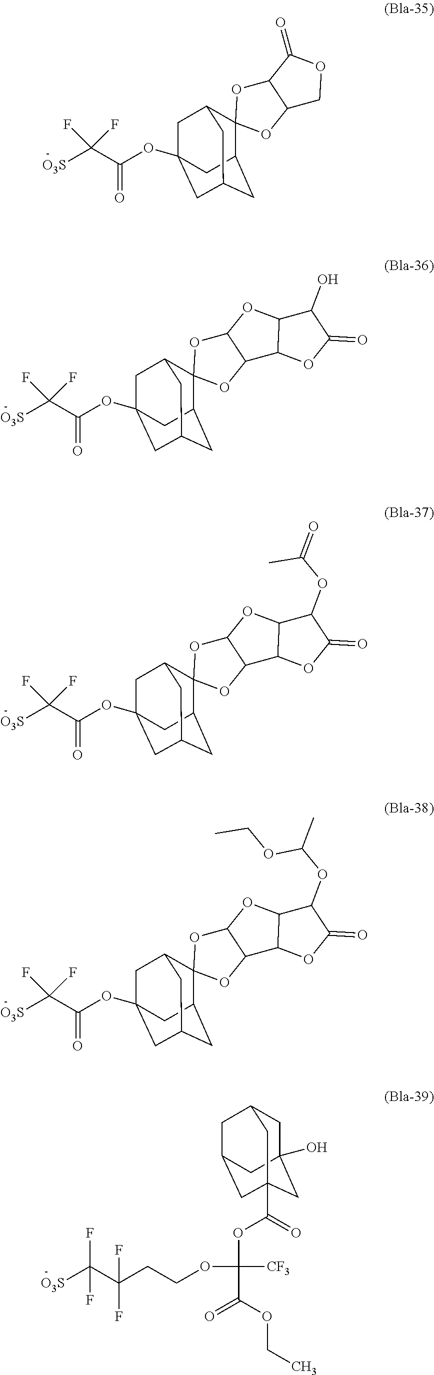

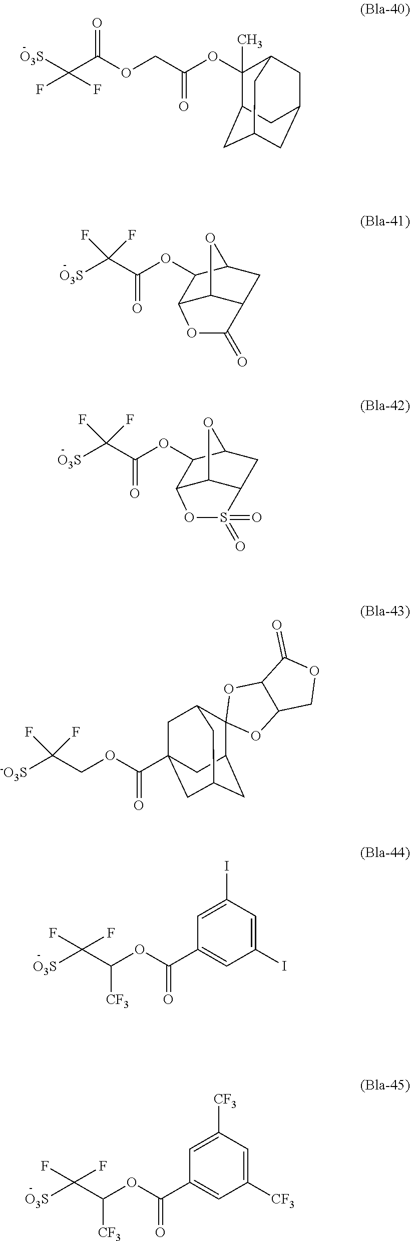

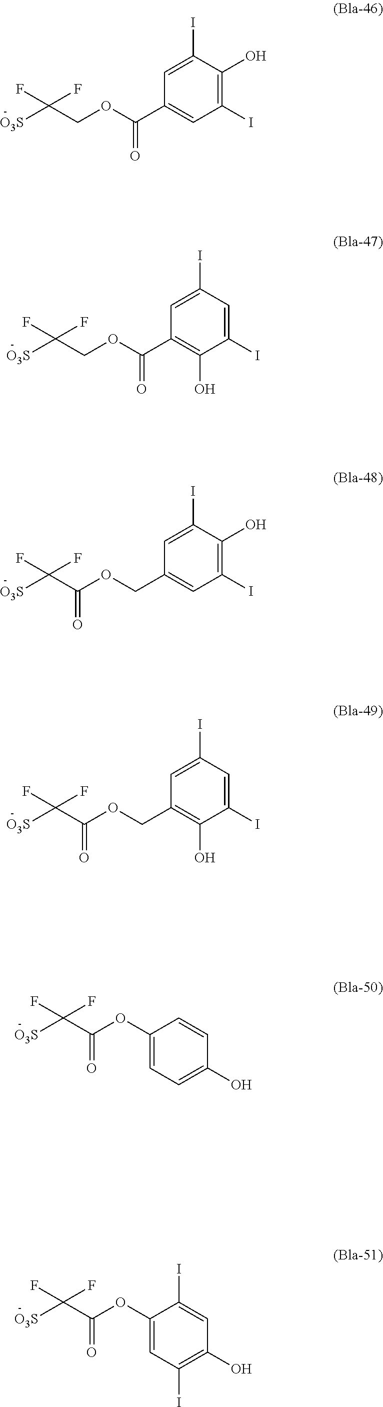

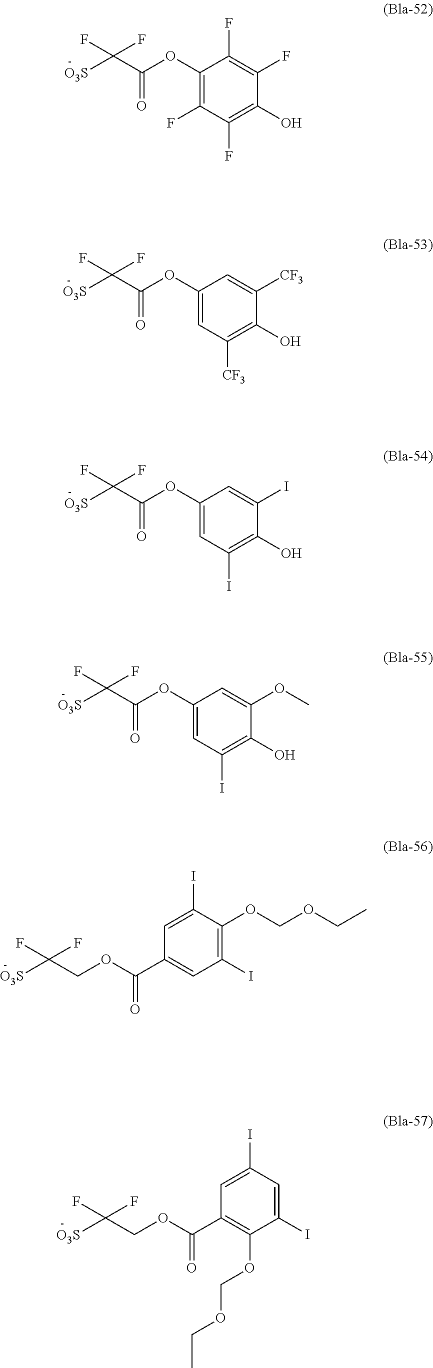

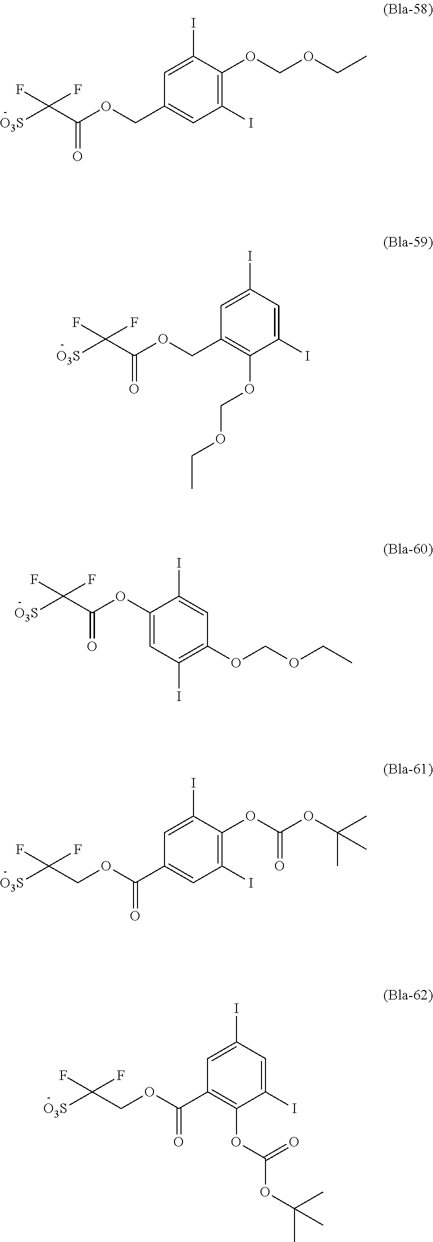

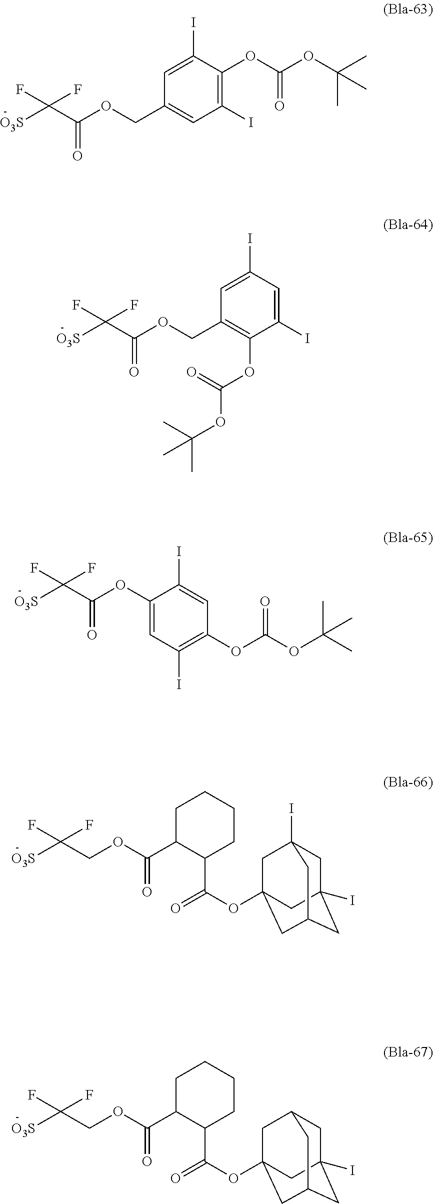

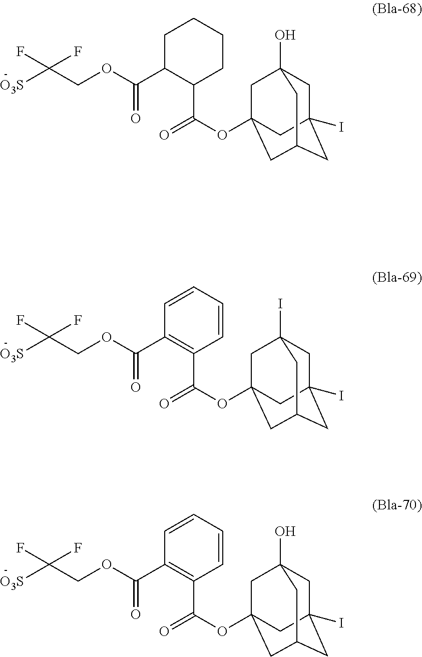

Of these, an anion represented by any one of formula (B1a-1) to formula (B1a-3), formula (B1a-7) to formula (B1a-19) and formula (B1a-22) to formula (B1a-70) is preferable.

The anion represented by formula (B1-2) is represented by the following formula:

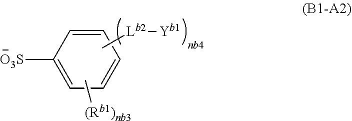

wherein, in formula (B1-2),
L^{b2} and Y^{b1} are the same as defined in formula (B1),
R^{b1} represents a halogen atom or an alkyl group having 1 to 6 carbon atoms, and —CH₂— included in the alkyl group may be replaced by —O— or —CO—,
nb4 represents an integer of 1 to 5, and when nb4 is 2 or more, a plurality of -L^{b4}-Y^{b2} may be the same or different from each other, and
nb3 represents an integer of 0 to 4, and when nb3 is 2 or more, a plurality of R^{b1} may be the same or different from each other,
in which nb1 and nb3 satisfy: 1≤nb1+nb3≤5.

In formula (B1-2), examples of the hydrocarbon group as for L^{b2} and the cyclic hydrocarbon group as for Y^{b1} include the same hydrocarbon groups as for L^{b2} and the same cyclic hydrocarbon groups as for Y^{b1} in formula (B1) as long as the upper limit of the number of carbon atoms permit. Examples of the substituent which may be possessed by the hydrocarbon group as for L^{b2}, and the methyl group and the cyclic hydrocarbon group as for Y^{b1} also include the same substituents which may be possessed by the hydrocarbon groups as for L^{b2}, and the methyl group and the cyclic hydrocarbon group as for Y^{b1} of formula (B1).

In formula (B1-2), L^{b2} is preferably an alkanediyl group having 1 to 12 carbon atoms, —CH₂— included in the alkanediyl group may be replaced by —O— or —CO—, and more preferably *—CO—O-L^{b41}- (L^{b41} is a single bond or a chain hydrocarbon group having 1 to 6 carbon atoms, —CH₂— included in the chain hydrocarbon group may be replaced by —O— or —CO—, * represents a bonding site to a phenyl group to which SO₃ is bonded, and L^{b41} is preferably a single bond or a chain hydrocarbon group having 1 to 3 carbon atoms).

In formula (B1-2), Y^{b1} is preferably an alicyclic hydrocarbon group having 3 to 18 carbon atoms (—CH₂— included in the chain hydrocarbon group may be replaced by —O— or —CO—), more preferably Y^{b1} of formula (B1-1) and an alicyclic hydrocarbon group mentioned as for L^{b1}, and still more preferably the following groups. R^{Yb} and R^{Yc} are as the same as defined above.

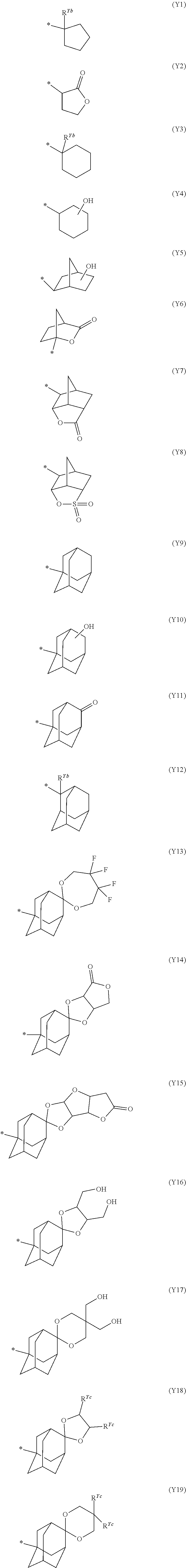

In formula (B1-2), n4b is preferably an integer of 1 to 4, more preferably an integer of 1 to 3, still more preferably 1 or 2, and yet more preferably 2. When n4b is 1 or 2, the bonding site of -L^{b2}-Y^{b1} is preferably the m-position of the benzene ring, with respect to the bonding site of SO₃, as shown in the following structures:

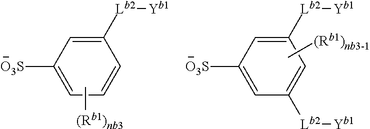

wherein, in the above formulas, L^{b2}, Y^{b1}, R^{b1} and nb3 are the same as defined in formula (B1-2).

In formula (B1-2), when nb4 is 2 or more, a plurality of L^{b2} and Y^{b1} preferably represent the same groups as each other.

Examples of the alkyl group having 1 to 6 carbon atoms as for R^{b1} include alkyl groups such as a methyl group, an ethyl group, a propyl group, an isopropyl group, a butyl group, an isobutyl group, a tert-butyl group, a pentyl group and a hexyl group. The number of carbon atoms of the alkyl group is preferably 1 to 4, and more preferably 1 to 3.

Examples of the halogen atom as for R^{b1} include a fluorine atom, a chlorine atom, a bromine atom and an iodine atom.

It is preferable that R^{b1} is each independently a halogen atom or an alkyl group having 1 to 4 carbon atoms (—CH₂— included in the alkyl group may be replaced by —O— or —CO—), more preferably a fluorine atom, an iodine atom or an alkyl group having 1 to 3 carbon atoms (—CH₂— included in the alkyl group may be replaced by —O— or —CO—), and still more preferably a fluorine atom, an iodine atom, a hydroxy group, a methoxy group or a methyl group.

nb3 is an integer of 0 to 4, preferably an integer of 0 to 3, and more preferably an integer of 0 to 2. In one embodiment, nb3 is preferably 0. In another embodiment, nb3 is preferably 1 or 2. When nb3 is 1, R^{b1} is preferably a halogen atom, and R^{b1} is more preferably a fluorine atom or an iodine atom. When nb3 is 2, it is preferable that one of R^{b1} is a halogen atom, and the other one is a halogen atom or an alkyl group having 1 to 4 carbon atoms, and it is more preferable that one of R^{b1} is a fluorine atom or an iodine atom, and the other one is a fluorine atom, an iodine atom or an alkyl group having 1 to 3 carbon atoms.

Examples of the anion (B1-2) include the following anions. Of these, anions represented by formula (B2a-1) to formula (B2a-20) are preferable, and anions represented by formula (B2a-1) to formula (B2a-11) and formula (B2a-16) to formula (B2a-20) are more preferable.

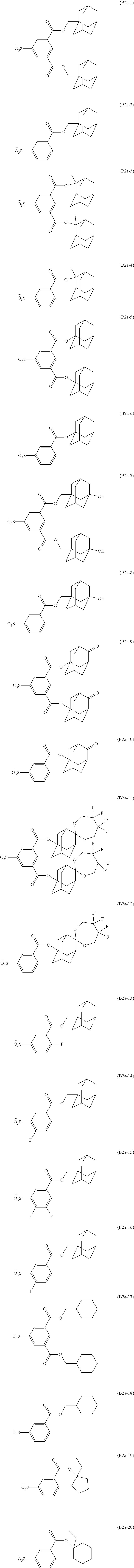

In another embodiment, the acid generator (B) may have a sulfonylimide anion or a sulfonylmethide anion, and it is also possible to use, as the acid generator (B), a salt in which the anion in the salt represented by formula (B1) is replaced by a sulfonylimide anion or a sulfonylmethide anion represented by formula (B1-A3) (hereinafter sometimes referred to as “anion (B1-A3)”).

The anion represented by formula (B1-A3) is represented by the following formula:

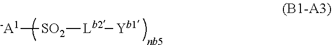

wherein, in formula (B1-A3),
A1 represents a nitrogen atom or a carbon atom,
L^{b2}′ represents a single bond or a divalent hydrocarbon group having 1 to 24 carbon atoms which may have a substituent, and —CH₂— included in the hydrocarbon group may be replaced by —O—, —S—, —CO— or —SO₂—,
Y^{b1}′ represents a methyl group which may have a substituent or a cyclic hydrocarbon group having 3 to 24 carbon atoms which may have a substituent, and —CH₂— included in the cyclic hydrocarbon group may be replaced by —O—, —S—, —SO₂— or —CO—, and two L^{b2}′-Y^{b1}′ may be bonded to each other to form a ring containing A¹, and
nb5 represents 2 or 3, and when nb5 is 2, A1 represents a nitrogen atom, and when nb5 is 3, A1 represents a carbon atom, and a plurality of groups in parentheses may be the same or different from each other.

In formula (B1-A3), examples of those as for L^{b2}‘ and Y^{b1}’ include the same groups as for L^{b2} and Y^{b1} of formula (B1-A1).

In formula (B1-A3), L^{b2}′ is preferably a single bond, *-L^{b23}-X²— or *-L^{b23}-X²—W²—X³— (L^{b23} represents an alkanediyl group having 1 to 6 carbon atoms which may have a fluorine atom, X² and X³ each independently represent —O—, —CO—O—, —O— CO—, —O—CO—O— or —O—, W² represents an alicyclic hydrocarbon group having 3 to 12 carbon atoms, —CH₂— included in the alicyclic hydrocarbon group may be replaced by —O— or —CO—, and * represents a bonding site to SO₂).

In formula (B1-A3), Y^{b1}′ is preferably a trifluoromethyl group, an alicyclic hydrocarbon group having 3 to 18 carbon atoms which may have a substituent, or a phenyl group which may have a substituent, and more preferably a trifluoromethyl group or groups represented by formula (Y1) to formula (Y36) in formula (B1-A1) is/are preferable.

nb5 is preferably 2 or 3.

In formula (B1-A3), when two L^{b2}′-Y^{b1}′ combine to form a ring containing A¹, examples include an anion represented by formula (B1-A3′):

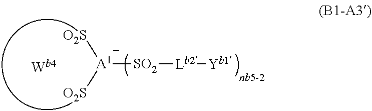

wherein, in formula (B1-A3′),
A¹, Y^{b1}′, L^{b2}′, nb5 are the same as defined in formula (B1-A3), and
W^{b4} represents a disulfonylimide ring or disulfonylmethide ring having 3 to 12 carbon atoms which may have a fluorine atom.

The disulfonylimide ring or disulfonylmethide ring as for W^{b4} preferably has 3 to 6 carbon atoms, and the hydrogen atom of the methylene group included in the ring is preferably substituted with a fluorine atom.

Examples of the anion represented by formula (B1-A3) include the following. Of these, an anion represented by formula (B1-a3-1), formula (B1-a3-2) is preferable.

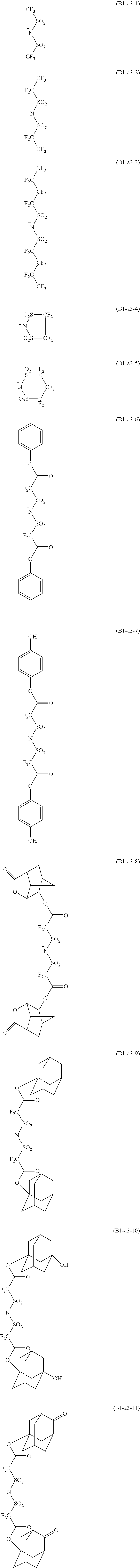

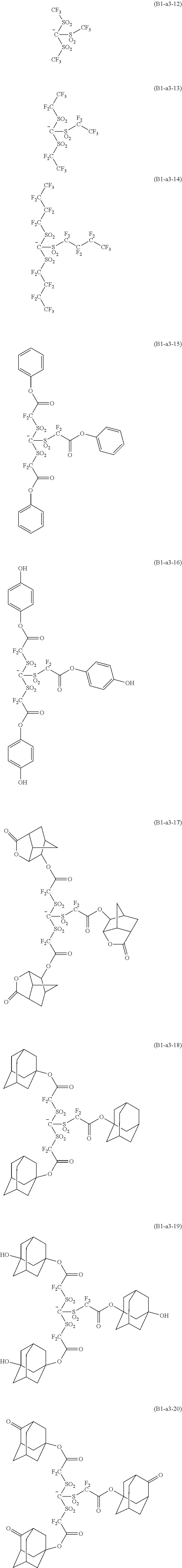

In another embodiment, the acid generator (B) also may have a carboxylic acid anion, and it is possible to use, as the acid generator (B), a salt in which the anion of the salt represented by formula (B1) is replaced by a carboxylic acid anion. Examples of the carboxylic acid anion include the following.

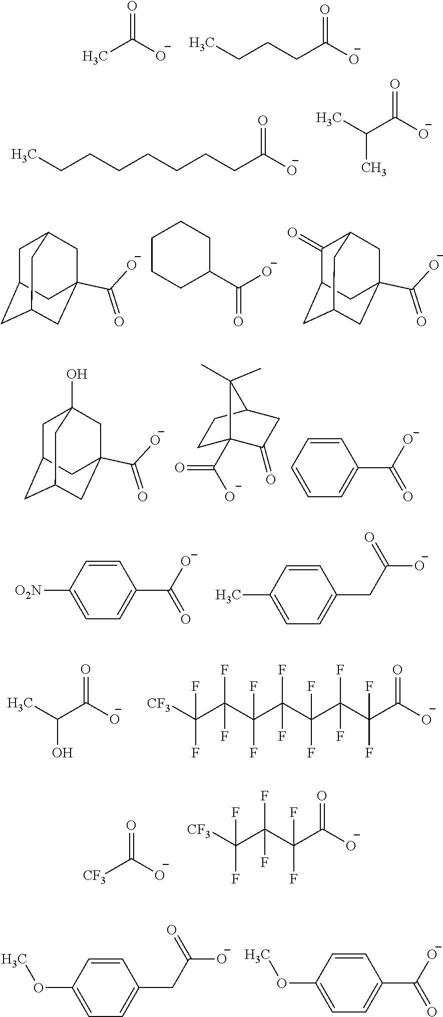

Examples of the organic cation as for Z1⁺ include the same organic cations as Z⁺ of the salt represented by formula (I).

The compound (B) is a combination of the anion mentioned above and the organic cation mentioned above, and these can be optionally combined. The acid generator (B) preferably includes a combination of an anion represented by any one of formula (B1a-1) to formula (B1a-3), formula (B1a-7) to formula (B1a-19), formula (B1a-22) to formula (B1a-70), or an anion represented by any one of formula (B2a-1) to formula (B2a-11), formula (B2a-16) to formula (B2a-20) and formula (B1-a3-1) to formula (B1-a3-20) with a cation (b2-1), a cation (b2-2), a cation (b2-3), a cation (b2-4) or a cation (b2-5).

The compound (B) preferably includes those represented by formula (B1-1) to formula (B1-105) and formula (B2-1) to formula (B2-20), formula (B3-1) to formula (B3-28), respectively. Of these, those containing an arylsulfonium cation are preferable and those represented by formula (B1-1) to formula (B1-3), formula (B1-5) to formula (B1-7), formula (B1-11) to formula (B1-14), formula (B1-20) to formula (B1-26), formula (B1-29), formula (B1-31) to formula (B1-105), and formula (B2-1) to formula (B2-20), formula (B3-1) to formula (B3-28) are particularly preferable.

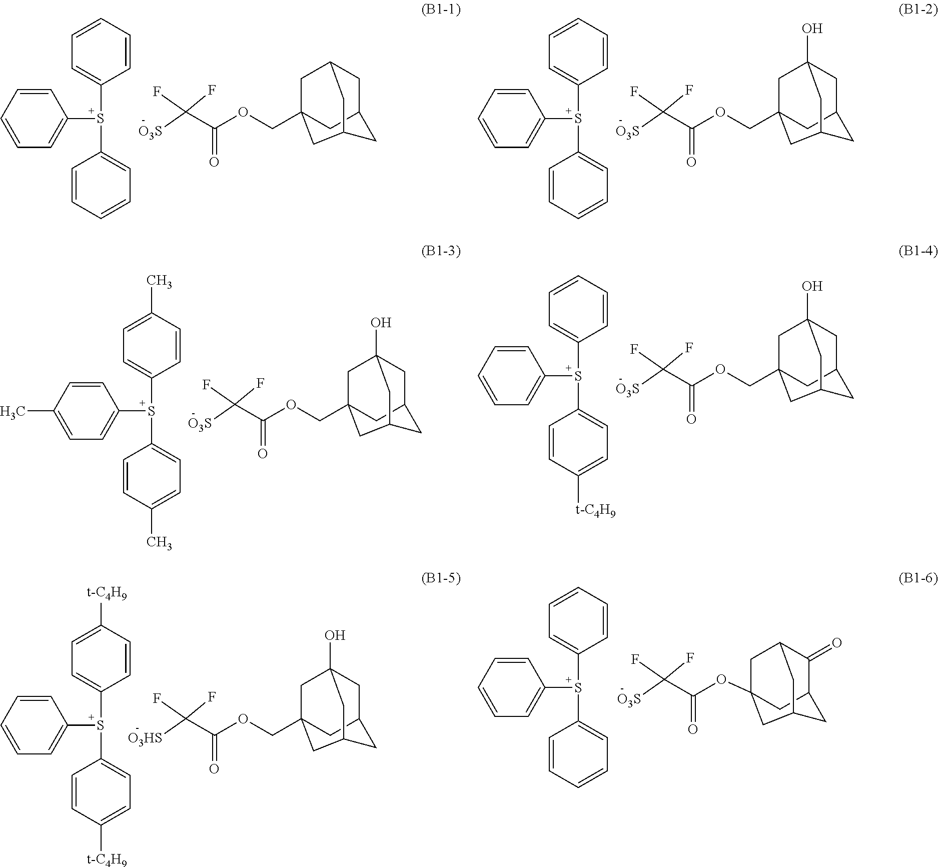

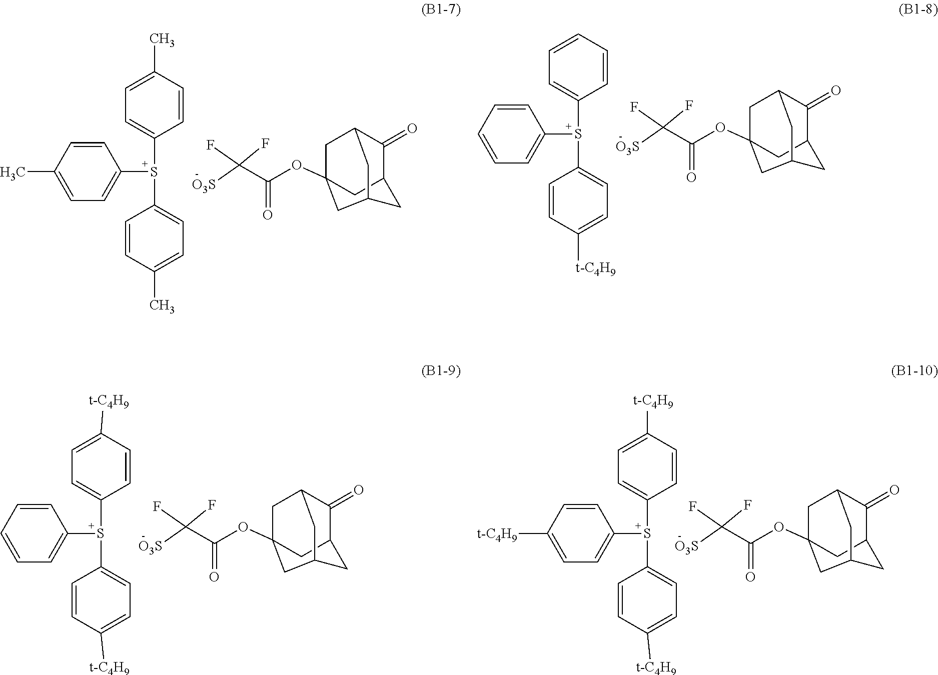

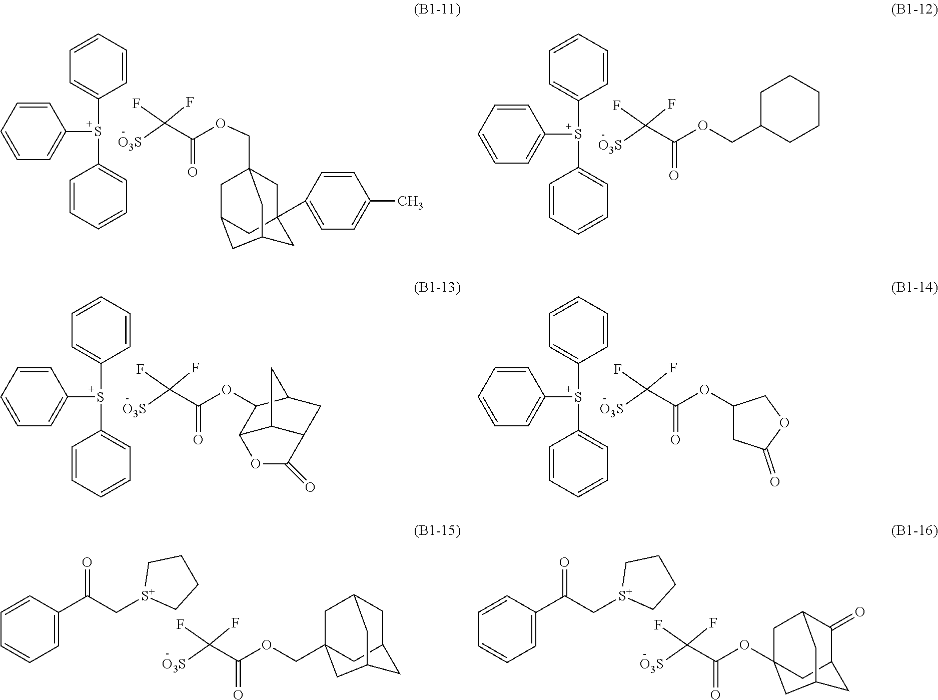

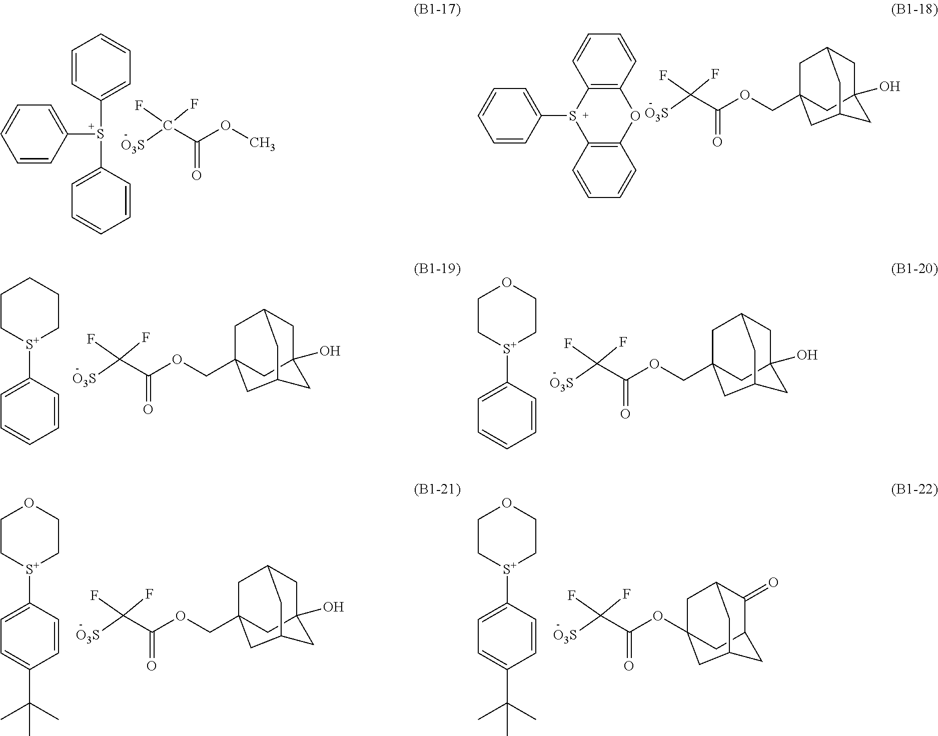

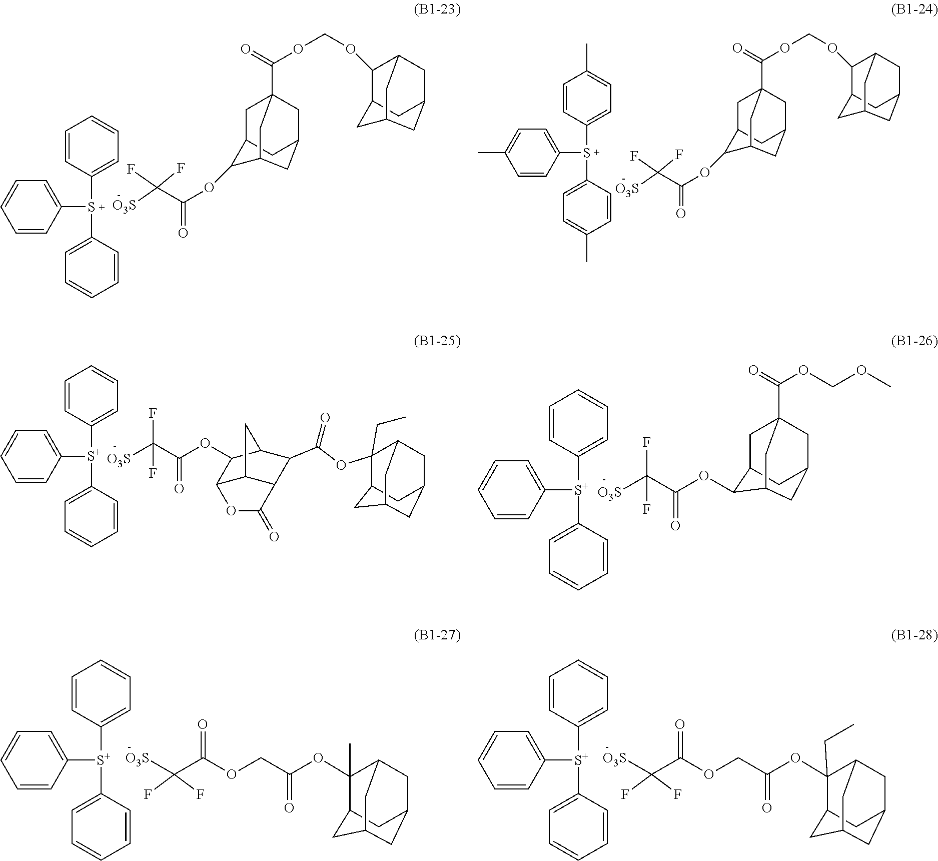

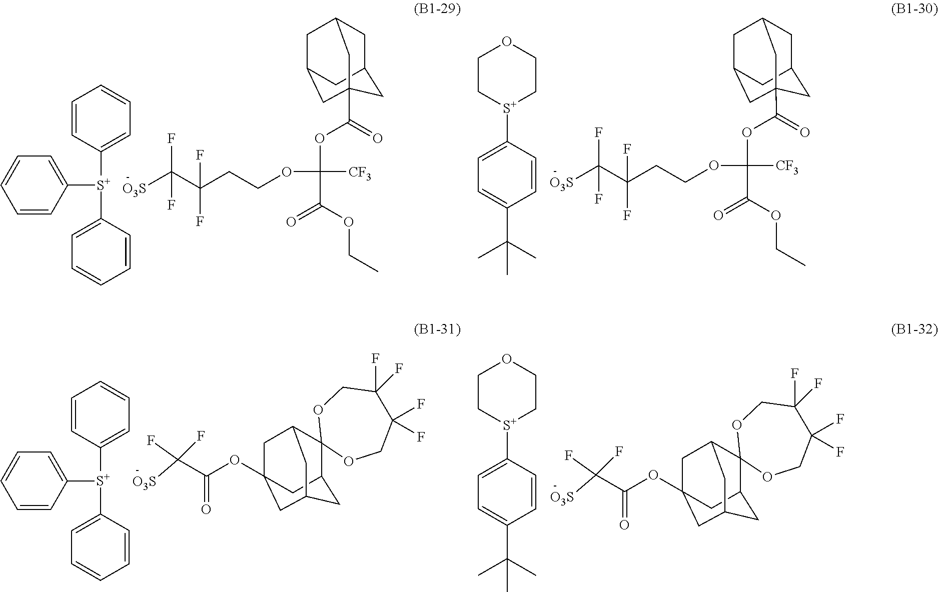

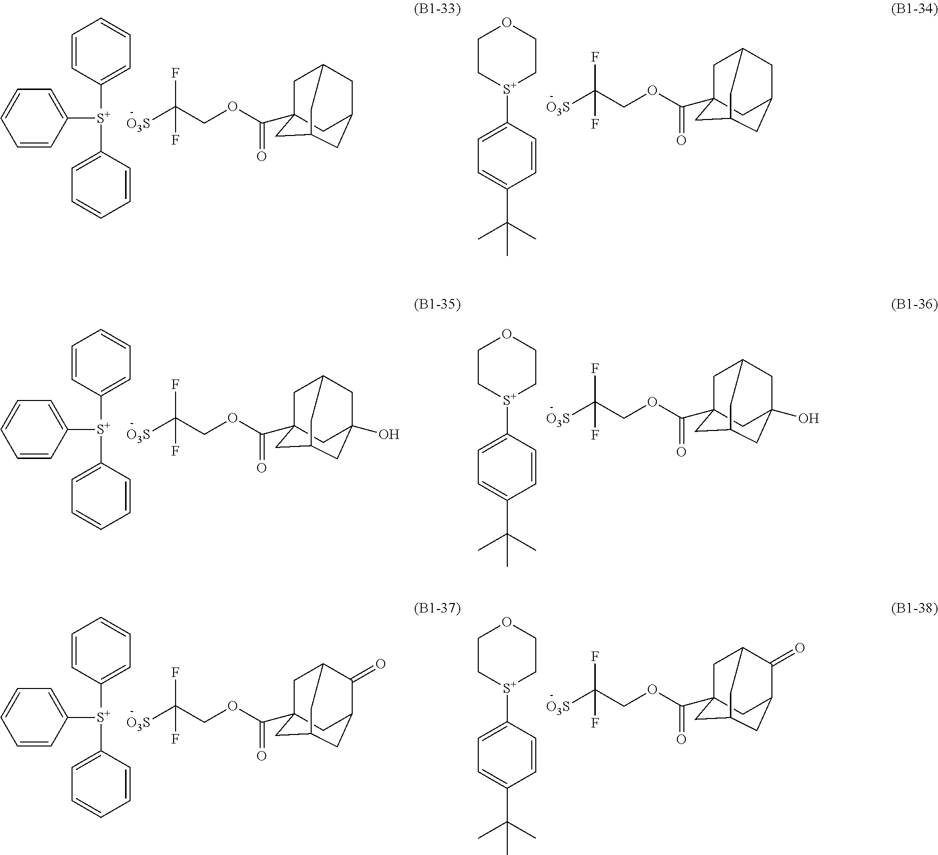

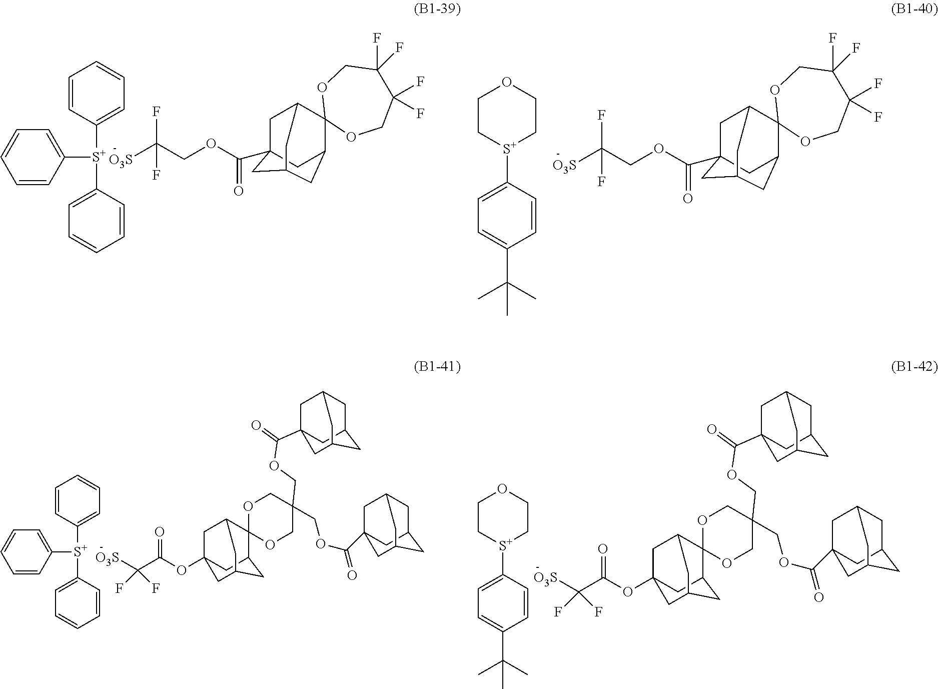

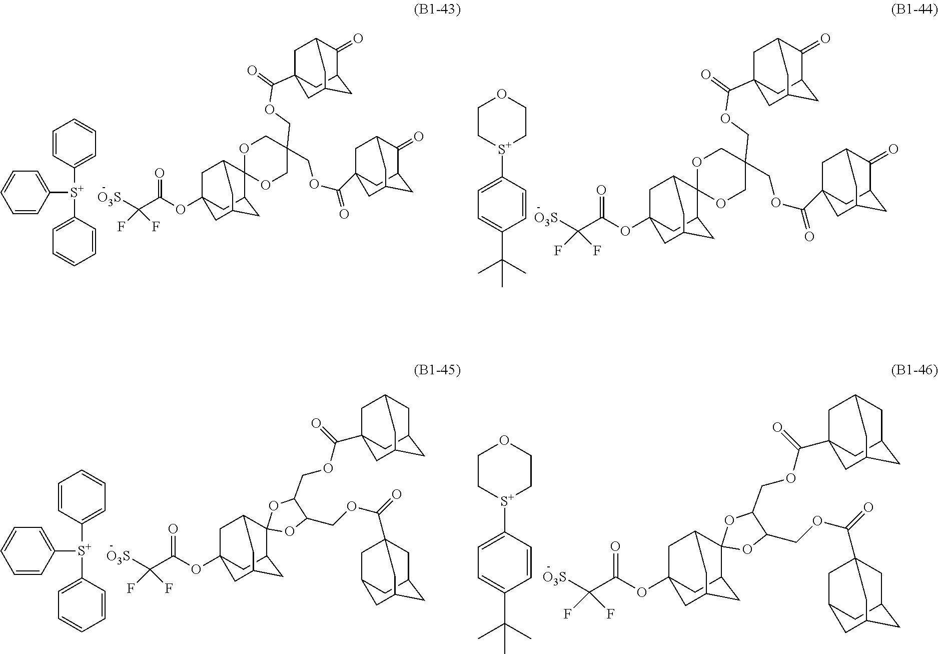

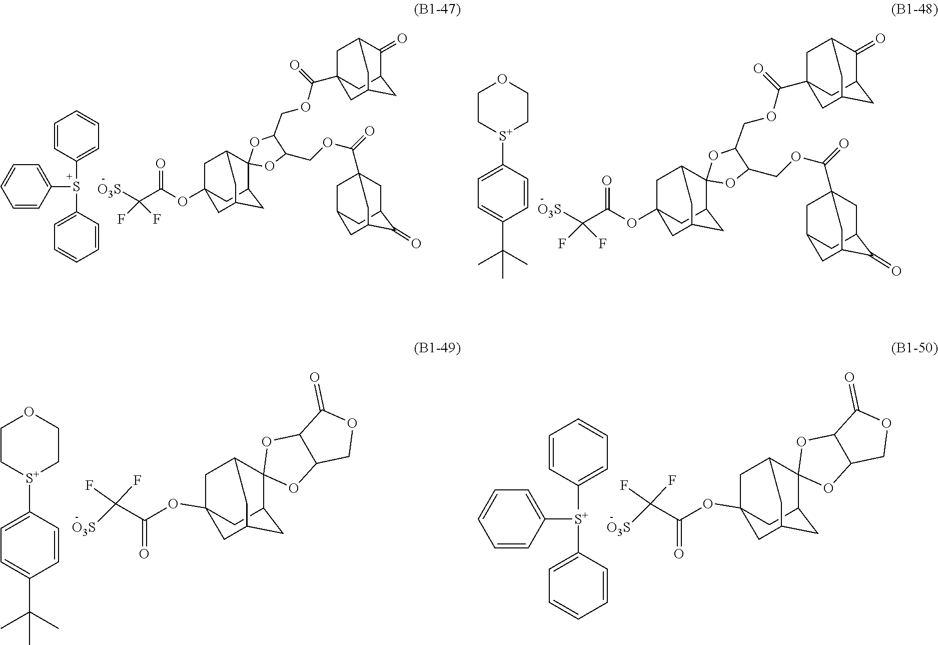

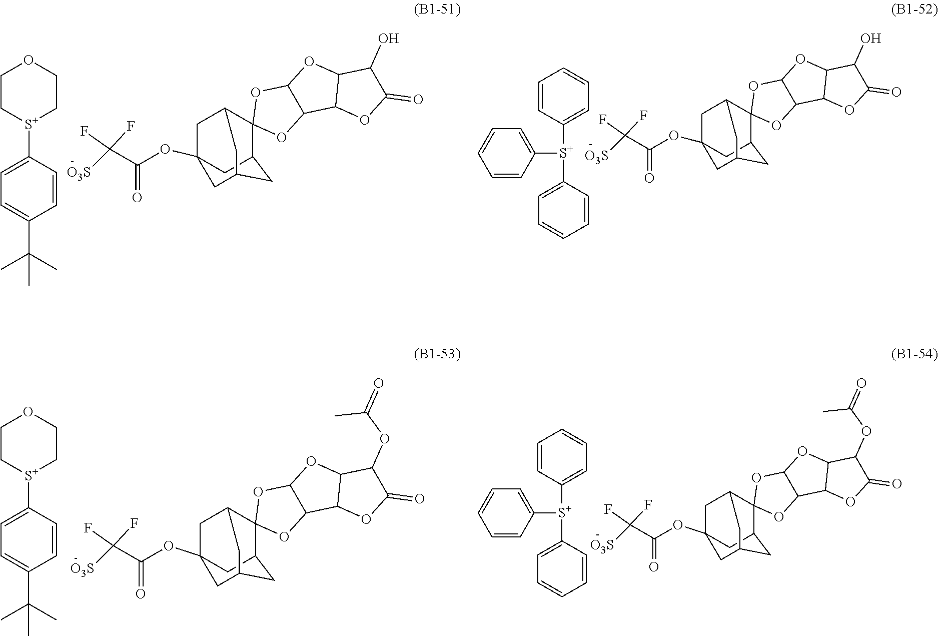

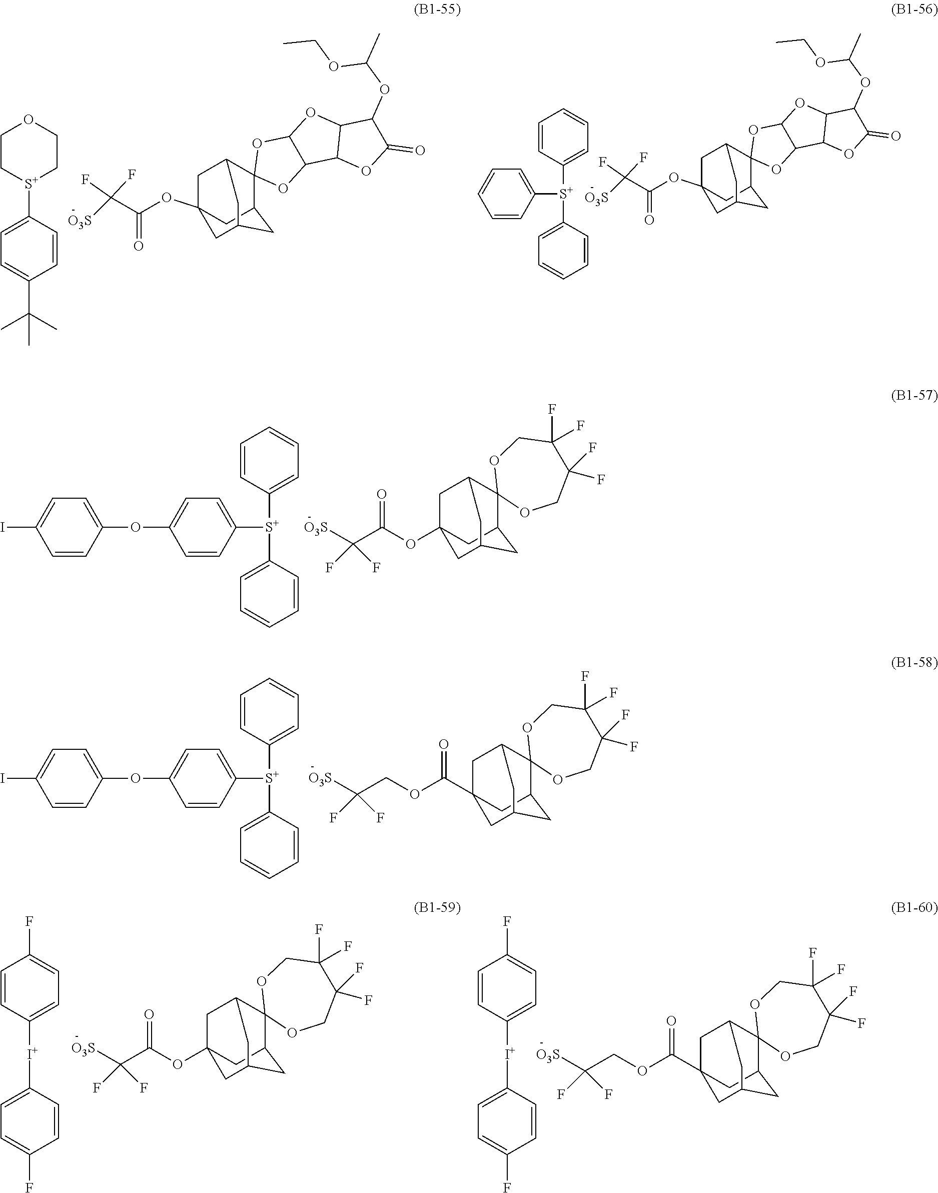

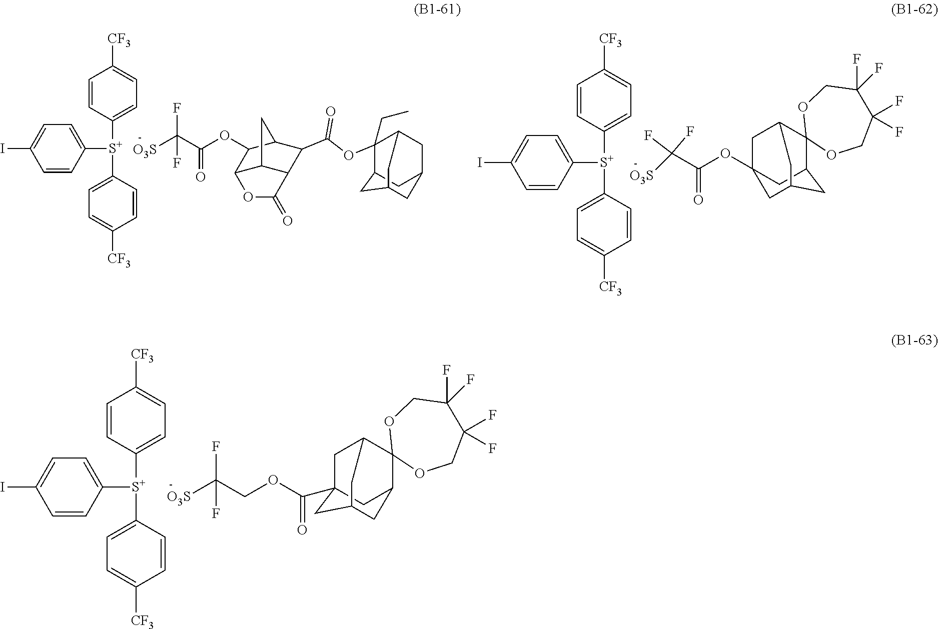

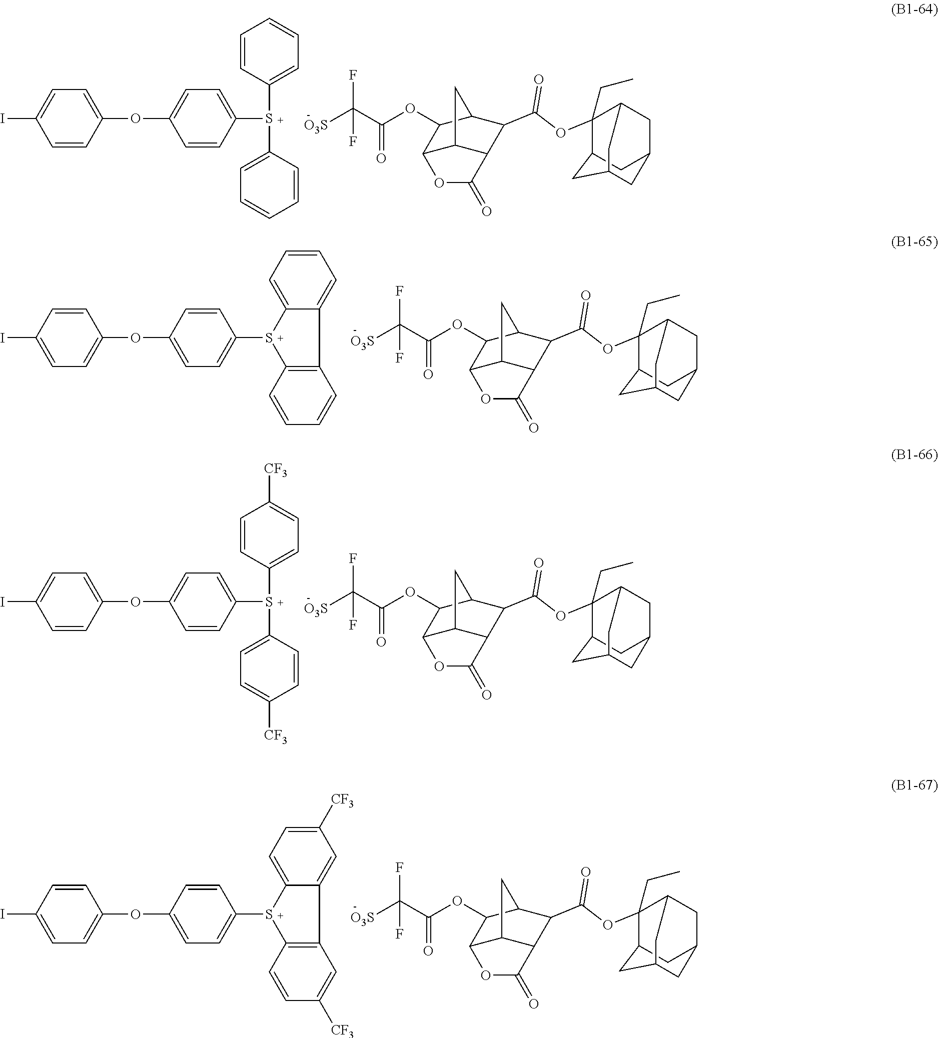

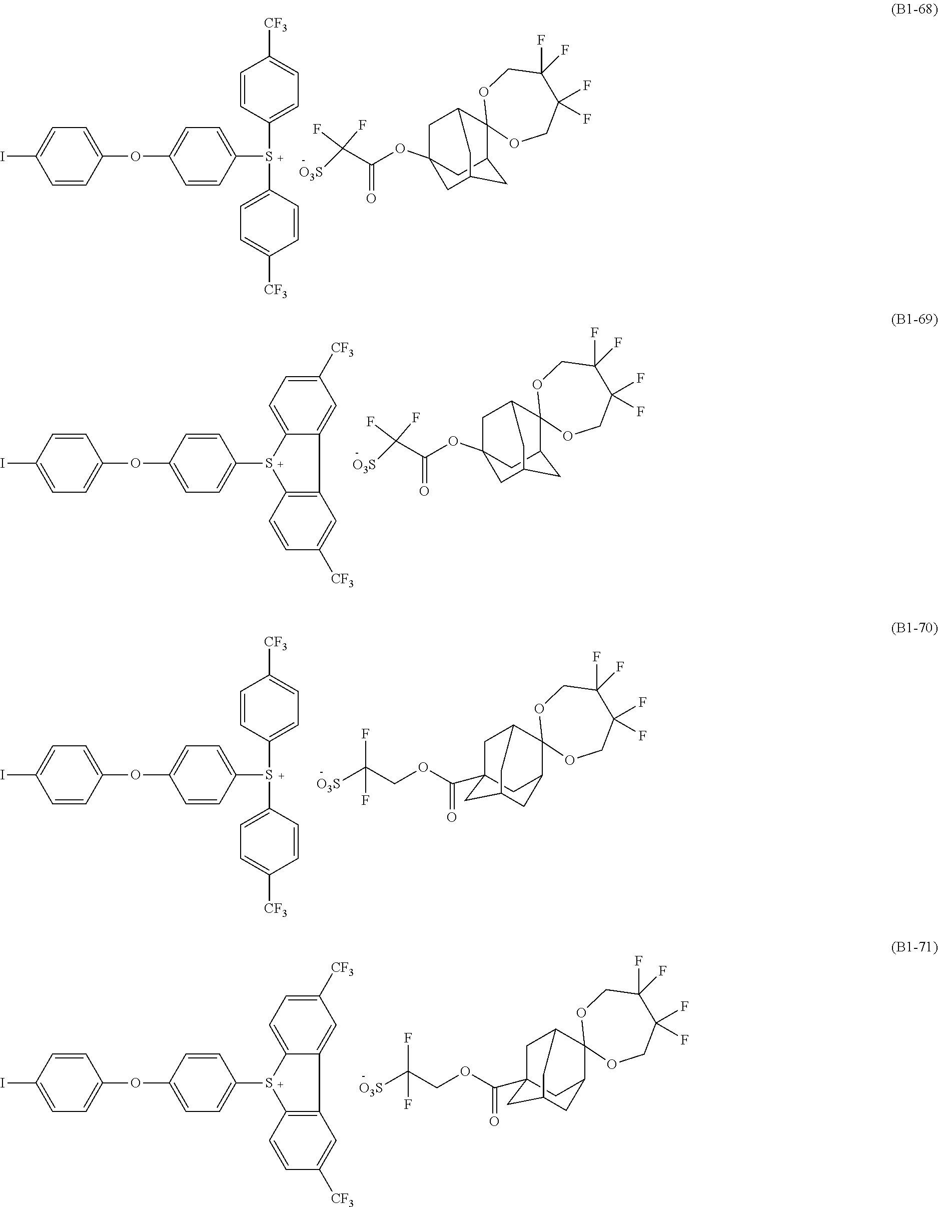

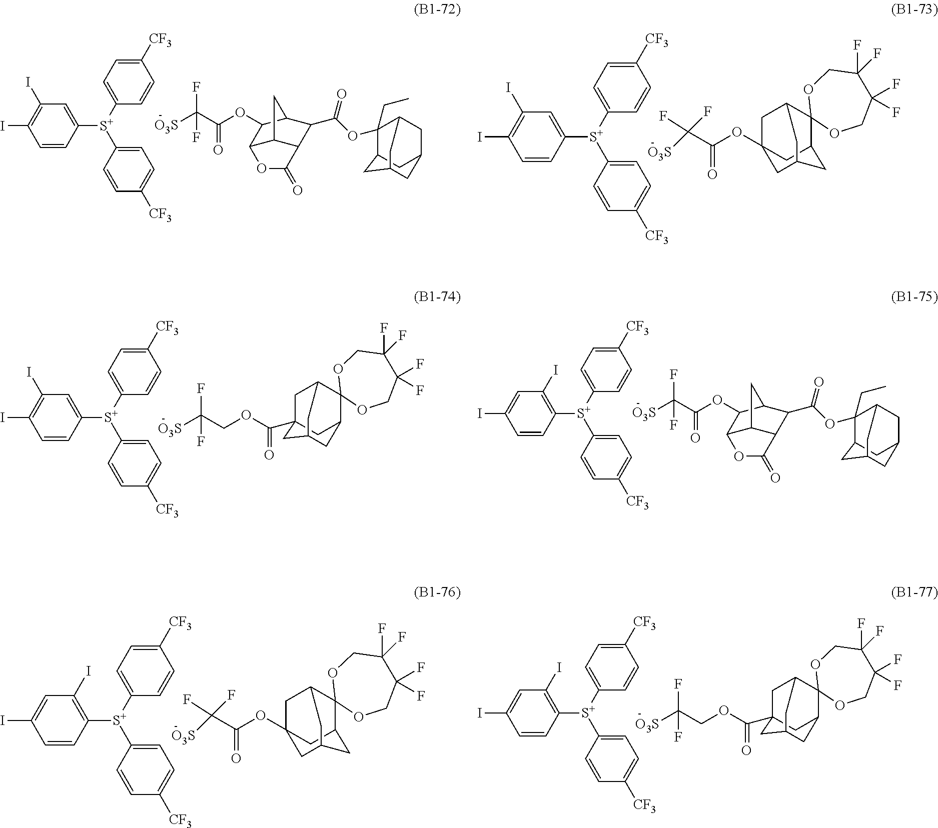

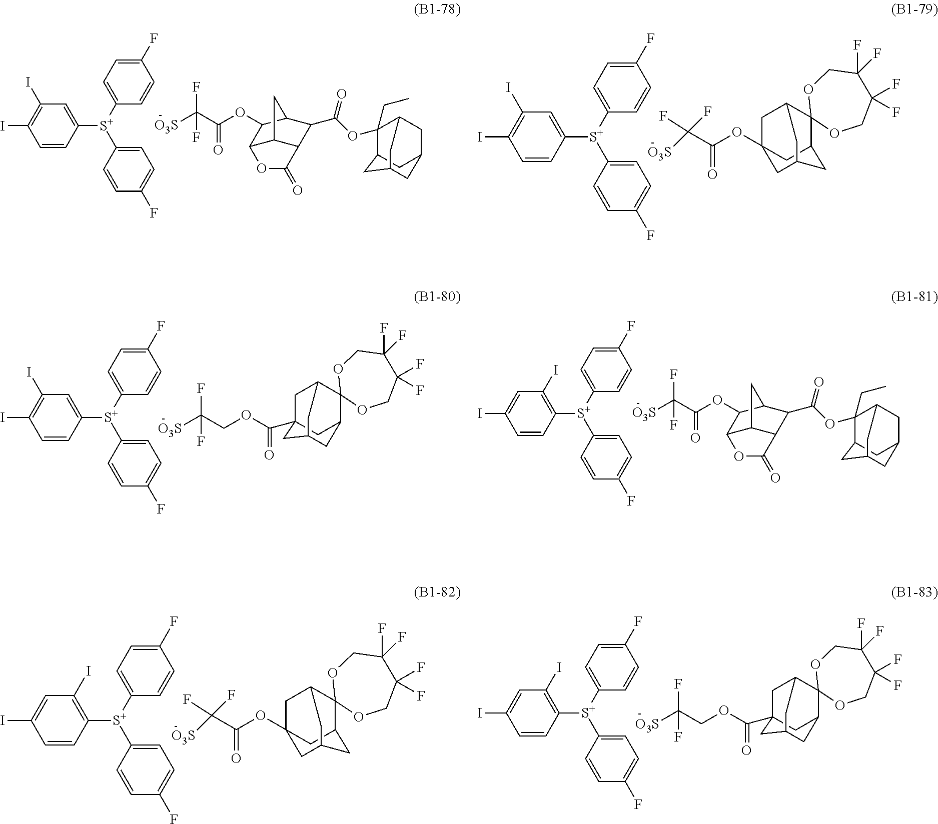

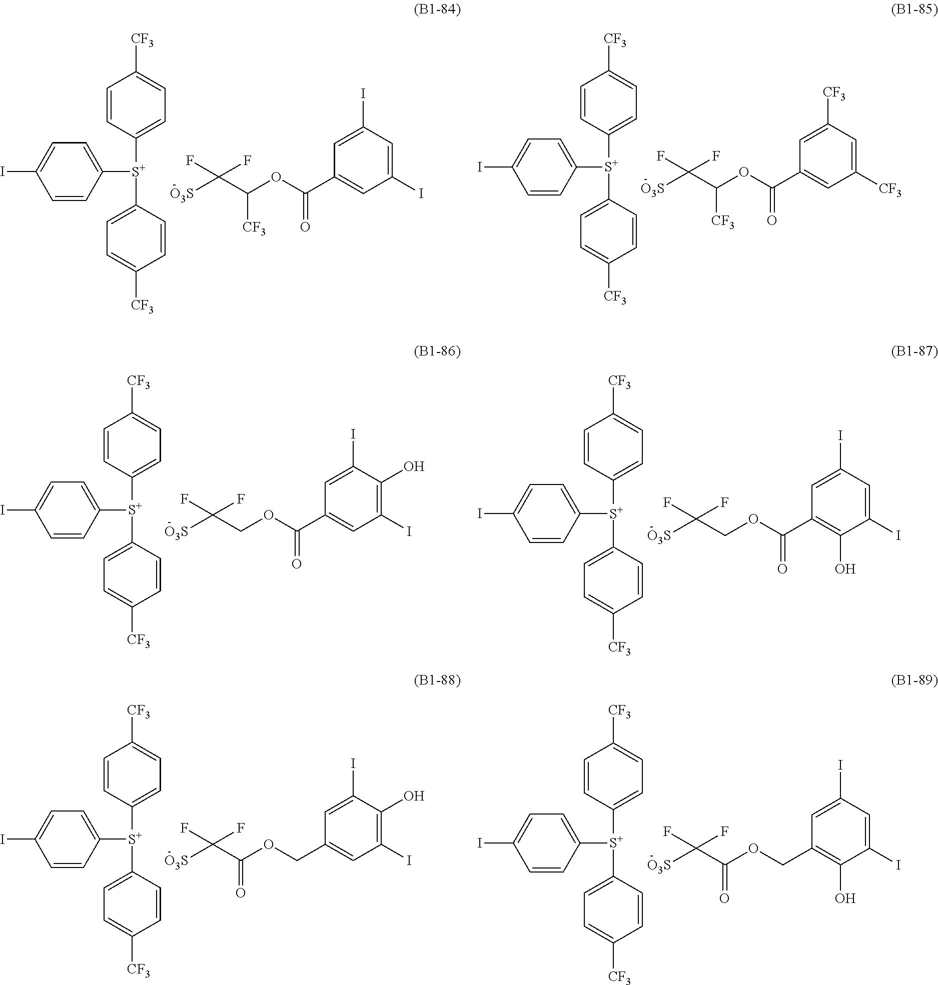

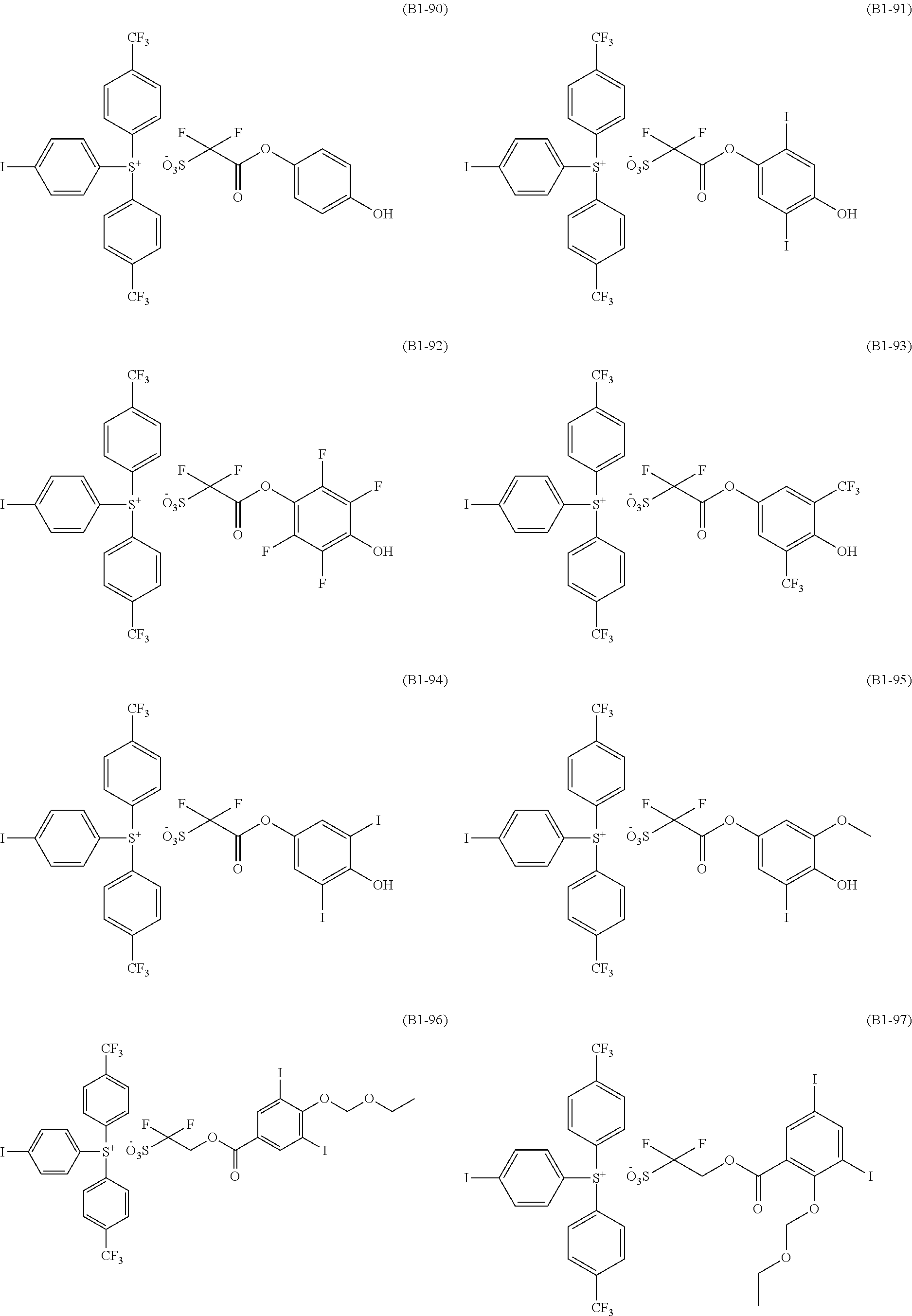

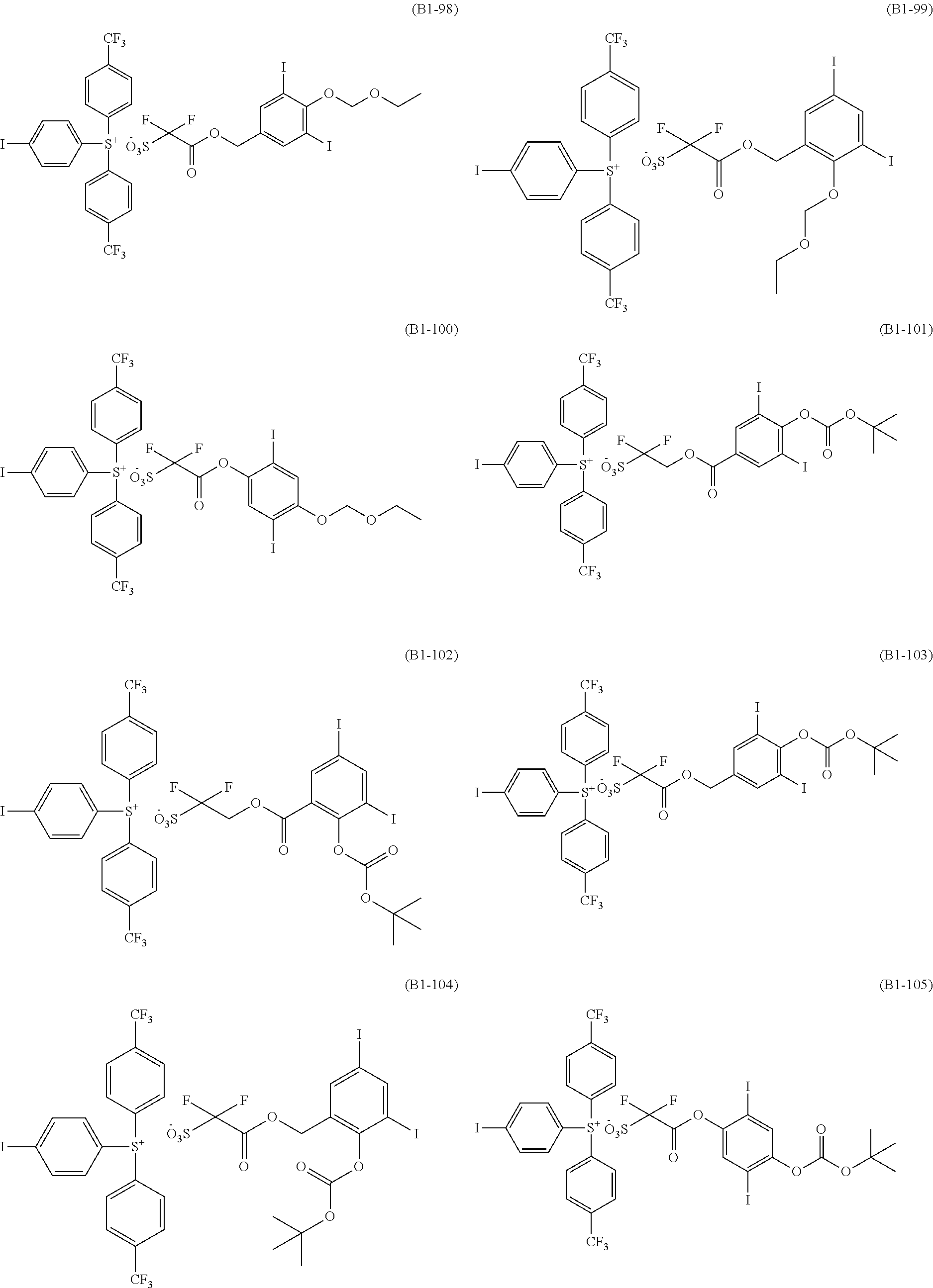

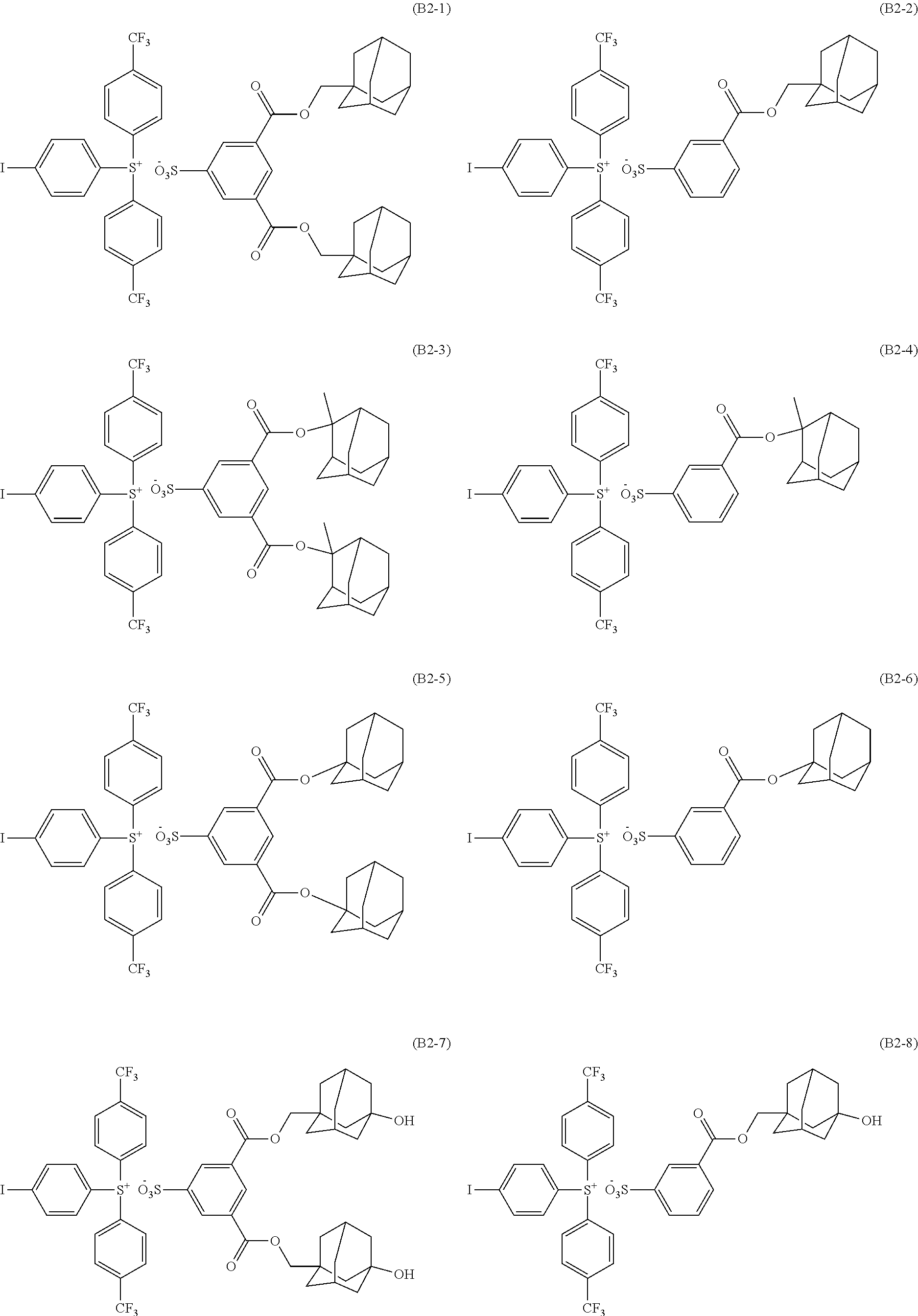

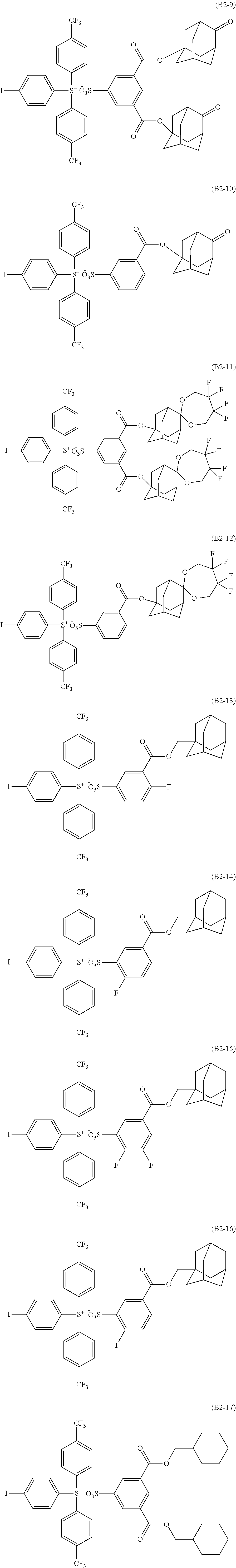

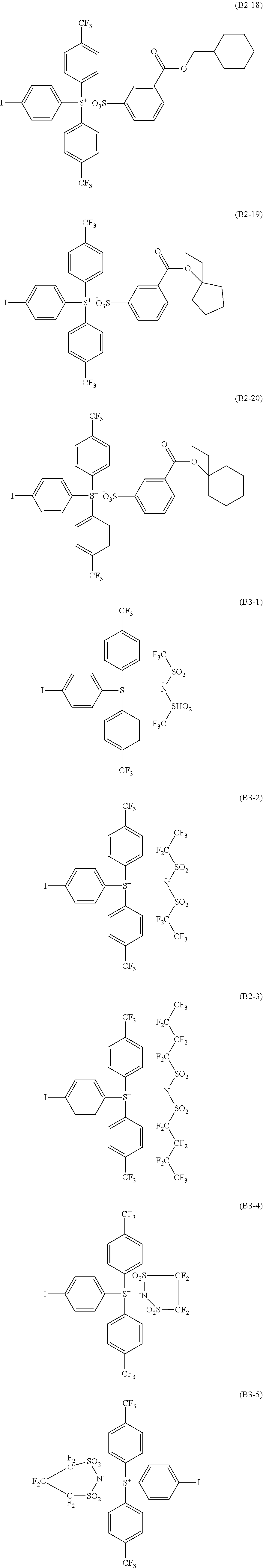

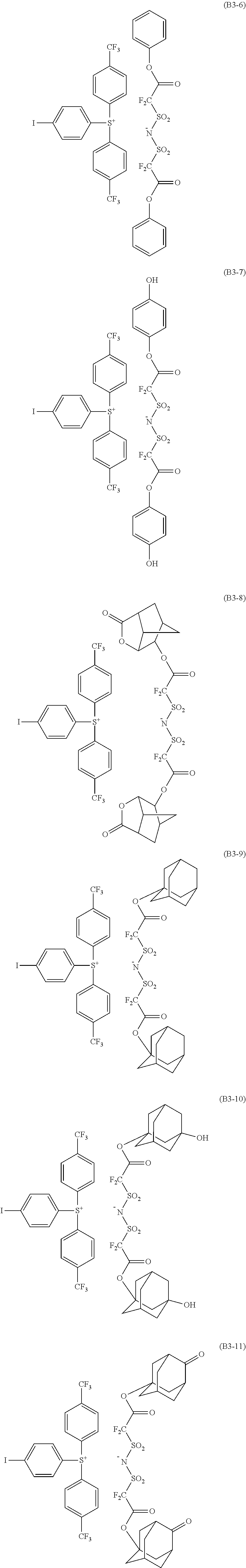

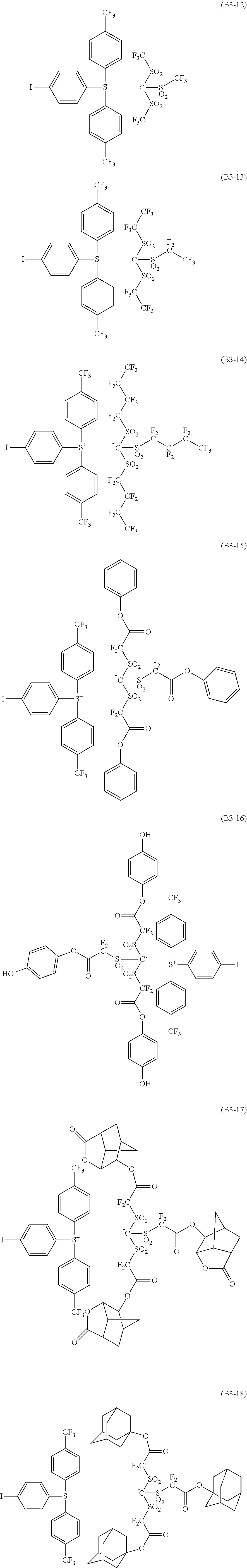

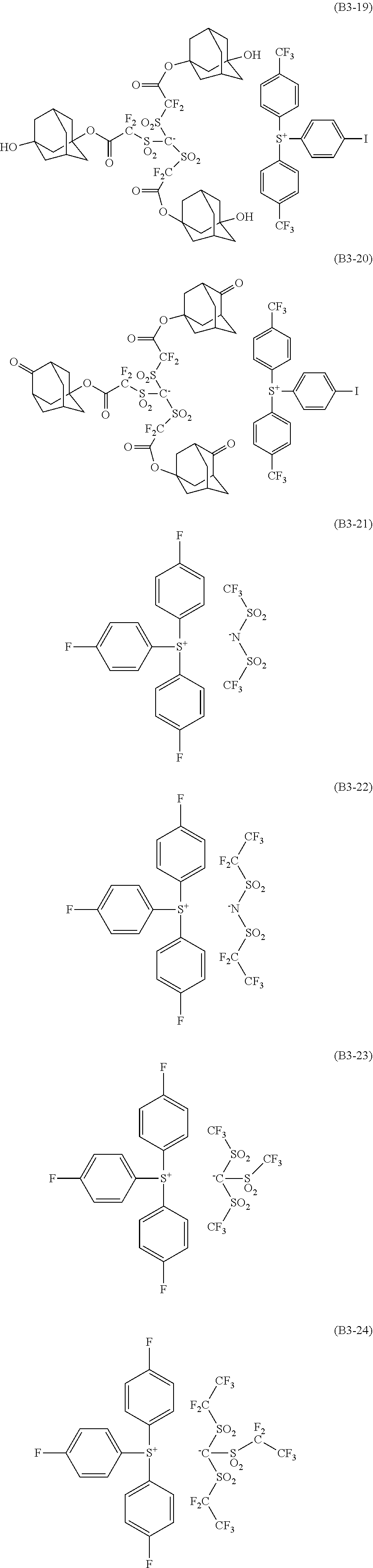

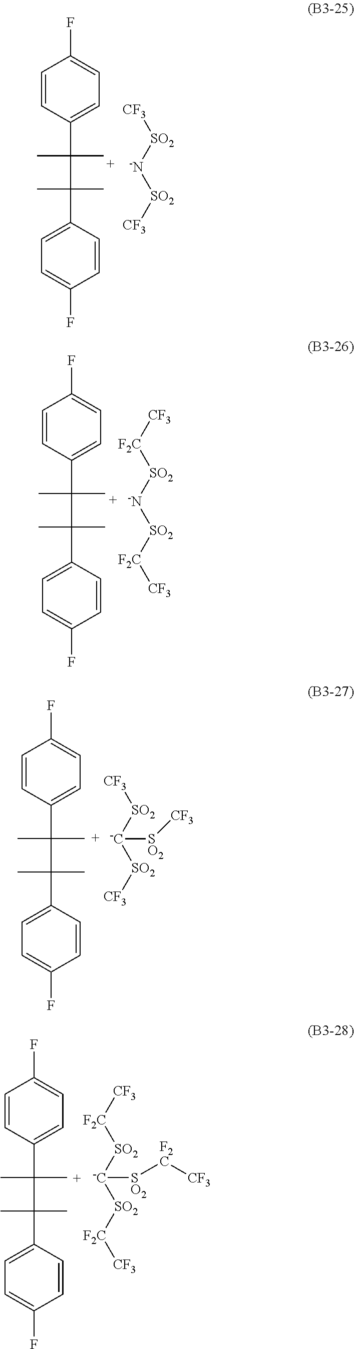

When including, as the acid generator, a salt (I) and a compound (B), a content ratio of the salt (I) to the compound (B) (mass ratio; salt (I):compound (B)) is usually 1:99 to 99:1, preferably 2:98 to 98:2, more preferably 5:95 to 95:5, still more preferably 10:90 to 90:10, and particularly preferably 15:85 to 85:15.

### <Solvent (E)>

The content of the solvent (E) in the resist composition is usually 90% by mass or more and 99.9% by mass or less, preferably 92% by mass or more and 99% by mass or less, and more preferably 94% by mass or more and 99% by mass or less. The content of the solvent (E) can be measured, for example, by a known analysis means such as liquid chromatography or gas chromatography.

Examples of the solvent (E) include glycol ether esters such as ethylcellosolve acetate, methylcellosolve acetate and propylene glycol monomethyl ether acetate; glycol ethers such as propylene glycol monomethyl ether; esters such as ethyl lactate, butyl acetate, amyl acetate and ethyl pyruvate; ketones such as acetone, methyl isobutyl ketone, 2-heptanone and cyclohexanone; and cyclic esters such as γ-butyrolactone. The solvent (E) may be used alone, or two or more solvents may be used.

### <Quencher (C)>

Examples of the quencher (C) include a basic nitrogen-containing organic compound, and a salt generating an acid having an acidity lower than that of an acid generated from an acid generator (e.g., salt (I) or compound (B)). When the resist composition includes the quencher (C), the content of the quencher (C) is preferably about 0.01 to 15% by mass, more preferably about 0.01 to 10% by mass, still more preferably about 0.1 to 5% by mass, and yet more preferably about 0.1 to 3% by mass, based on the amount of the solid component of the resist composition.

Examples of the basic nitrogen-containing organic compound include amine and an ammonium salt. Examples of the amine include an aliphatic amine and an aromatic amine. Examples of the aliphatic amine include a primary amine, a secondary amine and a tertiary amine.

Examples of the amine include 1-naphthylamine, 2-naphthylamine, aniline, diisopropylaniline, 2-, 3- or 4-methylaniline, 4-nitroaniline, N-methylaniline, N,N-dimethylaniline, diphenylamine, hexylamine, heptylamine, octylamine, nonylamine, decylamine, dibutylamine, dipentylamine, dihexylamine, diheptylamine, dioctylamine, dinonylamine, didecylamine, triethylamine, trimethylamine, tripropylamine, tributylamine, tripentylamine, trihexylamine, triheptylamine, trioctylamine, trinonylamine, tridecylamine, methyldibutylamine, methyldipentylamine, methyldihexylamine, methyldicyclohexylamine, methyldiheptylamine, methyldioctylamine, methyldinonylamine, methyldidecylamine, ethyldibutylamine, ethyldipentylamine, ethyldihexylamine, ethyldiheptylamine, ethyldioctylamine, ethyldinonylamine, ethyldidecylamine, dicyclohexylmethylamine, tris[2-(2-methoxyethoxy)ethyl]amine, triisopropanolamine, ethylenediamine, tetramethylenediamine, hexamethylenediamine, 4,4′-diamino-1,2-diphenylethane, 4,4′-diamino-3,3′-dimethyldiphenylmethane, 4,4′-diamino-3,3′-diethyldiphenylmethane, 2,2′-methylenebisaniline, imidazole, 4-methylimidazole, pyridine, 4-methylpyridine, 1,2-di(2-pyridyl)ethane, 1,2-di(4-pyridyl)ethane, 1,2-di(2-pyridyl)ethene, 1,2-di(4-pyridyl)ethene, 1,3-di(4-pyridyl)propane, 1,2-di(4-pyridyloxy)ethane, di(2-pyridyl)ketone, 4,4′-dipyridyl sulfide, 4,4′-dipyridyl disulfide, 2,2′-dipyridylamine, 2,2′-dipicolylamine, bipyridine and the like, preferably diisopropylaniline, and more preferably 2,6-diisopropylaniline.

Examples of the ammonium salt include tetramethylammonium hydroxide, tetraisopropylammonium hydroxide, tetrabutylammonium hydroxide, tetrahexylammonium hydroxide, tetraoctylammonium hydroxide, phenyltrimethylammonium hydroxide, 3-(trifluoromethyl)phenyltrimethylammonium hydroxide, tetra-n-butylammonium salicylate and choline.

The acidity in a salt generating an acid having an acidity lower than that of an acid generated from the acid generator (e.g., salt (I) or compound (B)) is indicated by the acid dissociation constant (pKa). Regarding the salt generating an acid having an acidity lower than that of an acid generated from the acid generator, the acid dissociation constant of an acid generated from the salt usually meets the following inequality: −3<pKa, preferably −1<pKa<7, and more preferably 0<pKa<5.

Examples of the salt generating an acid having an acidity lower than that of an acid generated from the acid generator include salts represented by the following formulas, a salt represented by formula (D) mentioned in JP 2015-147926 A (hereinafter sometimes referred to as “weak acid inner salt (D)”, and salts mentioned in JP 2012-229206 A, JP 2012-6908 A, JP 2012-72109 A, JP 2011-39502 A and JP 2011-191745 A. The salt generating an acid having an acidity lower than that of an acid generated from the acid generator is preferably a salt generating a carboxylic acid having an acidity lower than that of an acid generated from the acid generator (salt having a carboxylic acid anion), more preferably a weak acid inner salt (D), and still more preferably a diphenyliodonium salt in which a carboxylic acid anion has a substituted phenyl group of the weak acid inner salt (D).

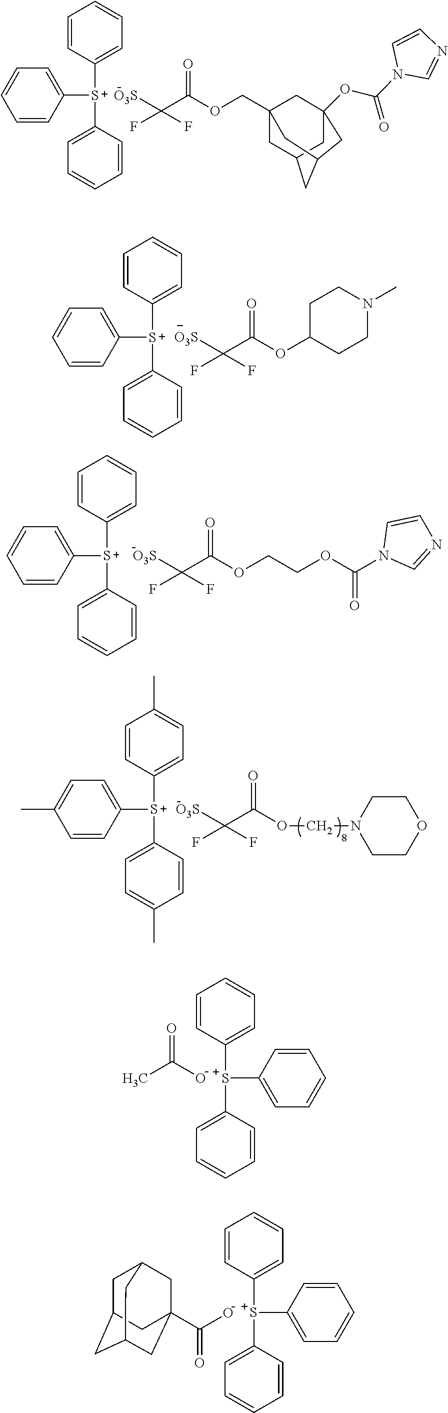

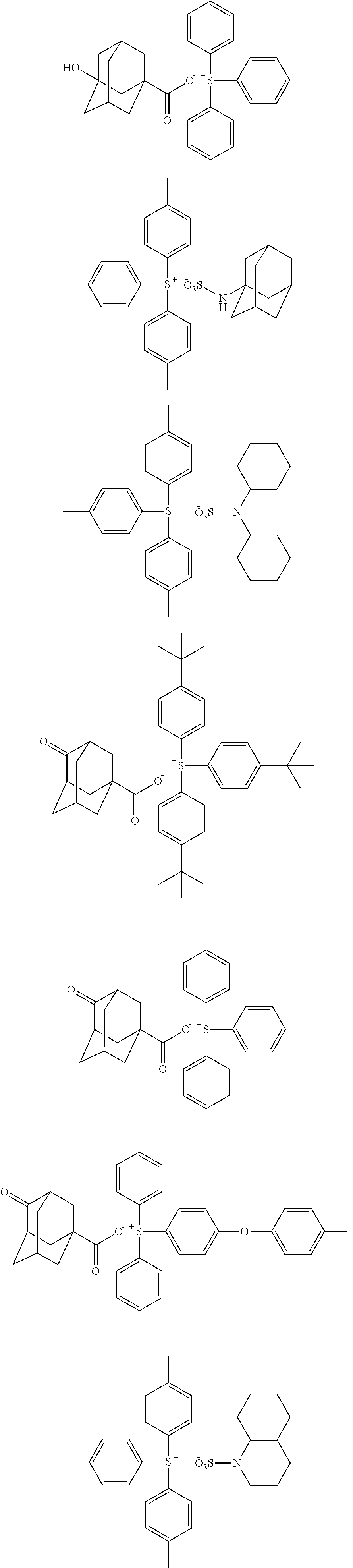

Examples of the weak acid inner salt (D) is preferably a diphenyliodonium salt having an iodonium cation to which two phenyl groups are bonded, and a carboxylic acid anion substituted with at least one phenyl group of two phenyl groups bonded to the iodonium cation, and specific examples thereof include a salt represented by the following formula:

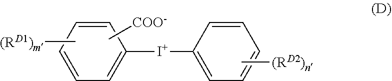

wherein, in formula (D),
R^{D1} and R^{D2} each independently represent a hydrocarbon group having 1 to 12 carbon atoms, an alkoxy group having 1 to 6 carbon atoms, an acyl group having 2 to 7 carbon atoms, an acyloxy group having 2 to 7 carbon atoms, an alkoxycarbonyl group having 2 to 7 carbon atoms, a nitro group or a halogen atom, and
m′ and n′ each independently represent an integer of 0 to 4, and when m′ is 2 or more, a plurality of R^{D1} may be the same or different, and when n′ is 2 or more, a plurality of R^{D2} may be the same or different.

Examples of the hydrocarbon group as for R^{D1} and R^{D2} include a chain hydrocarbon group, an alicyclic hydrocarbon group, an aromatic hydrocarbon group, and groups formed by combining these groups.

Examples of the chain hydrocarbon group include alkyl groups such as a methyl group, an ethyl group, a propyl group, an isopropyl group, a butyl group, an isobutyl group, a tert-butyl group, a pentyl group, a hexyl group, a nonyl group and the like.

The alicyclic hydrocarbon group may be either monocyclic or polycyclic, or may be either saturated or unsaturated. Examples thereof include cycloalkyl groups such as a cyclopropyl group, a cyclobutyl group, a cyclopentyl group, a cyclohexyl group, a cyclononyl group and a cyclododecyl group, a norbornyl group, an adamantyl group and the like.

Examples of the aromatic hydrocarbon group include aryl groups such as a phenyl group, a 1-naphthyl group, a 2-naphthyl group, a 2-methylphenyl group, a 3-methylphenyl group, a 4-methylphenyl group, a 4-ethylphenyl group, a 4-propylphenyl group, a 4-isopropylphenyl group, a 4-butylphenyl group, a 4-t-butylphenyl group, a 4-hexylphenyl group, a 4-cyclohexylphenyl group, an anthryl group, a p-adamantylphenyl group, a tolyl group, a xylyl group, a cumenyl group, a mesityl group, a biphenyl group, a phenanthryl group, a 2,6-diethylphenyl group and a 2-methyl-6-ethylphenyl group.

Examples of the groups formed by combining these groups include an alkyl-cycloalkyl group, a cycloalkyl-alkyl group, an aralkyl group (e.g., a phenylmethyl group, a 1-phenylethyl group, a 2-phenylethyl group, a 1-phenyl-1-propyl group, a 1-phenyl-2-propyl group, a 2-phenyl-2-propyl group, a 3-phenyl-1-propyl group, a 4-phenyl-1-butyl group, a 5-phenyl-1-pentyl group, a 6-phenyl-1-hexyl group, etc.) and the like.

Examples of the alkoxy group include a methoxy group, an ethoxy group and the like.

Examples of the acyl group include an acetyl group, a propanoyl group, a benzoyl group, a cyclohexanecarbonyl group and the like.

Examples of the acyloxy group include groups obtained by bonding an oxy group (—O—) to the above acyl group.

Examples of the alkoxycarbonyl group include groups obtained by bonding a carbonyl group (—CO—) to the above alkoxy group.

Examples of the halogen atom include a fluorine atom, a chlorine atom, a bromine atom and the like.

Preferably, R^{D1} and R^{D2} each independently represent an alkyl group having 1 to 8 carbon atoms, a cycloalkyl group having 3 to 10 carbon atoms, an alkoxy group having 1 to 6 carbon atoms, an acyl group having 2 to 4 carbon atoms, an acyloxy group having 2 to 4 carbon atoms, an alkoxycarbonyl group having 2 to 4 carbon atoms, a nitro group or a halogen atom.

Preferably, m′ and n′ are each independently an integer of 0 to 2, and more preferably 0, and when m′ is 2 or more, a plurality of R^{D1} may be the same or different, and when n′ is 2 or more, a plurality of R^{D2} may be the same or different.

More specifically, the following salts are exemplified.

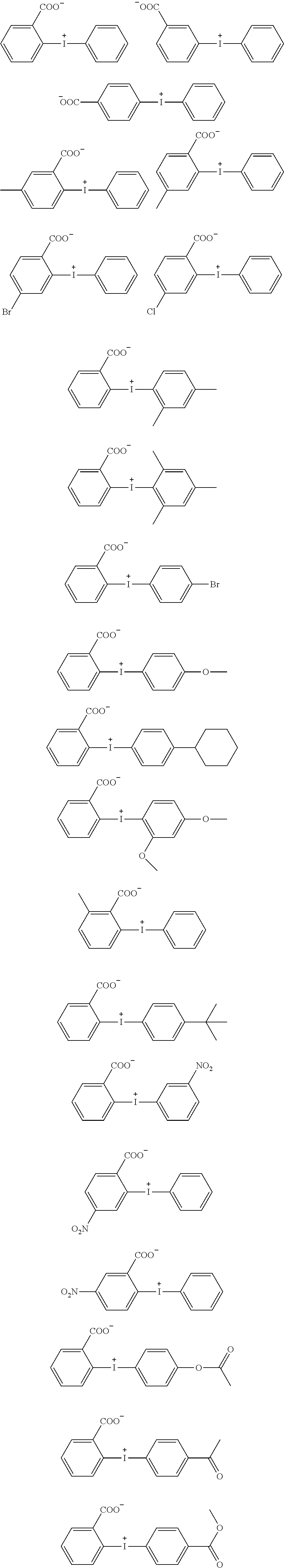

### <Other Components>

The resist composition of the present invention may also include components other than the components mentioned above (hereinafter sometimes referred to as “other components (F)”). The other components (F) are not particularly limited and it is possible to use various additives known in the resist field, for example, sensitizers, dissolution inhibitors, surfactants, stabilizers and dyes.

### <Preparation of Resist Composition>

The resist composition of the present invention can be prepared by mixing a salt (I) and/or a resin (Ap), and if necessary, an acid generator (B), a resin (A), resins other than the resin (A), a solvent (E), a quencher (C) and other components (F). The order of mixing these components is any order and is not particularly limited. It is possible to select, as the temperature during mixing, appropriate temperature from 10 to 40° C., according to the type of the resin, the solubility in the solvent (E) of the resin and the like. It is possible to select, as the mixing time, appropriate time from 0.5 to 24 hours according to the mixing temperature. The mixing means is not particularly limited and it is possible to use mixing with stirring.

After mixing the respective components, the mixture is preferably filtered through a filter having a pore diameter of about 0.003 to 0.2 μm.

### <Method for Producing Resist Pattern>

The method for producing a resist pattern of the present invention include:
(1) a step of applying the resist composition of the present invention on a substrate,
(2) a step of drying the applied composition to form a composition layer,
(3) a step of exposing the composition layer,
(4) a step of heating the exposed composition layer, and
(5) a step of developing the heated composition layer.

The resist composition can be usually applied on a substrate using a conventionally used apparatus, such as a spin coater. Examples of the substrate include inorganic substrates such as a silicon wafer, and organic substrates in which a resist film or the like is formed on the surface. Before applying the resist composition, the substrate may be washed, and an organic antireflection film may be formed on the substrate.

The solvent is removed by drying the applied composition to form a composition layer. Drying is performed by evaporating the solvent using a heating device such as a hot plate (so-called “prebake”), or a decompression device. The heating temperature is preferably 50 to 200° C. and the heating time is preferably 10 to 180 seconds. The pressure during drying under reduced pressure is preferably about 1 to 1.0×10⁵ Pa.

The composition layer thus obtained is usually exposed using an aligner. The aligner may be a liquid immersion aligner. It is possible to use, as an exposure source, various exposure sources, for example, exposure sources capable of emitting laser beam in an ultraviolet region such as KrF excimer laser (wavelength of 248 nm), ArF excimer laser (wavelength of 193 nm) and F₂ excimer laser (wavelength of 157 nm), an exposure source capable of emitting harmonic laser beam in a far-ultraviolet or vacuum ultra violet region by wavelength-converting laser beam from a solid-state laser source (YAG or semiconductor laser), an exposure source capable of emitting electron beam or extreme ultraviolet light (EUV) and the like. As used herein, such exposure to radiation is sometimes collectively referred to as “exposure”. The exposure is usually performed through a mask corresponding to a pattern to be required. When electron beam is used as the exposure source, exposure may be performed by direct writing without using the mask.

The exposed composition layer is subjected to a heat treatment (so-called “post-exposure bake”) to promote the deprotection reaction in an acid-labile group. The heating temperature is usually about 50 to 200° C., and preferably about 70 to 150° C. It is also possible to perform a chemical treatment (silylation) which adjusts the hydrophilicity or hydrophobicity of the resin on a surface side of the composition after heating. Before performing the development, the steps of application of the resist composition, drying, exposure and heating may be repeatedly performed on the exposed composition layer.

The heated composition layer is usually developed with a developing solution using a development apparatus. Examples of the developing method include a dipping method, a paddle method, a spraying method, a dynamic dispensing method and the like. The developing temperature is preferably, for example, 5 to 60° C. and the developing time is preferably, for example, 5 to 300 seconds. It is possible to produce a positive resist pattern or negative resist pattern by selecting the type of the developing solution as follows.

When the positive resist pattern is produced from the resist composition of the present invention, an alkaline developing solution is used as the developing solution. The alkaline developing solution may be various aqueous alkaline solutions used in this field. Examples thereof include aqueous solutions of tetramethylammonium hydroxide and (2-hydroxyethyl)trimethylammonium hydroxide (commonly known as choline). The surfactant may be contained in the alkaline developing solution.

It is preferable that the developed resist pattern is washed with ultrapure water and then water remaining on the substrate and the pattern is removed.

When the negative resist pattern is produced from the resist composition of the present invention, a developing solution containing an organic solvent (hereinafter sometimes referred to as “organic developing solution”) is used as the developing solution.

Examples of the organic solvent contained in the organic developing solution include ketone solvents such as 2-hexanone and 2-heptanone; glycol ether ester solvents such as propylene glycol monomethyl ether acetate; ester solvents such as butyl acetate; glycol ether solvents such as propylene glycol monomethyl ether; amide solvents such as N,N-dimethylacetamide; and aromatic hydrocarbon solvents such as anisole.

The content of the organic solvent in the organic developing solution is preferably 90% by mass or more and 100% by mass or less, more preferably 95% by mass or more and 100% by mass or less, and still more preferably the organic developing solution is substantially composed of the organic solvent.

Particularly, the organic developing solution is preferably a developing solution containing butyl acetate and/or 2-heptanone. The total content of butyl acetate and 2-heptanone in the organic developing solution is preferably 50% by mass or more and 100% by mass or less, more preferably 90% by mass or more and 100% by mass or less, and still more preferably the organic developing solution is substantially composed of butyl acetate and/or 2-heptanone.

The surfactant may be contained in the organic developing solution. A trace amount of water may be contained in the organic developing solution.

During development, the development may be stopped by replacing by a solvent with the type different from that of the organic developing solution.

The developed resist pattern is preferably washed with a rinsing solution. The rinsing solution is not particularly limited as long as it does not dissolve the resist pattern, and it is possible to use a solution containing an ordinary organic solvent which is preferably an alcohol solvent or an ester solvent.

After washing, the rinsing solution remaining on the substrate and the pattern is preferably removed.

### <Applications>

The resist composition of the present invention is suitable as a resist composition for exposure of KrF excimer laser, a resist composition for exposure of electron beam (EB) or a resist composition for exposure of EUV, particularly a resist composition for exposure of electron beam (EB) or a resist composition for exposure of EUV, and the resist composition is useful for fine processing of semiconductors.

### EXAMPLES

The present invention will be described more specifically by way of Examples. Percentages and parts expressing the contents or amounts used in the Examples are by mass unless otherwise specified.

The weight-average molecular weight is a value determined by gel permeation chromatography. Analysis conditions of gel permeation chromatography are as follows.

Column: TSKgel Multipore IIXL-M×3+guardcolumn (manufactured by TOSOH CORPORATION)
Eluent: tetrahydrofuran
Flow rate: 1.0 mL/min
Detector: RI detector
Column temperature: 40° C.
Injection amount: 100 μl
Molecular weight standards: polystyrene standard (manufactured by TOSOH CORPORATION)

Structures of compounds were confirmed by measuring a molecular ion peak using mass spectrometry (LC is Model 1100, manufactured by Agilent Technologies, Inc., and MASS is Model LC/MSD, manufactured by Agilent Technologies, Inc.). The value of this molecular ion peak in the following Examples is indicated by “MASS”.

### Example 1: Synthesis of Salt Represented by Formula (I-1)

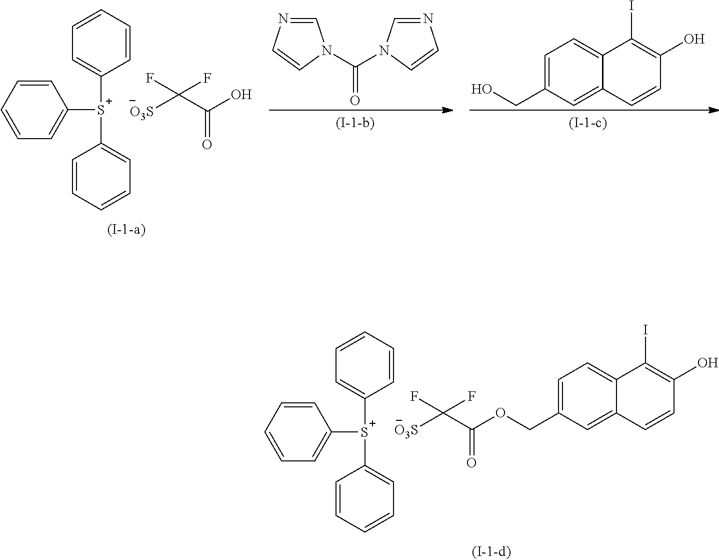

4.38 Parts of a salt represented by formula (I-1-a) and 30 parts of chloroform were mixed and, after stirring at 23° C. for 30 minutes, 1.62 parts of a compound represented by formula (I-1-b) was added, followed by temperature rise to 50° C. and further stirring at 50° C. for 2 hours. To the reaction mixture thus obtained, 3.00 parts of a compound represented by formula (I-1-c) was added, followed by stirring at 50° C. for 3 hours and further cooling to 23° C. To the mixture thus obtained, 10 parts of an aqueous 5% oxalic acid solution was added and, after stirring at 23° C. for 30 minutes, the organic layer was isolated through separation. To the organic layer thus obtained, 10 parts of ion-exchanged water was added and, after stirring at 23° C. for 30 minutes, the organic layer was isolated through separation. This water washing operation was repeated five times. The organic layer thus obtained was concentrated and then 30 parts of tert-butyl methyl ether was added to the concentrated residue and, after stirring at 23° C. for 30 minutes, the supernatant was removed, followed by concentration to obtain 6.02 parts of a salt represented by formula (I-1-d).

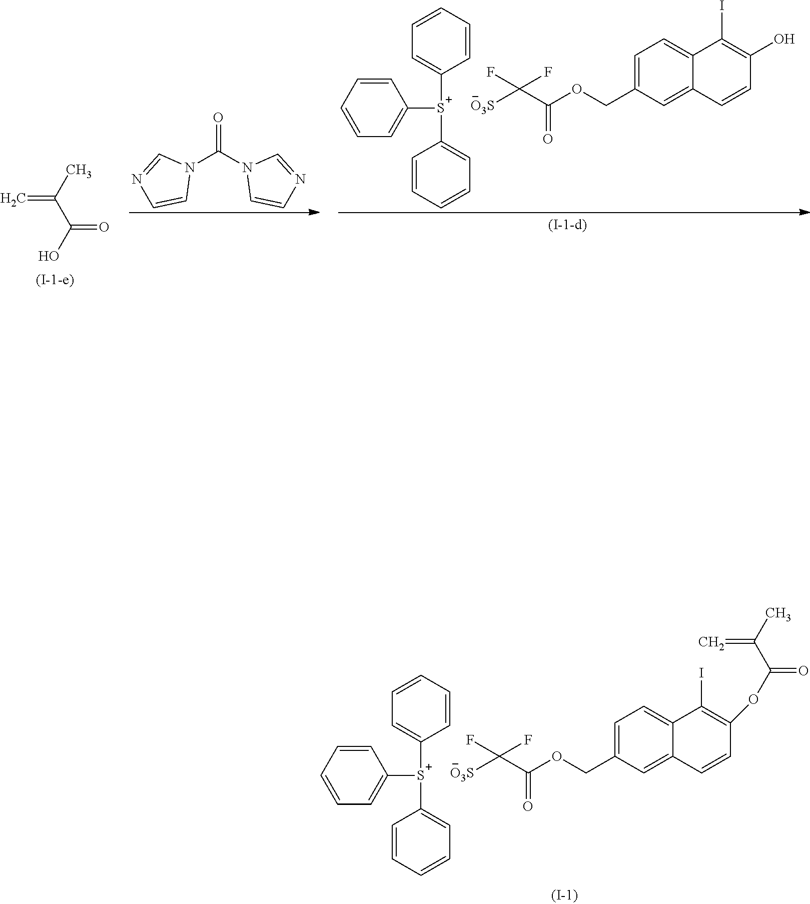

0.43 Part of a compound represented by formula (I-1-e) and 50 parts of chloroform were mixed, followed by stirring at 23° C. for 30 minutes. To the mixture thus obtained, 0.81 part of carbonyldiimidazole was added, followed by stirring at 50° C. for 2 hours. The mixture thus obtained was mixed with 3.61 parts of a salt represented by formula (I-1-d), followed nu stirring at 50° C. for 2 hours and further cooling to 23° C. To the mixture thus obtained, 20 parts of chloroform and 20 parts of an aqueous 5% oxalic acid solution were added and, after stirring at 23° C. for 30 minutes, the organic layer was isolated through separation. To the organic layer thus obtained, 20 parts of ion-exchanged water was added and, after stirring at 23° C. for 30 minutes, the organic layer was isolated through separation. This water washing operation was repeated five times. The organic layer thus obtained was concentrated and then 1 part of acetonitrile and 20 parts of tert-butyl methyl ether were added to the concentrated residue and, after stirring at 23° C. for 30 minutes, the supernatant was removed, followed by concentration to obtain 3.42 parts of a salt represented by formula (I-1).

MS (ESI (+) Spectrum): M⁺263.1

MS (ESI (−) Spectrum): M⁻ 524.9

### Example 2: Synthesis of Salt Represented by Formula (1-2)

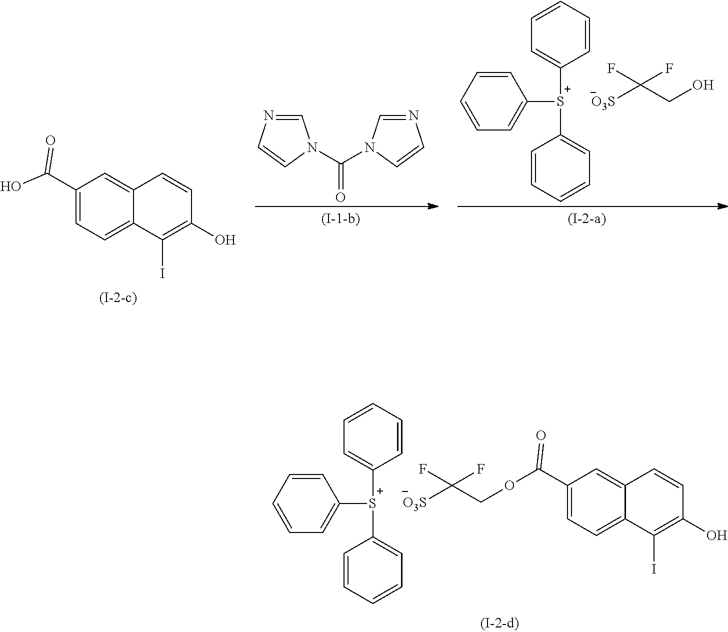

3.14 Parts of a compound represented by formula (I-2-c) and 30 parts of chloroform were mixed and, after stirring at 23° C. for 30 minutes, 1.62 parts of a compound represented by formula (I-1-b) was added, followed by temperature rise to 50° C. and further stirring at 50° C. for 2 hours. To the reaction mixture thus obtained, 4.24 parts of a salt represented by formula (I-2-a) was added, followed by stirring at 50° C. for 3 hours and further cooling to 23° C. To the mixture thus obtained, 10 parts of an aqueous 5% oxalic acid solution was added and, after stirring at 23° C. for 30 minutes, the organic layer was isolated through separation. To the organic layer thus obtained, 10 parts of ion-exchanged water was added and, after stirring at 23° C. for 30 minutes, the organic layer was isolated through separation. This water washing operation was repeated five times. The organic layer thus obtained was concentrated and then 30 parts of tert-butyl methyl ether was added to the concentrated residue and, after stirring at 23° C. for 30 minutes, the supernatant was removed, followed by concentration to obtain 5.77 parts of a salt represented by formula (I-2-d).

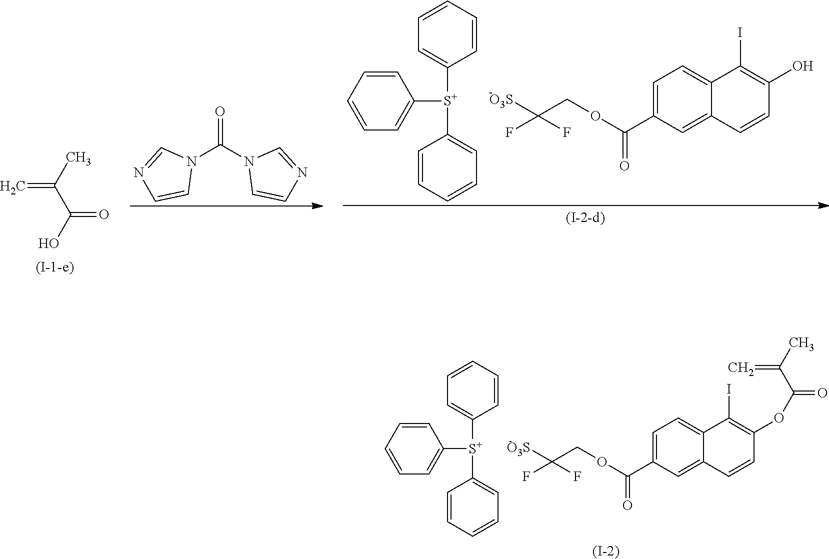

0.43 Part of a compound represented by formula (I-1-e) and 50 parts of chloroform were mixed, followed by stirring at 23° C. for 30 minutes. To the mixture thus obtained, 0.81 part of carbonyldiimidazole was added, followed by stirring at 50° C. for 2 hours. The mixture thus obtained was mixed with 3.61 parts of a salt represented by formula (I-2-d), followed by stirring at 50° C. for 2 hours and further cooling to 23° C. To the mixture thus obtained, 20 parts of chloroform and 20 parts of an aqueous 5% oxalic acid solution were added and, after stirring at 23° C. for 30 minutes, the organic layer was isolated through separation. To the organic layer thus obtained, 20 parts of ion-exchanged water was added and, after stirring at 23° C. for 30 minutes, the organic layer was isolated through separation. This water washing operation was repeated five times. The organic layer thus obtained was concentrated and then 1 part of acetonitrile and 20 parts of tert-butyl methyl ether were added to the concentrated residue and, after stirring at 23° C. for 30 minutes, the supernatant was removed, followed by concentration to obtain 3.59 parts of a salt represented by formula (I-2).

MS (ESI (+) Spectrum): M⁺263.1

MS (ESI (−) Spectrum): M⁻ 524.9

### Example 3: Synthesis of Salt Represented by Formula (I-3)

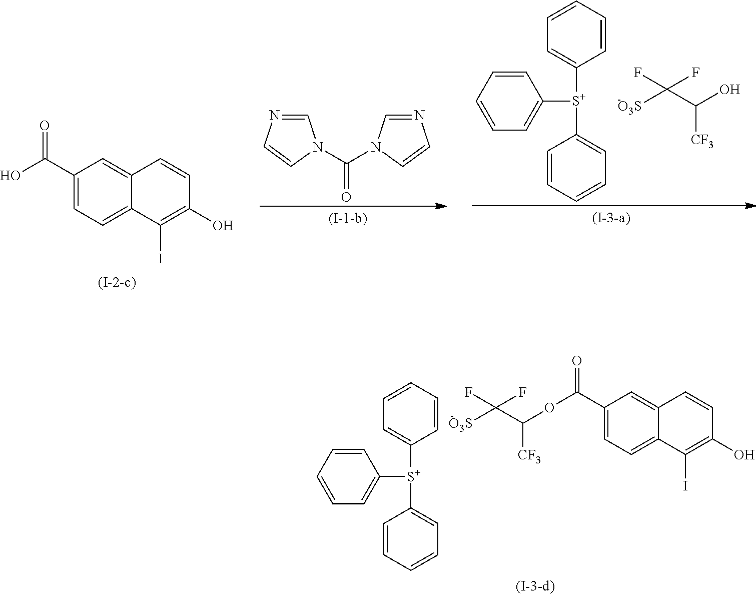

3.14 parts of a compound represented by formula (1-2-c) and 30 parts of chloroform were mixed and, after stirring at 23° C. for 30 minutes, 1.62 parts of a compound represented by formula (I-1-b) was added, followed by temperature rise to 50° C. and further stirring at 50° C. for 2 hours. To the reaction mixture thus obtained, 4.92 parts of a salt represented by formula (I-3-a) was added, followed by stirring at 50° C. for 3 hours and further cooling to 23° C. To the mixture thus obtained, 10 parts of an aqueous 5% oxalic acid solution was added and, after stirring at 23° C. for 30 minutes, the organic layer was isolated through separation. To the organic layer thus obtained, 10 parts of ion-exchanged water was added and, after stirring at 23° C. for 30 minutes, the organic layer was isolated through separation. This water washing operation was repeated five times. The organic layer thus obtained was concentrated and then 30 parts of tert-butyl methyl ether was added to the concentrated residue and, after stirring at 23° C. for 30 minutes, the supernatant was removed, followed by concentration to obtain 1.02 parts of a salt represented by formula (I-3-d).

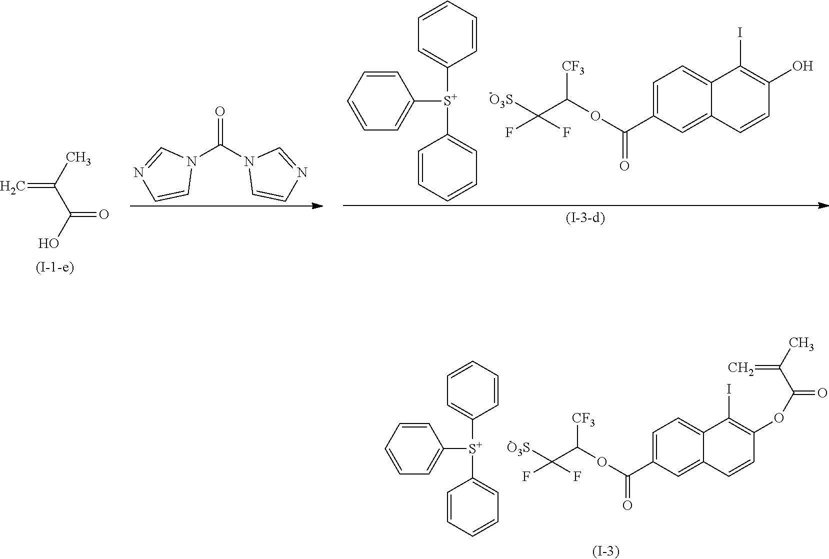

0.43 Part of a compound represented by formula (I-1-e) and 50 parts of chloroform were mixed, followed by stirring at 23° C. for 30 minutes. To the mixture thus obtained, 0.81 part of carbonyldiimidazole was added, followed by stirring at 50° C. for 2 hours. The mixture thus obtained was mixed with 3.94 parts of a salt represented by formula (I-3-d), followed by stirring at 50° C. for 2 hours and further cooling to 23° C. To the mixture thus obtained, 20 parts of chloroform and 20 parts of an aqueous 5% oxalic acid solution were added and, after stirring at 23° C. for 30 minutes, the organic layer was isolated through separation. To the organic layer thus obtained, 20 parts of ion-exchanged water was added and, after stirring at 23° C. for 30 minutes, the organic layer was isolated through separation. This water washing operation was repeated five times. The organic layer thus obtained was concentrated and then 1 part of acetonitrile and 20 parts of tert-butyl methyl ether were added to the concentrated residue and, after stirring at 23° C. for 30 minutes, the supernatant was removed, followed by concentration to obtain 3.11 parts of a salt represented by formula (I-3).

MS (ESI (+) Spectrum): M⁺263.1

MS (ESI (−) Spectrum): M⁻ 592.9

### Example 4: Synthesis of Salt Represented by Formula (I-24)

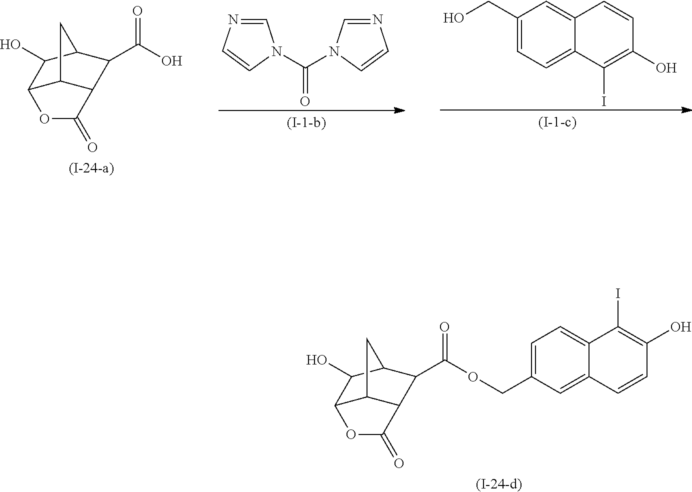

3.96 Parts of a compound represented by formula (I-24-a) and 40 parts of chloroform were mixed, followed by stirring at 23° C. for 30 minutes. To the mixed solution thus obtained, 3.24 parts of a compound represented by formula (I-1-b) was added, followed by stirring at 50° C. for 2 hours. To the mixed solution thus obtained, 6.00 parts of a compound represented by formula (I-1-c) was added, followed by stirring at 50° C. for 3 hours and further cooling to 23° C. To the mixture thus obtained, 20 parts of an aqueous 5% oxalic acid solution was added and, after stirring at 23° C. for 30 minutes, the organic layer was isolated through separation. To the organic layer thus obtained, 20 parts of ion-exchanged water was added and, after stirring at 23° C. for 30 minutes, the organic layer was isolated through separation. This water washing operation was repeated five times. The organic layer thus obtained was concentrated and then the concentrated mixture was isolated using a column (silica gel 60N (spherical, neutral) 100-210 μm; manufactured by Kanto Chemical Co., Inc., developing solvent: n-heptane/ethyl acetate=3/1) to obtain 7.02 parts of a compound represented by formula (I-24-d).

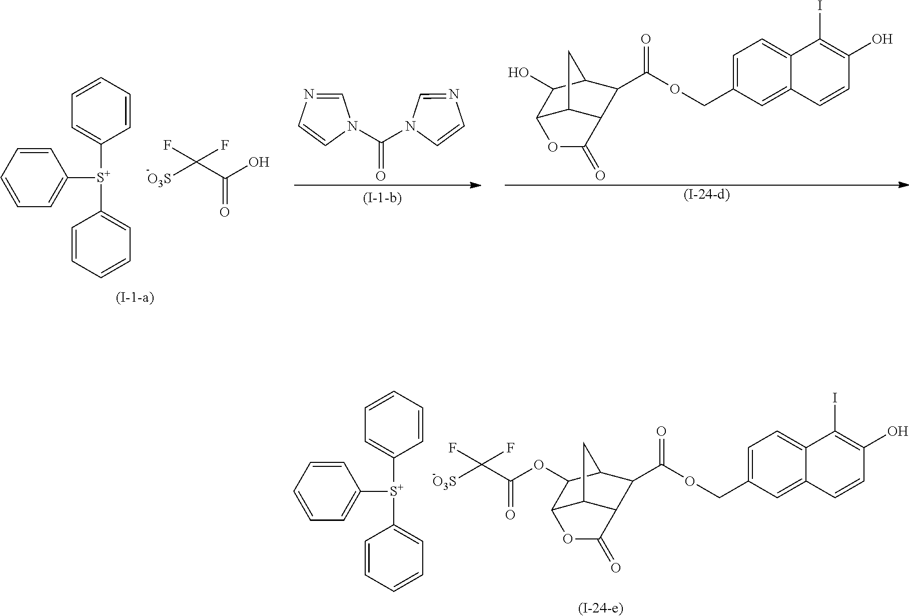

2.19 Parts of a salt represented by formula (I-1-a) and 30 parts of chloroform were mixed and, after stirring at 23° C. for 30 minutes, 0.81 part of a compound represented by formula (I-1-b) was added, followed by temperature rise to 50° C. and further stirring at 50° C. for 2 hours. To the reaction mixture thus obtained, 2.40 parts of a compound represented by formula (I-24-d) was added, followed by stirring at 50° C. for 3 hours and further cooling to 23° C. To the mixture thus obtained, 10 parts of an aqueous 5% oxalic acid solution was added and, after stirring at 23° C. for 30 minutes, the organic layer was isolated through separation. To the organic layer thus obtained, 10 parts of ion-exchanged water was added and, after stirring at 23° C. for 30 minutes, the organic layer was isolated through separation. This water washing operation was repeated five times. The organic layer thus obtained was concentrated and then the concentrated mixture was isolated using a column (silica gel 60N (spherical, neutral) 100-210 μm; manufactured by Kanto Chemical Co., Inc., developing solvent: methanol/chloroform=1/1) to obtain 2.24 parts of a salt represented by formula (I-24-e).

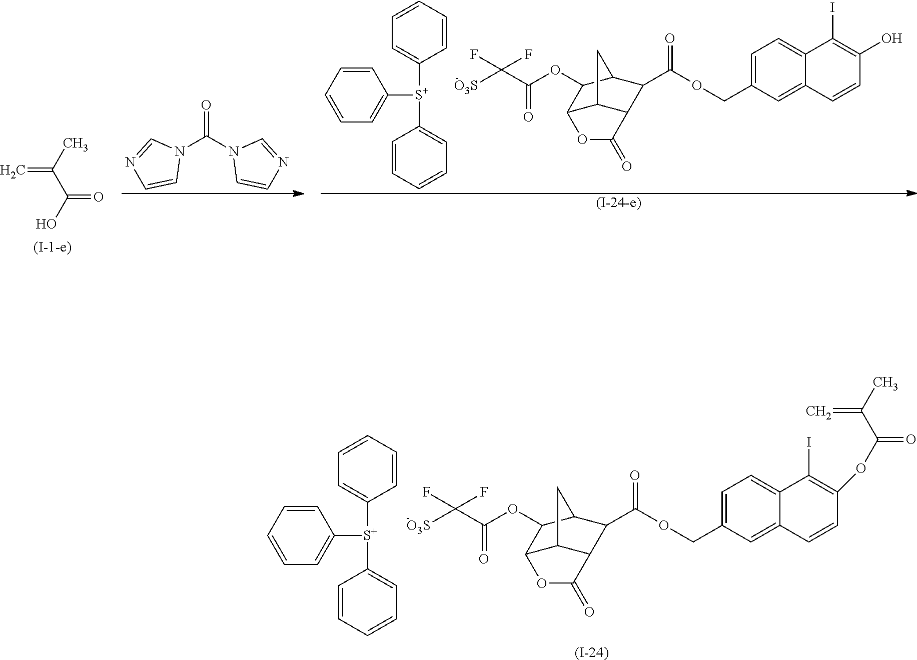

0.17 Part of a compound represented by formula (I-1-e) and 30 parts of chloroform were mixed, followed by stirring at 23° C. for 30 minutes. To the mixture thus obtained, 0.32 part of carbonyldiimidazole was added, followed by stirring at 50° C. for 2 hours. To the reaction mixture thus obtained, 1.80 parts of a salt represented by formula (I-24-e) was added, followed by stirring at 50° C. for 2 hours and further cooling to 23° C. To the mixture thus obtained, 20 parts of chloroform and 10 parts of an aqueous 5% oxalic acid solution were added and, after stirring at 23° C. for 30 minutes, the organic layer was isolated through separation. To the organic layer thus obtained, 20 parts of ion-exchanged water was added and, after stirring at 23° C. for 30 minutes, the organic layer was isolated through separation. This water washing operation was repeated five times. The organic layer thus obtained was concentrated and then 1 part of acetonitrile and 20 parts of tert-butyl methyl ether were added to the concentrated residue and, after stirring at 23° C. for 30 minutes, the supernatant was removed, followed by concentration to obtain 1.77 parts of a salt represented by formula (I-24).

MS (ESI (+) Spectrum): M⁺263.1

MS (ESI (−) Spectrum): M⁻ 705.0

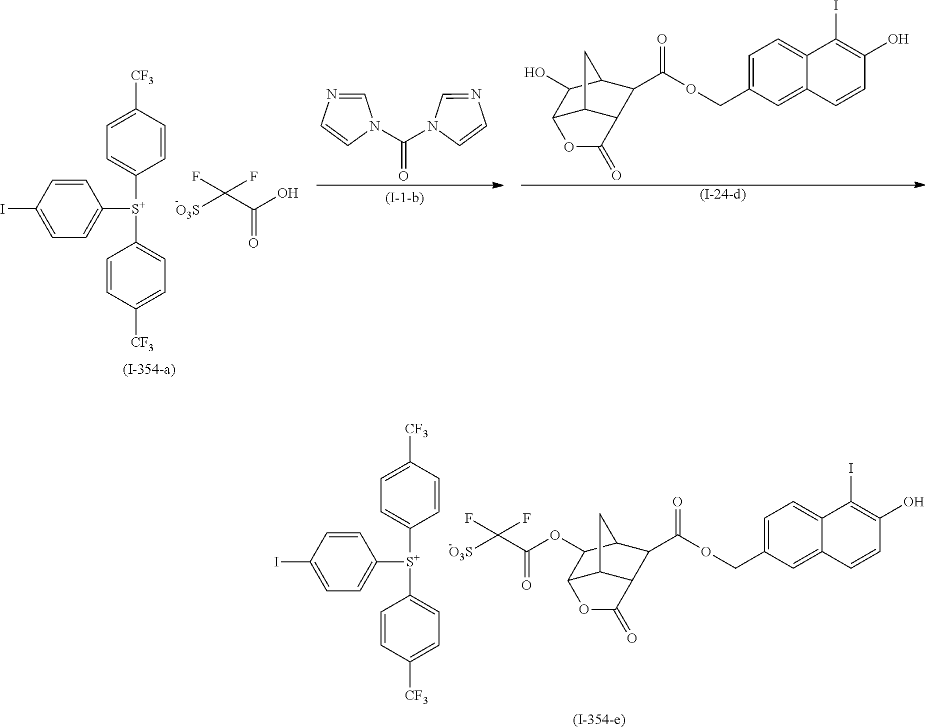

### Example 5: Synthesis of Salt Represented by Formula (I-354)

3.50 Parts of a salt represented by formula (I-354-a) and 30 parts of chloroform were mixed and, after stirring at 23° C. for 30 minutes, 0.81 part of a compound represented by formula (I-1-b) was added, followed by temperature rise to 50° C. and further stirring at 50° C. for 2 hours. To the reaction mixture thus obtained, 2.40 parts of a compound represented by formula (I-24-d) was added, followed by stirring at 50° C. for 3 hours and further cooling to 23° C. To the mixture thus obtained, 10 parts of an aqueous 5% oxalic acid solution was added and, after stirring at 23° C. for 30 minutes, the organic layer was isolated through separation. To the organic layer thus obtained, 10 parts of ion-exchanged water was added and, after stirring at 23° C. for 30 minutes, the organic layer was isolated through separation. This water washing operation was repeated five times. The organic layer thus obtained was concentrated and then 30 parts of tert-butyl methyl ether was added to the concentrated residue and, after stirring at 23° C. for 30 minutes, the supernatant was removed, followed by concentration to obtain 4.98 parts of a salt represented by formula (I-354-e).

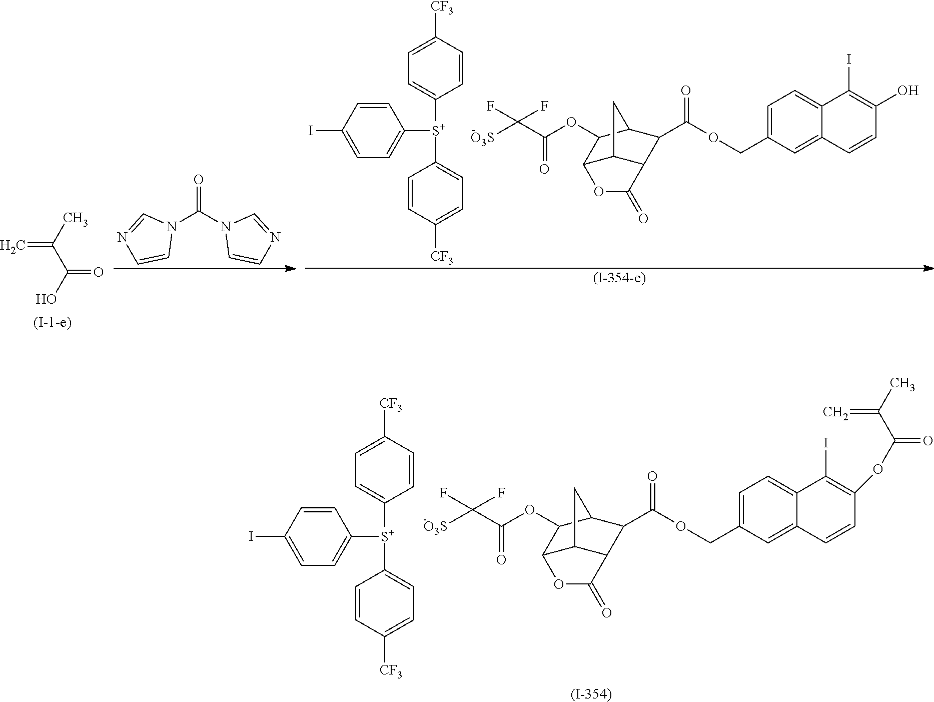

0.17 Part of a compound represented by formula (I-1-e) and 30 parts of chloroform were mixed, followed by stirring at 23° C. for 30 minutes. To the mixture thus obtained, 0.32 part of carbonyldiimidazole was added, followed by stirring at 50° C. for 2 hours. The mixture thus obtained was mixed with 2.33 parts of a salt represented by formula (I-354-e), followed by stirring at 50° C. for 2 hours and further cooling to 23° C. To the mixture thus obtained, 20 parts of chloroform and 10 parts of an aqueous 5% oxalic acid solution were added and, after stirring at 23° C. for 30 minutes, the organic layer was isolated through separation. To the organic layer thus obtained, 20 parts of ion-exchanged water was added and, after stirring at 23° C. for 30 minutes, the organic layer was isolated through separation. This water washing operation was repeated five times. The organic layer thus obtained was concentrated and then 1 part of acetonitrile and 20 parts of tert-butyl methyl ether were added to the concentrated residue and, after stirring at 23° C. for 30 minutes, the supernatant was removed, followed by concentration to obtain 2.24 parts of a salt represented by formula (I-354).

MS (ESI (+) Spectrum): M⁺525.0

MS (ESI (−) Spectrum): M⁻ 705.0

### Example 6: Synthesis of Salt Represented by Formula (I-1136)

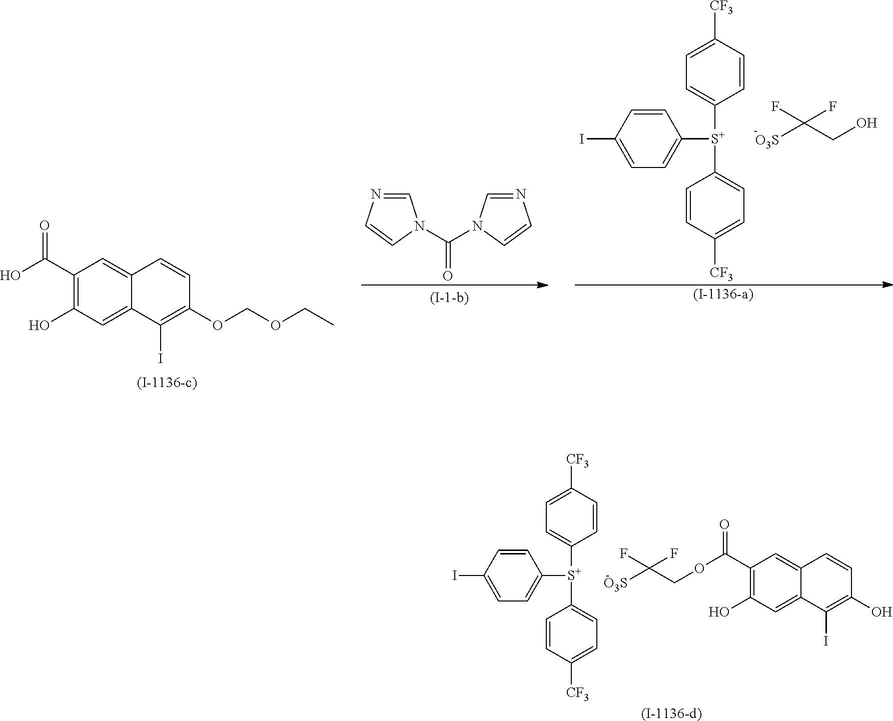

3.88 Parts of a compound represented by formula (I-1136-c) and 60 parts of chloroform were mixed and, after stirring at 23° C. for 30 minutes, 1.62 parts of a compound represented by formula (I-1-b) was added, followed by temperature rise to 50° C. and further stirring at 50° C. for 2 hours. To the reaction mixture thus obtained, 6.86 parts of a salt represented by formula (I-1136-a) was added, followed by stirring at 50° C. for 3 hours and further cooling to 23° C. To the mixture thus obtained, 30 parts of 1N hydrochloric acid was added and, after stirring at 23° C. for 30 minutes, the organic layer was isolated through separation. To the organic layer thus obtained, 30 parts of ion-exchanged water was added and, after stirring at 23° C. for 30 minutes, the organic layer was isolated through separation. This water washing operation was repeated five times. The organic layer thus obtained was concentrated and then 30 parts of tert-butyl methyl ether was added to the concentrated residue and, after stirring at 23° C. for 30 minutes, the supernatant was removed, followed by concentration to obtain 5.44 parts of a salt represented by formula (I-1136-d).

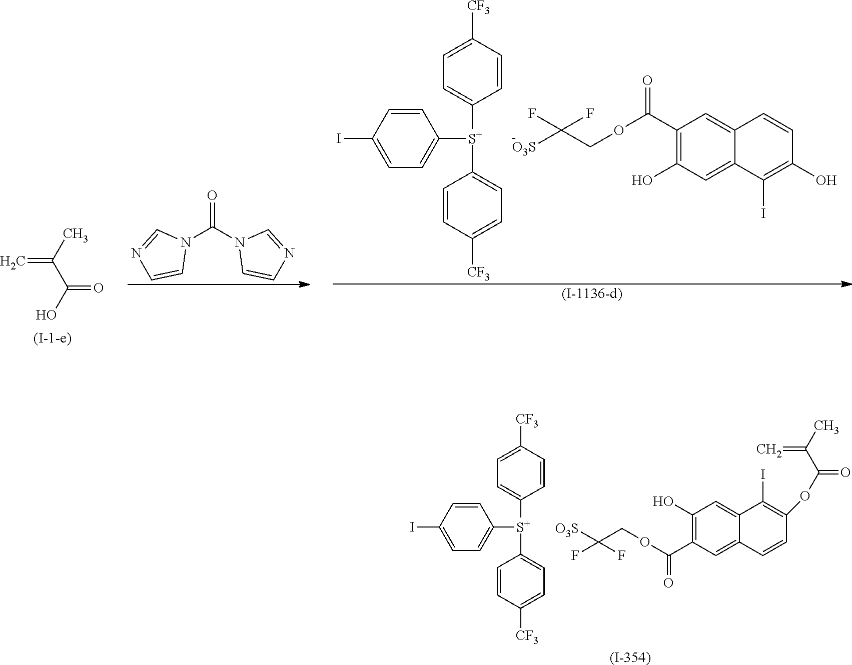

0.43 Part of a compound represented by formula (I-1-e) and 50 parts of chloroform were mixed, followed by stirring at 23° C. for 30 minutes. To the mixture thus obtained, 0.81 part of carbonyldiimidazole was added, followed by stirring at 50° C. for 2 hours. The mixture thus obtained was mixed with 4.99 parts of a salt represented by formula (I-1136-d), followed by stirring at 50° C. for 2 hours and further cooling to 23° C. To the mixture thus obtained, 20 parts of chloroform and 20 parts of an aqueous 5% oxalic acid solution were added and, after stirring at 23° C. for 30 minutes, the organic layer was isolated through separation. To the organic layer thus obtained, 20 parts of ion-exchanged water was added and, after stirring at 23° C. for 30 minutes, the organic layer was isolated through separation. This water washing operation was repeated five times. The organic layer thus obtained was concentrated and then 1 part of acetonitrile and 20 parts of tert-butyl methyl ether were added to the concentrated residue and, after stirring at 23° C. for 30 minutes, the supernatant was removed, followed by concentration to obtain 2.88 parts of a salt represented by formula (I-1136).

MS (ESI (+) Spectrum): M⁺525.0

MS (ESI (−) Spectrum): M⁻ 540.9

### Example 7: Synthesis of Salt Represented by Formula (I-1137)

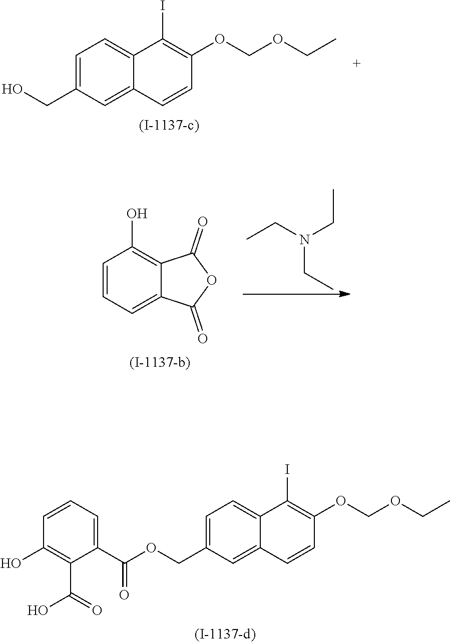

6.45 Parts of a compound represented by formula (I-1137-c), 0.45 part of dimethylaminopyridine, 2.19 parts of triethylamine and 40 parts of dimethylformamide were mixed and, after stirring at 23° C. for 30 minutes, 3.56 parts of a compound represented by formula (I-1137-b) and 20 parts of chloroform were added, followed by stirring at 50° C. for 5 hours. The mixture thus obtained was cooled to 23° C. and then 30 parts of an aqueous 5% oxalic acid solution were added, followed by stirring at 23° C. for 3 hours. The mixture thus obtained was cooled to 23° C. and then 30 parts of chloroform and 10 parts of ion-exchanged water were added and, after stirring at 23° C. for 30 minutes, the organic layer was isolated through separation. To the mixture thus obtained, 10 parts of ion-exchanged water was added and, after stirring at 23° C. for 30 minutes, the organic layer was isolated through separation. This water washing operation was repeated three times. The organic layer thus obtained was concentrated and then the concentrated mixture was isolated using a column (silica gel 60N (spherical, neutral) 100-210 μm; manufactured by Kanto Chemical Co., Inc., developing solvent: ethyl acetate) to obtain 5.72 parts of a salt represented by formula (I-1137-d).

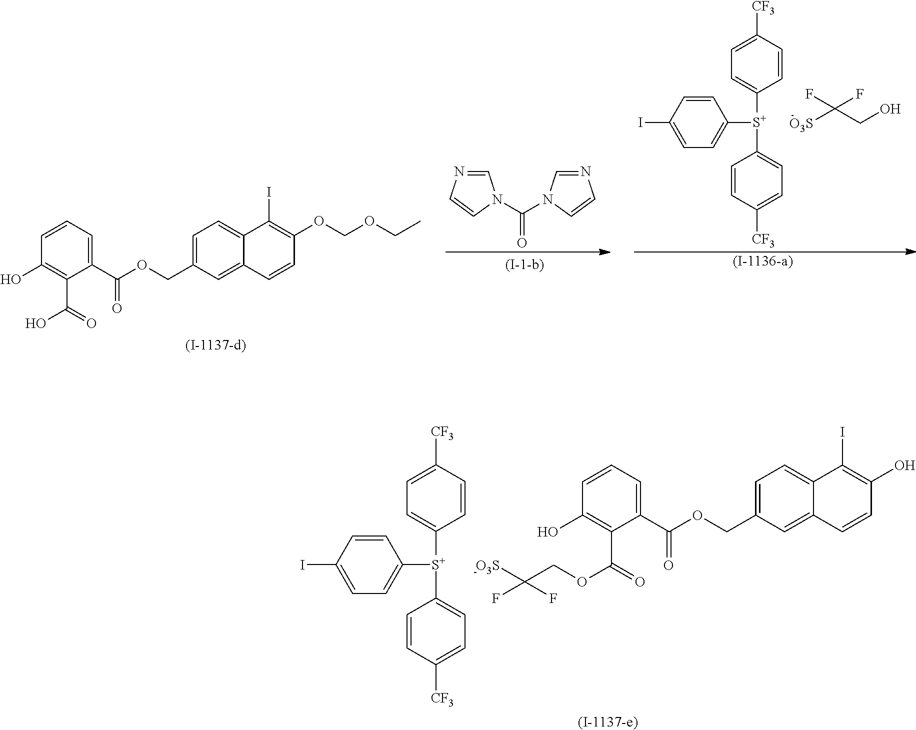

5.22 Parts of a compound represented by formula (I-1137-d) and 60 parts of chloroform were mixed and, after stirring at 23° C. for 30 minutes, 1.62 parts of a compound represented by formula (I-1-b) was added, followed by temperature rise to 50° C. and further stirring at 50° C. for 2 hours. To the reaction mixture thus obtained, 6.86 parts of a salt represented by formula (I-1136-a) was added, followed by stirring at 50° C. for 3 hours and further cooling to 23° C. To the mixture thus obtained, 30 parts of 1N hydrochloric acid was added and, after stirring at 23° C. for 30 minutes, the organic layer was isolated through separation. To the organic layer thus obtained, 30 parts of ion-exchanged water was added and, after stirring at 23° C. for 30 minutes, the organic layer was isolated through separation. This water washing operation was repeated five times. The organic layer thus obtained was concentrated and then 30 parts of tert-butyl methyl ether was added to the concentrated residue and, after stirring at 23° C. for 30 minutes, the supernatant was removed, followed by concentration to obtain 6.09 parts of a salt represented by formula (I-1137-e).

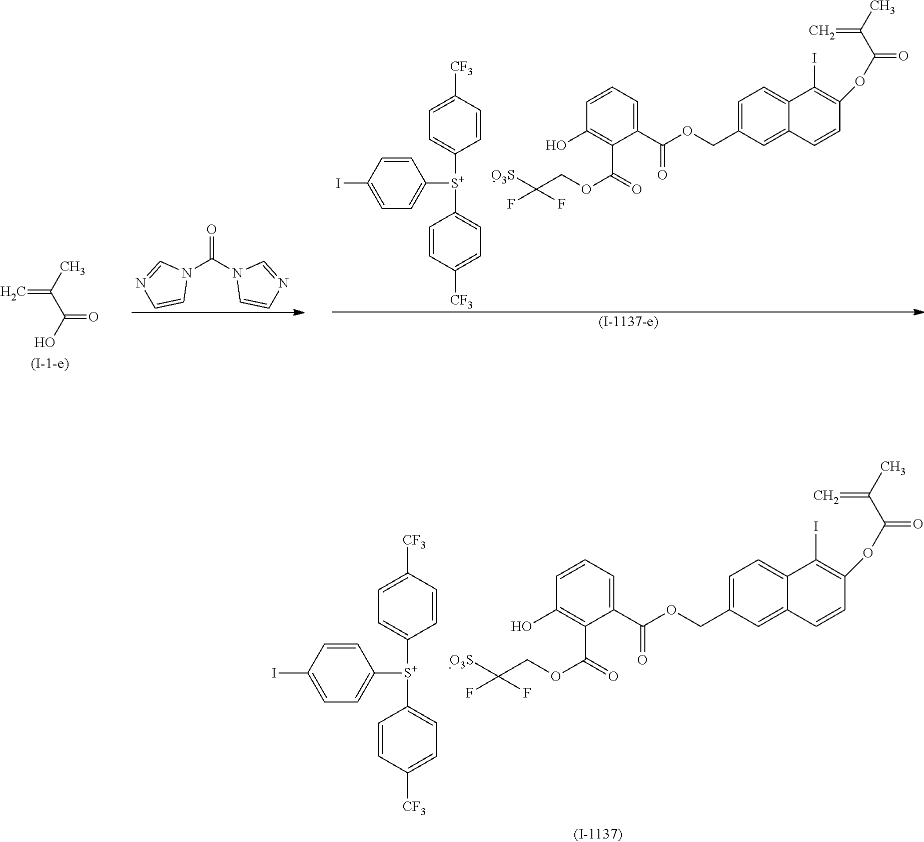

0.43 Part of a compound represented by formula (I-1-e) and 50 parts of chloroform 50 part were mixed, followed by stirring at 23° C. for 30 minutes. To the mixture thus obtained, 0.81 part of carbonyldiimidazole was added, followed by stirring at 50° C. for 2 hours. The mixture thus obtained was mixed with 5.66 parts of a salt represented by formula (I-1137-e), followed by stirring at 50° C. for 2 hours and further cooling to 23° C. To the mixture thus obtained, 20 parts of chloroform and 20 parts of an aqueous 5% oxalic acid solution were added and, after stirring at 23° C. for 30 minutes, the organic layer was isolated through separation. To the organic layer thus obtained, 20 parts of ion-exchanged water was added and, after stirring at 23° C. for 30 minutes, the organic layer was isolated through separation. This water washing operation was repeated five times. The organic layer thus obtained was concentrated and then 1 part of acetonitrile and 20 parts of tert-butyl methyl ether were added to the concentrated residue and, after stirring at 23° C. for 30 minutes, the supernatant was removed, followed by concentration to obtain 3.03 parts of a salt represented by formula (I-1137).

MS (ESI (+) Spectrum): M⁺525.0

MS (ESI (−) Spectrum): M⁻ 675.0

### Synthesis of Resin

Compounds (monomers) used in synthesis of a resin (A) are shown below. Hereinafter, these compounds are referred to as “monomer (a1-1-3)” or the like according to the formula number.

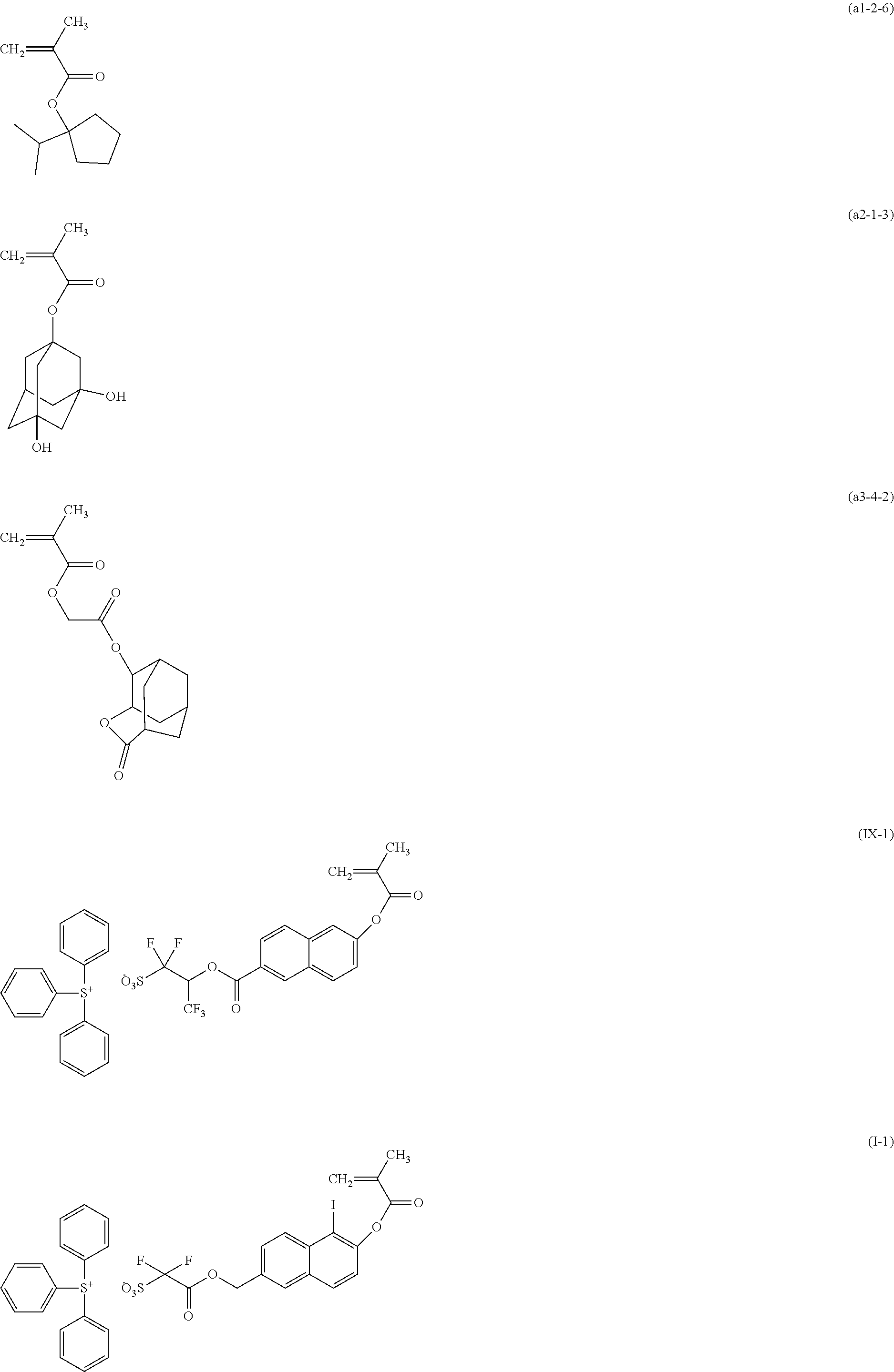

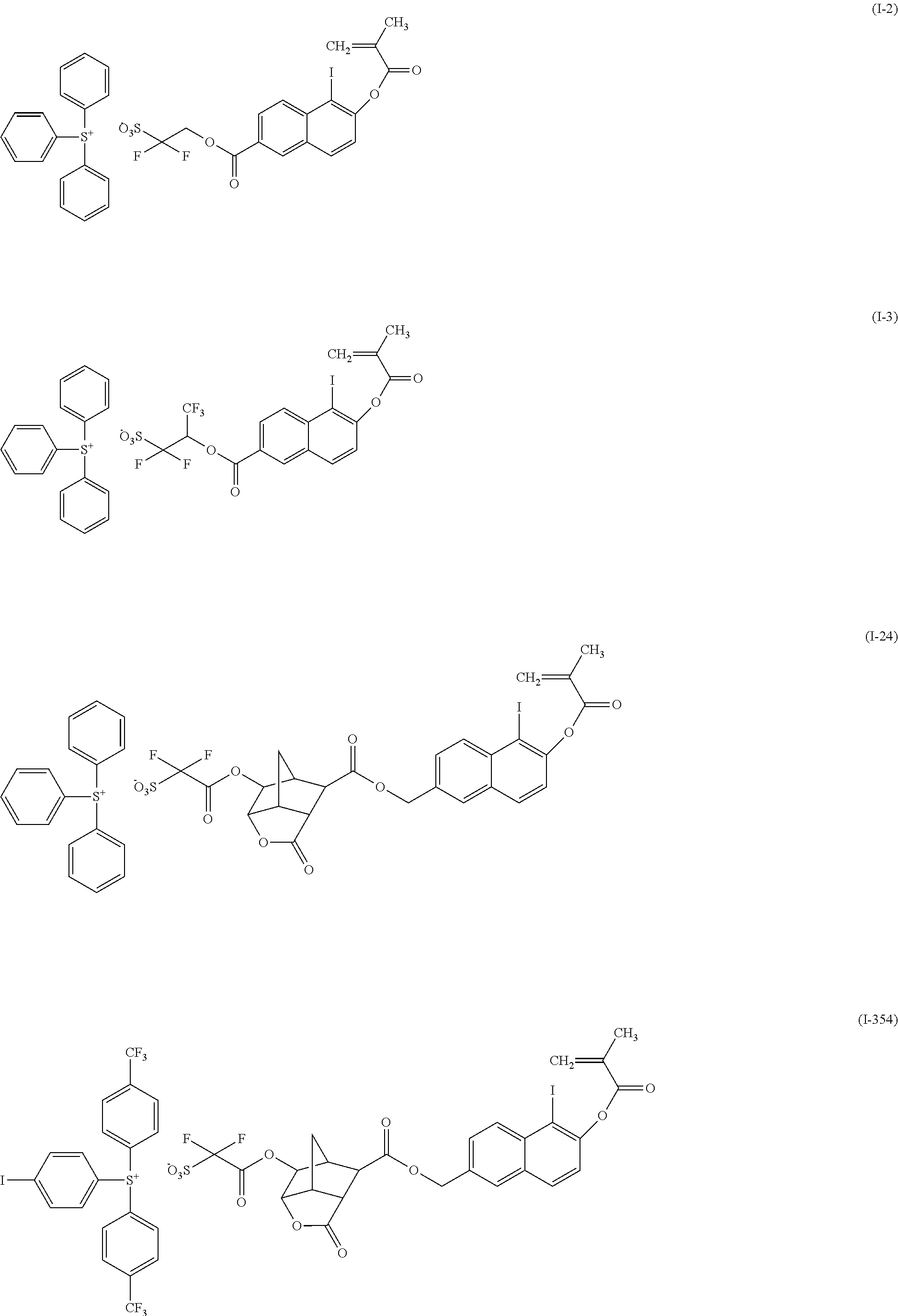

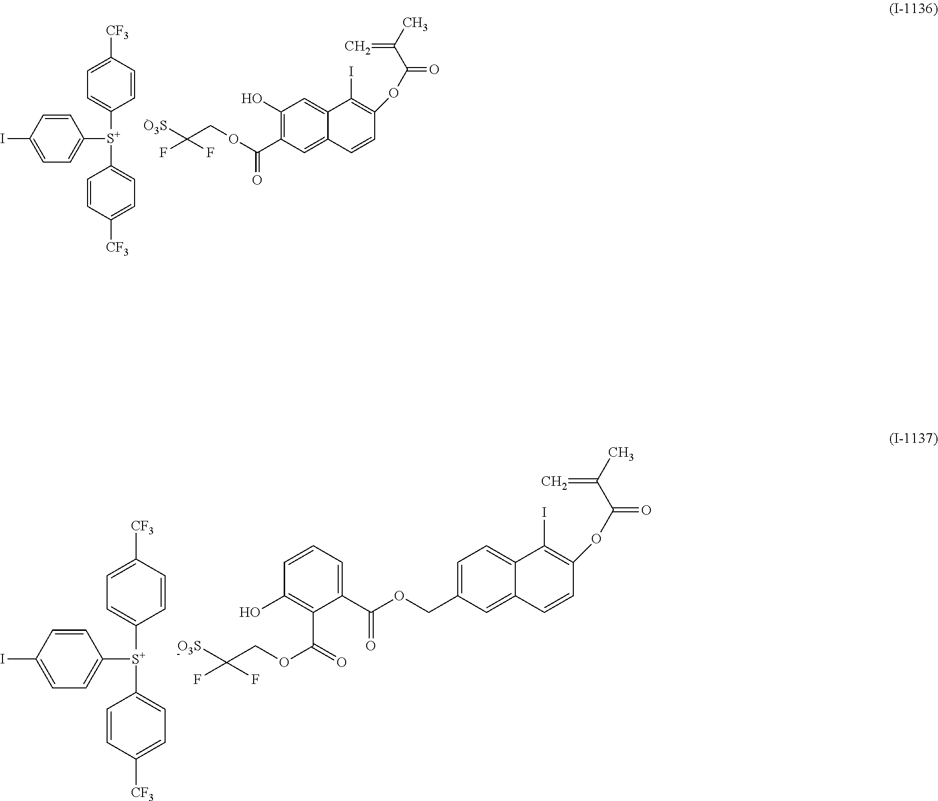

### Example 8 [Synthesis of Resin A1]

Using a monomer (a1-2-6), a monomer (a2-1-3), a monomer (a3-4-2), 4-acetoxystyrene and a monomer (I-1) as monomers, these monomers were mixed in a molar ratio of 55:3:12:25:5 [monomer (a1-2-6):monomer (a2-1-3):monomer (a3-4-2):4-acetoxystyrene:monomer (I-1)], and this monomer mixture was further mixed with propylene glycol monomethyl ether in the amount of 1.5 mass times the total mass of all monomers. To the mixture thus obtained, azobisisobutyronitrile and azobis(2,4-dimethylvaleronitrile) as initiators were respectively added in the amounts of 1.2 mol % and 3.6 mol % based on the total amount of monomers, followed by heating at 73° C. for about 5 hours. Thereafter, to the polymerization reaction solution thus obtained, an aqueous 25% tetramethylammonium hydroxide solution was added and, after stirring for 12 hours, the mixed solution was poured into a large amount of n-heptane to precipitate a resin, followed by filtration and recovery to obtain a resin A1 having a weight-average molecular weight of about 5.3×103 in a yield of 84%. This resin A1 has the following structural units.

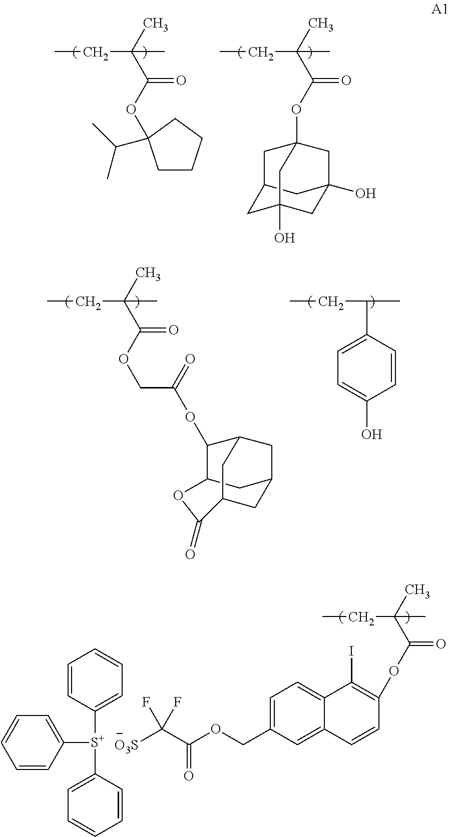

### Example 9 [Synthesis of Resin A2]

Using a monomer (a1-2-6), a monomer (a2-1-3), a monomer (a3-4-2), 4-acetoxystyrene and a monomer (I-2) as monomers, these monomers were mixed in a molar ratio of 55:3:12:25:5 [monomer (a1-2-6):monomer (a2-1-3):monomer (a3-4-2):4-acetoxystyrene:monomer (I-2)], and this monomer mixture was further mixed with propylene glycol monomethyl ether in the amount of 1.5 mass times the total mass of all monomers. To the mixture thus obtained, azobisisobutyronitrile and azobis(2,4-dimethylvaleronitrile) as initiators were respectively added in the amounts of 1.2 mol % and 3.6 mol % based on the total amount of monomers, followed by heating at 73° C. for about 5 hours. Thereafter, to the polymerization reaction solution thus obtained, an aqueous 25% tetramethylammonium hydroxide solution was added and, after stirring for 12 hours, the mixed solution was poured into a large amount of n-heptane to precipitate a resin, followed by filtration and recovery to obtain a resin A2 having a weight-average molecular weight of about 5.5×103 in a yield of 86%. This resin A2 has the following structural units.

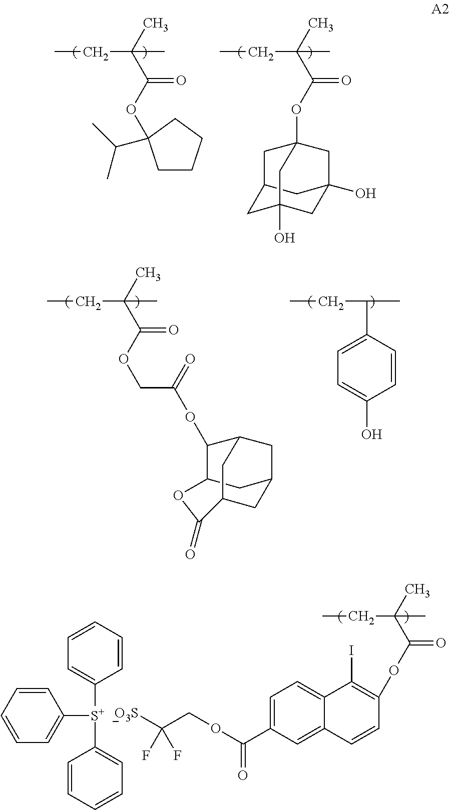

### Example 10 [Synthesis of Resin A3]

Using a monomer (a1-2-6), a monomer (a2-1-3), a monomer (a3-4-2), 4-acetoxystyrene and a monomer (I-3) as monomers, these monomers were mixed in a molar ratio of 55:3:12:25:5 [monomer (a1-2-6):monomer (a2-1-3):monomer (a3-4-2):4-acetoxystyrene:monomer (I-3)], and this monomer mixture was further mixed with propylene glycol monomethyl ether in the amount of 1.5 mass times the total mass of all monomers. To the mixture thus obtained, azobisisobutyronitrile and azobis(2,4-dimethylvaleronitrile) as initiators were respectively added in the amounts of 1.2 mol % and 3.6 mol % based on the total amount of monomers, followed by heating at 73° C. for about 5 hours. Thereafter, to the polymerization reaction solution thus obtained, an aqueous 25% tetramethylammonium hydroxide solution was added and, after stirring for 12 hours, the mixed solution was poured into a large amount of n-heptane to precipitate a resin, followed by filtration and recovery to obtain a resin A3 having a weight-average molecular weight of about 5.6×103 in a yield of 78%. This resin A3 has the following structural units.

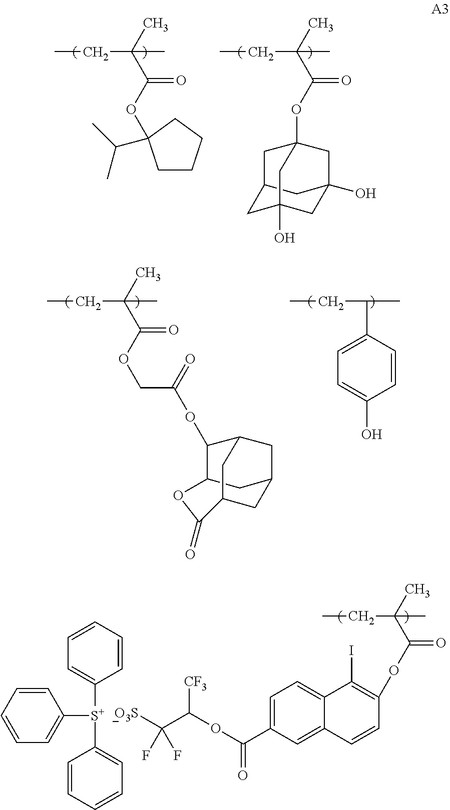

### Example 11 [Synthesis of Resin A4]

Using a monomer (a1-2-6), a monomer (a2-1-3), a monomer (a3-4-2), 4-acetoxystyrene and a monomer (I-24) as monomers, these monomers were mixed in a molar ratio of 55:3:12:25:5 [monomer (a1-2-6):monomer (a2-1-3):monomer (a3-4-2):4-acetoxystyrene:monomer (I-24)], and this monomer mixture was further mixed with propylene glycol monomethyl ether in the amount of 1.5 mass times the total mass of all monomers. To the mixture thus obtained, azobisisobutyronitrile and azobis(2,4-dimethylvaleronitrile) as initiators were respectively added in the amounts of 1.2 mol % and 3.6 mol % based on the total amount of monomers, followed by heating at 73° C. for about 5 hours. Thereafter, to the polymerization reaction solution thus obtained, an aqueous 25% tetramethylammonium hydroxide solution was added and, after stirring for 12 hours, the mixed solution was poured into a large amount of n-heptane to precipitate a resin, followed by filtration and recovery to obtain a resin A4 having a weight-average molecular weight of about 5.3×103 in a yield of 82%. This resin A4 has the following structural units.

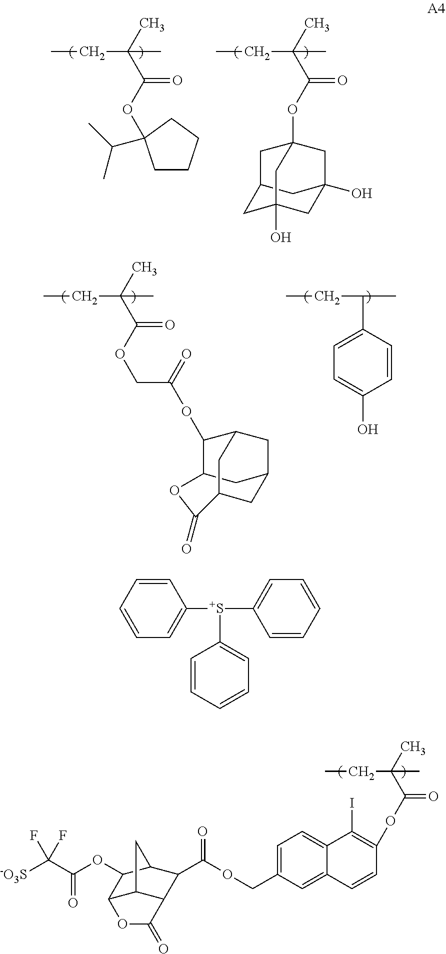

### Example 12 [Synthesis of Resin A5]

Using a monomer (a1-2-6), a monomer (a2-1-3), a monomer (a3-4-2), 4-acetoxystyrene and a monomer (I-354) as monomers, these monomers were mixed in a molar ratio of 55:3:12:25:5 [monomer (a1-2-6):monomer (a2-1-3):monomer (a3-4-2):4-acetoxystyrene:monomer (I-354)], and this monomer mixture was further mixed with propylene glycol monomethyl ether in the amount of 1.5 mass times the total mass of all monomers. To the mixture thus obtained, azobisisobutyronitrile and azobis(2,4-dimethylvaleronitrile) as initiators were respectively added in the amounts of 1.2 mol % and 3.6 mol % based on the total amount of monomers, followed by heating at 73° C. for about 5 hours. Thereafter, to the polymerization reaction solution thus obtained, an aqueous 25% tetramethylammonium hydroxide solution was added and, after stirring for 12 hours, the mixed solution was poured into a large amount of n-heptane to precipitate a resin, followed by filtration and recovery to obtain a resin A5 having a weight-average molecular weight of about 5.4×103 in a yield of 81%. This resin A5 has the following structural units.

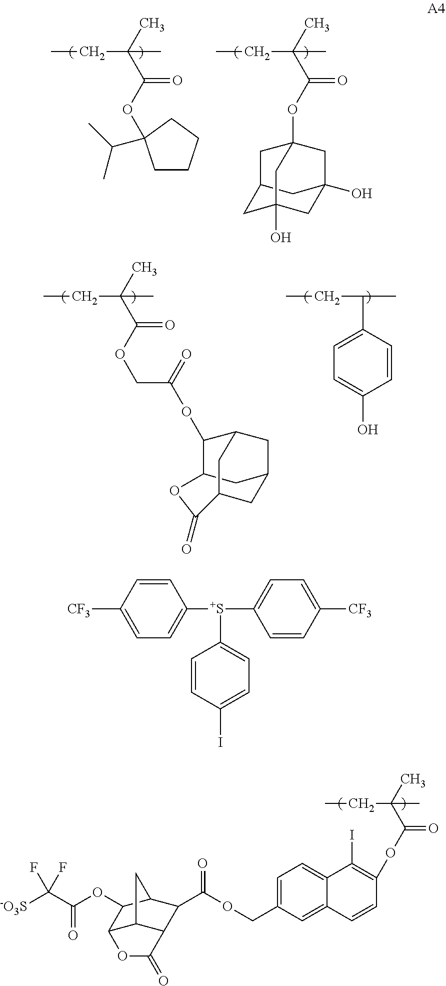

### Example 13 [Synthesis of Resin A6]

Using a monomer (a1-2-6), a monomer (a2-1-3), a monomer (a3-4-2), 4-acetoxystyrene and a monomer (I-1136) as monomers, these monomers were mixed in a molar ratio of 55:3:12:25:5 [monomer (a1-2-6):monomer (a2-1-3):monomer (a3-4-2):4-acetoxystyrene:monomer (I-1136)], and this monomer mixture was further mixed with propylene glycol monomethyl ether in the amount of 1.5 mass times the total mass of all monomers. To the mixture thus obtained, azobisisobutyronitrile and azobis(2,4-dimethylvaleronitrile) as initiators were respectively added in the amounts of 1.2 mol % and 3.6 mol % based on the total amount of monomers, followed by heating at 73° C. for about 5 hours. Thereafter, to the polymerization reaction solution thus obtained, an aqueous 25% tetramethylammonium hydroxide solution was added and, after stirring for 12 hours, the mixed solution was poured into a large amount of n-heptane to precipitate a resin, followed by filtration and recovery to obtain a resin A6 having a weight-average molecular weight of about 5.6×103 in a yield of 86%. This resin A6 has the following structural units.

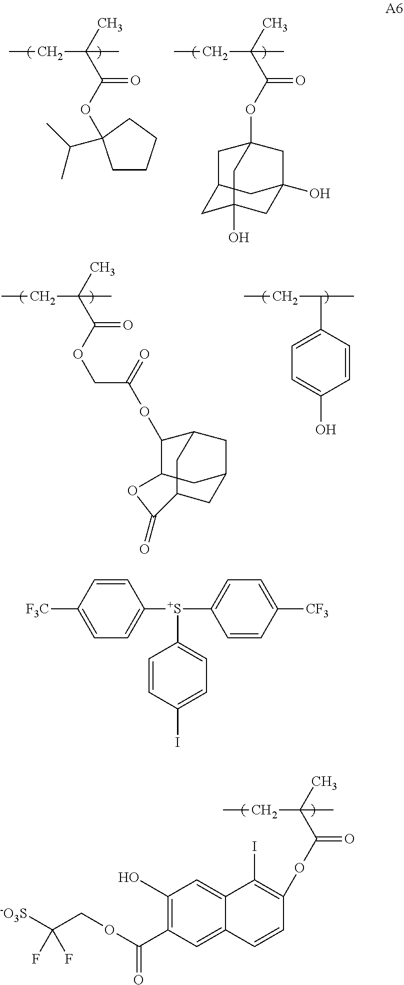

### Example 14 [Synthesis of Resin A7]

Using a monomer (a1-2-6), a monomer (a2-1-3), a monomer (a3-4-2), 4-acetoxystyrene and a monomer (I-1137) as monomers, these monomers were mixed in a molar ratio of 55:3:12:25:5 [monomer (a1-2-6):monomer (a2-1-3):monomer (a3-4-2):4-acetoxystyrene:monomer (I-1137)], and this monomer mixture was further mixed with propylene glycol monomethyl ether in the amount of 1.5 mass times the total mass of all monomers. To the mixture thus obtained, azobisisobutyronitrile and azobis(2,4-dimethylvaleronitrile) as initiators were respectively added in the amounts of 1.2 mol % and 3.6 mol % based on the total amount of monomers, followed by heating at 73° C. for about 5 hours. Thereafter, to the polymerization reaction solution thus obtained, an aqueous 25% tetramethylammonium hydroxide solution was added and, after stirring for 12 hours, the mixed solution was poured into a large amount of n-heptane to precipitate a resin, followed by filtration and recovery to obtain a resin A7 having a weight-average molecular weight of about 5.4×103 in a yield of 80%. This resin A7 has the following structural units.

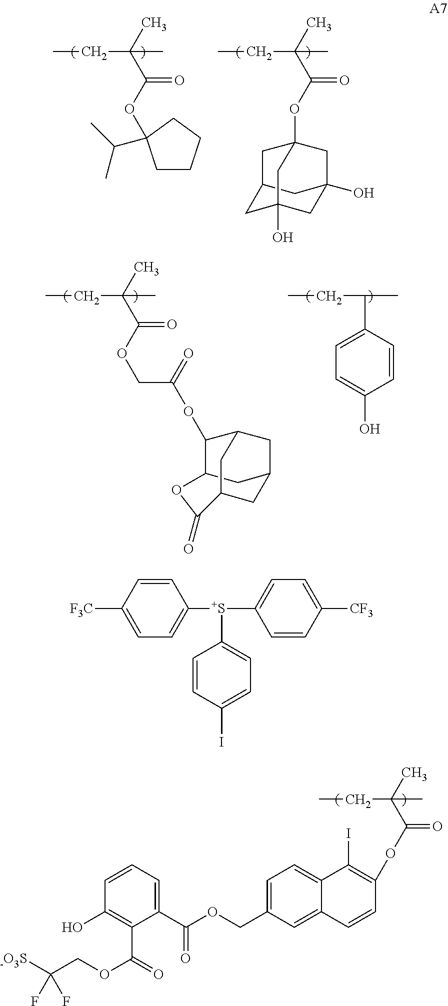

### Synthesis Example 1 [Synthesis of Resin AX1]

Using a monomer (a1-2-6), a monomer (a2-1-3), a monomer (a3-4-2), 4-acetoxystyrene and a monomer (IX-1) as monomers, these monomers were mixed in a molar ratio of 55:3:12:25:5 [monomer (a1-2-6):monomer (a2-1-3):monomer (a3-4-2):4-acetoxystyrene:monomer (IX-1)], and this monomer mixture was further mixed with propylene glycol monomethyl ether in the amount of 1.5 mass times the total mass of all monomers. To the mixture thus obtained, azobisisobutyronitrile and azobis(2,4-dimethylvaleronitrile) as initiators were respectively added in the amounts of 1.2 mol % and 3.6 mol % based on the total amount of monomers, followed by heating at 73° C. for about 5 hours. Thereafter, to the polymerization reaction solution thus obtained, an aqueous 25% tetramethylammonium hydroxide solution was added and, after stirring for 12 hours, the mixed solution was poured into a large amount of n-heptane to precipitate a resin, followed by filtration and recovery to obtain a resin AX1 having a weight-average molecular weight of about 5.7×103 in a yield of 78%. This resin AX1 has the following structural units.

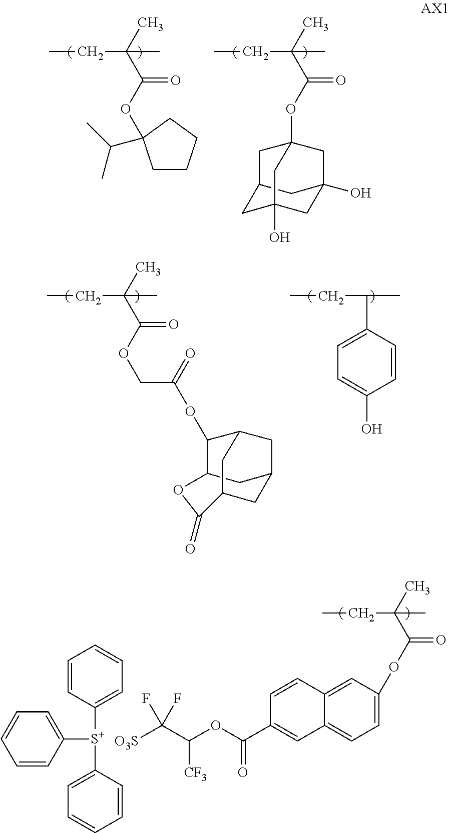

### Synthesis Example 2 [Synthesis of Resin AA1]

Using a monomer (a1-2-6), a monomer (a2-1-3), a monomer (a3-4-2) and 4-acetoxystyrene as monomers, these monomers were mixed in a molar ratio of 55:3:15:27 [monomer (a1-2-6):monomer (a2-1-3):monomer (a3-4-2):4-acetoxystyrene], and then this monomer mixture was mixed with methyl isobutyl ketone in the amount of 1.5 mass times the total mass of all monomers. To the mixture thus obtained, azobisisobutyronitrile and azobis(2,4-dimethylvaleronitrile) as initiators were respectively added in the amounts of 1.2 mol % and 3.6 mol % based on the amount of all monomers, followed by heating at 73° C. for about 5 hours. Thereafter, to the polymerization reaction solution thus obtained, an aqueous 25% tetramethylammonium hydroxide solution was added, followed by stirring for 12 hours and further isolation through separation. The organic layer thus recovered was poured into a large amount of n-heptane to precipitate a resin, followed by filtration and recovery to obtain a resin AA1 having a weight-average molecular weight of about 5.6×103 in a yield of 88%. This resin AA1 has the following structural units.

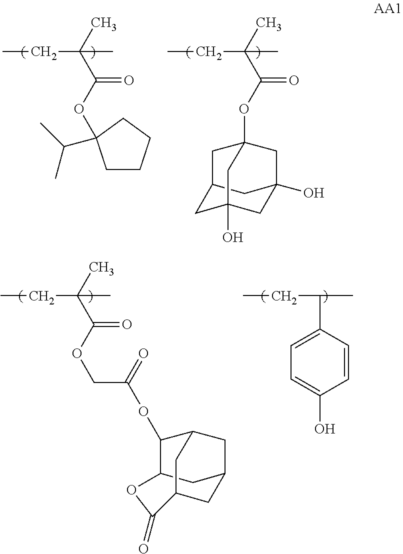

### <Preparation of Resist Composition>

As shown in Table 2, the respective components shown below were mixed and the mixture thus obtained was filtered through a fluororesin filter having a pore diameter of 0.2 μm to prepare resist compositions.

TABLE 2

| | | Acid | | | |
|---|---|---|---|---|---|
| Resist | | generator | | Quencher | |
| composition | Resin | (B) | Salt (I) | (C) | PB/PEB |
| Composition 1 | AA1 = | — | I-354 = | C1 = | 100° C./130° C. |
| | 10 parts | | 1.5 parts | 0.35 part | |
| Composition 2 | AA1 = | — | I-3 = | C1 = | 100° C./130° C. |
| | 10 parts | | 1.5 parts | 0.35 part | |
| Composition 3 | A1 = | B1-25 = | — | C1 = | 100° C./130° C. |
| | 10 parts | 0.5 part | | 0.35 part | |
| Composition 4 | A1 = | — | I-1 = | C1 = | 100° C./130° C. |
| | 10 parts | | 0.5 part | 0.35 part | |
| Composition 5 | A1 = | — | — | C1 = | 100° C./130° C. |
| | 10 parts | | | 0.35 part | |
| Composition 6 | A2 = | B1-25 = | — | C1 = | 100° C./130° C. |
| | 10 parts | 0.5 part | | 0.35 part | |
| Composition 7 | A2 = | — | I-2 = | C1 = | 100° C./130° C. |
| | 10 parts | | 0.5 part | 0.35 part | |
| Composition 8 | A2 = | — | — | C1 = | 100° C./130° C. |
| | 10 parts | | | 0.35 part | |
| Composition 9 | A3 = | B1-25 = | — | C1 = | 100° C./130° C. |
| | 10 parts | 0.5 part | | 0.35 part | |
| Composition | A3 = | — | I-3 | C1 = | 100° C./130° C. |
| 10 | 10 parts | | 0.5 part | 0.35 part | |
| Composition | A3 = | — | — | C1 = | 100° C./130° C. |
| 11 | 10 parts | | | 0.35 part | |
| Composition | A4 = | B1-25 = | — | C1 = | 100° C./130° C. |
| 12 | 10 parts | 0.5 part | | 0.35 part | |
| Composition | A4 = | — | I-24 = | C1 = | 100° C./130° C. |
| 13 | 10 parts | | 0.5 part | 0.35 part | |
| Composition | A4 = | — | — | C1 = | 100° C./130° C. |
| 14 | 10 parts | | | 0.35 part | |
| Composition | A5 = | B1-25 = | — | C1 = | 100° C./130° C. |
| 15 | 10 parts | 0.5 part | | 0.35 part | |
| Composition | A5 = | — | I-354 = | C1 = | 100° C./130° C. |
| 16 | 10 parts | | 0.5 part | 0.35 part | |
| Composition | A5 = | — | — | C1 = | 100° C./130° C. |
| 17 | 10 parts | | | 0.35 part | |
| Composition | A6 = | B1-25 = | — | C1 = | 100° C./130° C. |
| 18 | 10 parts | 0.5 part | | 0.35 part | |
| Composition | A6 = | — | I-1136 = | C1 = | 100° C./130° C. |
| 19 | 10 parts | | 0.5 part | 0.35 part | |
| Composition | A6 = | — | — | C1 = | 100° C./130° C. |
| 20 | 10 parts | | | 0.35 part | |
| Composition | A7 = | B1-25 = | — | C1 = | 100° C./130° C. |
| 21 | 10 parts | 0.5 part | | 0.35 part | |
| Composition | A7 = | — | I-1137 = | C1 = | 100° C./130° C. |
| 22 | 10 parts | | 0.5 part | 0.35 part | |
| Composition | A7 = | — | — | C1 = | 100° C./130° C. |
| 23 | 10 parts | | | 0.35 part | |
| Comparative | AA1 = | IX-1 = | — | C1 = | 100° C./130° C. |
| Composition 1 | 10 parts | 1.5 parts | | 0.35 part | |
| Comparative | AX1 = | B1-25 = | — | C1 = | 100° C./130° C. |
| Composition 2 | 10 parts | 0.5 part | | 0.35 part | |
| Comparative | AX1 = | IX-1 = | — | C1 = | 100° C./130° C. |
| Composition 3 | 10 parts | 0.5 part | | 0.35 part | |
| Comparative | AX1 = | — | — | C1 = | 100° C./130° C. |
| Composition 4 | 10 parts | | | 0.35 part | |

### <Resin>

A1 to A7, AA1, AX1: Resin A1 to Resin A7, Resin AA1, Resin AX1

### <Salt (I)>

I-1: Salt represented by Formula (I-1)
I-2: Salt represented by Formula (I-2)
I-3: Salt represented by Formula (I-3)
I-24: Salt represented by Formula (I-24)
I-354: Salt represented by Formula (I-354)
I-1136: Salt represented by Formula (I-1136)
I-1137: Salt represented by Formula (I-1137)

### <Acid Generator (B)>

B1-25: Salt represented by Formula (B1-25); synthesized by the method mentioned in JP 2011-126869 A

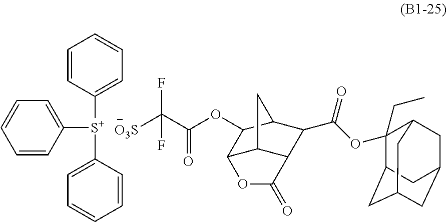

IX-1:

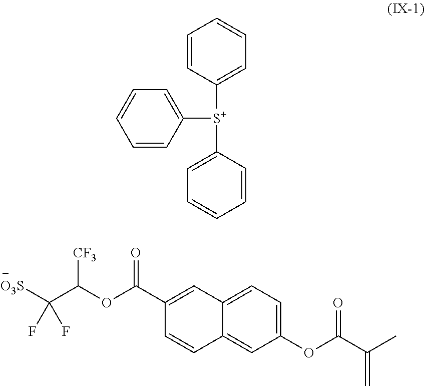

### <Quencher (C)>

C1: synthesized by the method mentioned in JP 2011-39502 A

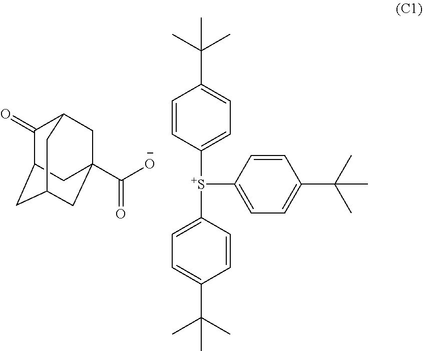 [SMILES: O=C1C2CC3CC1CC(C(=O)[O-])(C3)C2 ; CC(C)(C)c1ccc([S+](c2ccc(C(C)(C)C)cc2)c2ccc(C(C)(C)C)cc2)cc1]

### <Solvent>

| | |
|---|---|
| Methyl 2-hydroxyisobutyrate | 400 parts |
| Propylene glycol monomethyl ether | 100 parts |
| γ-Butyrolactone | 5 parts |

(Evaluation of Exposure of Resist Composition with Electron Beam)

Each 6 inch-diameter silicon wafer was treated with hexamethyldisilazane on a direct hot plate at 90° C. for 60 seconds. A resist composition was spin-coated on the silicon wafer in such a manner that the thickness of the composition layer became 0.04 μm. Then, the coated silicon wafer was prebaked on the direct hot plate at the temperature shown in the column “PB” of Table 2 for 60 seconds to form a composition layer. Using an electron-beam direct-write system (“ELS-F125 125 keV”, manufactured by ELIONIX INC.), contact hole patterns (hole pitch of 40 nm/hole diameter of 17 nm) were directly written on the composition layer formed on the wafer while changing the exposure dose stepwise.

After exposure, post-exposure baking was performed on the hot plate at the temperature shown in the column “PEB” of Table 2 for 60 seconds, followed by puddle-development with an aqueous 2.38% by mass tetramethylammonium hydroxide solution for 60 seconds to obtain resist patterns.

In the resist pattern obtained after development, the exposure dose at which the diameter of holes formed became 17 nm was defined as effective sensitivity.

### <Evaluation of CD Uniformity (CDU)>

In the effective sensitivity, the hole diameter of the pattern formed using a mask having a hole dimeter of 17 nm was determined by measuring 24 times per one hole and the average of the measured values was regarded as the average hole diameter of one hole. The standard deviation was determined under the conditions that the average diameter of 400 holes about the patterns formed using the mask having a hole dimeter of 17 nm in the same wafer was regarded to as population.

The results are shown in Table 3. The numerical value in the parenthesis represents the standard deviation (nm).

TABLE 3

| | Resist composition | CDU |
|---|---|---|
| Example 15 | Composition 1 | 2.69 |
| Example 16 | Composition 2 | 2.76 |
| Example 17 | Composition 3 | 2.64 |
| Example 18 | Composition 4 | 2.62 |
| Example 19 | Composition 5 | 2.70 |
| Example 20 | Composition 6 | 2.62 |
| Example 21 | Composition 7 | 2.60 |
| Example 22 | Composition 8 | 2.67 |
| Example 23 | Composition 9 | 2.70 |
| Example 24 | Composition 10 | 2.72 |
| Example 25 | Composition 11 | 2.79 |
| Example 26 | Composition 12 | 2.48 |
| Example 27 | Composition 13 | 2.45 |
| Example 28 | Composition 14 | 2.50 |
| Example 29 | Composition 15 | 2.36 |
| Example 30 | Composition 16 | 2.33 |
| Example 31 | Composition 17 | 2.39 |
| Example 32 | Composition 18 | 2.29 |
| Example 33 | Composition 19 | 2.25 |
| Example 34 | Composition 20 | 2.31 |
| Example 35 | Composition 21 | 2.24 |
| Example 36 | Composition 22 | 2.21 |
| Example 37 | Composition 23 | 2.26 |
| Comparative Example 1 | Comparative Composition 1 | 2.93 |
| Comparative Example 2 | Comparative Composition 2 | 2.86 |
| Comparative Example 3 | Comparative Composition 3 | 2.93 |
| Comparative Example 4 | Comparative Composition 4 | 3.01 |

As compared with Comparative Compositions 1 to 4, Compositions 1 to 23 exhibited small standard deviation and satisfactory evaluation of CD uniformity (CDU).

### INDUSTRIAL APPLICABILITY

A salt and a resist composition including the salt of the present invention exhibit satisfactory CD uniformity (CDU), and are therefore useful for fine processing of semiconductors.

1. A salt represented by formula (I): 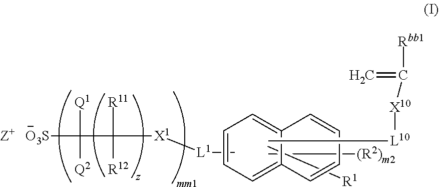
wherein, in formula (I),
Q¹ and Q² each independently represent a hydrogen atom, a fluorine atom, an alkyl group having 1 to 6 carbon atoms, or a perfluoroalkyl group having 1 to 6 carbon atoms,
R¹¹ and R¹² each independently represent a hydrogen atom, a fluorine atom, an alkyl group having 1 to 6 carbon atoms, or a perfluoroalkyl group having 1 to 6 carbon atoms,
z represents an integer of 0 to 6, and when z is 2 or more, a plurality of R¹¹ and R¹² may be the same or different from each other,
X¹ represents *—CO—O—, *—O—CO—, *—O—CO—O— or *—O—, and * represents a bonding site to C(R¹¹) (R¹²) or C(Q¹) (Q²),
mm1 represents 0 or 1,
L¹ represents a single bond or a hydrocarbon group having 1 to 40 carbon atoms which may have a substituent, and —CH₂— included in the hydrocarbon group may be replaced by —O—, —S—, —SO₂— or —CO—,
R¹ represents a halogen atom or a haloalkyl group having 1 to 12 carbon atoms,
R² represents a halogen atom, a hydroxy group, a haloalkyl group having 1 to 12 carbon atoms, or an alkyl group having 1 to 12 carbon atoms, and —CH₂— included in the haloalkyl group and the alkyl group may be replaced by —O— or —CO—,
m2 represents an integer of 0 to 5, and when m2 is 2 or more, a plurality of R² may be the same or different from each other,
R^{bb1} represents a hydrogen atom, a halogen atom, or an alkyl group having 1 to 6 carbon atoms which may have a halogen atom,
X¹⁰ represents a single bond, *—O—**, *—CO—O—**, *—O—CO—O—**, *—CO—NR³—**, *—NR³—CO—O—**, *—O—CO—NR³—** or *-Ax-Ph-Ay-**,
R³ represents a hydrogen atom or an alkyl group having 1 to 6 carbon atoms,
Ph represents a phenylene group which may have a substituent,
Ax and Ay each independently represent one or more groups selected from the group consisting of a single bond, an ether bond, a thioether bond, an ester bond, an amide bond and a carbonic acid ester bond,
* and ** represent a bonding site, and * represents a bonding site to carbon atoms to which R^{bb1} is bonded,
L¹⁰ represents a single bond or a hydrocarbon group having 1 to 36 carbon atoms which may have a substituent, and —CH₂— included in the hydrocarbon group may be replaced by —O—, —S—, —SO₂— or —CO—, and
Z⁺ represents an organic cation.

2. The salt according to claim 1, wherein X¹⁰ is a single bond or a group represented by any one of formula (X¹⁰-1) to formula (X¹⁰-10): 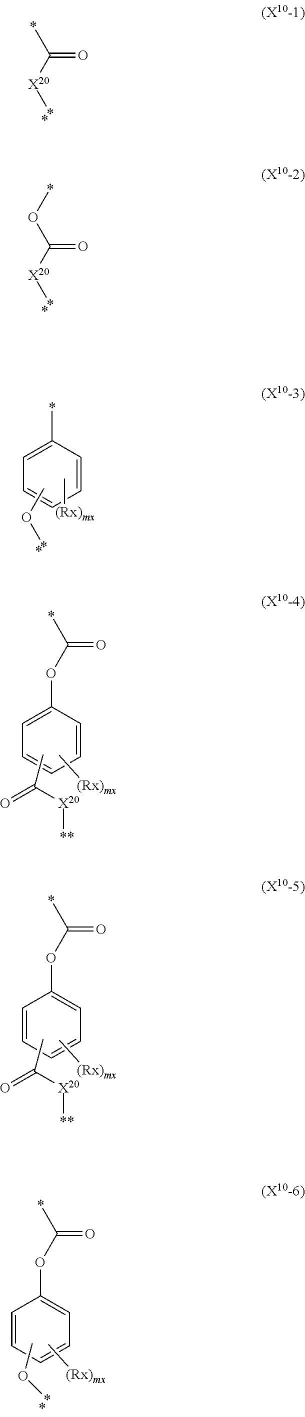 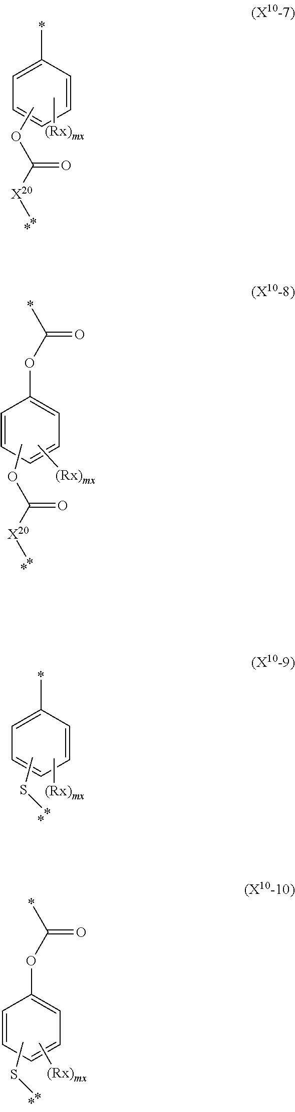
wherein, in formula (X¹⁰-1) to formula (X¹⁰-10),
* and ** are bonding sites, and * represents a bonding site to carbon atoms to which R^{bb1} is bonded,
Rx represents a halogen atom, a hydroxy group, an alkyl fluoride group having 1 to 6 carbon atoms, an alkyl group having 1 to 18 carbon atoms or an alkoxy group having 1 to 6 carbon atoms,
mx represents an integer of 0 to 4,
X²⁰ represents —O— or —NR³—, and
R³ represents a hydrogen atom or an alkyl group having 1 to 6 carbon atoms.

3. The salt according to claim 1, wherein R¹ is an iodine atom, a fluorine atom or a perfluoroalkyl group having 1 to 3 carbon atoms.

4. The salt according to claim 1, wherein L′ is a single bond, *-L²- or *-L²-X³-L³_(L² represents a chain hydrocarbon group having 1 to 10 carbon atoms which may have a substituent, a cyclic hydrocarbon group having 3 to 24 carbon atoms which may have a substituent, or groups obtained by combining a cyclic hydrocarbon group having 3 to 24 carbon atoms which may have a substituent with a chain hydrocarbon group having 1 to 8 carbon atoms which may have a substituent, —CH₂— included in the cyclic hydrocarbon group may be replaced by —O—, —S—, —CO— or —SO₂—, —CH₂— included in the chain hydrocarbon group may be replaced by —O— or —CO—, * represents a bonding site to L², X³ represents **—CO—O—, **—O—CO—, **—O—CO—O— or **—O—, ** represents a bonding site to L², L³ represents a single bond or a chain hydrocarbon group having 1 to 8 carbon atoms which may have a substituent, and —CH₂— included in the chain hydrocarbon group may be replaced by —O— or —CO—).

5. The salt according to claim 1, wherein when m2 is an integer of 1 or more, R² is an iodine atom, a fluorine atom, a hydroxy group, a haloalkyl group having 1 to 6 carbon atoms or an alkyl group having 1 to 6 carbon atoms (—CH₂— included in the haloalkyl group and the alkyl group may be replaced by —O— or —CO—).

6. A resin comprising a structural unit represented by formula (IP): 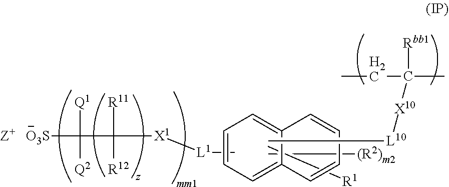
wherein, in formula (IP),
Q¹ and Q² each independently represent a hydrogen atom, a fluorine atom, an alkyl group having 1 to 6 carbon atoms or a perfluoroalkyl group having 1 to 6 carbon atoms,
R¹¹ and R¹² each independently represent a hydrogen atom, a fluorine atom, an alkyl group having 1 to 6 carbon atoms, or a perfluoroalkyl group having 1 to 6 carbon atoms,
z represents an integer of 0 to 6, and when z is 2 or more, a plurality of R¹¹ and R¹² may be the same or different from each other,
X¹ represents *—CO—O—, *—O—CO—, *—O—CO—O— or *—O—, and * represents a bonding site to C(R¹¹) (R¹²) or C(Q¹) (Q²),
mm1 represents 0 or 1,
L¹ represents a single bond or a hydrocarbon group having 1 to 40 carbon atoms which may have a substituent, and —CH₂— included in the hydrocarbon group may be replaced by —O—, —S—, —SO₂— or —CO—,
R¹ represents a halogen atom or a haloalkyl group having 1 to 12 carbon atoms,
R² represents a halogen atom, a hydroxy group, a haloalkyl group having 1 to 12 carbon atoms or an alkyl group having 1 to 12 carbon atoms, and —CH₂— included in the haloalkyl group and the alkyl group may be replaced by —O— or —CO—,
m2 represents an integer of 0 to 5, and when m2 is 2 or more, a plurality of R² may be the same or different from each other,
R^{bb1} represents a hydrogen atom, a halogen atom, or an alkyl group having 1 to 6 carbon atoms which may have a halogen atom,
X¹⁰ represents a single bond, *—O—**, *—CO—O—**, *—O—CO—O—**, *—CO—NR³—**, *—NR³—CO—O—**, *—O—CO—NR³—** or *-Ax-Ph-Ay-**,
R³ represents a hydrogen atom or an alkyl group having 1 to 6 carbon atoms,
Ph represents a phenylene group which may have a substituent,
Ax and Ay each independently represent one or more bonds selected from the group consisting of a single bond, an ether bond, a thioether bond, an ester bond, an amide bond and a carbonic acid ester bond,
* and ** represent a bonding site, and * represents a bonding site to carbon atoms to which R^{bb1} is bonded,
L¹⁰ represents a single bond or a hydrocarbon group having 1 to 36 carbon atoms which may have a substituent, and —CH₂— included in the hydrocarbon group may be replaced by —O—, —S—, —SO₂— or —CO—, and
Z⁺ represents an organic cation.

7. The resin according to claim 6, further including a structural unit having an acid-labile group.

8. An acid generator comprising the salt according to claim 1.

9. An acid generator comprising the resin according to claim 6.

10. A resist composition comprising:
the salt according to claim 1, and
a quencher.

11. A resist composition comprising:
the resin according to claim 6, and
a quencher.

12. A resist composition comprising:
the salt according to claim 1, and
a resin comprising a structural unit represented by formula (IP); 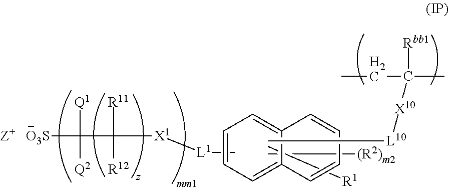
wherein, in formula (IP),
Q¹ and Q² each independently represent a hydrogen atom, a fluorine atom, an alkyl group having 1 to 6 carbon atoms or a perfluoroalkyl group having 1 to 6 carbon atoms,
R¹¹ and R¹² each independently represent a hydrogen atom, a fluorine atom, an alkyl group having 1 to 6 carbon atoms, or a perfluoroalkyl group having 1 to 6 carbon atoms,
z represents an integer of 0 to 6, and when z is 2 or more, a plurality of R¹¹ and R¹² may be the same or different from each other,
X¹ represents *—CO—O—, *—O—CO—, *—O—CO—O— or *—O—, and * represents a bonding site to C(R¹¹) (R¹²) or C(Q¹) (02),
mm1 represents 0 or 1,
L¹ represents a single bond or a hydrocarbon group having 1 to 40 carbon atoms which may have a substituent, and —CH₂— included in the hydrocarbon group may be replaced by —O—, —S—, —SO₂— or —CO—,
R¹ represents a halogen atom or a haloalkyl group having 1 to 12 carbon atoms,
R² represents a halogen atom, a hydroxy group, a haloalkyl group having 1 to 12 carbon atoms or an alkyl group having 1 to 12 carbon atoms, and —CH₂— included in the haloalkyl group and the alkyl group may be replaced by —O— or —CO—,
m2 represents an integer of 0 to 5, and when m2 is 2 or more, a plurality of R² may be the same or different from each other,
R^{bb1} represents a hydrogen atom, a halogen atom, or an alkyl group having 1 to 6 carbon atoms which may have a halogen atom,
X¹⁰ represents a single bond, *—O—**, *—CO—O—**, *—O—CO—O—**, *—CO—NR³-**, *—NR³—CO—O—**, *—O—CO—NR³—** or *-Ax-Ph-Ay-**,
R³ represents a hydrogen atom or an alkyl group having 1 to 6 carbon atoms,
Ph represents a phenylene group which may have a substituent,
Ax and Ay each independently represent one or more bonds selected from the group consisting of a single bond, an ether bond, a thioether bond, an ester bond, an amide bond and a carbonic acid ester bond,
* and ** represent a bonding site, and * represents a bonding site to carbon atoms to which R^{bb1} is bonded,
L¹⁰ represents a single bond or a hydrocarbon group having 1 to 36 carbon atoms which may have a substituent, and —CH₂— included in the hydrocarbon group may be replaced by —O—, —S—, —SO₂— or —CO—, and
Z⁺ represents an organic cation.

13. A resist composition comprising the resin according to claim 7 and a quencher.

14. The resist composition according to claim 13, wherein the structural unit having an acid-labile group includes at least one selected from the group consisting of a structural unit represented by formula (a1-0), a structural unit represented by formula (a1-1), a structural unit represented by formula (a1-2), a structural unit represented by formula (a1-4), a structural unit represented by formula (a1-5) and a structural unit represented by formula (a1-6): 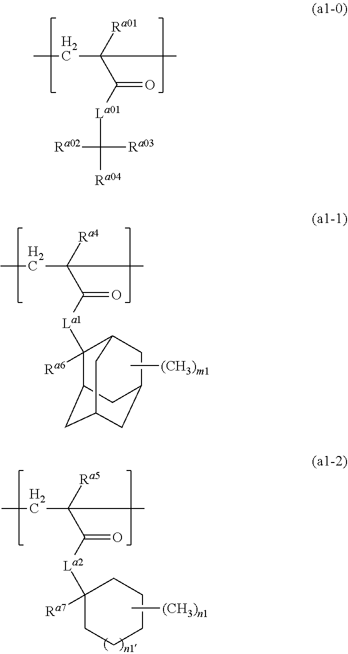
wherein, in formula (a1-0), formula (a1-1) and formula (a1-2),
L^{a01}, L^{a1} and L^{a2} each independently represent-O— or *—O—(CH₂)ₖ₁—CO—O—, k1 represents an integer of 1 to 7, and * represents a bonding site to —CO—,
R^{a01}, R^{a4} and R^{a5} each independently represent a hydrogen atom, a halogen atom, or an alkyl group having 1 to 6 carbon atoms which may have a halogen atom,
R^{a02}, R^{a03} and R^{a04} each independently represent an alkyl group having 1 to 8 carbon atoms, an alicyclic hydrocarbon group having 3 to 18 carbon atoms, an aromatic hydrocarbon group having 6 to 18 carbon atoms, or groups obtained by combining these groups, and the alkyl group, the alicyclic hydrocarbon group and the aromatic hydrocarbon group may have a halogen atom,
R^{a6} and R^{a7} each independently represent an alkyl group having 1 to 8 carbon atoms, an alkenyl group having 2 to 8 carbon atoms, an alicyclic hydrocarbon group having 3 to 18 carbon atoms, an aromatic hydrocarbon group having 6 to 18 carbon atoms, or groups formed by combining these groups, and the alkyl group, the alicyclic hydrocarbon group and the aromatic hydrocarbon group may have a halogen atom,
m1′ represents an integer of 0 to 14,
n1 represents an integer of 0 to 10, and
n1′ represents an integer of 0 to 3: 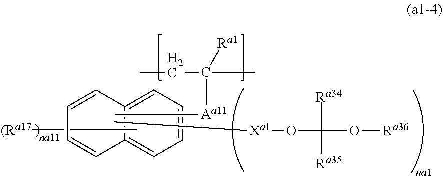
wherein, in formula (a1-4),
R^{a1} represents a hydrogen atom, a halogen atom, or an alkyl group having 1 to 6 carbon atoms which may have a halogen atom,
R^{a17} represents a halogen atom, a hydroxy group, a carboxy group, an alkyl group having 1 to 6 carbon atoms which may have a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group or a methacryloyloxy group,
A^{a11} represents a single bond or an alkanediyl group having 1 to 12 carbon atoms, and —CH₂— included in the alkanediyl group may be replaced by —O—, —CO— or —NR^{a18}—,
R^{a18} represents a hydrogen atom or an alkyl group having 1 to 6 carbon atoms,
X^{a1} represents a single bond or a carbonyl group,
R^{a34} and R^{a35} each independently represent a hydrogen atom or a hydrocarbon group having 1 to 12 carbon atoms, R^{a36} represents a hydrocarbon group having 1 to 20 carbon atoms, or R^{a35} and R^{a36} may be bonded to each other to form a divalent hydrocarbon group having 2 to 20 carbon atoms together with —C—O— to which R^{a35} and R^{a36} are bonded, and —CH₂— included in the hydrocarbon group and the divalent hydrocarbon group may be replaced by —O— or —S—,
na1 represents an integer of 1 to 4, and when na1 is 2 or more, a plurality of groups in parentheses may be the same or different from each other,
na11 represents an integer of 0 to 4 and satisfies 1≤na1+na11≤5, and when na11 is 2 or more, a plurality of R^{a17} may be the same or different from each other, and
mc represents an integer of 0 to 2: 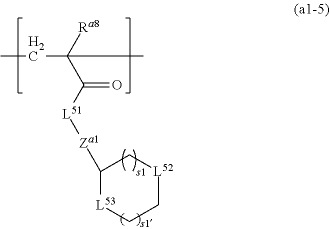
wherein, in formula (a1-5),
R^{a8} represents an alkyl group having 1 to 6 carbon atoms which may have a halogen atom, a hydrogen atom or a halogen atom,
Z^{a1} represents a single bond or *—(CH₂)ₕ₃—CO-L⁵⁴-, h3 represents an integer of 1 to 4, and * represents a bonding site to L⁵¹,
L⁵¹, L⁵², L⁵³ and L⁵⁴ each independently represent-O— or —S—,
s1 represents an integer of 1 to 3, and
s1′ represents an integer of 0 to 3, and: 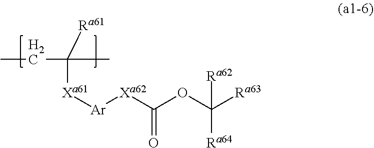
wherein, in formula (a1-6),
R^{a61} represents a hydrogen atom, a halogen atom, or an alkyl group having 1 to 6 carbon atoms which may have a halogen atom,
R^{a62}, R^{a63} and R^{a64} each independently represent an alkyl group having 1 to 6 carbon atoms or a cyclic hydrocarbon group having 3 to 18 carbon atoms which may have a substituent, or R^{a62} and R^{a63} may be bonded to each other to form a ring having 3 to 20 carbon atoms together with carbon atoms to which R^{a62} and R^{a63} are bonded,
X^{a61} represents a single bond, —CO—O—* or —CO—NR⁵—*, * represents a bonding site to —Ar, and R⁵ represents a hydrogen atom or an alkyl group having 1 to 4 carbon atoms,
X^{a62} represents a single bond, *—O-L^{a61}- or *—CO—O-L^{a62}-, * represents a bonding site to —Ar, and L^{a61} and L^{a62} each independently represent an alkanediyl group having 1 to 4 carbon atoms, and
Ar represents an aromatic hydrocarbon group having 6 to 20 carbon atoms which may have a substituent.

15. The resist composition according to claim 14, wherein the resin includes a structural unit represented by formula (a2-A): 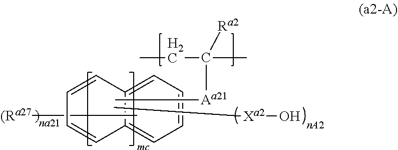
wherein, in formula (a2-A),
R^{a2} represents a hydrogen atom, a halogen atom, or an alkyl group having 1 to 6 carbon atoms which may have a halogen atom,
R^{a27} represents a halogen atom, an alkyl group having 1 to 6 carbon atoms which may have a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group or a methacryloyloxy group,
A^{a21} represents a single bond or an alkanediyl group having 1 to 12 carbon atoms, and —CH₂— included in the alkanediyl group may be replaced by —O—, —CO— or —NR^{a28}—,
R^{a28} represents a hydrogen atom or an alkyl group having 1 to 6 carbon atoms,
X^{a2} represents a single bond or a carbonyl group,
nA2 represents an integer of 1 to 4, and when nA2 is 2 or more, a plurality of groups in parentheses may be the same or different from each other,
na21 represents an integer of 0 to 4 and satisfies 1≤nA2+na21≤5, and when na21 is 2 or more, a plurality of R^{a27} may be the same or different from each other, and
mc represents an integer of 0 to 2.

16. The resist composition according to claim 13, wherein the resin further includes a structural unit represented by formula (a3-1), formula (a3-2), formula (a3-3) or formula (a3-4): 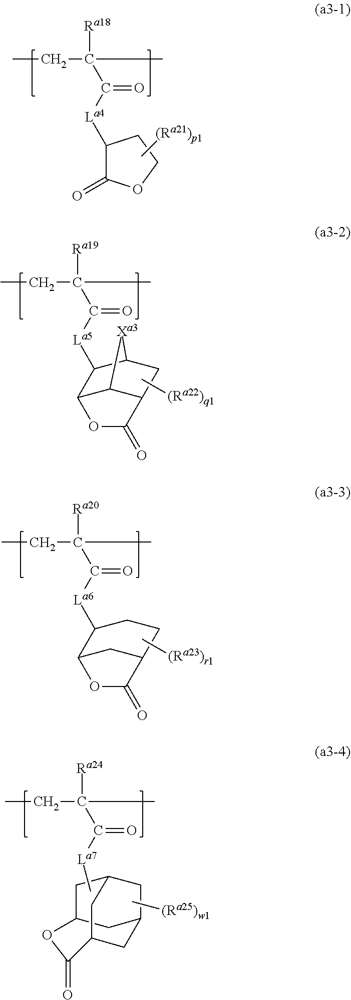
wherein, in formula (a3-1), formula (a3-2), formula (a3-3) and formula (a3-4),
L^{a4}, L^{a5} and L^{a6} each independently represent a group represented by —O— or *—O— (CH₂)ₖ₃—CO—O— (k3 represents an integer of 1 to 7),
L^{a7} represents —O—, *—O-L^{a8}-O—, *—O-L^{a8}-CO—O—, *—O-L^{a8}-CO—O-L^{a9}-CO—O— or *—O— L^{a8}-O—CO-L^{a9}-O—,
L^{a8} and L^{a9} each independently represent an alkanediyl group having 1 to 6 carbon atoms,
* represents a bonding site to a carbonyl group,
R^{a18}, R^{a19}, R^{a20} and R^{a24} each independently represent an alkyl group having 1 to 6 carbon atoms which may have a halogen atom, a hydrogen atom or a halogen atom,
X^{a3} represents —CH₂— or an oxygen atom,
R^{a21} represents an aliphatic hydrocarbon group having 1 to 4 carbon atoms,
R^{a22}, R^{a23} and R^{a25} each independently represent a carboxy group, a cyano group or an aliphatic hydrocarbon group having 1 to 4 carbon atoms,
p1 represents an integer of 0 to 5,
q1 represents an integer of 0 to 3,
r1 represents an integer of 0 to 3,
w1 represents an integer of 0 to 8, and
when p1, q1, r1 and/or w1 is/are 2 or more, a plurality of R^{a21}, R^{a22}, R^{a23} and/or R^{a25} may be the same or different from each other.

17. The resist composition according to claim 10, wherein the quencher (C) further includes a salt generating an acid having an acidity lower than that of an acid generated from an acid generator.

18. A method for producing a resist pattern, which comprises:
(1) a step of applying the resist composition according to claim 10 on a substrate,
(2) a step of drying the applied resist composition to form a composition layer,
(3) a step of exposing the composition layer,
(4) a step of heating the exposed composition layer, and
(5) a step of developing the heated composition layer.

19. A method for producing a resist pattern, which comprises:
(1) a step of applying the resist composition according to claim 11 on a substrate,
(2) a step of drying the applied resist composition to form a composition layer,
(3) a step of exposing the composition layer,
(4) a step of heating the exposed composition layer, and
(5) a step of developing the heated composition layer.
